(19) United States
(12) Patent Application Publication
(10) Pub. No.: US 2017/0007615 A1
(43) Pub. Date: Jan. 12, 2017

(54) Antibacterial Compounds

(71) Applicant: BIOTA EUROPE LTD, Yarnton, Oxfordshire (GB)

(72) Inventors: Ian Collins, Yarnton (GB); David John Haydon, Yarnton (GB); Helena Thomaides-Brears, Yarnton (GB); James T Palmer, Victoria (AU); Christopher James Lunniss, Victoria (AU)

(21) Appl. No.: 15/116,150

(22) PCT Filed: Feb. 3, 2015
(86) PCT No.: PCT/IB2015/000177

§ 371 Date: Aug. 2, 2016

(30) Foreign Application Priority Data
Feb. 3, 2014 (AU) 2014900308
Jun. 12, 2014 (AU) 2014902238

(51) Int. Cl. A61K 31/5383; A61K 38/12; A61K 31/444; A61K 31/506; A61K 31/497; A61K 31/496; A61K 31/5377; A61K 31/4439; A61K 31/428

(52) U.S. Cl. CPC A61K 31/5383; A61K 31/4439; A61K 38/12; A61K 31/444; A61K 31/428; A61K 31/497; A61K 31/496; A61K 31/5377; A61K 31/506

(57) ABSTRACT

The present disclosure relates to a novel combination of compounds, their use as antibacterials, compositions comprising them and methods for treating or preventing bacterial infections, more particularly, bacterial infections caused by Gram-negative pathogens and/or drug resistant Gram-negative bacteria.

### CROSS-REFERENCE TO RELATED APPLICATIONS

The present application claims priority from Australian Provisional Patent Application No 2014900308 filed on 3 Feb. 2014, Australian Provisional Patent Application No 2014902238 filed on 12 Jun. 2014, the contents of which are incorporated herein by reference.

### TECHNICAL FIELD

The present disclosure relates to a novel combination of compounds, their use as antibacterials, compositions comprising them and methods for treating or preventing bacterial infections, more particularly, bacterial infections caused by Gram-negative pathogens and/or drug resistant Gram-negative bacteria.

### BACKGROUND

The Global Risks 2013, Eighth Edition Insight Report by the World Economic Forum considers that emerging resistance to our current arsenal of clinically approved antibiotics is of great concern to human health. The report provides that “[a]lthough several new compounds for fighting bacteria are in development, experts caution that we are decades behind in comparison with the historical rate at which we have discovered and developed new antibiotics. More, worryingly, none of the drugs currently in the development pipeline would be effective against certain killer bacteria, which have newly emerging resistance to our strongest antibiotics (carbapenems) and fatality rates of up to 50%.” Further, the Report highlights that in addition to treating bacterial infections, antibiotics are also used to guard against, that is with the aim of preventing, bacterial infections during medical procedures making otherwise impossible or risky surgeries possible. As resistance to antibiotics increases due in part to the over-use both in humans and animals and the development of new antibiotics slows, the situation remains dire. As FIG. 18 of the Report highlights, there have been no new classes of antibiotic compounds discovered since the lipopeptides in 1987, thereby resulting in a “discovery void.”

Type II topoisomerases have been the target of a number of antibacterial agents. The most prominent of these agents are the quinolones. The original quinolone antibiotics included nalidixic acid, cinoxacin and oxolinic acid. The addition of fluorine yielded a new class of drugs, the fluoroquinolones, which have a broader antimicrobial spectrum and improved pharmacokinetic properties. The fluoroquinolones include norfloxacin, ciprofloxacin, second generation fluoroquinolones such as ofloxacin and fourth generation quinolones gatifloxacin and moxifloxacin. The coumarins and the cyclothialidines are further classes of antibiotics that inhibit type II topoisomerases however they are not widely used because of poor permeability in bacteria, eukaryotic toxicity, and low water solubility. Examples of such antibiotics include novobiocin, coumermycin A1, cyclothialidine, cinodine, and clerocidin.

Ideally, an antibiotic based on the inhibition of bacterial type II topoisomerases would be selective for the bacterial enzymes and be relatively inactive against the eukaryotic type II isomerases. The type II topoisomerases are highly conserved enzymes allowing the design of broad-spectrum inhibitors. Furthermore, the GyrB and ParE subunits are functionally similar, having an ATPase domain in the N-terminal domain and a C-terminal domain that interacts with the other subunit (GyrA and ParC respectively) and the DNA. The conservation between the gyrase and topoisomerase IV active sites suggests that inhibitors of the sites might simultaneously target both type II topoisomerases. Such dual-targeting inhibitors are attractive because they have the potential to reduce the development of target-based resistance.

Polymyxins, a class of compounds unrelated to the bacterial type II topoisomerase inhibitors described above, are cyclic lipodecapeptides which were first discovered in the late-1940s. Two notable examples of polymyxins are colistin and polymyxin B (PMB) which were discovered in the mid-1950s and originally used as intravenously administered antibacterials. Colistin is typically administered in a prodrug form, specifically as its methanesulfonate, colistin methanesulfonate (CMS). However, an undesirable side effect associated with the therapeutic use of these compounds was their toxicity. The emergence of polymyxin resistant strains is also now of some growing concern.

The need for new antibiotics is undisputed, however more pertinent is the need for new classes of antibiotics, particularly those with an ability to treat antibiotic resistant species and/or strains of bacteria. One such group of bacterial pathogens for which emerging resistance is of great concern is the Gram-positive and Gram-negative pathogens, Enterococcus faecium, Staphylococcus aureus, Klebsiella pneumonia, Acinetobacter baumannii, Pseudomonas aeruginosa and Enterobacter species, sometimes referred to by the acronym ESKAPE pathogens (Rice, L. B., J. Infect. Dis., 2008, 197:1079-81). This group of difficult to treat pathogens was subsequently modified to also include Clostridium difficile and additional Enterobacteriaceae, such as Escherichia coli, sometimes referred to by the acronym ESCAPE pathogens (Peterson, L. R., Gin. Infect. Dis., 2009, 49:992-3). While vancomycin resistance and multidrug resistant species of Gram-positive bacteria such as E. faecium (VRE) and S. aureus (MRSA), may have a high profile amongst the general public, it is the emerging resistance in the Gram-negative bacterial species of the ESKAPE and ESCAPE groups which is of increasing concern and presents additional challenges not faced by Gram-positive species. This may be due in part to the inherent differences in the cellular make-up of Gram-positive and Gram-negative bacteria and the added complexities of developing a clinically effective treatment as a result.

The inventors have discovered a novel drug combination which has potent activity against Gram-negative pathogens and/or drug resistant Gram-negative bacteria.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### SUMMARY

According to one aspect there is provided a composition comprising a bacterial type II topoisomerase inhibitor and a polymyxin or polymyxin derivative wherein the bacterial type II topoisomerase inhibitor has on-target enzyme activity against DNA gyrase and optionally on-target enzyme activity against topoisomerase IV.

According another aspect there is provided an antibacterial agent comprising a bacterial type II topoisomerase inhibitor and a polymyxin or polymyxin derivative wherein the bacterial type II topoisomerase inhibitor has on-target enzyme activity against DNA gyrase and optionally on-target enzyme activity against topoisomerase IV.

According to another aspect there is provided a method for the treatment or prevention of a bacterial infection comprising administration of a bacterial type II topoisomerase inhibitor in combination with a polymyxin or polymyxin derivative to a subject suffering from infection or at risk of infection, wherein the bacterial infection is caused by a Gram-negative bacteria or drug resistant Gram-negative bacteria and the bacterial type II topoisomerase inhibitor has on-target enzyme activity against DNA gyrase and optionally on-target enzyme activity against topoisomerase IV.

In one embodiment, the combination is administered as a composition.

In another embodiment, the combination is administered as an antibacterial agent.

In another aspect there is provided use of a bacterial type II topoisomerase inhibitor in combination with a polymyxin or polymyxin derivative in the treatment or prevention of a bacterial infection wherein the bacterial type II topoisomerase inhibitor has on-target enzyme activity against DNA gyrase and optionally on-target enzyme activity against topoisomerase IV and wherein the bacterial infection is caused by a Gram-negative bacteria or drug resistant Gram-negative bacteria.

In another aspect there is provided the use of a bacterial type II topoisomerase inhibitor in combination with a polymyxin or polymyxin derivative in the preparation of a medicament for the treatment or prevention of a bacterial infection wherein the bacterial type II topoisomerase inhibitor has on-target enzyme activity against DNA gyrase and optionally on-target enzyme activity against topoisomerase IV and wherein the bacterial infection is caused by a Gram-negative bacteria or drug resistant Gram-negative bacteria.

In another aspect there is provided a method of improving the antibacterial activity of a bacterial type II topoisomerase inhibitor wherein the method comprises the step of administration of the bacterial type II topoisomerase inhibitor with a polymyxin or polymyxin derivative to a subject suffering from an bacterial infection or at risk of a bacterial infection wherein the bacterial type II topoisomerase inhibitor has on-target enzyme activity against DNA gyrase and optionally on-target enzyme activity against topoisomerase IV and wherein the bacterial infection is caused by a Gram-negative bacteria or drug resistant Gram-negative bacteria.

In another aspect there is provided a method of improving the antibacterial efficacy of a bacterial type II topoisomerase inhibitor wherein the method comprises the step of administration of the bacterial type II topoisomerase inhibitor with a polymyxin or polymyxin derivative to a subject suffering from an bacterial infection or at risk of a bacterial infection wherein the bacterial type II topoisomerase inhibitor has on-target enzyme activity against DNA gyrase and optionally on-target enzyme activity against topoisomerase IV and wherein the bacterial infection is caused by a Gram-negative bacteria or drug resistant Gram-negative bacteria.

In one embodiment, the bacterial type II topoisomerase inhibitor has on-target enzyme activity against DNA gyrase and on-target enzyme activity against topoisomerase IV.

In one embodiment, the bacterial type II topoisomerase inhibitor is a GyrB/ParE inhibitor.

In one embodiment, the combination may be administered concurrently, sequentially or separately to a patient suffering from infection or at risk of infection.

In one embodiment, the Gram-negative bacteria or drug resistant Gram-negative bacteria comprises a lipopolysaccharide (LPS) layer.

In another embodiment, the Gram-negative bacteria or drug resistant Gram-negative bacteria comprises a lipooligosaccharide (LOS) layer.

In one embodiment, the bacterial type II topoisomerase inhibitor is a compound of Formula (I) as defined herein or a salt, racemate, diastereomer, enantiomer, ester, carbamate, phosphate, sulfate, deuterated form or prodrug thereof.

In one embodiment, the bacterial type II topoisomerase inhibitor is a compound of Formula (II) as defined herein or a salt, racemate, diastereomer, enantiomer, ester, carbamate, phosphate, sulfate, deuterated form or prodrug thereof.

In another aspect there is provided a compound of Formula (II) as defined herein or a salt, racemate, diastereomer, enantiomer, ester, carbamate, phosphate, sulfate, deuterated form or prodrug thereof.

In another aspect there is provided a process for the manufacture of a compound of Formula (II) as defined herein or a salt, racemate, diastereomer, enantiomer, ester, carbamate, phosphate, sulfate, deuterated form or prodrug thereof.

In one embodiment, the bacterial type II topoisomerase inhibitor is a compound of Formula (III) as defined herein or a salt, racemate, diastereomer, enantiomer, ester, carbamate, phosphate, sulfate, deuterated form or prodrug thereof.

In another aspect there is provided a compound of Formula (III) as defined herein or a salt, racemate, diastereomer, enantiomer, ester, carbamate, phosphate, sulfate, deuterated form or prodrug thereof.

In another aspect there is provided a process for the manufacture of a compound of Formula (III) as defined herein or a salt, racemate, diastereomer, enantiomer, ester, carbamate, phosphate, sulfate, deuterated form or prodrug thereof.

In one embodiment, the bacterial type II topoisomerase inhibitor is a compound of Formula (IV) as defined herein or a salt, racemate, diastereomer, enantiomer, ester, carbamate, phosphate, sulfate, deuterated form or prodrug thereof.

In another aspect there is provided a compound of Formula (IV) as defined herein or a salt, racemate, diastereomer, enantiomer, ester, carbamate, phosphate, sulfate, deuterated form or prodrug thereof.

In another aspect there is provided a process for the manufacture of a compound of Formula (IV) as defined herein or a salt, racemate, diastereomer, enantiomer, ester, carbamate, phosphate, sulfate, deuterated form or prodrug thereof.

### BRIEF DESCRIPTION OF THE FIGURES

FIG. 1: Shows in vivo efficacy of a compound of Formula (I) in combination with a polymyxin derivative in accordance with an embodiment of the present disclosure in the E. coli septicaemia model of infection. Groups of mice were inoculated intraperitoneally (IP) with a lethal dose of E. coli and survival was monitored for five (5) days. The groups of mice tested were as follows:

Vehicle control (results represented by thin dotted line);

Polymyxin B nonapeptide (PMBN) (50 mg/kg) administered subcutaneously (SC) at 1 and 3 hours post infection (results represented by thin dashed line);

Compound of Formula (I) (Example 152 of WO2013/138860) (100 mg/kg) administered intravenously (IV) at 1 and 3 hours post infection (results represented by thick dotted line);

Compound of Formula (I) (Example 152 of WO2013/138860) (100 mg/kg) administered intravenously (IV) plus PMBN (50 mg/kg) administered subcutaneously (SC) at 1 hour post infection (results represented by thick dashed line);

Compound of Formula (I) (Example 152 WO2013/138860) (100 mg/kg) administered intravenously (IV) plus PMBN (50 mg/kg) administered subcutaneously (SC) at 1 and 3 hours post infection (results represented by thick solid line).

### DETAILED DESCRIPTION

The present disclosure is predicated on the discovery of a novel combination of a bacterial type II topoisomerase inhibitor and polymyxin or a polymyxin derivative. This novel combination shows potent activity against bacterial infections caused by a Gram-negative bacteria or drug resistant Gram-negative bacteria, particularly when compared to the antibacterial activity of the bacterial type II topoisomerase inhibitor or polymyxin or polymyxin derivative alone.

The polymyxin and polymyxin derivatives of the combination may be selected from antibacterial polymyxins, antibacterial polymyxin derivatives, non-antibacterial polymyxins, non-antibacterial polymyxin derivatives, and polymyxins and polymyxin derivatives which may act as an antibacterial or non-antibacterial agent depending on the amount or dosage to be administered.

In another embodiment the polymyxin or polymyxin derivative may be provided in a therapeutically effective antibacterial amount or dosage.

In another embodiment the polymyxin or polymyxin derivative may be an antibacterial polymyxin or antibacterial polymyxin derivative.

In another embodiment the antibacterial polymyxin or antibacterial polymyxin derivative may be provided in a sub-inhibitory MIC amount or dosage, that is, a non-therapeutically effective antibacterial amount or dosage.

In another embodiment the polymyxin or polymyxin derivative may be a non-antibacterial polymyxin or a non-antibacterial polymyxin derivative.

Examples of Polymyxins useful in the novel combination include Polymyxin B (PMB) and colistin (Polymyxin E). PMB and colistin are examples of antibacterial polymyxins which may act as either an antibacterial or non-antibacterial agent depending on the amount or dosage to be administered.

Examples of Polymyxin derivatives may be useful in the novel combination therapy include nonapeptide derivatives such as Polymyxin B nonapeptide (PMBN) and prodrug forms of colistin. For example, the produce form of colistin may be colistin methanesulfonate (CMS). PMBN is an example of a non-antibacterial agent. CMS is also an example of a non-antibacterial agent although as it is a prodrug of colistin, the amount or dosage of CMS to be administered will determine whether or not the amount or dosage of colistin when released in vivo from its prodrug form will act as an antibacterial or non-antibacterial agent.

In one embodiment the polymyxin may be colistin (Polymyxin E). In a further embodiment colistin may be administered in an antibacterially effective amount or dosage. In another embodiment colistin may be administered in a non-antibacterially effective amount or dosage.

In one embodiment the polymyxin derivative may be a prodrug of colistin. In a further embodiment the prodrug of colistin may be administered in an amount or dosage to provide an antibacterially effective amount or dosage of colistin. In another embodiment the prodrug of colistin may be administered in an amount or dosage to provide a non-antibacterially effective amount or dosage of colistin.

In a further embodiment the prodrug of colistin may be colistin methanesulfonate (CMS).

In one embodiment the polymyxin may be Polymyxin B (PMB). In a further embodiment PMB may be administered in an antibacterially effective amount or dosage. In another embodiment PMB may be administered in a non-antibacterially effective amount or dosage.

In one embodiment the polymyxin derivative may be Polymyxin B nonapeptide (PMBN).

The bacterial type II topoisomerase inhibitors for use in the novel combination therapy have on-target enzyme activity against DNA gyrase and optionally on-target enzyme activity against topoisomerase IV. Examples of bacterial type II topoisomerase inhibitors that may be useful in the novel combination therapy include, though are not limited to compounds described in applicant's earlier filed applications WO2007/148093, WO2009/074812, WO2009/074810, WO2012/045124 and WO2013/138860. Examples of compounds of Formula (I) and or Formula (II) as described herein may be useful as bacterial type II topoisomerase inhibitors for use in the novel combination therapy.

Other examples of bacterial type II topoisomerase inhibitors that have demonstrated on-target enzyme activity against DNA gyrase which may also be useful in the novel combination therapy include, though are not limited to compounds described in WO2001/052845 (Vertex Pharmaceuticals Incorporated, Charifson P. et. al.); WO2001/052846 (Vertex Pharmaceuticals Incorporated, Charifson P. et. al.); WO2002/060879 (Vertex Pharmaceuticals Incorporated, Grillot, A. et. al.); WO2003/105846 (Vertex Pharmaceuticals Incorporated, Charifson P. et. al.); WO2005/012292 (Vertex Pharmaceuticals Incorporated, Charifson P. et. al.); WO2006/022773 (Vertex Pharmaceuticals Incorporated, Charifson P. et. al.); WO2007/056330 (Vertex Pharmaceuticals Incorporated, Charifson P. et. al.); WO2009/061875 (Vertex Pharmaceuticals Incorporated, Forslund, R. et. al.); WO2009/076200 (Vertex Pharmaceuticals Incorporated, Alargova, R. et. al.); WO2012/097269 (Vertex Pharmaceuticals Incorporated, Le Tiran, A. et. al.); WO2012/097270 (Vertex Pharmaceuticals Incorporated, Shannon, D. et. al.); WO2012/097273 (Vertex Pharmaceuticals Incorporated, Shannon, D. et. al.); WO2012/097274 (Vertex Pharmaceuticals Incorporated, Shannon, D. et. al.); WO2012/177707 (Vertex Pharmaceuticals Incorporated, Bennani, Y. L. et. al.); WO2014/014845 (Vertex Pharmaceuticals Incorporated, Locher, C. P, et. al.); WO2014/015105 (Vertex Pharmaceuticals Incorporated, O'Dowd, H. et. al.); WO2011/032050 (Trius Therapeutics, Inc., Creighton, C. et. al.); and WO2012/125746 (Trius Therapeutics, Inc., Bensen, D. et. al.).

In one embodiment the bacterial type II topoisomerase may be a compound of Formula (I):

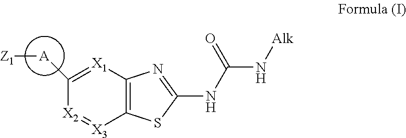

and; salts, racemates, diastereomers, enantiomers, deuterated forms, hydrates, solvates and prodrugs thereof wherein Alk, A, X₁, X₂, X₃ and Z₁ may be as follows.

Alk may be an optionally substituted C₁₋₆alkyl, C₂₋₆alkenyl, C₂₋₆alkynyl, or C₃₋₆cycloalkyl. For example, Alk may be an optionally substituted C₁₋₆alkyl. In one example, Alk may be an unsubstituted C₁₋₆alkyl. In one example, Alk may be an ethyl. Accordingly, any compound according to Formula (I), Alk may be an ethyl.

A represents “Ring A” which may be selected from saturated or unsaturated monocyclic C₃₋₇ cycloalkyl, saturated or unsaturated monocyclic 3-7 membered heterocycle, saturated or unsaturated fused bicyclic C₈₋₁₀cycloalkyl, saturated or unsaturated fused bicyclic 8-10 membered-heterocyclyl, C₆₋₁₀aryl and 5-10 membered heteroaryl and may be optionally substituted. For example, Ring A may be an optionally substituted 5-6-membered hetero-monocyclic ring or an 8-10-membered fused hetero-bicyclic ring. In another example, Ring A may be an optionally substituted 5-6-membered heteroaryl ring. In another example, Ring A may be an optionally substituted 6-membered heteroaryl ring.

X₁ may be a CH, —N═ or C—R₁, where R₁ may be selected from OH, optionally substituted C₁₋₃alkyl, optionally substituted C₂₋₃alkenyl, optionally substituted C₂₋₃alkynyl, optionally substituted C₁₋₃alkoxyl, halo, haloC₁₋₃alkyl, NH₂, optionally substituted NHC₁₋₃alkyl, optionally substituted N(C₁₋₃alkyl)₂, optionally substituted SC₁₋₃alkyl and CN.

X₂ may be a CH, —N═ or C—R₂, where R₂ may be selected from OH, optionally substituted C₁₋₆alkyl, optionally substituted C₂₋₆alkenyl, optionally substituted C₂₋₆alkynyl, optionally substituted (CH₂)ₘOC₁₋₆alkyl, optionally substituted (CH₂)ₘSC₁₋₆alkyl, optionally substituted (CH₂)ₘS(═O)C₁₋₆alkyl, optionally substituted (CH₂)ₘO(CH₂)ₘC₃₋₇cycloalkyl, optionally substituted (CH₂)ₘC₃₋₇cycloalkyl, optionally substituted (CH₂)ₘO(CH₂)ₘphenyl, optionally substituted (CH₂)ₘphenyl, optionally substituted (CH₂)ₘO(CH₂)ₘ-5-10-membered heterocycle, optionally substituted (CH₂)ₘ-5-10-membered heterocyclyl, halo, optionally substituted haloC₁₋₃alkyl, CN and optionally substituted (CH₂)ₘNR^{a}R^{b}.

X₃ may be a CH, —N═ or C—R₃, where R₃ may be selected from OH, optionally substituted C₁₋₆alkyl, optionally substituted C₂₋₆alkenyl, optionally substituted C₂₋₆alkynyl, optionally substituted (CH₂)ₘOC₁₋₆alkyl, optionally substituted (CH₂)ₘSC₁₋₆alkyl, optionally substituted (CH₂)ₘS(═O)C₁₋₆alkyl, optionally substituted (CH₂)ₘO(CH₂)ₘC₃₋₇cycloalkyl, optionally substituted (CH₂)ₘC₃₋₇cycloalkyl, optionally substituted (CH₂)ₘO(CH₂)ₘphenyl, optionally substituted (CH₂)ₘphenyl, optionally substituted (CH₂)ₘO(CH₂)ₘ-5-10-membered heterocycle, optionally substituted (CH₂)ₘ-5-10-membered heterocyclyl, halo, optionally substituted haloC₁₋₃alkyl, CN and optionally substituted (CH₂)ₘNR^{a}R^{b};

In one example, R₃ may be an optionally substituted 5-membered or 6-membered (CH₂)ₘheterocyclic ring, wherein the optional substituents may be one or more substituents independently selected from OH, optionally substituted C₁₋₆alkyl, optionally substituted C₂₋₆alkenyl, optionally substituted C₂₋₆alkynyl, optionally substituted (CH₂)ₘOC₁₋₆alkyl, optionally substituted (CH₂)ₘSC₁₋₆alkyl, optionally substituted (CH₂)ₘS(═O)C₁₋₆alkyl, halo, optionally substituted haloC₁₋₃alkyl, CN, optionally substituted (CH₂)ₘNR^{a}R^{b}, optionally substituted (CH₂)ₚ-4-6-membered heterocyclic ring, optionally substituted (CH₂)ₚ-spiro-bicyclic-7-11-membered heterocyclic ring and optionally substituted

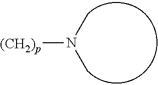

where p may be an integer selected from 0, 1, 2 and 3 and

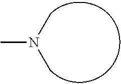

may represent an optionally substituted 4-6 membered heterocyclic ring or an optionally substituted spiro bicyclic 7-11-membered heterocyclic ring;

In another example, R₃ may be an optionally substituted 6-membered heteroaryl ring selected from pyridinyl, pyrimidinyl, pyridazinyl and pyrazinyl.

Each R^{a} and R^{b} may be independently selected from H, optionally substituted C₁₋₆alkyl, optionally substituted C₃₋₆cycloalkyl and optionally substituted 4-6-membered heterocyclyl or R^{a} and R^{b} join together to form an optionally substituted 4-6-membered heterocyclyl.

Each m may be an integer independently selected from 0, 1, 2 and 3. For example, m may be 0 or 1. In one example, m may be 0.

Z₁ may be selected from H, halo, C₁₋₆alkyl, a 5-membered heterocyclic ring including 5-membered heteroaryl rings, a 6-membered heterocyclic ring including 6-membered heteroaryl rings, OH, OC₁₋₆alkyl, C₁₋₆alkoxyl, cyano (CN), a carbonyl moiety (═O), C(═O)OC₁₋₆alkyl, NH₂, NH—C₁₋₆alkyl, N(C₁₋₆alkyl)₂, and C(═O)NH—C₁₋₆alkyl.

In another example, Z₁ may be a carbonyl containing group of general formula —(Y)_{q}B(R₄)—C(═O)—W—R₅;

wherein:

q may be an integer 0 or 1;

Y may be attached to Ring A and when q is 0 then Y may be a covalent bond, a spiro ring centre, or a fused ring bond. For example, Y may be a covalent bond when q may be 0. In one example, q is 1 then Y may be selected from optionally substituted C₁₋₃alkylene, optionally substituted C₂₋₃alkenylene and optionally substituted C₂₋₃alkynylene and wherein each carbon atom in C₁₋₃alkylene may be optionally replaced by an oxygen or nitrogen heteroatom or C(═O). In another example, Y may be selected from the group consisting of —C(O)NH— or —NHC(O)—, —NH—, —CH₂NH—, —NHCH₂—, —N(CH₃)—, —CH₂N(CH₃)—, —N(CH₃)CH₂—, methylene, ethylene, propylene and C═O. In another example, Y may be selected from methylene, NH, N(CH₃) and C(═O) when q is 1.

B represents “Ring B” and may be selected from saturated or unsaturated monocyclic C₃₋₇cycloalkyl, saturated or unsaturated monocyclic 3-7 membered heterocycle, saturated or unsaturated fused bicyclic C₈₋₁₀cycloalkyl, saturated or unsaturated fused bicyclic 8-12 membered heterocyclyl, C₆₋₁₀aryl, 5-10 membered heteroaryl, and a spiro bicyclic 8-12 membered heterocyclic ring system. In one example, Ring B may be optionally substituted. In another example, Ring B may join together with Ring A to form a saturated or unsaturated fused bicyclic C₈₋₁₀cycloalkyl, a saturated or unsaturated fused bicyclic 8-10 membered heterocyclyl and a spiro bicyclic 8-12 membered heterocyclic ring system. In one example, Ring B may be an optionally substituted C₃₋₇cycloalkyl or an optionally substituted 4-, 5-, 6- or 7-membered heterocyclic group. For example, an optionally substituted C₅₋₆cycloalkyl. For example, cyclohexyl or an optionally substituted 5- or 6-membered heterocyclic group. For example, a 6-membered. In one example, Ring B may be a heterocyclic group containing nitrogen and/or oxygen and includes dioxane, piperidinyl, pyrrolidinyl, azepane, isoxazolyl and morpholinyl. In one example, Ring B may be selected from piperidinyl, pyrrolidinyl, azepane, isoxazolyl and morpholinyl. In one example, Ring B may be piperidinyl.

R₄ may be joined to the same Ring B atom as the —C(═O)—W—R₅ moiety and may be selected from C₁₋₆alkyl, C₂₋₆alkenyl, C₂₋₆alkynyl, (C₁₋₆alkyl)ₜC₃₋₇cycloalkyl, (C₁₋₆alkyl)ₜaryl, (C₁₋₆ alkyl)ₜheterocyclyl, (C₁₋₆alkyl)ₜheteroaryl, NH₂, NH(C₁₋₆alkyl), N(C₁₋₆alkyl)₂, CN, OH, C₁₋₆alkoxy, SO₂H, SO₂C₁₋₆alkyl, SH, SC₁₋₆alkyl, halo, haloC₁₋₆alkyl, —NH(C═O)OC₁₋₆alkyl, —NH(C═O)OC(C₁₋₃alkyl)₃, and wherein C₁₋₃alkyl, C₁₋₆alkyl, C₂₋₆ alkenyl, C₂₋₆alkynyl, C₃₋₇cycloalkyl, aryl and heterocyclyl in each case may be further optionally substituted, with, for example, one or more substituents selected from NH₂, NH(C₁₋₆alkyl), N(C₁₋₆alkyl)₂, CN, OH, C₁₋₆alkoxy, SO₂H, SO₂C₁₋₆alkyl, SH, SC₁₋₆alkyl and halo or R₄ may be a chain of 3 or 4 carbon atoms or carbon and heteroatoms which joins with an adjacent B ring atom to form a fused carbocyclylic or heterocyclic ring which is optionally further substituted. In one example, R₄ may be a C₁₋₆alkyl or C₃₋₇cycloalkyl. In another example, R₄ may be a C₁₋₃alkyl or cyclopropyl. In another example, R₄ may be a methyl, ethyl, n-propyl and iso-propyl. In one example, R₄ may be a methyl or ethyl.

The —C(═O)—W—R₅ moiety may be joined to the same Ring B atom as R₄; wherein W may be a O, NH or N(C₁₋₆alkyl). In one example, W may be O; and R₅ may be selected from H, C₁₋₆alkyl, C₂₋₆alkenyl, C₂₋₆alkynyl, S(O)₂OH, S(O)₂—C₁₋₆alkyl, or M where M represents a monovalent or divalent cation selected from the group comprising pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. In one example, R₅ may be H or C₁₋₃alkyl selected from methyl, ethyl, propyl and iso-propyl. In another example, R₅ may be H.

In another example, Z₁ is an alcohol containing group of general formula (CH₂)ₛC(OH)(R₆)(R₇) or an ester, carbamate, phosphate, sulfate or prodrug thereof; wherein the OH, R₆ and R₇ groups are each attached to the same carbon atom; and wherein:

s may be an integer selected from 0, 1, 2 and 3. In one example, s may be 0 or 1. In one example, s is 0.

R₆ may be H or may be selected from optionally substituted C₁₋₆alkyl, optionally substituted C₂₋₆alkenyl, optionally substituted C₂₋₆alkynyl, optionally substituted (CH₂)ₜOC₁₋₆alkyl, optionally substituted (CH₂)ₜOC(═O)C₁₋₆alkyl, optionally substituted (CH₂)ₜSC₁₋₆alkyl, optionally substituted (CH₂)ₜS(═O)C₁₋₆alkyl, halo, optionally substituted haloC₁₋₃alkyl and optionally substituted (CH₂)ₜNR^{a}R^{b}. For example, R₆ may be H or optionally substituted C₁₋₃alkyl. For example, methyl and ethyl.

R₇ may be selected from optionally substituted C₁₋₆alkyl, optionally substituted C₂₋₆alkenyl, optionally substituted C₂₋₆alkynyl, optionally substituted C₃₋₇cycloalkyl ring, optionally substituted phenyl, optionally substituted 4-6-membered heterocyclyl ring, optionally substituted 5-6-membered heteroaryl ring, optionally substituted (CH₂)ₜOC₁₋₆alkyl, optionally substituted (CH₂)ₜOC(═O)C₁₋₆ alkyl, optionally substituted (CH₂)ₜSC₁₋₆alkyl, optionally substituted (CH₂)ₜS(═O)C₁₋₆alkyl, halo, optionally substituted haloC₁₋₃alkyl and optionally substituted (CH₂)ₜNR^{a}R^{b}. For example, R₇ may be selected from optionally substituted C₁₋₃alkyl. For example, methyl and ethyl, optionally substituted haloC₁₋₃alkyl. In one example, R₇ may be selected from CHF₂, CH₂CHF₂, CF₃ and CH₂CF₃. In one example, R₇ may be an optionally substituted C₃₋₇cycloalkyl ring. In one example, R₇ may be selected from cyclopropyl, cyclobutyl, cyclopentyl and cyclohexyl. In another example, R₇ may be an optionally substituted 4-6-membered heterocyclyl ring (e.g. morpholinyl), optionally substituted 5-6-membered heteroaryl ring (e.g. containing at least one nitrogen heteroatom such as imidazolyl and pyridinyl).

t may be an integer selected from 1, 2, 3, 4, 5 and 6. For example, t may be an integer selected from 1, 2 or 3.

In another example, R₆ and R₇ together with the carbon atom to which they are attached form an optionally substituted 4-6-membered heterocyclic ring or C₃₋₇cycloalkyl ring.

In one example, the prodrug may be selected from an ester, carbamate, phosphate or sulfate formed from the hydroxyl moiety.

In another example, Z₁ may be a sulfonamide containing group of general formula (CH₂)ᵥNRS(═O)₂R₈ or (CH₂)ᵥS(═O)₂NR₉R₁₀ or a sulfamide containing group of general formula (CH₂)ᵥNRS(═O)₂NR₉R₁₀; wherein

v may be an integer 0, 1, 2 or 3. For example, v may be 0 or 1.

R may be H or an optionally substituted C₁₋₆alkyl. For example, R may be H; and

R₈, R₉ and R₁₀ are each independently selected from H, C₁₋₆alkyl, C₂₋₆alkenyl, C₂-6alkynyl, C₃₋₇cycloalkyl, phenyl, benzyl, a 3-10-membered heterocyclic ring, a 5-10-membered heteroaryl ring and further wherein each C₁₋₆alkyl, C₂₋₆alkenyl, C₂₋₆alkynyl, C₃₋₇cycloalkyl, phenyl, benzyl, a 3-10-membered heterocyclic ring, a 5-10-membered heteroaryl ring may be optionally substituted;

or R₉ and R₁₀ may join to form an optionally substituted 3-6-membered heterocyclic ring together with the nitrogen to which they are attached.

In one embodiment of Formula (I):

Alk may be an unsubstituted C₁₋₆alkyl. For example, Alk may be an ethyl.

Ring A may be an optionally substituted 5-6-membered hetero-monocyclic ring or an 8-10-membered fused hetero-bicyclic ring. In one example, Ring A may be an optionally substituted 5-6-membered heteroaryl ring. In one example, Ring A may be an optionally substituted 6-membered heteroaryl ring.

X₁ may be an CH, —N═ or C—R₁ where R₁ may be selected from OH, optionally substituted C₁₋₃alkyl, optionally substituted C₂₋₃alkenyl, optionally substituted C₂₋₃alkynyl, optionally substituted C₁₋₃alkoxyl, halo, haloC₁₋₃alkyl, NH₂, optionally substituted NHC₁₋₃alkyl, optionally substituted N(C₁₋₃alkyl)₂, optionally substituted SC₁₋₃alkyl and CN. In one example, R₁ may be a halo or C₁₋₃alkyl. In one example, X₁ may be a CH.

X₂ may be a CH or —N═.

X₃ may be a CH, —N═ or C—R₃. In one example, X₃ may be C—R₃ where R₃ may be halo or an optionally substituted 5-membered or 6-membered heteroaryl ring. In one example, X₃ may be an optionally substituted 6-membered heteroaryl ring; and

Z₁ may be selected from H, halo, C₁₋₆alkyl, a 5-membered heterocyclic ring including 5-membered heteroaryl rings, a 6-membered heterocyclic ring including 6-membered heteroaryl rings, OH, OC₁₋₆alkyl, C₁₋₆alkoxyl, cyano (CN), a carbonyl moiety (═O), C(═O)OC₁₋₆alkyl, NH₂, NH—C₁₋₆alkyl, N(C₁₋₆alkyl)₂, and C(═O)NH—C₁₋₆alkyl; and wherein Z₁ may be further optionally substituted.

In one example, optional substituents for Ring A, R₃ and/or Z₁ include but are not limited to one or more substituents independently selected from halo, C₁₋₆alkyl, a 5-membered heterocyclic ring including 5-membered heteroaryl rings, a 6-membered heterocyclic ring including 6-membered heteroaryl rings, OH, OC₁₋₆alkyl, C₁₋₆alkoxyl, cyano (CN), a carbonyl moiety (═O), C(═O)OC₁₋₆alkyl, NH₂, NH—C₁₋₆alkyl, N(C₁₋₆alkyl)₂, and C(═O)NH—C₁₋₆alkyl.

Suitable compounds according to this embodiment where X₂ may be CH includes but is not limited to, any one of compound examples 1 to 179 as previously disclosed in WO2007/148093:

| No. | Structure |
|---|---|
| 1 | 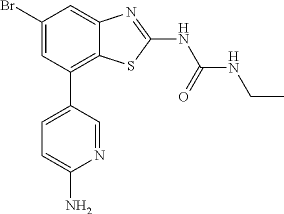 [SMILES: CCNC(=O)Nc1nc2cc(Br)cc(-c3ccc(N)nc3)c2s1] |
| 2 | 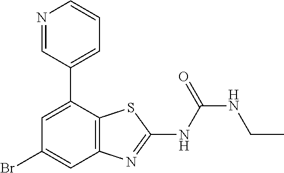 [SMILES: CCNC(=O)Nc1nc2cc(Br)cc(-c3cccnc3)c2s1] |
| 3 | 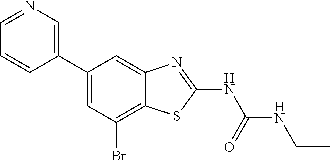 [SMILES: CCNC(=O)Nc1nc2cc(-c3cccnc3)cc(Br)c2s1] |
| 4 | 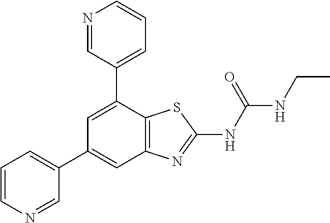 [SMILES: CCNC(=O)Nc1nc2cc(-c3cccnc3)cc(-c3cccnc3)c2s1] |
| 5 | 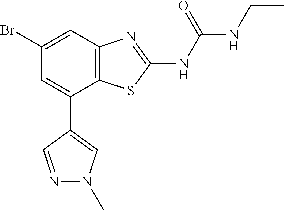 [SMILES: CCNC(=O)Nc1nc2cc(Br)cc(-c3cnn(C)c3)c2s1] |
| 6 | 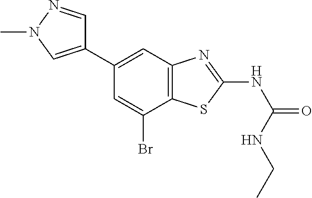 [SMILES: CCNC(=O)Nc1nc2cc(-c3cnn(C)c3)cc(Br)c2s1] |
| 7 | 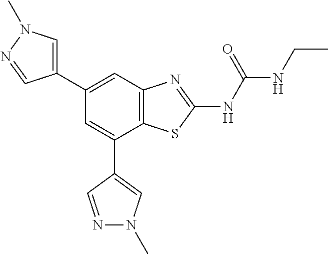 [SMILES: CCNC(=O)Nc1nc2cc(-c3cnn(C)c3)cc(-c3cnn(C)c3)c2s1] |
| 8 | 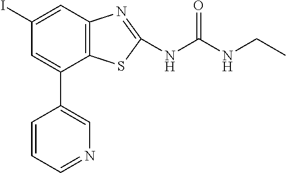 [SMILES: CCNC(=O)Nc1nc2cc(I)cc(-c3cccnc3)c2s1] |
| 9 | 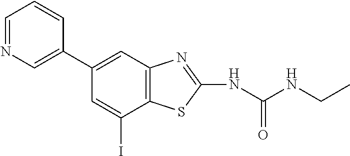 [SMILES: CCNC(=O)Nc1nc2cc(-c3cccnc3)cc(I)c2s1] |
| 10 | 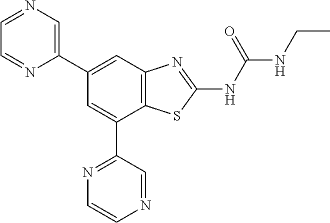 [SMILES: CCNC(=O)Nc1nc2cc(-c3cnccn3)cc(-c3cnccn3)c2s1] |
| 11 | 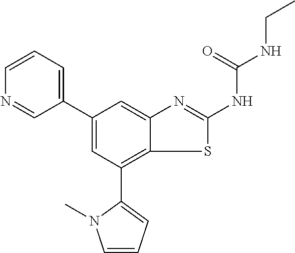 [SMILES: CCNC(=O)Nc1nc2cc(-c3cccnc3)cc(-c3cccn3C)c2s1] |
| 12 | 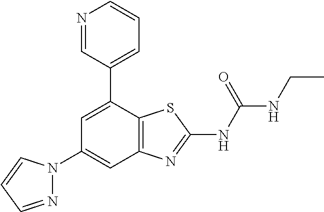 [SMILES: CCNC(=O)Nc1nc2cc(-n3cccn3)cc(-c3cccnc3)c2s1] |
| 13 | 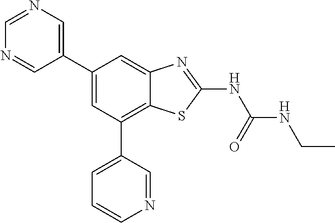 [SMILES: CCNC(=O)Nc1nc2cc(-c3cncnc3)cc(-c3cccnc3)c2s1] |
| 14 | 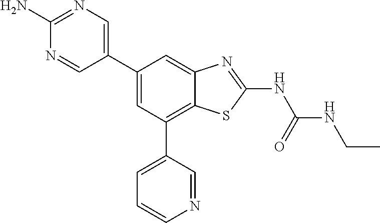 [SMILES: CCNC(=O)Nc1nc2cc(-c3cnc(N)nc3)cc(-c3cccnc3)c2s1] |
| 15 | 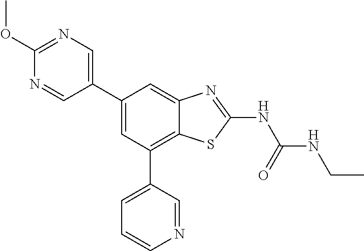 [SMILES: CCNC(=O)Nc1nc2cc(-c3cnc(OC)nc3)cc(-c3cccnc3)c2s1] |
| 16 | 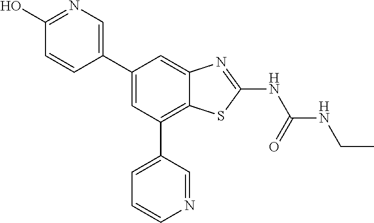 [SMILES: CCNC(=O)Nc1nc2cc(-c3ccc(O)nc3)cc(-c3cccnc3)c2s1] |
| 17 | 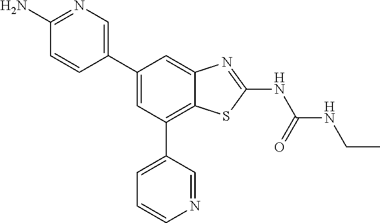 [SMILES: CCNC(=O)Nc1nc2cc(-c3ccc(N)nc3)cc(-c3cccnc3)c2s1] |
| 18 | 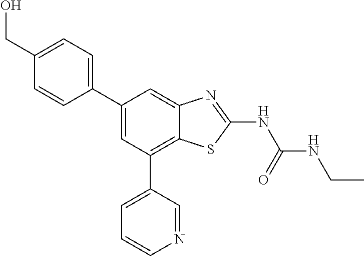 [SMILES: CCNC(=O)Nc1nc2cc(-c3ccc(CO)cc3)cc(-c3cccnc3)c2s1] |
| 19 | 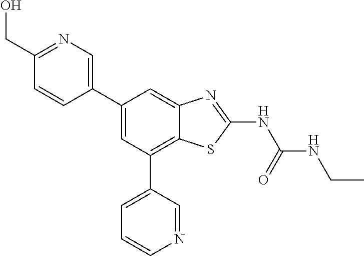 [SMILES: CCNC(=O)Nc1nc2cc(-c3ccc(CO)nc3)cc(-c3cccnc3)c2s1] |
| 20 | 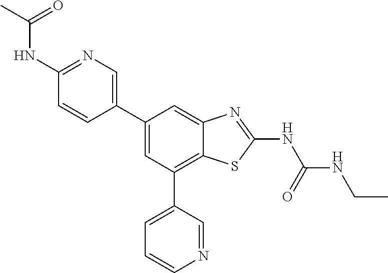 [SMILES: CCNC(=O)Nc1nc2cc(-c3ccc(NC(C)=O)nc3)cc(-c3cccnc3)c2s1] |
| 21 | 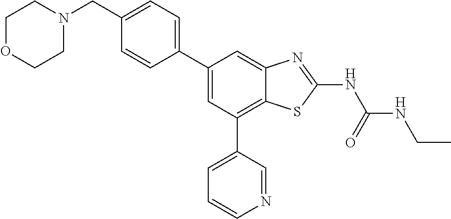 [SMILES: CCNC(=O)Nc1nc2cc(-c3ccc(CN4CCOCC4)cc3)cc(-c3cccnc3)c2s1] |
| 22 | 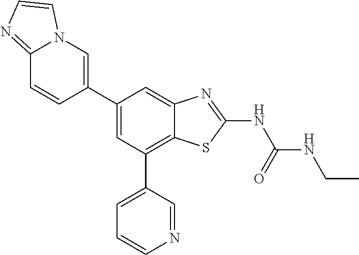 [SMILES: CCNC(=O)Nc1nc2cc(-c3ccc4nccn4c3)cc(-c3cccnc3)c2s1] |
| 23 | 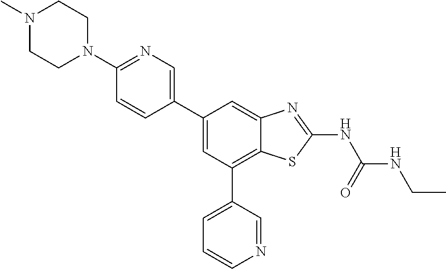 [SMILES: CCNC(=O)Nc1nc2cc(-c3ccc(N4CCN(C)CC4)nc3)cc(-c3cccnc3)c2s1] |
| 24 | 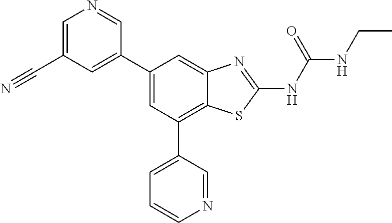 [SMILES: CCNC(=O)Nc1nc2cc(-c3cncc(C#N)c3)cc(-c3cccnc3)c2s1] |
| 25 | 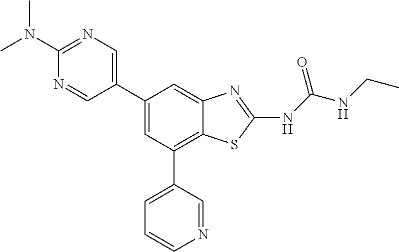 [SMILES: CCNC(=O)Nc1nc2cc(-c3cnc(N(C)C)nc3)cc(-c3cccnc3)c2s1] |
| 26 | 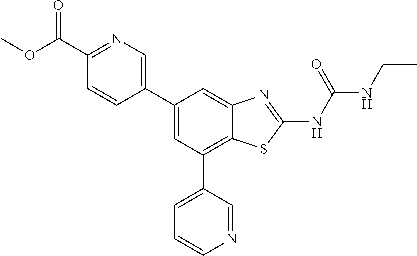 [SMILES: CCNC(=O)Nc1nc2cc(-c3ccc(C(=O)OC)nc3)cc(-c3cccnc3)c2s1] |
| 27 | 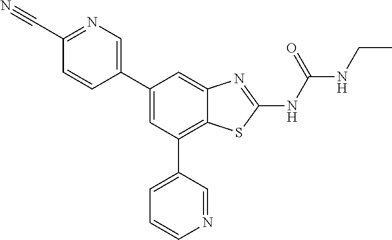 [SMILES: CCNC(=O)Nc1nc2cc(-c3ccc(C#N)nc3)cc(-c3cccnc3)c2s1] |
| 28 | 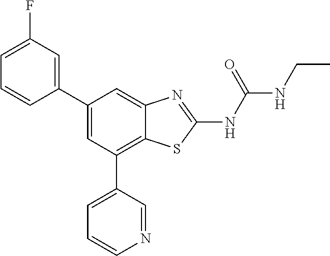 [SMILES: CCNC(=O)Nc1nc2cc(-c3cccc(F)c3)cc(-c3cccnc3)c2s1] |
| 29 | 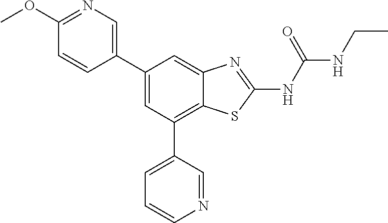 [SMILES: CCNC(=O)Nc1nc2cc(-c3ccc(OC)nc3)cc(-c3cccnc3)c2s1] |
| 30 | 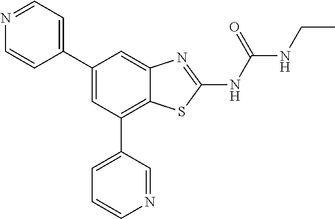 [SMILES: CCNC(=O)Nc1nc2cc(-c3ccncc3)cc(-c3cccnc3)c2s1] |
| 31 | 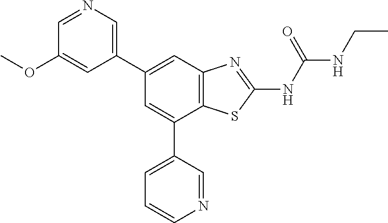 [SMILES: CCNC(=O)Nc1nc2cc(-c3cncc(OC)c3)cc(-c3cccnc3)c2s1] |
| 32 | 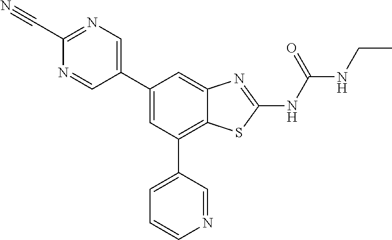 [SMILES: CCNC(=O)Nc1nc2cc(-c3cnc(C#N)nc3)cc(-c3cccnc3)c2s1] |
| 33 | 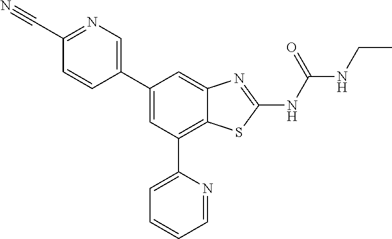 [SMILES: CCNC(=O)Nc1nc2cc(-c3ccc(C#N)nc3)cc(-c3ccccn3)c2s1] |
| 34 | 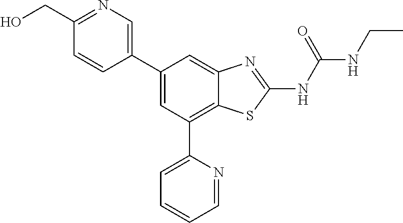 [SMILES: CCNC(=O)Nc1nc2cc(-c3ccc(CO)nc3)cc(-c3ccccn3)c2s1] |
| 35 | 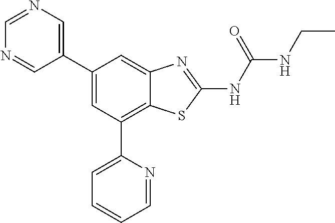 [SMILES: CCNC(=O)Nc1nc2cc(-c3cncnc3)cc(-c3ccccn3)c2s1] |
| 36 | 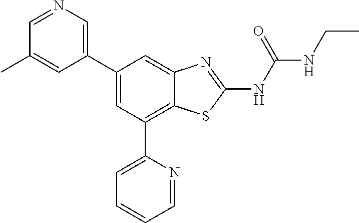 [SMILES: CCNC(=O)Nc1nc2cc(-c3cncc(C)c3)cc(-c3ccccn3)c2s1] |
| 37 | 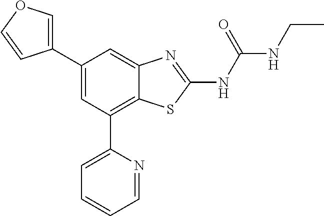 [SMILES: CCNC(=O)Nc1nc2cc(-c3ccoc3)cc(-c3ccccn3)c2s1] |
| 38 | 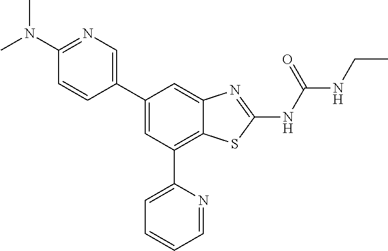 [SMILES: CCNC(=O)Nc1nc2cc(-c3ccc(N(C)C)nc3)cc(-c3ccccn3)c2s1] |
| 39 | 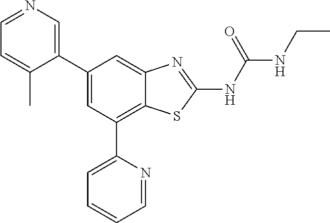 [SMILES: CCNC(=O)Nc1nc2cc(-c3cnccc3C)cc(-c3ccccn3)c2s1] |
| 40 | 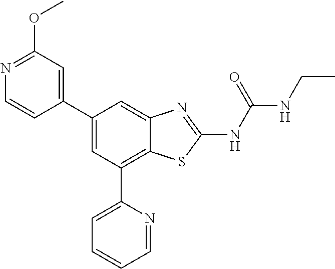 [SMILES: CCNC(=O)Nc1nc2cc(-c3ccnc(OC)c3)cc(-c3ccccn3)c2s1] |
| 41 | 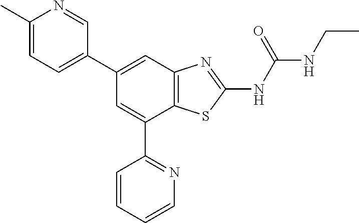 [SMILES: CCNC(=O)Nc1nc2cc(-c3ccc(C)nc3)cc(-c3ccccn3)c2s1] |
| 42 | 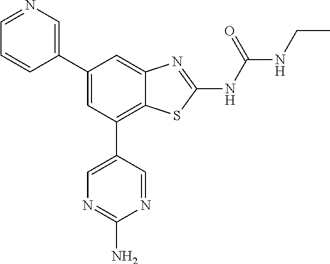 [SMILES: CCNC(=O)Nc1nc2cc(-c3cccnc3)cc(-c3cnc(N)nc3)c2s1] |
| 43 | 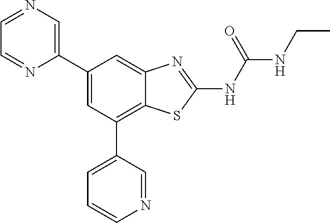 [SMILES: CCNC(=O)Nc1nc2cc(-c3cnccn3)cc(-c3cccnc3)c2s1] |
| 44 | 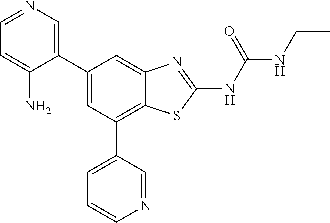 [SMILES: CCNC(=O)Nc1nc2cc(-c3cnccc3N)cc(-c3cccnc3)c2s1] |
| 45 | 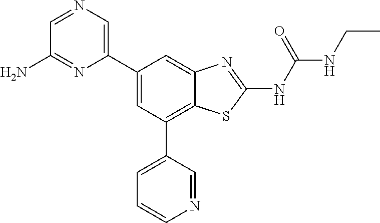 [SMILES: CCNC(=O)Nc1nc2cc(-c3cncc(N)n3)cc(-c3cccnc3)c2s1] |
| 46 | 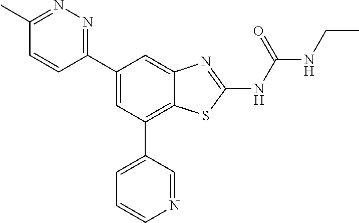 [SMILES: CCNC(=O)Nc1nc2cc(-c3ccc(C)nn3)cc(-c3cccnc3)c2s1] |
| 47 | 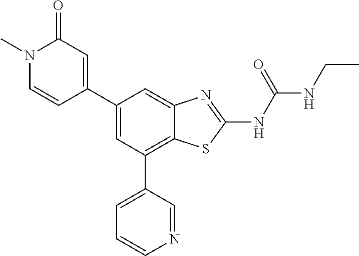 [SMILES: CCNC(=O)Nc1nc2cc(-c3ccn(C)c(=O)c3)cc(-c3cccnc3)c2s1] |
| 48 | 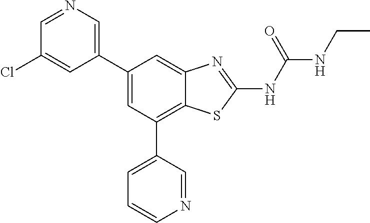 [SMILES: CCNC(=O)Nc1nc2cc(-c3cncc(Cl)c3)cc(-c3cccnc3)c2s1] |
| 49 | 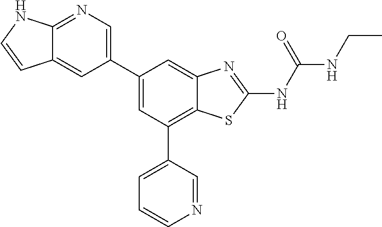 [SMILES: CCNC(=O)Nc1nc2cc(-c3cnc4[nH]ccc4c3)cc(-c3cccnc3)c2s1] |
| 50 | 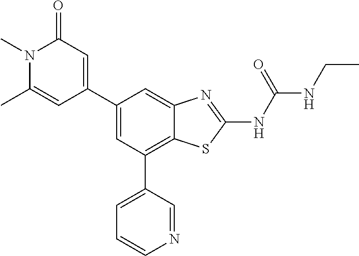 [SMILES: CCNC(=O)Nc1nc2cc(-c3cc(C)n(C)c(=O)c3)cc(-c3cccnc3)c2s1] |
| 51 | 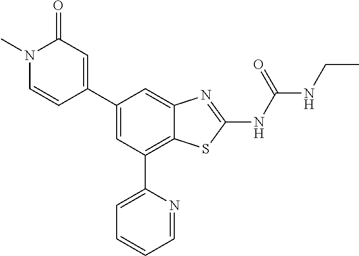 [SMILES: CCNC(=O)Nc1nc2cc(-c3ccn(C)c(=O)c3)cc(-c3ccccn3)c2s1] |
| 52 | 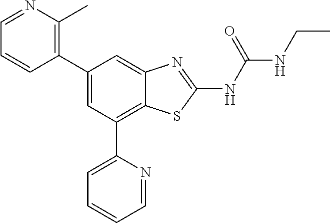 [SMILES: CCNC(=O)Nc1nc2cc(-c3cccnc3C)cc(-c3ccccn3)c2s1] |
| 53 | 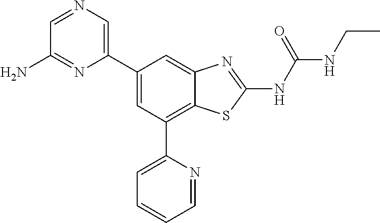 [SMILES: CCNC(=O)Nc1nc2cc(-c3cncc(N)n3)cc(-c3ccccn3)c2s1] |
| 54 | 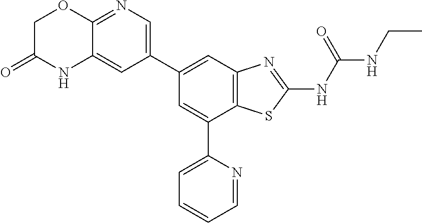 [SMILES: CCNC(=O)Nc1nc2cc(-c3cnc4c(c3)NC(=O)CO4)cc(-c3ccccn3)c2s1] |
| 55 | 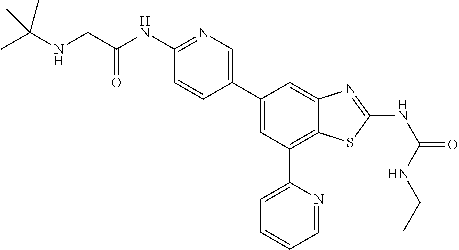 [SMILES: CCNC(=O)Nc1nc2cc(-c3ccc(NC(=O)CNC(C)(C)C)nc3)cc(-c3ccccn3)c2s1] |
| 56 | 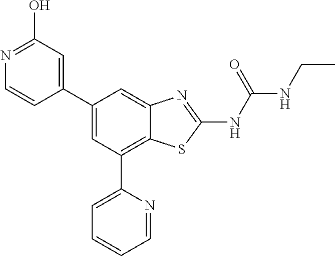 [SMILES: CCNC(=O)Nc1nc2cc(-c3ccnc(O)c3)cc(-c3ccccn3)c2s1] |
| 57 | 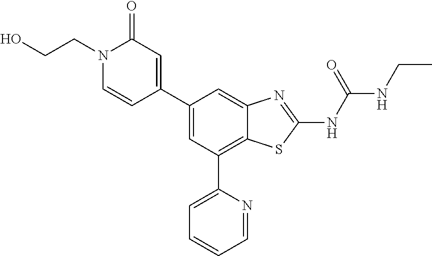 [SMILES: CCNC(=O)Nc1nc2cc(-c3ccn(CCO)c(=O)c3)cc(-c3ccccn3)c2s1] |
| 58 | 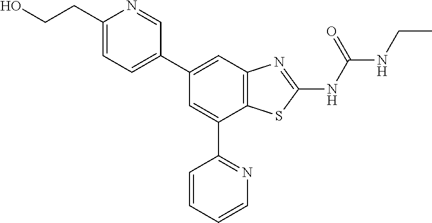 [SMILES: CCNC(=O)Nc1nc2cc(-c3ccc(CCO)nc3)cc(-c3ccccn3)c2s1] |
| 59 | 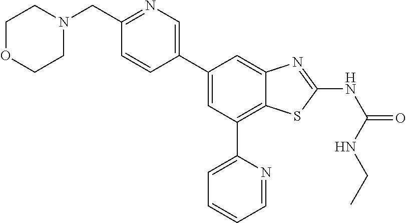 [SMILES: CCNC(=O)Nc1nc2cc(-c3ccc(CN4CCOCC4)nc3)cc(-c3ccccn3)c2s1] |
| 60 | 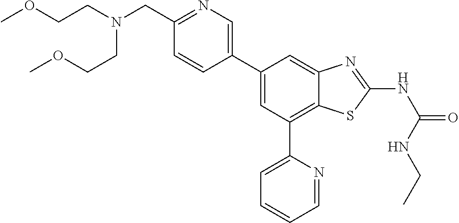 [SMILES: CCNC(=O)Nc1nc2cc(-c3ccc(CN(CCOC)CCOC)nc3)cc(-c3ccccn3)c2s1] |
| 61 | 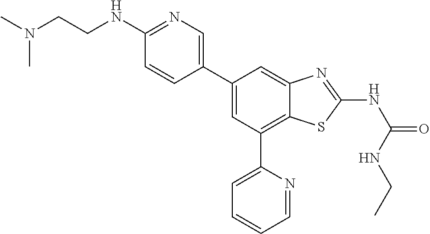 [SMILES: CCNC(=O)Nc1nc2cc(-c3ccc(NCCN(C)C)nc3)cc(-c3ccccn3)c2s1] |
| 62 | 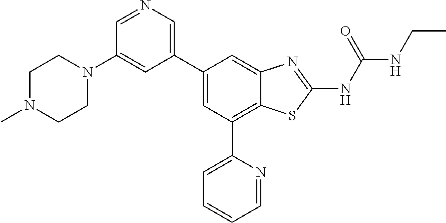 [SMILES: CCNC(=O)Nc1nc2cc(-c3cncc(N4CCN(C)CC4)c3)cc(-c3ccccn3)c2s1] |
| 63 | 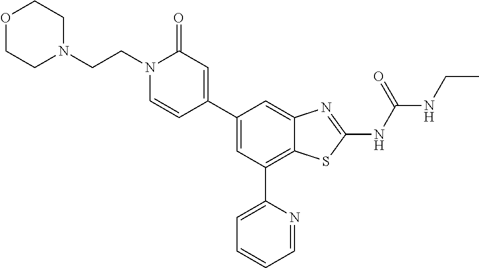 [SMILES: CCNC(=O)Nc1nc2cc(-c3ccn(CCN4CCOCC4)c(=O)c3)cc(-c3ccccn3)c2s1] |
| 64 | 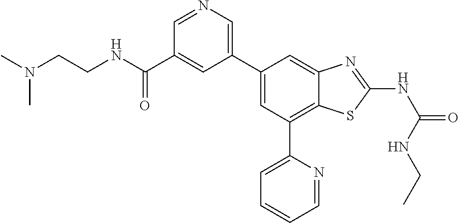 [SMILES: CCNC(=O)Nc1nc2cc(-c3cncc(C(=O)NCCN(C)C)c3)cc(-c3ccccn3)c2s1] |
| 65 | 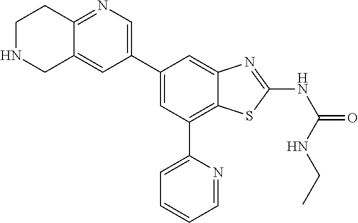 [SMILES: CCNC(=O)Nc1nc2cc(-c3cnc4c(c3)CNCC4)cc(-c3ccccn3)c2s1] |
| 66 | 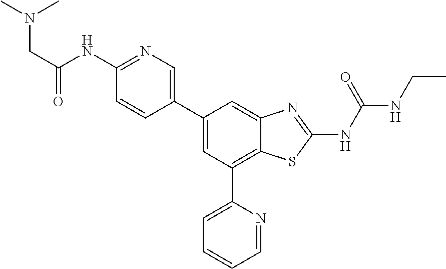 [SMILES: CCNC(=O)Nc1nc2cc(-c3ccc(NC(=O)CN(C)C)nc3)cc(-c3ccccn3)c2s1] |
| 67 | 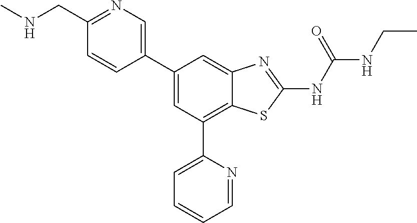 [SMILES: CCNC(=O)Nc1nc2cc(-c3ccc(CNC)nc3)cc(-c3ccccn3)c2s1] |
| 68 | 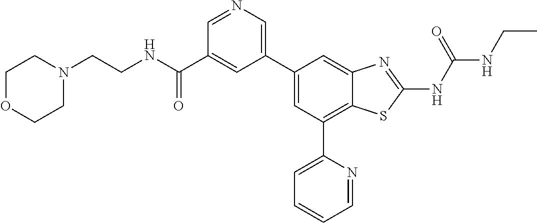 [SMILES: CCNC(=O)Nc1nc2cc(-c3cncc(C(=O)NCCN4CCOCC4)c3)cc(-c3ccccn3)c2s1] |
| 69 | 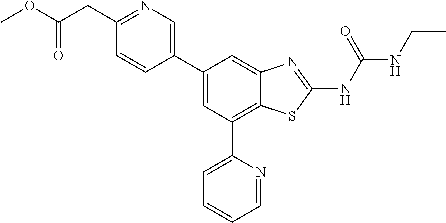 [SMILES: CCNC(=O)Nc1nc2cc(-c3ccc(CC(=O)OC)nc3)cc(-c3ccccn3)c2s1] |
| 70 | 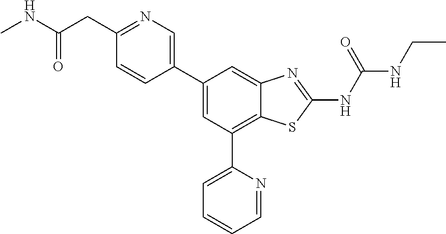 [SMILES: CCNC(=O)Nc1nc2cc(-c3ccc(CC(=O)NC)nc3)cc(-c3ccccn3)c2s1] |
| 71 | 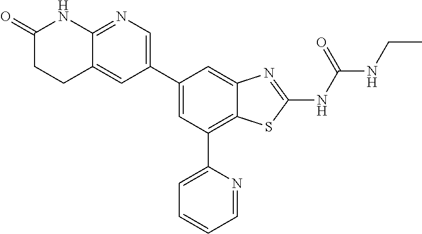 [SMILES: CCNC(=O)Nc1nc2cc(-c3cnc4c(c3)CCC(=O)N4)cc(-c3ccccn3)c2s1] |
| 72 | 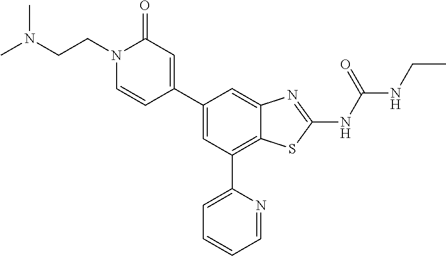 [SMILES: CCNC(=O)Nc1nc2cc(-c3ccn(CCN(C)C)c(=O)c3)cc(-c3ccccn3)c2s1] |
| 73 | 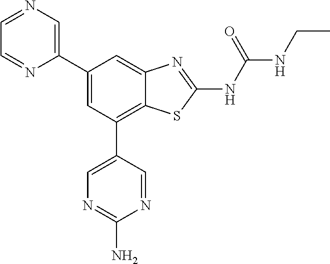 [SMILES: CCNC(=O)Nc1nc2cc(-c3cnccn3)cc(-c3cnc(N)nc3)c2s1] |
| 74 | 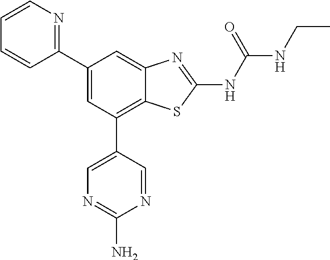 [SMILES: CCNC(=O)Nc1nc2cc(-c3ccccn3)cc(-c3cnc(N)nc3)c2s1] |
| 75 | 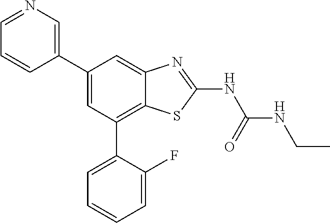 [SMILES: CCNC(=O)Nc1nc2cc(-c3cccnc3)cc(-c3ccccc3F)c2s1] |
| 76 | 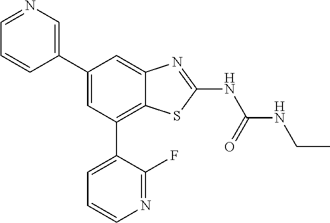 [SMILES: CCNC(=O)Nc1nc2cc(-c3cccnc3)cc(-c3cccnc3F)c2s1] |
| 77 | 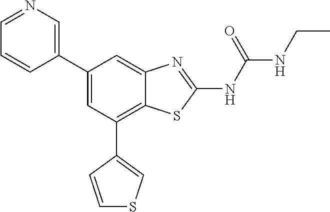 [SMILES: CCNC(=O)Nc1nc2cc(-c3cccnc3)cc(-c3ccsc3)c2s1] |
| 78 | 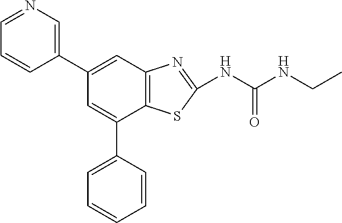 [SMILES: CCNC(=O)Nc1nc2cc(-c3cccnc3)cc(-c3ccccc3)c2s1] |
| 79 | 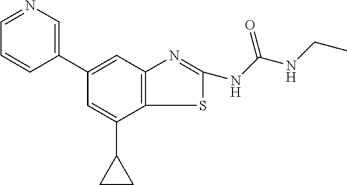 [SMILES: CCNC(=O)Nc1nc2cc(-c3cccnc3)cc(C3CC3)c2s1] |
| 80 | 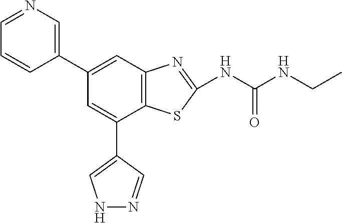 [SMILES: CCNC(=O)Nc1nc2cc(-c3cccnc3)cc(-c3cn[nH]c3)c2s1] |
| 81 | 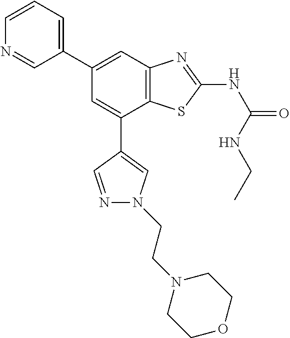 [SMILES: CCNC(=O)Nc1nc2cc(-c3cccnc3)cc(-c3cnn(CCN4CCOCC4)c3)c2s1] |
| 82 | 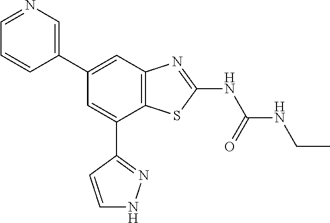 [SMILES: CCNC(=O)Nc1nc2cc(-c3cccnc3)cc(-c3cc[nH]n3)c2s1] |
| 83 | 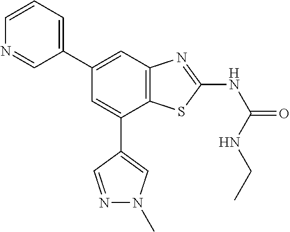 [SMILES: CCNC(=O)Nc1nc2cc(-c3cccnc3)cc(-c3cnn(C)c3)c2s1] |
| 84 | 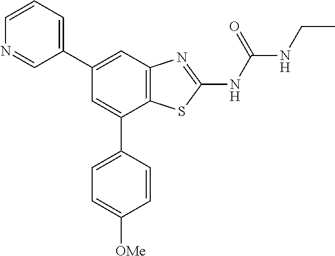 [SMILES: CCNC(=O)Nc1nc2cc(-c3cccnc3)cc(-c3ccc(OC)cc3)c2s1] |
| 85 | 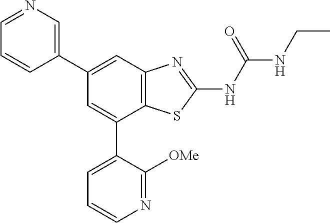 [SMILES: CCNC(=O)Nc1nc2cc(-c3cccnc3)cc(-c3cccnc3OC)c2s1] |
| 86 | 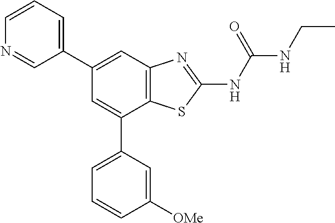 [SMILES: CCNC(=O)Nc1nc2cc(-c3cccnc3)cc(-c3cccc(OC)c3)c2s1] |
| 87 | 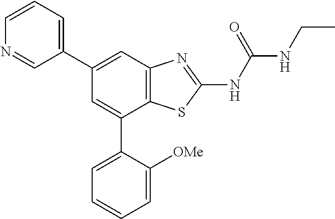 [SMILES: CCNC(=O)Nc1nc2cc(-c3cccnc3)cc(-c3ccccc3OC)c2s1] |
| 88 | 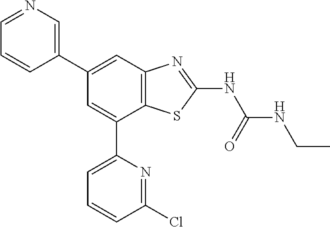 [SMILES: CCNC(=O)Nc1nc2cc(-c3cccnc3)cc(-c3cccc(Cl)n3)c2s1] |
| 89 | 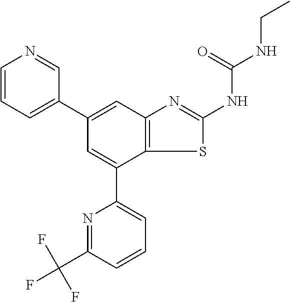 [SMILES: CCNC(=O)Nc1nc2cc(-c3cccnc3)cc(-c3cccc(C(F)(F)F)n3)c2s1] |
| 90 | 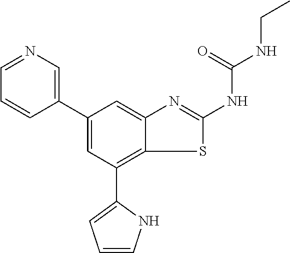 [SMILES: CCNC(=O)Nc1nc2cc(-c3cccnc3)cc(-c3ccc[nH]3)c2s1] |
| 91 | 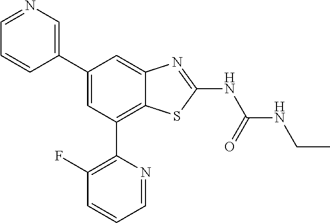 [SMILES: CCNC(=O)Nc1nc2cc(-c3cccnc3)cc(-c3ncccc3F)c2s1] |
| 92 | 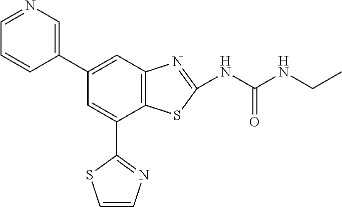 [SMILES: CCNC(=O)Nc1nc2cc(-c3cccnc3)cc(-c3nccs3)c2s1] |
| 93 | 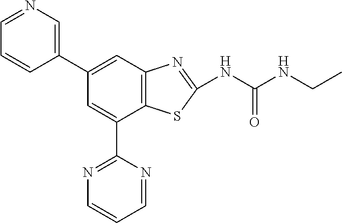 [SMILES: CCNC(=O)Nc1nc2cc(-c3cccnc3)cc(-c3ncccn3)c2s1] |
| 94 | 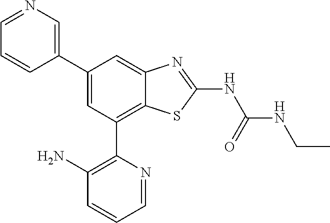 [SMILES: CCNC(=O)Nc1nc2cc(-c3cccnc3)cc(-c3ncccc3N)c2s1] |
| 95 | 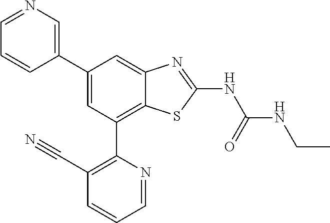 [SMILES: CCNC(=O)Nc1nc2cc(-c3cccnc3)cc(-c3ncccc3C#N)c2s1] |
| 96 | 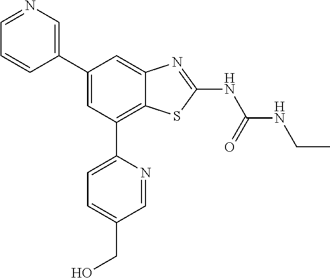 [SMILES: CCNC(=O)Nc1nc2cc(-c3cccnc3)cc(-c3ccc(CO)cn3)c2s1] |
| 97 | 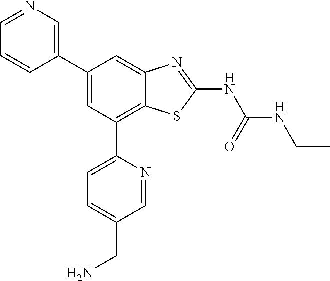 [SMILES: CCNC(=O)Nc1nc2cc(-c3cccnc3)cc(-c3ccc(CN)cn3)c2s1] |
| 98 | 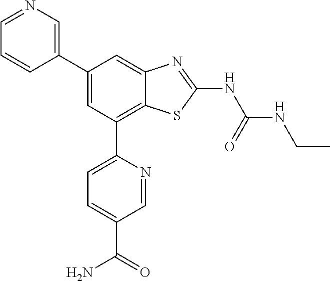 [SMILES: CCNC(=O)Nc1nc2cc(-c3cccnc3)cc(-c3ccc(C(N)=O)cn3)c2s1] |
| 99 | 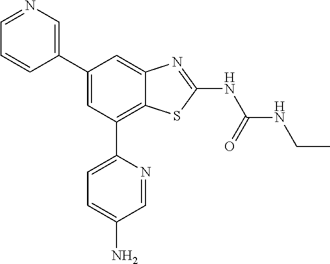 [SMILES: CCNC(=O)Nc1nc2cc(-c3cccnc3)cc(-c3ccc(N)cn3)c2s1] |
| 100 | 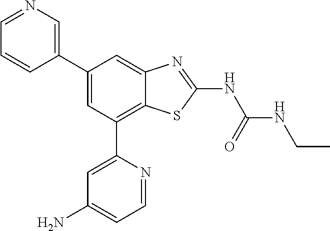 [SMILES: CCNC(=O)Nc1nc2cc(-c3cccnc3)cc(-c3cc(N)ccn3)c2s1] |
| 101 | 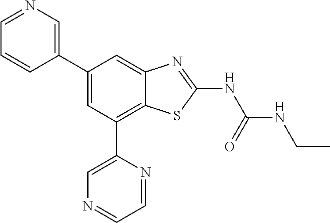 [SMILES: CCNC(=O)Nc1nc2cc(-c3cccnc3)cc(-c3cnccn3)c2s1] |
| 102 | 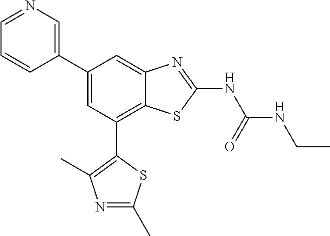 [SMILES: CCNC(=O)Nc1nc2cc(-c3cccnc3)cc(-c3sc(C)nc3C)c2s1] |
| 103 | 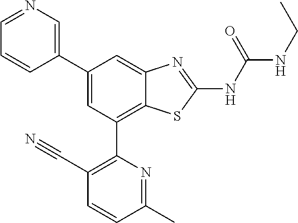 [SMILES: CCNC(=O)Nc1nc2cc(-c3cccnc3)cc(-c3nc(C)ccc3C#N)c2s1] |
| 104 | 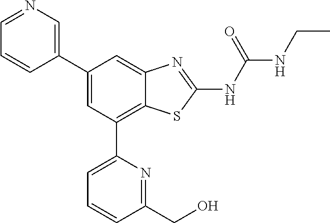 [SMILES: CCNC(=O)Nc1nc2cc(-c3cccnc3)cc(-c3cccc(CO)n3)c2s1] |
| 105 | 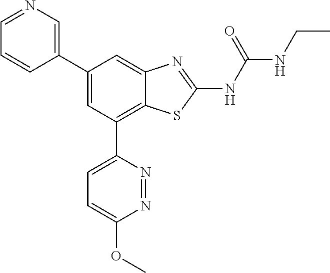 [SMILES: CCNC(=O)Nc1nc2cc(-c3cccnc3)cc(-c3ccc(OC)nn3)c2s1] |
| 106 | 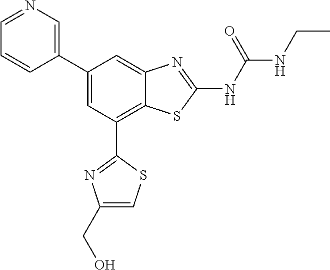 [SMILES: CCNC(=O)Nc1nc2cc(-c3cccnc3)cc(-c3nc(CO)cs3)c2s1] |
| 107 | 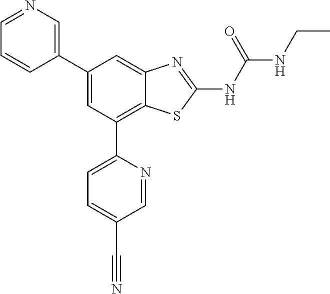 [SMILES: CCNC(=O)Nc1nc2cc(-c3cccnc3)cc(-c3ccc(C#N)cn3)c2s1] |
| 108 | 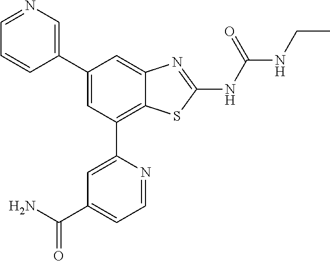 [SMILES: CCNC(=O)Nc1nc2cc(-c3cccnc3)cc(-c3cc(C(N)=O)ccn3)c2s1] |
| 109 | 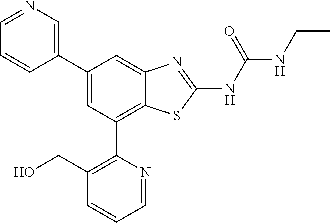 [SMILES: CCNC(=O)Nc1nc2cc(-c3cccnc3)cc(-c3ncccc3CO)c2s1] |
| 110 | 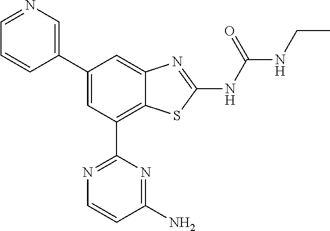 [SMILES: CCNC(=O)Nc1nc2cc(-c3cccnc3)cc(-c3nccc(N)n3)c2s1] |
| 111 | 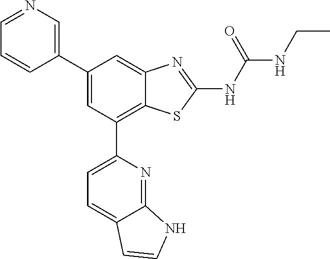 [SMILES: CCNC(=O)Nc1nc2cc(-c3cccnc3)cc(-c3ccc4cc[nH]c4n3)c2s1] |
| 112 | 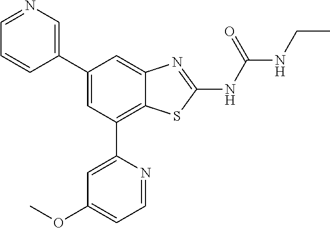 [SMILES: CCNC(=O)Nc1nc2cc(-c3cccnc3)cc(-c3cc(OC)ccn3)c2s1] |
| 113 | 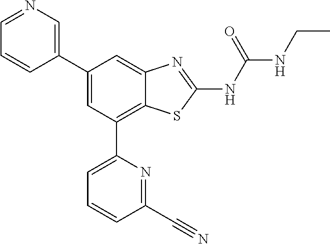 [SMILES: CCNC(=O)Nc1nc2cc(-c3cccnc3)cc(-c3cccc(C#N)n3)c2s1] |
| 114 | 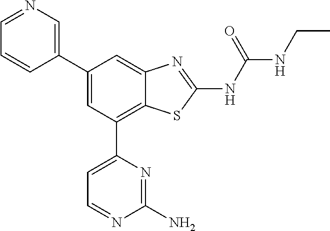 [SMILES: CCNC(=O)Nc1nc2cc(-c3cccnc3)cc(-c3ccnc(N)n3)c2s1] |
| 115 | 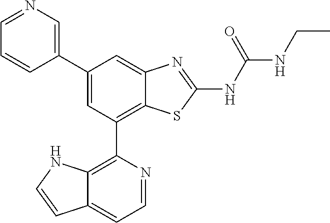 [SMILES: CCNC(=O)Nc1nc2cc(-c3cccnc3)cc(-c3nccc4cc[nH]c34)c2s1] |
| 116 | 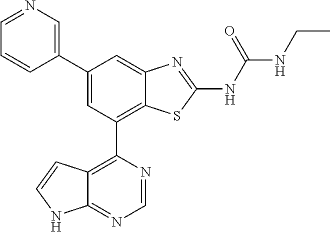 [SMILES: CCNC(=O)Nc1nc2cc(-c3cccnc3)cc(-c3ncnc4[nH]ccc34)c2s1] |
| 117 | 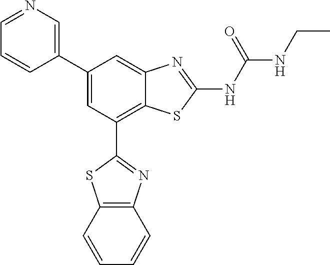 [SMILES: CCNC(=O)Nc1nc2cc(-c3cccnc3)cc(-c3nc4ccccc4s3)c2s1] |
| 118 | 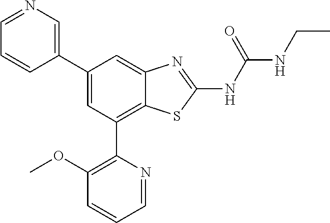 [SMILES: CCNC(=O)Nc1nc2cc(-c3cccnc3)cc(-c3ncccc3OC)c2s1] |
| 119 | 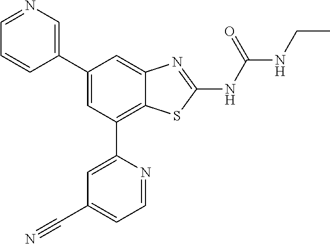 [SMILES: CCNC(=O)Nc1nc2cc(-c3cccnc3)cc(-c3cc(C#N)ccn3)c2s1] |
| 120 | 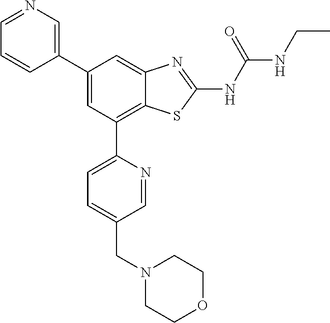 [SMILES: CCNC(=O)Nc1nc2cc(-c3cccnc3)cc(-c3ccc(CN4CCOCC4)cn3)c2s1] |
| 121 | 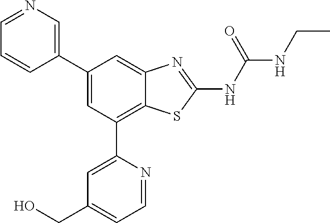 [SMILES: CCNC(=O)Nc1nc2cc(-c3cccnc3)cc(-c3cc(CO)ccn3)c2s1] |
| 122 | 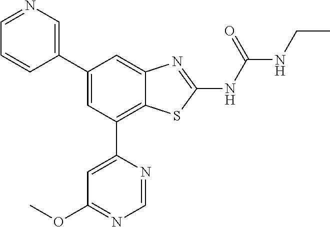 [SMILES: CCNC(=O)Nc1nc2cc(-c3cccnc3)cc(-c3cc(OC)ncn3)c2s1] |
| 123 | 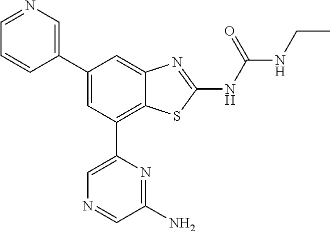 [SMILES: CCNC(=O)Nc1nc2cc(-c3cccnc3)cc(-c3cncc(N)n3)c2s1] |
| 124 | 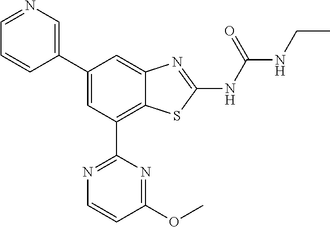 [SMILES: CCNC(=O)Nc1nc2cc(-c3cccnc3)cc(-c3nccc(OC)n3)c2s1] |
| 125 | 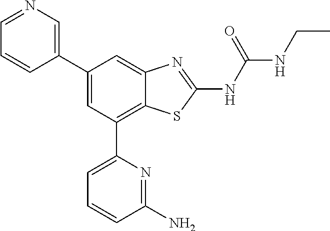 [SMILES: CCNC(=O)Nc1nc2cc(-c3cccnc3)cc(-c3cccc(N)n3)c2s1] |
| 126 | 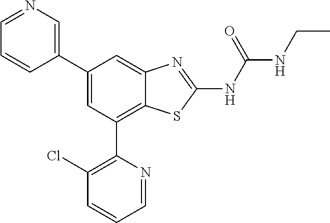 [SMILES: CCNC(=O)Nc1nc2cc(-c3cccnc3)cc(-c3ncccc3Cl)c2s1] |
| 127 | 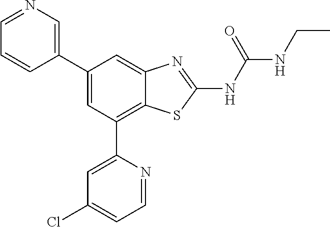 [SMILES: CCNC(=O)Nc1nc2cc(-c3cccnc3)cc(-c3cc(Cl)ccn3)c2s1] |
| 128 | 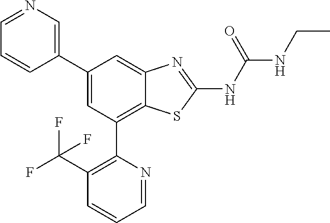 [SMILES: CCNC(=O)Nc1nc2cc(-c3cccnc3)cc(-c3ncccc3C(F)(F)F)c2s1] |
| 129 | 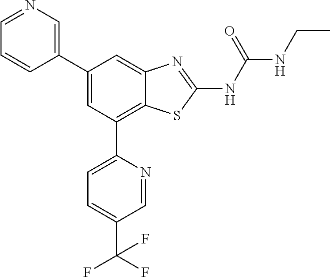 [SMILES: CCNC(=O)Nc1nc2cc(-c3cccnc3)cc(-c3ccc(C(F)(F)F)cn3)c2s1] |
| 130 | 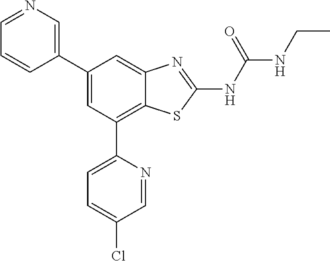 [SMILES: CCNC(=O)Nc1nc2cc(-c3cccnc3)cc(-c3ccc(Cl)cn3)c2s1] |
| 131 | 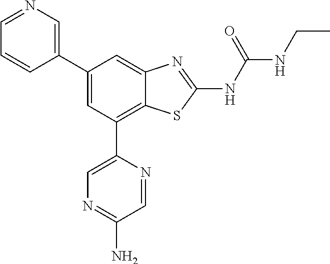 [SMILES: CCNC(=O)Nc1nc2cc(-c3cccnc3)cc(-c3cnc(N)cn3)c2s1] |
| 132 | 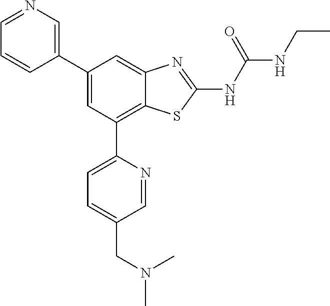 [SMILES: CCNC(=O)Nc1nc2cc(-c3cccnc3)cc(-c3ccc(CN(C)C)cn3)c2s1] |
| 133 | 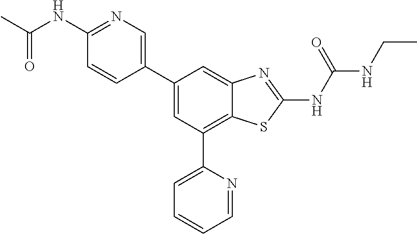 [SMILES: CCNC(=O)Nc1nc2cc(-c3ccc(NC(C)=O)nc3)cc(-c3ccccn3)c2s1] |
| 134 | 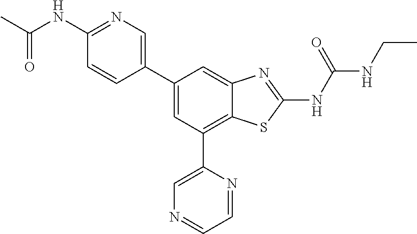 [SMILES: CCNC(=O)Nc1nc2cc(-c3ccc(NC(C)=O)nc3)cc(-c3cnccn3)c2s1] |
| 135 | 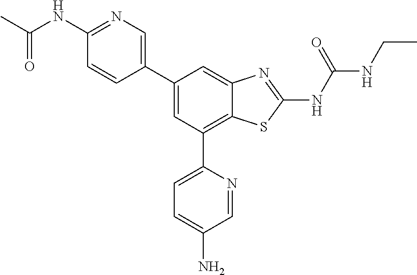 [SMILES: CCNC(=O)Nc1nc2cc(-c3ccc(NC(C)=O)nc3)cc(-c3ccc(N)cn3)c2s1] |
| 136 | 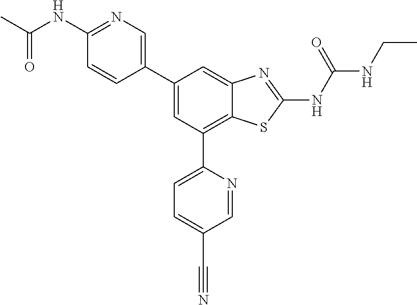 [SMILES: CCNC(=O)Nc1nc2cc(-c3ccc(NC(C)=O)nc3)cc(-c3ccc(C#N)cn3)c2s1] |
| 137 | 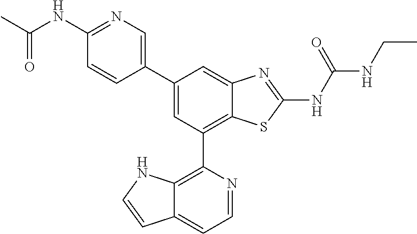 [SMILES: CCNC(=O)Nc1nc2cc(-c3ccc(NC(C)=O)nc3)cc(-c3nccc4cc[nH]c34)c2s1] |
| 138 | 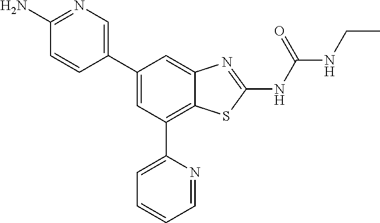 [SMILES: CCNC(=O)Nc1nc2cc(-c3ccc(N)nc3)cc(-c3ccccn3)c2s1] |
| 139 | 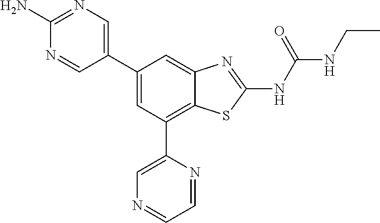 [SMILES: CCNC(=O)Nc1nc2cc(-c3cnc(N)nc3)cc(-c3cnccn3)c2s1] |
| 140 | 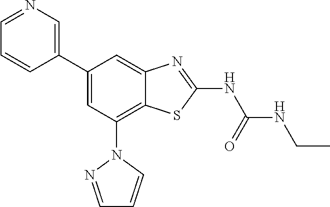 [SMILES: CCNC(=O)Nc1nc2cc(-c3cccnc3)cc(-n3cccn3)c2s1] |
| 141 | 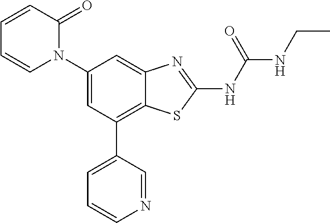 [SMILES: CCNC(=O)Nc1nc2cc(-n3ccccc3=O)cc(-c3cccnc3)c2s1] |
| 142 | 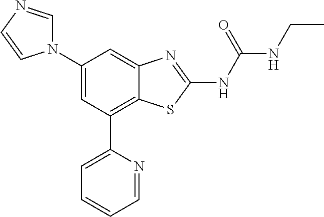 [SMILES: CCNC(=O)Nc1nc2cc(-n3ccnc3)cc(-c3ccccn3)c2s1] |
| 143 | 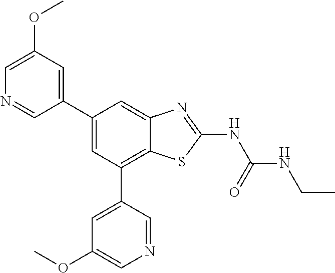 [SMILES: CCNC(=O)Nc1nc2cc(-c3cncc(OC)c3)cc(-c3cncc(OC)c3)c2s1] |
| 144 | 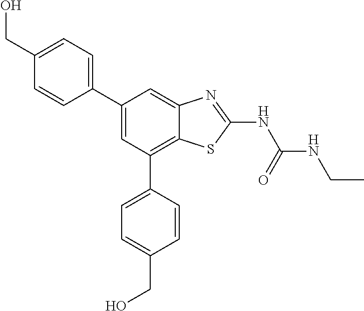 [SMILES: CCNC(=O)Nc1nc2cc(-c3ccc(CO)cc3)cc(-c3ccc(CO)cc3)c2s1] |
| 145 | 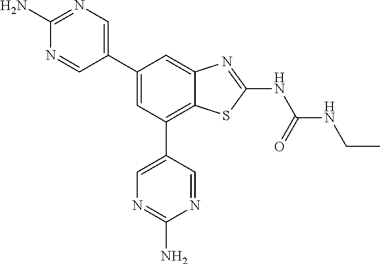 [SMILES: CCNC(=O)Nc1nc2cc(-c3cnc(N)nc3)cc(-c3cnc(N)nc3)c2s1] |
| 146 | 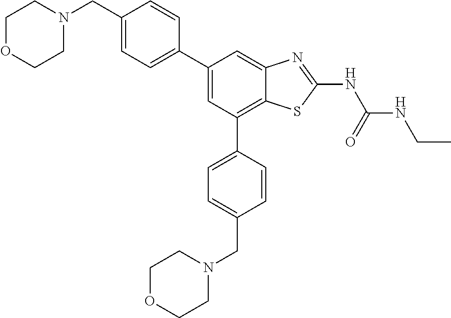 [SMILES: CCNC(=O)Nc1nc2cc(-c3ccc(CN4CCOCC4)cc3)cc(-c3ccc(CN4CCOCC4)cc3)c2s1] |
| 147 | 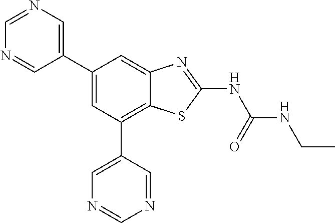 [SMILES: CCNC(=O)Nc1nc2cc(-c3cncnc3)cc(-c3cncnc3)c2s1] |
| 148 | 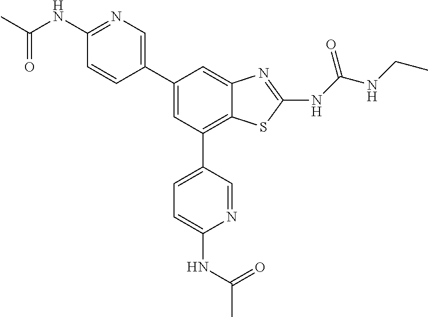 [SMILES: CCNC(=O)Nc1nc2cc(-c3ccc(NC(C)=O)nc3)cc(-c3ccc(NC(C)=O)nc3)c2s1] |
| 149 | 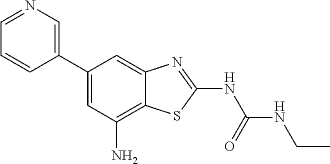 [SMILES: CCNC(=O)Nc1nc2cc(-c3cccnc3)cc(N)c2s1] |
| 150 | 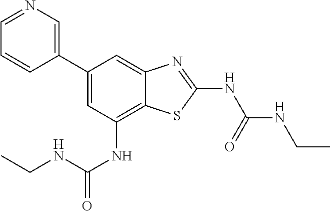 [SMILES: CCNC(=O)Nc1nc2cc(-c3cccnc3)cc(NC(=O)NCC)c2s1] |
| 151 | 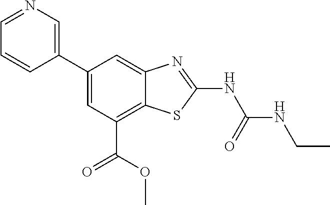 [SMILES: CCNC(=O)Nc1nc2cc(-c3cccnc3)cc(C(=O)OC)c2s1] |
| 152 | 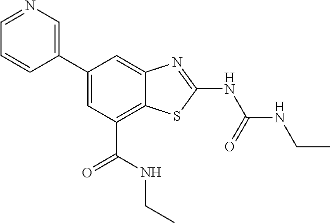 [SMILES: CCNC(=O)Nc1nc2cc(-c3cccnc3)cc(C(=O)NCC)c2s1] |
| 153 | 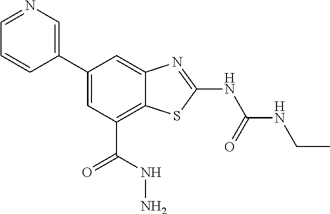 [SMILES: CCNC(=O)Nc1nc2cc(-c3cccnc3)cc(C(=O)NN)c2s1] |
| 154 | 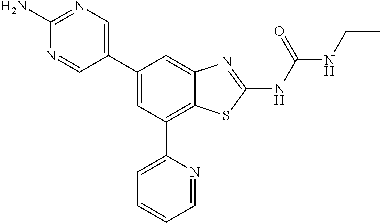 [SMILES: CCNC(=O)Nc1nc2cc(-c3cnc(N)nc3)cc(-c3ccccn3)c2s1] |
| 155 | 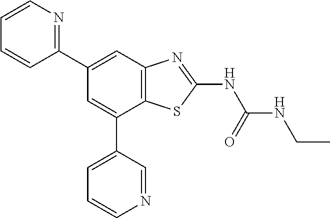 [SMILES: CCNC(=O)Nc1nc2cc(-c3ccccn3)cc(-c3cccnc3)c2s1] |
| 156 | 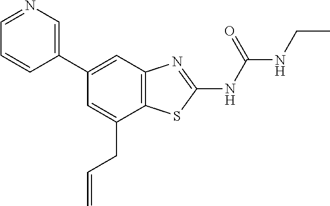 [SMILES: C=CCc1cc(-c2cccnc2)cc2nc(NC(=O)NCC)sc12] |
| 157 | 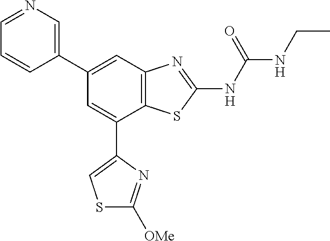 [SMILES: CCNC(=O)Nc1nc2cc(-c3cccnc3)cc(-c3csc(OC)n3)c2s1] |
| 158 | 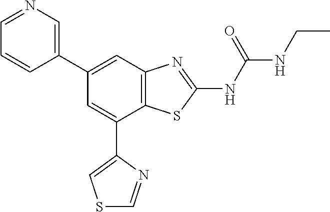 [SMILES: CCNC(=O)Nc1nc2cc(-c3cccnc3)cc(-c3cscn3)c2s1] |
| 159 | 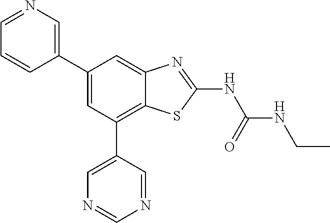 [SMILES: CCNC(=O)Nc1nc2cc(-c3cccnc3)cc(-c3cncnc3)c2s1] |
| 160 | 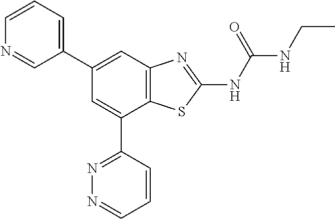 [SMILES: CCNC(=O)Nc1nc2cc(-c3cccnc3)cc(-c3cccnn3)c2s1] |
| 161 | 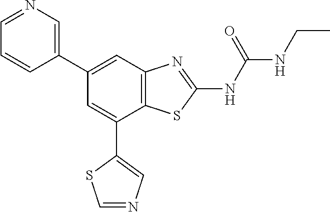 [SMILES: CCNC(=O)Nc1nc2cc(-c3cccnc3)cc(-c3cncs3)c2s1] |
| 162 | 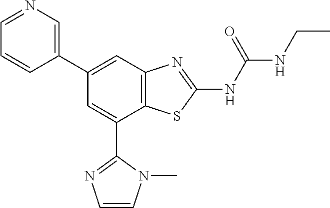 [SMILES: CCNC(=O)Nc1nc2cc(-c3cccnc3)cc(-c3nccn3C)c2s1] |
| 163 | 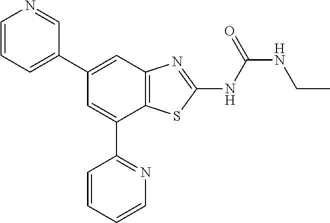 [SMILES: CCNC(=O)Nc1nc2cc(-c3cccnc3)cc(-c3ccccn3)c2s1] |
| 164 | 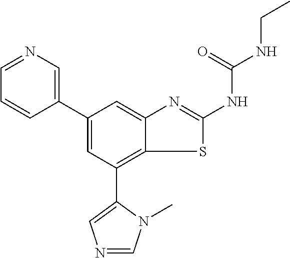 [SMILES: CCNC(=O)Nc1nc2cc(-c3cccnc3)cc(-c3cncn3C)c2s1] |
| 165 | 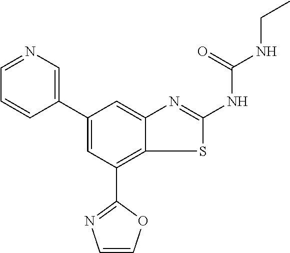 [SMILES: CCNC(=O)Nc1nc2cc(-c3cccnc3)cc(-c3ncco3)c2s1] |
| 166 | 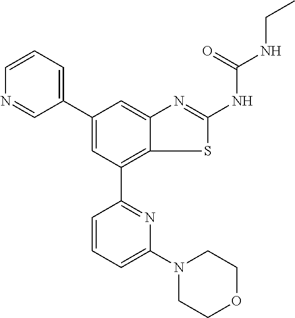 [SMILES: CCNC(=O)Nc1nc2cc(-c3cccnc3)cc(-c3cccc(N4CCOCC4)n3)c2s1] |
| 167 | 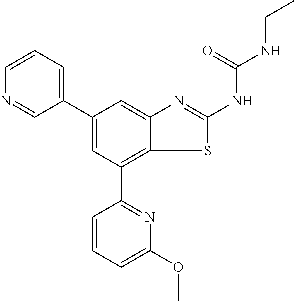 [SMILES: CCNC(=O)Nc1nc2cc(-c3cccnc3)cc(-c3cccc(OC)n3)c2s1] |
| 168 | 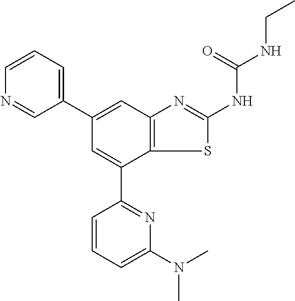 [SMILES: CCNC(=O)Nc1nc2cc(-c3cccnc3)cc(-c3cccc(N(C)C)n3)c2s1] |
| 169 | 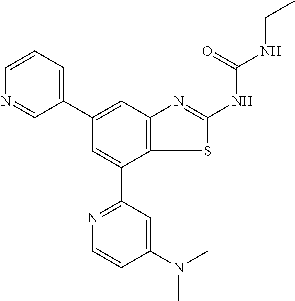 [SMILES: CCNC(=O)Nc1nc2cc(-c3cccnc3)cc(-c3cc(N(C)C)ccn3)c2s1] |
| 170 | 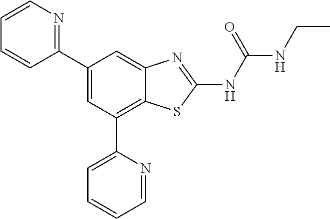 [SMILES: CCNC(=O)Nc1nc2cc(-c3ccccn3)cc(-c3ccccn3)c2s1] |
| 171 | 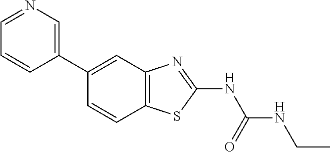 [SMILES: CCNC(=O)Nc1nc2cc(-c3cccnc3)ccc2s1] |
| 172 | 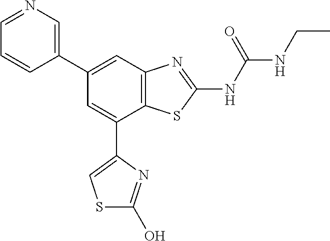 [SMILES: CCNC(=O)Nc1nc2cc(-c3cccnc3)cc(-c3csc(O)n3)c2s1] |
| 173 | 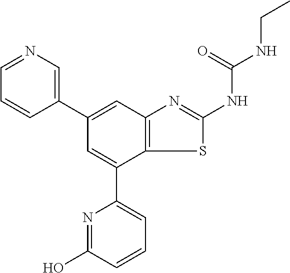 [SMILES: CCNC(=O)Nc1nc2cc(-c3cccnc3)cc(-c3cccc(O)n3)c2s1] |
| 174 | 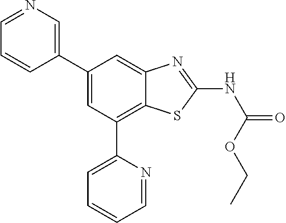 [SMILES: CCOC(=O)Nc1nc2cc(-c3cccnc3)cc(-c3ccccn3)c2s1] |
| 175 | 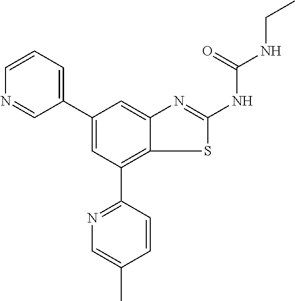 [SMILES: CCNC(=O)Nc1nc2cc(-c3cccnc3)cc(-c3ccc(C)cn3)c2s1] |
| 176 | 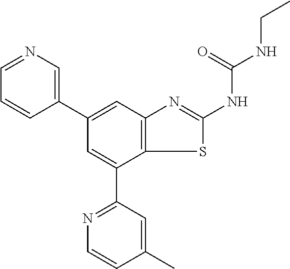 [SMILES: CCNC(=O)Nc1nc2cc(-c3cccnc3)cc(-c3cc(C)ccn3)c2s1] |
| 177 | 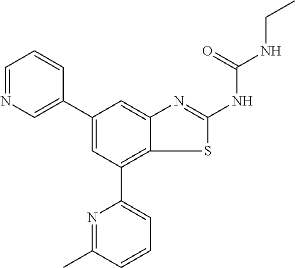 [SMILES: CCNC(=O)Nc1nc2cc(-c3cccnc3)cc(-c3cccc(C)n3)c2s1] |
| 178 | 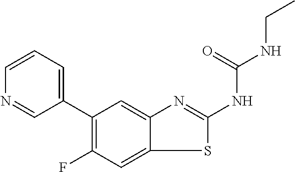 [SMILES: CCNC(=O)Nc1nc2cc(-c3cccnc3)c(F)cc2s1] |
| 179 | 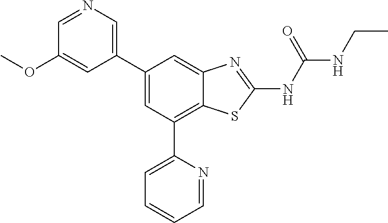 [SMILES: CCNC(=O)Nc1nc2cc(-c3cncc(OC)c3)cc(-c3ccccn3)c2s1] |

and; salts, racemates, diastereomers, enantiomers, deuterated forms, hydrates, solvates and prodrugs thereof.

Suitable compounds according to this embodiment where X₂ may be —N═ includes but is not limited to, any one of compound Examples 1 and 2 as previously disclosed in WO2009/074810:
1) 1-(4,6-di-pyridin-3-yl-thiazolo[5,4-c]pyridine-2-yl)-3-ethylurea; and
2) 1-(4,6-di(pyrazin-2-yl)-thiazolo[5,4-c]pyridine-2-yl)-3-ethylurea; and
   and; salts, racemates, diastereomers, enantiomers, deuterated forms, hydrates, solvates and prodrugs thereof.

In one embodiment of Formula (I):

Alk may be unsubstituted C₁₋₆alkyl. For example, Alk may be ethyl.

Ring A may be an optionally substituted 5-6-membered hetero-monocyclic ring or an 8-10-membered fused hetero-bicyclic ring. For example, Ring A may be an optionally substituted 5-6-membered heteroaryl ring. In one example, Ring A may be an optionally substituted 6-membered heteroaryl ring.

X₁ may be an CH, —N═ or C—R₁ where R₁ may be selected from H, OH, optionally substituted C₁₋₃alkyl, optionally substituted C₂₋₃alkenyl, optionally substituted C₂₋₃alkynyl, optionally substituted C₁₋₃alkoxyl, halo, haloC₁₋₃alkyl, NH₂, optionally substituted NHC₁₋₃alkyl, optionally substituted N(C₁₋₃alkyl)₂, optionally substituted SC₁₋₃alkyl and CN. For example, R₁ may be a halo or C₁₋₃alkyl. In one example, X₁ may be CH.

X₂ may be CH, —N═ or C—R₂. For example, X₂ may be CH or C—R₂ where R₂ may be selected from halo, OH, optionally substituted C₁₋₆alkyl, optionally substituted OC₁₋₆alkyl and optionally substituted C₁₋₆alkoxyl.

X₃ may be CH, —N═ or C—R₃ where R₃ may be halo, or an optionally substituted 5-membered or 6-membered heteroaryl ring. For example, an optionally substituted 6-membered heteroaryl ring. For example, X₃ may be CH or —N═.

Z₁ may be selected from H, halo, C₁₋₆alkyl, a 5-membered heterocyclic ring including 5-membered heteroaryl rings, a 6-membered heterocyclic ring including 6-membered heteroaryl rings, OH, OC₁₋₆alkyl, C₁₋₆alkoxyl, cyano (CN), a carbonyl moiety (═O), C(═O)OC₁₋₆alkyl, NH₂, NH—C₁₋₆alkyl, N(C₁₋₆alkyl)₂, and C(═O)NH—C₁₋₆alkyl; and wherein Z₁ may be further optionally substituted.

In one embodiment, optional substituents for Ring A, R₂, R₃ and/or Z₁ include but are not limited to one or more substituents independently selected from halo, C₁₋₆alkyl, a 5-membered heterocyclic ring including 5-membered heteroaryl rings, a 6-membered heterocyclic ring including 6-membered heteroaryl rings, OH, OC₁₋₆alkyl, C₁₋₆alkoxyl, cyano (CN), a carbonyl moiety (═O), C(═O)OC₁₋₆alkyl, NH₂, NH—C₁₋₆alkyl, N(C₁₋₆alkyl)₂, and C(═O)NH—C₁₋₆alkyl.

In one embodiment X₂ may be CH and X₃ may be CH.

In one embodiment X₂ may be CH and X₃ may be N.

In one embodiment X₂ may be N and X₃ may be CH.

In one embodiment X₂ may be N and X₃ may be N.

In another embodiment X₂ may be C—R₂ and X₃ may be CH.

In another embodiment X₂ may be C—R₂ and X₃ may be N.

In yet another embodiment X₂ may be C—R₂ and X₃ may be C—R₃.

Suitable examples of compounds according to this embodiment includes but is not limited to, any one of compound examples 1 to 79 as previously disclosed in WO2009/074812:
1) 1-ethyl-3-(5-pyridin-3-yl-benzothiazol-2-yl)-urea;
2) 2-{5-[2-(3-ethyl-ureido)-benzothiazol-5-yl]-pyridin-2-yl}-N-methyl-acetamide;
3) 1-ethyl-3-[5-(1-methyl-2-oxo-1,2-dihydro-pyridin-4-yl)-benzothiazol-2-yl]-urea;
4) 1-ethyl-3-(6-fluoro-5-pyridin-3-yl-benzothiazol-2-yl)-urea;
5) 1-(5-([1,2,4]triazolo[4,3-a]pyridin-6-yl)benzo[d]thiazol-2-yl)-3-ethylurea;
6) 1-ethyl-3-(5-(imidazo[1,2-a]pyridin-6-yl)benzo[d]thiazol-2-yl)urea;
7) 1-ethyl-3-(5-(tetrazolo[1,5-a]pyridin-6-yl)benzo[d]thiazol-2-yl)urea;
8) 1-(5-(3,4-dihydro-2H-pyrido[3,2-b][1,4]oxazin-7-yl)benzo[d]thiazol-2-yl)-3-ethylurea;
9) 1-ethyl-3-(5-(6-(hydroxymethyl)pyridin-3-yl)benzo[d]thiazol-2-yl)urea;
10) 1-ethyl-3-(5-(5-(2-oxopyridin-1(2H)-yl)pyrazin-2-yl)benzo[d]thiazol-2-yl)urea;
11) 1-ethyl-3-(5-(2-(hydroxymethyl)-1-methyl-1H-imidazol-5-yl)benzo[d]thiazol-2-yl)urea;
12) Ethyl 2-(5-(2-(3-ethylureido)benzo[d]thiazol-5-yl)-2-oxopyridin-1(2H)-yl)acetate;
13) 1-(5-(1-(2-ethoxyethyl)-6-oxo-1,6-dihydropyridin-3-yl)benzo[d]thiazol-2-yl)-3-ethylurea;
14) 1-ethyl-3-(5-(6-methyl-2-oxo-2H-pyran-4-yl)benzo[d]thiazol-2-yl)urea;
15) Methyl 5-(2-(3-ethylureido)benzo[d]thiazol-5-yl)picolinate;
16) 1-ethyl-3-(5-(1-(2-morpholinoethyl)-1H-pyrazol-4-yl)benzo[d]thiazol-2-yl)urea;
17) 1-(5-(1H-pyrazol-3-yl)benzo[d]thiazol-2-yl)-3-ethylurea;
18) 1-ethyl-3-(5-(1-methyl-1H-pyrazol-4-yl)benzo[d]thiazol-2-yl)urea;
19) 1-ethyl-3-(5-(4-methyl-3,4-dihydro-2H-pyrido[3,2-b][1,4]oxazin-7-yl)benzo[d]thiazol-2-yl)urea;
20) 1-ethyl-3-(5-(isoquinolin-4-yl)benzo[d]thiazol-2-yl)urea;
21) 1-(5-(1H-pyrazol-4-yl)benzo[d]thiazol-2-yl)-3-ethylurea;
22) 1-ethyl-3-(5-(2-methoxythiazol-5-yl)benzo[d]thiazol-2-yl)urea;
23) 1-ethyl-3-(5-(2-hydroxythiazol-5-yl)benzo[d]thiazol-2-yl)urea;
24) 1-ethyl-3-(5-(imidazo[1,2-a]pyridin-3-yl)benzo[d]thiazol-2-yl)urea;
25) 1-(5-([1,2,4]triazolo[1,5-a]pyridin-6-yl)benzo[d]thiazol-2-yl)-3-ethylurea;
26) N-(5-(2-(3-ethylureido)benzo[d]thiazol-5-yl)pyridin-2-yl)acetamide;
27) 1-ethyl-3-(5-(6-morpholinopyridin-3-yl)benzo[d]thiazol-2-yl)urea;
28) 1-(5-(2-(1H-imidazol-1-yl)pyrimidin-5-yl)benzo[d]thiazol-2-yl)-3-ethylurea;
29) 1-ethyl-3-(5-(2-oxo-1,2-dihydropyridin-4-yl)benzo[d]thiazol-2-yl)urea;
30) 1-ethyl-3-(5-(6-(2-methyl-2H-tetrazol-5-yl)pyridin-3-yl)benzo[d]thiazol-2-yl)urea;
31) 1-ethyl-3-(5-(1-(2-methoxyethyl)-6-methyl-2-oxo-1,2-dihydropyridin-4-yl)benzo[d]thiazol-2-yl)urea;
32) 1-ethyl-3-(5-(1-(2-hydroxyethyl)-6-methyl-2-oxo-1,2-dihydropyridin-4-yl)benzo[d]thiazol-2-yl)urea;
33) 1-ethyl-3-(5-(6-methyl-1-((6-methylpyridin-2-yl)methyl)-2-oxo-1,2-dihydropyridin-4-yl)benzo[d]thiazol-2-yl)urea;
34) 1-ethyl-3-(5-(6-methyl-2-oxo-1-(pyridin-3-ylmethyl)-1,2-dihydropyridin-4-yl)benzo[d]thiazol-2-yl)urea;
35) 1-ethyl-3-(5-(6-methyl-2-oxo-1-(pyridin-2-ylmethyl)-1,2-dihydropyridin-4-yl)benzo[d]thiazol-2-yl)urea;
36) 1-ethyl-3-(5-(6-methyl-2-oxo-1-(1-(pyridin-2-yl)ethyl)-1,2-dihydropyridin-4-yl)benzo[d]thiazol-2-yl)urea;
37) Ethyl 7-(2-(3-ethylureido)benzo[d]thiazol-5-yl)-1-methyl-4-oxo-1,4-dihydroquinoline-3-carboxylate;
38) N-(5-(2-(3-ethylureido)-6-fluorobenzo[d]thiazol-5-yl)pyridin-2-yl)acetamide;
39) Ethyl 2-(4-(2-(3-ethylureido)-6-fluorobenzo[d]thiazol-5-yl)-1H-pyrazol-1-yl)acetate;
40) 1-ethyl-3-(6-fluoro-5-(6-methyl-2-oxo-2H-pyran-4-yl)benzo[d]thiazol-2-yl)urea;
41) 1-ethyl-3-(6-fluoro-5-(2-(4-(2-hydroxyethyl)piperazin-1-yl)pyrimidin-5-yl)benzo[d]thiazol-2-yl)urea
42) 1-ethyl-3-(6-fluoro-5-(2-(piperazin-1-yl)pyrimidin-5-yl)benzo[d]thiazol-2-yl)urea hydrochloride;
43) Methyl 1-(5-(2-(3-ethylureido)-6-fluorobenzo[d]thiazol-5-yl)pyrimidin-2-yl)piperidine-4-carboxylate;
44) 1-(5-(2-(3-ethylureido)-6-fluorobenzo[d]thiazol-5-yl)pyrimidin-2-yl)piperidine-4-carboxylic acid;
45) Methyl 1-(5-(2-(3-ethylureido)-6-fluorobenzo[d]thiazol-5-yl)pyrimidin-2-yl)piperidine-3-carboxylate;
46) 1-(5-(2-(3-ethylureido)-6-fluorobenzo[d]thiazol-5-yl)pyrimidin-2-yl)piperidine-3-carboxylic acid;
47) Methyl 2-(4-(2-(3-ethylureido)-6-fluorobenzo[d]thiazol-5-yl)-1H-pyrazol-1-yl)propanoate;
48) 1-ethyl-3-(6-fluoro-5-(1H-pyrrolo[2,3-b]pyridin-3-yl)benzo[d]thiazol-2-yl)urea;
49) 1-ethyl-3-(6-fluoro-5-(6-methyl-2-oxo-1-(pyridin-3-ylmethyl)-1,2-dihydropyridin-4-yl)benzo[d]thiazol-2-yl)urea;
50) 1-ethyl-3-(6-fluoro-5-(6-methyl-1-((1-methylpyrrolidin-3-yl)methyl)-2-oxo-1,2-dihydropyridin-4-yl)benzo[d]thiazol-2-yl)urea;
51) 1-ethyl-3-(6-fluoro-5-(6-methyl-1-((1-methylpiperidin-2-yl)methyl)-2-oxo-1,2-dihydropyridin-4-yl)benzo[d]thiazol-2-yl)urea;
52) 1-ethyl-3-(6-fluoro-5-(6-methyl-1-((1-methyl-1H-imidazol-4-yl)methyl)-2-oxo-1,2-dihydropyridin-4-yl)benzo[d]thiazol-2-yl)urea;
53) Tert-butyl 2-((4-(2-(3-ethylureido)-6-fluorobenzo[d]thiazol-5-yl)-6-methyl-2-oxopyridin-1(2H)-yl)methyl)pyrrolidine-1-carboxylate;
54) 1-(5-(1-(3-(dimethylamino)propyl)-6-methyl-2-oxo-1,2-dihydropyridin-4-yl)-6-fluorobenzo[d]thiazol-2-yl)-3-ethylurea;
55) 1-ethyl-3-(6-fluoro-5-(6-(5-methyl-1,2,4-oxadiazol-3-yl)pyridin-3-yl)benzo[d]thiazol-2-yl)urea;
56) 1-ethyl-3-[6-fluoro-5-(4-methoxy-pyridin-2-yl)-benzothiazol-2-yl]-urea;
57) 1-ethyl-3-(6-fluoro-5-(2-(4-methylpiperazin-1-yl)pyrimidin-5-yl)benzo[d]thiazol-2-yl)urea;
58) 1-ethyl-3-(6-fluoro-5-(5-methoxypyridin-3-yl)benzo[d]thiazol-2-yl)urea;
59) 1-ethyl-3-(6-fluoro-5-(5-hydroxypyridin-3-yl)benzo[d]thiazol-2-yl)urea;
60) 5-(2-(3-ethylureido)-6-fluorobenzo[d]thiazol-5-yl)-N′-hydroxypicolinimidamide;
61) 1-ethyl-3-(6-fluoro-5-(1-methyl-2-oxo-1,2-dihydropyridin-4-yl)benzo[d]thiazol-2-yl)urea;
62) 1-ethyl-3-(6-fluoro-5-(1-(2-morpholinoethyl)-2-oxo-1,2-dihydropyridin-4-yl)benzo[d]thiazol-2-yl)urea;
63) 1-(5-(1-(2-(dimethylamino)ethyl)-2-oxo-1,2-dihydropyridin-4-yl)-6-fluorobenzo[d]thiazol-2-yl)-3-ethylurea;
64) Tert-butyl 3-((4-(2-(3-ethylureido)-6-fluorobenzo[d]thiazol-5-yl)-2,2′-dioxo-2H-1,4′-bipyridin-1′(2′H)-yl)methyl)piperidine-1-carboxylate;
65) 1-ethyl-3-(6-fluoro-5-(6-(hydroxymethyl)pyridin-3-yl)benzo[d]thiazol-2-yl)urea;
66) 1-ethyl-3-(6-fluoro-5-(6-(morpholinomethyl)pyridin-3-yl)benzo[d]thiazol-2-yl)urea;
67) 1-ethyl-3-(6-fluoro-5-(2-oxo-1-(pyrrolidin-3-yl)-1,2-dihydropyridin-4-yl)benzo[d]thiazol-2-yl)urea;
68) 2-{5-[2-(3-ethyl-ureido)-6-fluoro-benzothiazol-5-yl]-pyridin-2-yl}-N-methyl-acetamide;
69) 1-ethyl-3-(6-fluoro-5-(thiazol-5-yl)benzo[d]thiazol-2-yl)urea;
70) 1-ethyl-3-(6-fluoro-5-(2-(methylsulfonyl)pyrimidin-5-yl)benzo[d]thiazol-2-yl)urea;
71) 1-ethyl-3-(6-methoxy-5-(pyridin-3-yl)benzo[d]thiazol-2-yl)urea;
72) 1-ethyl-3-(6-methoxy-5-(1-methyl-2-oxo-1,2-dihydropyridin-4-yl)benzo[d]thiazol-2-yl)urea;
73) 1-ethyl-3-(6-methyl-5-(pyridin-3-yl)benzo[d]thiazol-2-yl)urea;
74) 1-ethyl-3-(6-methyl-5-(1-methyl-2-oxo-1,2-dihydropyridin-4-yl)benzo[d]thiazol-2-yl)urea;
75) 1-ethyl-3-(6-(pyridin-3-yl)thiazolo[5,4-b]pyridin-2-yl)urea;
76) N-(5-(2-(3-ethylureido)thiazolo[5,4-b]pyridin-6-yl)pyridin-2-yl) acetamide;
77) 1-ethyl-3-(6-(6-methyl-2-oxo-2H-pyran-4-yl) thiazolo[5,4-b]pyridin-2-yl)urea;
78) 1-ethyl-3-(6-fluoro-5-{6-methyl-1-[1-(6-methyl-pyridin-3-yl)-ethyl]-2-oxo-1,2-dihydro-pyridin-4-yl}-benzothiazol-2-yl)-urea; and
79) 1-ethyl-3-{6-fluoro-5-[1-(3-methoxy-pyridin-2-ylmethyl)-2-oxo-1,2-dihydro-pyridin-4-yl]-benzothiazol-2-yl}-urea; and
   and; salts, racemates, diastereomers, enantiomers, deuterated forms, hydrates, solvates and prodrugs thereof.

In one embodiment of Formula (I):

Alk, Ring A, X₁ and X₂ are as previously defined;

X₃ may be CH, —N═ or C—(Y₁)ₙR₃ wherein R₃ may be as previously defined. In one example, R₃ may be an optionally substituted 5-membered or 6-membered heteroaryl ring. In another example, R₃ may be an optionally substituted 6-membered heteroaryl ring.

n may be an integer 0 or 1 and when n is 0 then Y₁ may be a covalent bond or may be absent and when n is 1 then Y₁ may be selected from O, C(═O). For example, Y₁ may be C₁₋₆alkylene. In one example, Y₁ may be C₁₋₃alkylene. In one example, Y₁ may be —CH₂—, C₁₋₆alkylO—, for example, C₁₋₃alkylO— such as —CH₂O—. In one example, Y₁ may be C₁₋₆ alkylNH—. In another example, Y₁ may be C₁₋₃alkylNH—, such as —CH₂NH—. In another example, Y₁ may be C₁₋₆alkylN(C₁₋₃alkyl)-. For example, Y₁ may be C₁₋₃alkylN(C₁₋₃alkyl)-. In one example, Y₁ may be —CH₂N(Me)—. In another example, Y₁ may be C₂₋₆alkenylene. For example, Y₁ may be C₂₋₃alkenylene. In another example, Y₁ may be C₂₋₆alkynylene. In one example, Y₁ may be C₂₋₃alkynylene, —CH₂N(C₁₋₃alkyl)-, NH, N(C₁₋₃alkyl). In one example, Y₁ may be N(Me), —C(O)NH—, —C(O)N(C₁₋₃alkyl)-. In one example, Y₁ may be —C(O)N(Me)—, —NHC(O)—, —C(C₁₋₃alkyl)=N—O— and —CH═N—O—. In one example, Y₁ may be —C(Et)=N—O— or C(Me)=N—O—.

Z₁ may be a carbonyl containing group of general formula —(Y)_{q}B(R₄)—C(═O)—W—R₅ wherein:

q may be an integer 0 or 1.

Y is attached to Ring A and when q is 0 then Y is a covalent bond, a spiro ring centre, or a fused ring bond; and in a one example Y is a covalent bond when q is 0; or when q is 1 then Y is selected from optionally substituted C₁₋₃alkylene, optionally substituted C₂₋₃alkenylene and optionally substituted C₂₋₃alkynylene and wherein each carbon atom in C₁₋₃alkylene may be optionally replaced by an oxygen or nitrogen heteroatom or C(═O). In one example, Y may be selected from the group comprising of —C(O)NH— or —NHC(O)—, —NH—, —CH₂NH—, —NHCH₂—, —N(CH₃)—, —CH₂N(CH₃)—, —N(CH₃)CH₂—, methylene, ethylene, propylene and C═O. In one example, Y may be selected from methylene, NH, N(CH₃) and C(═O) when q is 1.

B represents “Ring B” and may be selected from saturated or unsaturated monocyclic C₃₋₇cycloalkyl, saturated or unsaturated monocyclic 3-7 membered heterocycle, saturated or unsaturated fused bicyclic C₈₋₁₀cycloalkyl, saturated or unsaturated fused bicyclic 8-12 membered heterocyclyl, C₆₋₁₀aryl, 5-10 membered heteroaryl, and a spiro bicyclic 8-12 membered heterocyclic ring system; and further Ring B may be optionally substituted; or Ring B may join together with Ring A to form a saturated or unsaturated fused bicyclic C₈₋₁₀cycloalkyl, a saturated or unsaturated fused bicyclic 8-10 membered heterocyclyl and a spiro bicyclic 8-12 membered heterocyclic ring system. For example, Ring B may be an optionally substituted C₃₋₇cycloalkyl or an optionally substituted 4-, 5-, 6- or 7-membered heterocyclic group. For example, Ring B may be an optionally substituted C₅₋₆cycloalkyl. In one example, Ring B may be a cyclohexyl or an optionally substituted 5- or 6-membered heterocyclic group. In one example, Ring B may be a 6-membered heterocyclic group.

In one example, Ring B may be a heterocylic group containing nitrogen and/or oxygen and includes dioxane, piperidinyl, pyrrolidinyl, azepane, isoxazolyl and morpholinyl.

In one example, Ring B may be selected from piperidinyl, pyrrolidinyl, azepane, isoxazolyl and morpholinyl. For example, Ring B may be piperidinyl.

R₄ may be joined to the same Ring B atom as the —C(═O)—W—R₅ moiety and may be selected from C₁₋₆alkyl, C₂₋₆alkenyl, C₂₋₆alkynyl, (C₁₋₆alkyl)ₜC₃₋₇cycloalkyl, (C₁₋₆alkyl)ₜaryl, (C₁₋₆ alkyl)ₜheterocyclyl, (C₁₋₆alkyl)ₜheteroaryl, NH₂, NH(C₁₋₆alkyl), N(C₁₋₆alkyl)₂, CN, OH, C₁₋₆alkoxy, SO₂H, SO₂C₁₋₆alkyl, SH, SC₁₋₆alkyl, halo, haloC₁₋₆alkyl, —NH(C═O)OC₁₋₆alkyl, —NH(C═O)OC(C₁₋₃alkyl)₃, and wherein C₁₋₃alkyl, C₁₋₆alkyl, C₂₋₆alkenyl, C₂₋₆alkynyl, C₃₋₇cycloalkyl, aryl and heterocyclyl in each case may be further optionally substituted, for example, with one or more substituents selected from NH₂, NH(C₁₋₆alkyl), N(C₁₋₆alkyl)₂, CN, OH, C₁₋₆alkoxy, SO₂H, SO₂C₁₋₆alkyl, SH, SC₁₋₆alkyl and halo or R₄ is a chain of 3 or 4 carbon atoms or carbon and heteroatoms which joins with an adjacent B ring atom to form a fused carbocyclylic or heterocyclic ring which is optionally further substituted. For example, R₄ may be a C₁₋₆alkyl or C₃₋₇cycloalkyl. In one example, R₄ may be a C₁₋₃alkyl or cyclopropyl. In one example, R₄ may be a methyl, ethyl, n-propyl and iso-propyl. In one example, R₄ may be a methyl or ethyl.

The —C(═O)—W—R₅ moiety may be joined to the same Ring B atom as R₄; wherein W may be O, NH or N(C₁₋₆alkyl). For example, W may be O; and R₅ may be selected from H, C₁₋₆alkyl, C₂₋₆alkenyl, C₂₋₆alkynyl, S(O)₂OH, S(O)₂—C₁₋₆alkyl, or M where M may represent a monovalent or divalent cation selected from the group comprising pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. For example, R₅ may be a H or C₁₋₃alkyl selected from methyl, ethyl, propyl and iso-propyl. In one example, R₅ may be H.

In one embodiment Ring A and/or Ring B may be optionally substituted with one or more substituents. For example, one or two optional substituents independently selected from C₁₋₃alkyl, for example methyl, OH, ═O, halo, for example F and C₁₋₃alkoxy, for example methoxy.

Examples of suitable compounds according to this embodiment includes but is not limited to, any one of compound examples 1 to 234 as previously disclosed in WO2012/045124:
1) 1-[5-[2-(ethylcarbamoylamino)-7-(2-pyridyl)-1,3-benzothiazol-5-yl]pyrimidin-2-yl]-4-methyl-piperidine-4-carboxylic acid;
2) 1-[5-[2-(ethylcarbamoylamino)-7-(2-pyridyl)-1,3-benzothiazol-5-yl]pyrimidin-2-yl]-2-methyl-piperidine-2-carboxylic acid;
3) 1-[5-[2-(ethylcarbamoylamino)-7-(2-pyridyl)-1,3-benzothiazol-5-yl]pyrimidin-2-yl]-4-hydroxy-3-methyl-piperidine-3-carboxylic acid;
4) 1-[5-[2-(ethylcarbamoylamino)-7-(2-pyridyl)-1,3-benzothiazol-5-yl]-2-pyridyl]-4-methyl-piperidine-4-carboxylic acid;
5) 1-[5-[2-(ethylcarbamoylamino)-7-(2-pyridyl)-1,3-benzothiazol-5-yl]-3-methyl-2-pyridyl]-4-methyl-piperidine-4-carboxylic acid;
6) 1-[5-[2-(ethylcarbamoylamino)-7-(4-methyl-2-pyridyl)-1,3-benzothiazol-5-yl]pyrimidin-2-yl]-4-methyl-piperidine-4-carboxylic acid;
7) 1-[5-[7-(5,6-dimethoxy-2-pyridyl)-2-(ethylcarbamoylamino)-1,3-benzothiazol-5-yl]pyrimidin-2-yl]-4-methyl-piperidine-4-carboxylic acid;
8) 1-[5-[2-(ethylcarbamoylamino)-7-(4-methoxypyrimidin-2-yl)-1,3-benzothiazol-5-yl]pyrimidin-2-yl]-4-methyl-piperidine-4-carboxylic acid;
9) 1-[5-[2-(ethylcarbamoylamino)-7-(2-pyridyl)-1,3-benzothiazol-5-yl]pyrimidin-2-yl]-3,4-dimethyl-piperidine-4-carboxylic acid;
10) 1-[5-[2-(ethylcarbamoylamino)-7-(4-fluoro-2-pyridyl)-1,3-benzothiazol-5-yl]pyrimidin-2-yl]-4-methyl-piperidine-4-carboxylic acid;
11) 1-[5-[2-(ethylcarbamoylamino)-7-pyrazin-2-yl-1,3-benzothiazol-5-yl]pyrimidin-2-yl]-4-methyl-piperidine-4-carboxylic acid;
12) 1-[5-[2-(ethylcarbamoylamino)-7-(4-methylpyrimidin-2-yl)-1,3-benzothiazol-5-yl]pyrimidin-2-yl]-4-methyl-piperidine-4-carboxylic acid;
13) 1-[5-[7-(3-cyano-2-pyridyl)-2-(ethylcarbamoylamino)-1,3-benzothiazol-5-yl]pyrimidin-2-yl]-4-methyl-piperidine-4-carboxylic acid;
14) 1-[5-[7-(5-cyano-2-pyridyl)-2-(ethylcarbamoylamino)-1,3-benzothiazol-5-yl]pyrimidin-2-yl]-4-methyl-piperidine-4-carboxylic acid;
15) 1-[5-[2-(ethylcarbamoylamino)-7-(6-methoxypyrimidin-4-yl)-1,3-benzothiazol-5-yl]pyrimidin-2-yl]-4-methyl-piperidine-4-carboxylic acid;
16) 1-[5-[7-(4-cyano-2-pyridyl)-2-(ethylcarbamoylamino)-1,3-benzothiazol-5-yl]pyrimidin-2-yl]-4-methyl-piperidine-4-carboxylic acid;
17) 1-[5-[2-(ethylcarbamoylamino)-7-(4-methoxy-2-pyridyl)-1,3-benzothiazol-5-yl]pyrimidin-2-yl]-4-methyl-piperidine-4-carboxylic acid;
18) 1-[5-[2-(ethylcarbamoylamino)-7-(3-fluoro-2-pyridyl)-1,3-benzothiazol-5-yl]pyrimidin-2-yl]-4-methyl-piperidine-4-carboxylic acid;
19) 1-[5-[2-(ethylcarbamoylamino)-7-oxazol-2-yl-1,3-benzothiazol-5-yl]pyrimidin-2-yl]-4-methyl-piperidine-4-carboxylic acid;
20) 1-[5-[2-(ethylcarbamoylamino)-7-thiazol-5-yl-1,3-benzothiazol-5-yl]pyrimidin-2-yl]-4-methyl-piperidine-4-carboxylic acid;
21) 1-[5-[2-(ethylcarbamoylamino)-7-thiazol-4-yl-1,3-benzothiazol-5-yl]pyrimidin-2-yl]-4-methyl-piperidine-4-carboxylic acid;
22) 1-[5-[2-(ethylcarbamoylamino)-7-hex-1-ynyl-1,3-benzothiazol-5-yl]pyrimidin-2-yl]-4-methyl-piperidine-4-carboxylic acid;
23) 1-[5-[2-(ethylcarbamoylamino)-7-(2-methoxythiazol-4-yl)-1,3-benzothiazol-5-yl]pyrimidin-2-yl]-4-methyl-piperidine-4-carboxylic acid;
24) 1-[5-[2-(ethylcarbamoylamino)-7-(1-methylpyrazol-4-yl)-1,3-benzothiazol-5-yl]pyrimidin-2-yl]-4-methyl-piperidine-4-carboxylic acid;
25) 1-[5-[2-(ethylcarbamoylamino)-7-(1H-pyrazol-4-yl)-1,3-benzothiazol-5-yl]pyrimidin-2-yl]-4-methyl-piperidine-4-carboxylic acid;
26) 1-[5-[2-(ethylcarbamoylamino)-7-hydroxy-1,3-benzothiazol-5-yl]pyrimidin-2-yl]-4-methyl-piperidine-4-carboxylic acid;
27) 1-[5-[2-(ethylcarbamoylamino)-7-[4-(hydroxymethyl)thiazol-2-yl]-1,3-benzothiazol-5-yl]pyrimidin-2-yl]-4-methyl-piperidine-4-carboxylic acid;
28) 1-[5-[2-(ethylcarbamoylamino)-7-ethynyl-1,3-benzothiazol-5-yl]pyrimidin-2-yl]-4-methyl-piperidine-4-carboxylic acid;
29) 1-[5-[2-(ethylcarbamoylamino)-7-pyrazol-1-yl-1,3-benzothiazol-5-yl]pyrimidin-2-yl]-4-methyl-piperidine-4-carboxylic acid;
30) 1-[5-[2-(ethylcarbamoylamino)-7-(1-methylpyrrol-2-yl)-1,3-benzothiazol-5-yl]pyrimidin-2-yl]-4-methyl-piperidine-4-carboxylic acid;
31) 1-[5-[2-(ethylcarbamoylamino)-7-(5-methyl-2-pyridyl)-1,3-benzothiazol-5-yl]pyrimidin-2-yl]-4-methyl-piperidine-4-carboxylic acid;
32) 1-[5-[7-(6-cyano-2-pyridyl)-2-(ethylcarbamoylamino)-1,3-benzothiazol-5-yl]pyrimidin-2-yl]-4-methyl-piperidine-4-carboxylic acid;
33) 1-[5-[2-(ethylcarbamoylamino)-7-imidazol-1-yl-1,3-benzothiazol-5-yl]pyrimidin-2-yl]-4-methyl-piperidine-4-carboxylic acid;
34) 1-[5-[2-(ethylcarbamoylamino)-7-(5-fluoropyrimidin-2-yl)-1,3-benzothiazol-5-yl]pyrimidin-2-yl]-4-methyl-piperidine-4-carboxylic acid;
35) 1-[5-[2-(ethylcarbamoylamino)-7-[5-(trifluoromethyl)-2-pyridyl]-1,3-benzothiazol-5-yl]pyrimidin-2-yl]-4-methyl-piperidine-4-carboxylic acid;
36) 1-[5-[2-(ethylcarbamoylamino)-7-(2-pyridyl)-1,3-benzothiazol-5-yl]-3-fluoro-2-pyridyl]-4-methyl-piperidine-4-carboxylic acid;
37) 1-[5-[7-ethyl-2-(ethylcarbamoylamino)-1,3-benzothiazol-5-yl]pyrimidin-2-yl]-4-methyl-piperidine-4-carboxylic acid;
38) 1-[5-[2-(ethylcarbamoylamino)-7-methyl-1,3-benzothiazol-5-yl]pyrimidin-2-yl]-4-methyl-piperidine-4-carboxylic acid;
39) 1-[5-[7-cyclohexyl-2-(ethylcarbamoylamino)-1,3-benzothiazol-5-yl]pyrimidin-2-yl]-4-methyl-piperidine-4-carboxylic acid;
40) 1-[5-[2-(ethylcarbamoylamino)-7-(2-pyridyl)-1,3-benzothiazol-5-yl]pyrazin-2-yl]-4-methyl-piperidine-4-carboxylic acid;
41) 1-[5-[2-(ethylcarbamoylamino)-7-(1-phenyltriazol-4-yl)-1,3-benzothiazol-5-yl]pyrimidin-2-yl]-4-methyl-piperidine-4-carboxylic acid;
42) 1-[5-[2-(ethylcarbamoylamino)-7-(2-methoxypyrimidin-4-yl)-1,3-benzothiazol-5-yl]pyrimidin-2-yl]-4-methyl-piperidine-4-carboxylic acid;
43) 1-[5-[2-(ethylcarbamoylamino)-7-(2-methylpyrimidin-4-yl)-1,3-benzothiazol-5-yl]pyrimidin-2-yl]-4-methyl-piperidine-4-carboxylic acid;
44) 1-[5-[7-(2-cyanopyrimidin-4-yl)-2-(ethylcarbamoylamino)-1,3-benzothiazol-5-yl]pyrimidin-2-yl]-4-methyl-piperidine-4-carboxylic acid;
45) 1-[5-[2-(ethylcarbamoylamino)-7-(6-methylpyrimidin-4-yl)-1,3-benzothiazol-5-yl]pyrimidin-2-yl]-4-methyl-piperidine-4-carboxylic acid;
46) 1-[5-[2-(ethylcarbamoylamino)-1,3-benzothiazol-5-yl]pyrimidin-2-yl]-4-methyl-piperidine-4-carboxylic acid;
47) 1-[5-[2-(ethylcarbamoylamino)-7-tetrahydropyran-4-yl-1,3-benzothiazol-5-yl]pyrimidin-2-yl]-4-methyl-piperidine-4-carboxylic acid;
48) 1-[5-[2-(ethylcarbamoylamino)-7-(6-isopropoxypyrimidin-4-yl)-1,3-benzothiazol-5-yl]pyrimidin-2-yl]-4-methyl-piperidine-4-carboxylic acid;
49) 1-[5-[2-(ethylcarbamoylamino)-7-[6-(2-methoxyethoxy)-2-methyl-pyrimidin-4-yl]-1,3-benzothiazol-5-yl]pyrimidin-2-yl]-4-methyl-piperidine-4-carboxylic acid;
50) 1-[5-[2-(ethylcarbamoylamino)-7-(1-methyltriazol-4-yl)-1,3-benzothiazol-5-yl]pyrimidin-2-yl]-4-methyl-piperidine-4-carboxylic acid;
51) 1-[5-[2-(ethylcarbamoylamino)-7-(5-methoxypyrimidin-2-yl)-1,3-benzothiazol-5-yl]pyrimidin-2-yl]-4-methyl-piperidine-4-carboxylic acid;
52) 1-[5-[2-(ethylcarbamoylamino)-7-(6-methoxy-2-pyridyl)-1,3-benzothiazol-5-yl]pyrimidin-2-yl]-4-methyl-piperidine-4-carboxylic acid;
53) 1-[5-[2-(ethylcarbamoylamino)-7-(5-fluoro-6-methyl-2-pyridyl)-1,3-benzothiazol-5-yl]pyrimidin-2-yl]-4-methyl-piperidine-4-carboxylic acid;
54) 1-[5-[7-(3,5-difluoro-2-pyridyl)-2-(ethylcarbamoylamino)-1,3-benzothiazol-5-yl]pyrimidin-2-yl]-4-methyl-piperidine-4-carboxylic acid;
55) 1-[5-[2-(ethylcarbamoylamino)-7-(3-fluoro-4-methoxy-2-pyridyl)-1,3-benzothiazol-5-yl]pyrimidin-2-yl]-4-methyl-piperidine-4-carboxylic acid;
56) 1-[5-[2-(ethylcarbamoylamino)-7-(5-methoxypyrazin-2-yl)-1,3-benzothiazol-5-yl]pyrimidin-2-yl]-4-methyl-piperidine-4-carboxylic acid;
57) 1-[5-[2-(ethylcarbamoylamino)-7-(1-isopropyl-6-oxo-pyrimidin-4-yl)-1,3-benzothiazol-5-yl]pyrimidin-2-yl]-4-methyl-piperidine-4-carboxylic acid;
58) 1-[5-[7-(5-cyano-6-methyl-2-pyridyl)-2-(ethylcarbamoylamino)-1,3-benzothiazol-5-yl]pyrimidin-2-yl]-4-methyl-piperidine-4-carboxylic acid;
59) 1-[5-[2-(ethylcarbamoylamino)-7-(5-methylpyrimidin-2-yl)-1,3-benzothiazol-5-yl]pyrimidin-2-yl]-4-methyl-piperidine-4-carboxylic acid;
60) 1-[5-[2-(ethylcarbamoylamino)-7-(5-ethylpyrimidin-2-yl)-1,3-benzothiazol-5-yl]pyrimidin-2-yl]-4-methyl-piperidine-4-carboxylic acid;
61) 1-[5-[2-(ethylcarbamoylamino)-7-(3-methoxy-2-pyridyl)-1,3-benzothiazol-5-yl]pyrimidin-2-yl]-4-methyl-piperidine-4-carboxylic acid;
62) 1-[5-[7-(3-amino-6-methoxy-2-pyridyl)-2-(ethylcarbamoylamino)-1,3-benzothiazol-5-yl]pyrimidin-2-yl]-4-methyl-piperidine-4-carboxylic acid;
63) 1-[5-[7-(5-cyano-3-fluoro-2-pyridyl)-2-(ethylcarbamoylamino)-1,3-benzothiazol-5-yl]pyrimidin-2-yl]-4-methyl-piperidine-4-carboxylic acid;
64) 1-[5-[2-(ethylcarbamoylamino)-7-(3-methoxy-6-methyl-2-pyridyl)-1,3-benzothiazol-5-yl]pyrimidin-2-yl]-4-methyl-piperidine-4-carboxylic acid;
65) 1-[5-[7-(3-cyano-5-methyl-2-pyridyl)-2-(ethylcarbamoylamino)-1,3-benzothiazol-5-yl]pyrimidin-2-yl]-4-methyl-piperidine-4-carboxylic acid;
66) 1-[5-[2-(ethylcarbamoylamino)-7-(5-methylpyrazin-2-yl)-1,3-benzothiazol-5-yl]pyrimidin-2-yl]-4-methyl-piperidine-4-carboxylic acid;
67) 1-[5-[2-(ethylcarbamoylamino)-7-pyrimidin-4-yl-1,3-benzothiazol-5-yl]pyrimidin-2-yl]-4-methyl-piperidine-4-carboxylic acid;
68) 1-[5-[7-(4-ethoxy-2-pyridyl)-2-(ethylcarbamoylamino)-1,3-benzothiazol-5-yl]pyrimidin-2-yl]-4-methyl-piperidine-4-carboxylic acid;
69) 1-[5-[7-(5-cyano-3-methyl-2-pyridyl)-2-(ethylcarbamoylamino)-1,3-benzothiazol-5-yl]pyrimidin-2-yl]-4-methyl-piperidine-4-carboxylic acid;
70) 1-[5-[2-(ethylcarbamoylamino)-7-furo[3,2-c]pyridin-4-yl-1,3-benzothiazol-5-yl]pyrimidin-2-yl]-4-methyl-piperidine-4-carboxylic acid;
71) 1-[5-[7-[3-cyano-4-(dimethylamino)-2-pyridyl]-2-(ethylcarbamoylamino)-1,3-benzothiazol-5-yl]pyrimidin-2-yl]-4-methyl-piperidine-4-carboxylic acid;
72) 1-[5-[2-(ethylcarbamoylamino)-7-[(E)-methoxyiminomethyl]-1,3-benzothiazol-5-yl]pyrimidin-2-yl]-4-methyl-piperidine-4-carboxylic acid;
73) 1-[5-[7-(6-tert-butoxypyrazin-2-yl)-2-(ethylcarbamoylamino)-1,3-benzothiazol-5-yl]pyrimidin-2-yl]-4-methyl-piperidine-4-carboxylic acid;
74) 1-[5-[7-(1-acetyl-4-piperidyl)-2-(ethylcarbamoylamino)-1,3-benzothiazol-5-yl]pyrimidin-2-yl]-4-methyl-piperidine-4-carboxylic acid;
75) 1-[5-[7-[2-(dimethylamino)thiazol-5-yl]-2-(ethylcarbamoylamino)-1,3-benzothiazol-5-yl]pyrimidin-2-yl]-4-methyl-piperidine-4-carboxylic acid;
76) 1-[5-[2-(ethylcarbamoylamino)-7-(2-morpholinothiazol-5-yl)-1,3-benzothiazol-5-yl]pyrimidin-2-yl]-4-methyl-piperidine-4-carboxylic acid;
77) 1-[5-[2-(ethylcarbamoylamino)-7-(2-ethylthiazol-5-yl)-1,3-benzothiazol-5-yl]pyrimidin-2-yl]-4-methyl-piperidine-4-carboxylic acid;
78) 1-[5-[7-(2-ethoxythiazol-5-yl)-2-(ethylcarbamoylamino)-1,3-benzothiazol-5-yl]pyrimidin-2-yl]-4-methyl-piperidine-4-carboxylic acid;
79) 1-[5-[2-(ethylcarbamoylamino)-7-(1-methyl-2-oxo-4-pyridyl)-1,3-benzothiazol-5-yl]pyrimidin-2-yl]-4-methyl-piperidine-4-carboxylic acid;
80) 1-[5-[2-(ethylcarbamoylamino)-7-[1-(2-methoxyethyl)-6-oxo-pyrimidin-4-yl]-1,3-benzothiazol-5-yl]pyrimidin-2-yl]-4-methyl-piperidine-4-carboxylic acid;
81) 1-[5-[2-(ethylcarbamoylamino)-7-(6-methoxypyrazin-2-yl)-1,3-benzothiazol-5-yl]pyrimidin-2-yl]-4-methyl-piperidine-4-carboxylic acid;
82) 1-[5-[7-[5-(cyanomethyl)-2-pyridyl]-2-(ethylcarbamoylamino)-1,3-benzothiazol-5-yl]pyrimidin-2-yl]-4-methyl-piperidine-4-carboxylic acid;
83) 1-[5-[2-(ethylcarbamoylamino)-7-(5-ethylpyrazin-2-yl)-1,3-benzothiazol-5-yl]pyrimidin-2-yl]-4-methyl-piperidine-4-carboxylic acid;
84) 1-[5-[2-(ethylcarbamoylamino)-7-(3-methylpyrazin-2-yl)-1,3-benzothiazol-5-yl]pyrimidin-2-yl]-4-methyl-piperidine-4-carboxylic acid;
85) 1-[5-[2-(ethylcarbamoylamino)-7-(5-fluoro-4-methyl-2-pyridyl)-1,3-benzothiazol-5-yl]pyrimidin-2-yl]-4-methyl-piperidine-4-carboxylic acid;
86) 1-[5-[2-(ethylcarbamoylamino)-7-(3-methoxypyrazin-2-yl)-1,3-benzothiazol-5-yl]pyrimidin-2-yl]-4-methyl-piperidine-4-carboxylic acid;
87) 1-[5-[7-cyclopropyl-2-(ethylcarbamoylamino)-1,3-benzothiazol-5-yl]pyrimidin-2-yl]-4-methyl-piperidine-4-carboxylic acid;
88) 1-[5-[2-(ethylcarbamoylamino)-7-[(E)-N-methoxy-C-methyl-carbonimidoyl]-1,3-benzothiazol-5-yl]pyrimidin-2-yl]-4-methyl-piperidine-4-carboxylic acid;
89) 1-[5-[2-(ethylcarbamoylamino)-7-(4-pyridyl)-1,3-benzothiazol-5-yl]pyrimidin-2-yl]-4-methyl-piperidine-4-carboxylic acid;
90) 1-[5-[7-(3-cyano-4-methoxy-2-pyridyl)-2-(ethylcarbamoylamino)-1,3-benzothiazol-5-yl]pyrimidin-2-yl]-4-methyl-piperidine-4-carboxylic acid;
91) 1-[5-[2-(ethylcarbamoylamino)-7-[ethyl(methyl)carbamoyl]-1,3-benzothiazol-5-yl]pyrimidin-2-yl]-4-methyl-piperidine-4-carboxylic acid;
92) 1-[5-[7-(5-cyano-4-methyl-2-pyridyl)-2-(ethylcarbamoylamino)-1,3-benzothiazol-5-yl]pyrimidin-2-yl]-4-methyl-piperidine-4-carboxylic acid;
93) 1-[5-[2-(ethylcarbamoylamino)-7-(3-fluoro-5-methyl-2-pyridyl)-1,3-benzothiazol-5-yl]pyrimidin-2-yl]-4-methyl-piperidine-4-carboxylic acid;
94) 1-[5-[2-(ethylcarbamoylamino)-7-[(E)-C-methyl-N-(2,2,2-trifluoroethoxy)carbonimidoyl]-1,3-benzothiazol-5-yl]pyrimidin-2-yl]-4-methyl-piperidine-4-carboxylic acid;
95) 1-[5-[2-(ethylcarbamoylamino)-7-[(E)-N-hydroxy-C-methyl-carbonimidoyl]-1,3-benzothiazol-5-yl]pyrimidin-2-yl]-4-methyl-piperidine-4-carboxylic acid;
96) 1-[5-[7-[(E)-N-ethoxy-C-methyl-carbonimidoyl]-2-(ethylcarbamoylamino)-1,3-benzothiazol-5-yl]pyrimidin-2-yl]-4-methyl-piperidine-4-carboxylic acid;
97) 1-[5-[7-(ethylcarbamoyl)-2-(ethylcarbamoylamino)-1,3-benzothiazol-5-yl]pyrimidin-2-yl]-4-methyl-piperidine-4-carboxylic acid;
98) 4-ethyl-1-[5-[2-(ethylcarbamoylamino)-7-(2-pyridyl)-1,3-benzothiazol-5-yl]pyrimidin-2-yl]piperidine-4-carboxylic acid;
99) 4-ethyl-1-[5-[2-(ethylcarbamoylamino)-7-pyrazin-2-yl-1,3-benzothiazol-5-yl]pyrimidin-2-yl]piperidine-4-carboxylic acid;
100) 1-[5-[7-(4-cyano-2-pyridyl)-2-(ethylcarbamoylamino)-1,3-benzothiazol-5-yl]pyrimidin-2-yl]-4-ethyl-piperidine-4-carboxylic acid;
101) 4-ethyl-1-[5-[2-(ethylcarbamoylamino)-7-(4-methylpyrimidin-2-yl)-1,3-benzothiazol-5-yl]pyrimidin-2-yl]piperidine-4-carboxylic acid;
102) 4-ethyl-1-[5-[2-(ethylcarbamoylamino)-7-(4-methoxy-2-pyridyl)-1,3-benzothiazol-5-yl]pyrimidin-2-yl]piperidine-4-carboxylic acid;
103) 4-ethyl-1-[5-[2-(ethylcarbamoylamino)-7-(2-pyridyl)-1,3-benzothiazol-5-yl]-2-pyridyl]piperidine-4-carboxylic acid;
104) 1-[5-[7-(3-cyano-2-pyridyl)-2-(ethylcarbamoylamino)-1,3-benzothiazol-5-yl]pyrimidin-2-yl]-4-ethyl-piperidine-4-carboxylic acid;
105) 1-[5-[7-(5-cyano-2-pyridyl)-2-(ethylcarbamoylamino)-1,3-benzothiazol-5-yl]pyrimidin-2-yl]-4-ethyl-piperidine-4-carboxylic acid;
106) 4-ethyl-1-[5-[2-(ethylcarbamoylamino)-7-(7H-pyrrolo[2,3-d]pyrimidin-4-yl)-1,3-benzothiazol-5-yl]pyrimidin-2-yl]piperidine-4-carboxylic acid;
107) 1-[5-[7-bromo-2-(ethylcarbamoylamino)-1,3-benzothiazol-5-yl]pyrimidin-2-yl]-4-ethyl-piperidine-4-carboxylic acid;
108) 4-ethyl-1-[5-[2-(ethylcarbamoylamino)-7-(2-pyridyl)-1,3-benzothiazol-5-yl]pyrazin-2-yl]piperidine-4-carboxylic acid;
109) 4-ethyl-1-[5-[2-(ethylcarbamoylamino)-7-(4-methoxypyrimidin-2-yl)-1,3-benzothiazol-5-yl]pyrimidin-2-yl]piperidine-4-carboxylic acid;
110) 4-ethyl-1-[5-[2-(ethylcarbamoylamino)-7-(6-methoxypyrimidin-4-yl)-1,3-benzothiazol-5-yl]pyrimidin-2-yl]piperidine-4-carboxylic acid;
111) 4-ethyl-1-[5-[2-(ethylcarbamoylamino)-7-(6-oxo-1H-pyrimidin-4-yl)-1,3-benzothiazol-5-yl]pyrimidin-2-yl]piperidine-4-carboxylic acid;
112) 4-ethyl-1-[5-[2-(ethylcarbamoylamino)-7-(4-methylpyrimidin-2-yl)-1,3-benzothiazol-5-yl]pyrazin-2-yl]piperidine-4-carboxylic acid;
113) 4-ethyl-1-[5-[2-(ethylcarbamoylamino)-1,3-benzothiazol-5-yl]pyrimidin-2-yl]piperidine-4-carboxylic acid;
114) 4-ethyl-1-[5-[2-(ethylcarbamoylamino)-7-morpholino-1,3-benzothiazol-5-yl]pyrimidin-2-yl]piperidine-4-carboxylic acid;
115) 4-ethyl-1-[5-[2-(ethylcarbamoylamino)-7-(4-fluorophenyl)-1,3-benzothiazol-5-yl]pyrimidin-2-yl]piperidine-4-carboxylic acid;
116) 4-ethyl-1-[5-[2-(ethylcarbamoylamino)-7-[(E)-N-methoxy-C-methyl-carbonimidoyl]-1,3-benzothiazol-5-yl]pyrimidin-2-yl]piperidine-4-carboxylic acid;
117) 1-[5-[2-(ethylcarbamoylamino)-7-(2-pyridyl)-1,3-benzothiazol-5-yl]pyrimidin-2-yl]-4-propyl-piperidine-4-carboxylic acid;
118) 1-[5-[2-(ethylcarbamoylamino)-7-pyrazin-2-yl-1,3-benzothiazol-5-yl]pyrimidin-2-yl]-4-propyl-piperidine-4-carboxylic acid;
119) 1-[5-[7-bromo-2-(ethylcarbamoylamino)-1,3-benzothiazol-5-yl]pyrimidin-2-yl]-4-propyl-piperidine-4-carboxylic acid;
120) 1-[5-[2-(ethylcarbamoylamino)-7-(4-methylpyrimidin-2-yl)-1,3-benzothiazol-5-yl]pyrimidin-2-yl]-4-propyl-piperidine-4-carboxylic acid;
121) 1-[5-[2-(ethylcarbamoylamino)-7-(6-methoxypyrimidin-4-yl)-1,3-benzothiazol-5-yl]pyrimidin-2-yl]-4-propyl-piperidine-4-carboxylic acid;
122) 1-[5-[2-(ethylcarbamoylamino)-7-(4-methoxy-2-pyridyl)-1,3-benzothiazol-5-yl]pyrimidin-2-yl]-4-propyl-piperidine-4-carboxylic acid;
123) 1-[5-[7-(4-cyano-2-pyridyl)-2-(ethylcarbamoylamino)-1,3-benzothiazol-5-yl]pyrimidin-2-yl]-4-propyl-piperidine-4-carboxylic acid;
124) 1-[5-[7-(5-cyano-2-pyridyl)-2-(ethylcarbamoylamino)-1,3-benzothiazol-5-yl]pyrimidin-2-yl]-4-propyl-piperidine-4-carboxylic acid;
125) 1-[5-[2-(ethylcarbamoylamino)-7-(7H-pyrrolo[2,3-d]pyrimidin-4-yl)-1,3-benzothiazol-5-yl]pyrimidin-2-yl]-4-propyl-piperidine-4-carboxylic acid;
126) 1-[5-[7-(3-cyano-2-pyridyl)-2-(ethylcarbamoylamino)-1,3-benzothiazol-5-yl]pyrimidin-2-yl]-4-propyl-piperidine-4-carboxylic acid;
127) 1-[5-[2-(ethylcarbamoylamino)-7-(4-methoxypyrimidin-2-yl)-1,3-benzothiazol-5-yl]pyrimidin-2-yl]-4-propyl-piperidine-4-carboxylic acid;
128) 4-amino-1-[5-[2-(ethylcarbamoylamino)-7-(2-pyridyl)-1,3-benzothiazol-5-yl]pyrimidin-2-yl]piperidine-4-carboxylic acid;
129) 1-[5-[2-(ethylcarbamoylamino)-7-(2-pyridyl)-1,3-benzothiazol-5-yl]pyrimidin-2-yl]-4-phenyl-piperidine-4-carboxylic acid;
130) 4-cyano-1-[5-[2-(ethylcarbamoylamino)-7-(2-pyridyl)-1,3-benzothiazol-5-yl]pyrimidin-2-yl]piperidine-4-carboxylic acid;
131) 1-[5-[2-(ethylcarbamoylamino)-7-(2-pyridyl)-1,3-benzothiazol-5-yl]pyrimidin-2-yl]-4-hydroxy-piperidine-4-carboxylic acid;
132) 1-[5-[2-(ethylcarbamoylamino)-7-(2-pyridyl)-1,3-benzothiazol-5-yl]pyrimidin-2-yl]-4-methoxy-piperidine-4-carboxylic acid;
133) 1-[5-[2-(ethylcarbamoylamino)-7-(2-pyridyl)-1,3-benzothiazol-5-yl]pyrimidin-2-yl]-4-methylsulfonyl-piperidine-4-carboxylic acid;
134) 4-benzyl-1-[5-[2-(ethylcarbamoylamino)-7-(2-pyridyl)-1,3-benzothiazol-5-yl]pyrimidin-2-yl]piperidine-4-carboxylic acid;
135) 1-[5-[2-(ethylcarbamoylamino)-7-(2-pyridyl)-1,3-benzothiazol-5-yl]pyrimidin-2-yl]-4-fluoro-piperidine-4-carboxylic acid;
136) 4-allyl-1-[5-[2-(ethylcarbamoylamino)-7-(2-pyridyl)-1,3-benzothiazol-5-yl]pyrimidin-2-yl]piperidine-4-carboxylic acid;
137) 1-[5-[2-(ethylcarbamoylamino)-7-(2-pyridyl)-1,3-benzothiazol-5-yl]pyrimidin-2-yl]-4-isopropyl-piperidine-4-carboxylic acid;
138) 1-[5-[2-(ethylcarbamoylamino)-7-(2-pyridyl)-1,3-benzothiazol-5-yl]pyrimidin-2-yl]-4-methylsulfanyl-piperidine-4-carboxylic acid;
139) 4-allyl-1-[5-[2-(ethylcarbamoylamino)-7-pyrazin-2-yl-1,3-benzothiazol-5-yl]pyrimidin-2-yl]piperidine-4-carboxylic acid;
140) 1-[5-[2-(ethylcarbamoylamino)-7-(2-pyridyl)-1,3-benzothiazol-5-yl]pyrimidin-2-yl]-4-(2,2,2-trifluoroethyl)piperidine-4-carboxylic acid;
141) 1-[5-[2-(ethylcarbamoylamino)-7-(2-pyridyl)-1,3-benzothiazol-5-yl]pyrimidin-2-yl]-4-(methoxymethyl)piperidine-4-carboxylic acid;
142) 1-[5-[2-(ethylcarbamoylamino)-7-(2-pyridyl)-1,3-benzothiazol-5-yl]pyrimidin-2-yl]-4-(trifluoromethyl)piperidine-4-carboxylic acid;
143) 4-cyclopropyl-1-[5-[2-(ethylcarbamoylamino)-7-(2-pyridyl)-1,3-benzothiazol-5-yl]pyrimidin-2-yl]piperidine-4-carboxylic acid;
144) 1-[5-[2-(ethylcarbamoylamino)-7-[2-(3-methylimidazol-4-yl)ethynyl]-1,3-benzothiazol-5-yl]pyrimidin-2-yl]-4-methyl-piperidine-4-carboxylic acid;
145) 1-[5-[2-(ethylcarbamoylamino)-7-(thiazol-2-ylcarbamoyl)-1,3-benzothiazol-5-yl]pyrimidin-2-yl]-4-methyl-piperidine-4-carboxylic acid;
146) 1-[5-[2-(ethylcarbamoylamino)-7-(pyrimidine-2-carbonylamino)-1,3-benzothiazol-5-yl]pyrimidin-2-yl]-4-methyl-piperidine-4-carboxylic acid;
147) 1-[5-[2-(ethylcarbamoylamino)-7-[[methyl(pyrimidin-2-yl)amino]methyl]-1,3-benzothiazol-5-yl]pyrimidin-2-yl]-4-methyl-piperidine-4-carboxylic acid;
148) 1-[5-[2-(ethylcarbamoylamino)-7-[2-(3-pyridyl)ethynyl]-1,3-benzothiazol-5-yl]pyrimidin-2-yl]-4-methyl-piperidine-4-carboxylic acid;
149) 1-[5-[2-(ethylcarbamoylamino)-7-(pyridine-2-carbonylamino)-1,3-benzothiazol-5-yl]pyrimidin-2-yl]-4-methyl-piperidine-4-carboxylic acid;
150) 1-[5-[7-(2-cyclopentylethynyl)-2-(ethylcarbamoylamino)-1,3-benzothiazol-5-yl]pyrimidin-2-yl]-4-methyl-piperidine-4-carboxylic acid;
151) 1-[5-[2-(ethylcarbamoylamino)-7-[2-(4-pyridyl)ethynyl]-1,3-benzothiazol-5-yl]pyrimidin-2-yl]-4-methyl-piperidine-4-carboxylic acid;
152) 1-[5-[2-(ethylcarbamoylamino)-7-[2-(6-methoxy-2-pyridyl)ethynyl]-1,3-benzothiazol-5-yl]pyrimidin-2-yl]-4-methyl-piperidine-4-carboxylic acid;
153) 1-[5-[2-(ethylcarbamoylamino)-7-[2-(5-methyl-1,3,4-thiadiazol-2-yl)ethynyl]-1,3-benzothiazol-5-yl]pyrimidin-2-yl]-4-methyl-piperidine-4-carboxylic acid;
154) 1-[5-[2-(ethylcarbamoylamino)-7-(2-pyrimidin-2-ylethynyl)-1,3-benzothiazol-5-yl]pyrimidin-2-yl]-4-methyl-piperidine-4-carboxylic acid;
155) 1-[5-[2-(ethylcarbamoylamino)-7-(2-pyrimidin-4-ylethynyl)-1,3-benzothiazol-5-yl]pyrimidin-2-yl]-4-methyl-piperidine-4-carboxylic acid;
156) 1-[5-[2-(ethylcarbamoylamino)-7-(2-thiazol-2-ylethynyl)-1,3-benzothiazol-5-yl]pyrimidin-2-yl]-4-methyl-piperidine-4-carboxylic acid;
157) 1-[5-[2-(ethylcarbamoylamino)-7-(2-pyrazin-2-ylethynyl)-1,3-benzothiazol-5-yl]pyrimidin-2-yl]-4-methyl-piperidine-4-carboxylic acid;
158) 1-[5-[2-(ethylcarbamoylamino)-7-(morpholinomethyl)-1,3-benzothiazol-5-yl]pyrimidin-2-yl]-4-methyl-piperidine-4-carboxylic acid;
159) 1-[5-[2-(ethylcarbamoylamino)-7-(morpholine-4-carbonyl)-1,3-benzothiazol-5-yl]pyrimidin-2-yl]-4-methyl-piperidine-4-carboxylic acid;
160) 1-[[5-[2-(ethylcarbamoylamino)-7-(2-pyridyl)-1,3-benzothiazol-5-yl]pyrimidin-2-yl]methyl]-4-methyl-piperidine-4-carboxylic acid;
161) 1-[5-[2-(ethylcarbamoylamino)-4-fluoro-7-(2-pyridyl)-1,3-benzothiazol-5-yl]pyrimidin-2-yl]-4-methyl-piperidine-4-carboxylic acid;
162) 1-[5-[2-(ethylcarbamoylamino)-4-(2-pyridyl)thiazolo[5,4-c]pyridin-6-yl]pyrimidin-2-yl]-4-methyl-piperidine-4-carboxylic acid;
163) 1-[5-[2-(ethylcarbamoylamino)-7-(2-pyridyl)-1,3-benzothiazol-5-yl]pyrimidin-2-yl]-2-methyl-pyrrolidine-2-carboxylic acid;
164) 1-[5-[2-(ethylcarbamoylamino)-7-(2-pyridyl)-1,3-benzothiazol-5-yl]pyrimidin-2-yl]-3-(trifluoromethyl)pyrrolidine-3-carboxylic acid;
165) 2-[5-[2-(ethylcarbamoylamino)-7-(2-pyridyl)-1,3-benzothiazol-5-yl]pyrimidin-2-yl]-1,3,4,5,6,6a-hexahydrocyclopenta[c]pyrrole-3a-carboxylic acid;
166) 1-[5-[2-(ethylcarbamoylamino)-7-(2-pyridyl)-1,3-benzothiazol-5-yl]pyrimidin-2-yl]-4-methyl-azepane-4-carboxylic acid;
167) methyl 4-(tert-butoxycarbonylamino)-1-[5-[2-(ethylcarbamoylamino)-7-(2-pyridyl)-1,3-benzothiazol-5-yl]pyrimidin-2-yl]piperidine-4-carboxylate;
168) methyl 4-amino-1-[5-[2-(ethylcarbamoylamino)-7-(2-pyridyl)-1,3-benzothiazol-5-yl]pyrimidin-2-yl]piperidine-4-carboxylate;
169) methyl 1-[5-[2-(ethylcarbamoylamino)-7-(2-pyridyl)-1,3-benzothiazol-5-yl]pyrimidin-2-yl]-4-methyl-piperidine-4-carboxylate;
170) methyl 1-[5-[2-(ethylcarbamoylamino)-7-(2-pyridyl)-1,3-benzothiazol-5-yl]pyrimidin-2-yl]-3-methyl-4-oxo-piperidine-3-carboxylate;
171) ethyl 1-[5-[2-(ethylcarbamoylamino)-7-(2-pyridyl)-1,3-benzothiazol-5-yl]pyrimidin-2-yl]-4-methyl-piperidine-4-carboxylate;
172) methyl 1-[5-[2-(ethylcarbamoylamino)-7-(2-pyridyl)-1,3-benzothiazol-5-yl]pyrimidin-2-yl]-4-hydroxy-3-methyl-piperidine-3-carboxylate;
173) methyl 1-[5-[2-(ethylcarbamoylamino)-7-(2-pyridyl)-1,3-benzothiazol-5-yl]pyrimidin-2-yl]-4-hydroxy-piperidine-4-carboxylate;
174) ethyl 4-cyano-1-[5-[2-(ethylcarbamoylamino)-7-(2-pyridyl)-1,3-benzothiazol-5-yl]-pyrimidin-2-yl]piperidine-4-carboxylate;
175) methyl 1-[5-[2-(ethylcarbamoylamino)-7-(2-pyridyl)-1,3-benzothiazol-5-yl]pyrimidin-2-yl]-4-methoxy-piperidine-4-carboxylate;
176) ethyl 1-[5-[2-(ethylcarbamoylamino)-7-(2-pyridyl)-1,3-benzothiazol-5-yl]pyrimidin-2-yl]-4-methylsulfonyl-piperidine-4-carboxylate;
177) ethyl 4-benzyl-1-[5-[2-(ethylcarbamoylamino)-7-(2-pyridyl)-1,3-benzothiazol-5-yl]-pyrimidin-2-yl]piperidine-4-carboxylate;
178) ethyl 1-[5-[2-(ethylcarbamoylamino)-7-tetrahydropyran-4-yl-1,3-benzothiazol-5-yl]pyrimidin-2-yl]-4-methyl-piperidine-4-carboxylate;
179) ethyl 1-[5-[7-(1-acetyl-4-piperidyl)-2-(ethylcarbamoylamino)-1,3-benzothiazol-5-yl]pyrimidin-2-yl]-4-methyl-piperidine-4-carboxylate;
180) methyl 1-[5-[2-(ethylcarbamoylamino)-7-(2-pyridyl)-1,3-benzothiazol-5-yl]pyrimidin-2-yl]-3-(trifluoromethyl)pyrrolidine-3-carboxylate;
181) 1-[5-[2-(ethylcarbamoylamino)-7-(2-pyridyl)-1,3-benzothiazol-5-yl]-3-pyridyl]-4-methyl-piperidine-4-carboxylic acid;
182) 1-[4-[2-(ethylcarbamoylamino)-7-(2-pyridyl)-1,3-benzothiazol-5-yl]-2-pyridyl]-4-methyl-piperidine-4-carboxylic acid;
183) 3-[5-[2-(ethylcarbamoylamino)-7-(2-pyridyl)-1,3-benzothiazol-5-yl]-2-pyridyl]-5-methyl-4H-isoxazole-5-carboxylic acid;
184) ethyl 3-[5-[2-(ethylcarbamoylamino)-7-(2-pyridyl)-1,3-benzothiazol-5-yl]-2-pyridyl]-5-methyl-4H-isoxazole-5-carboxylate;
185) 1-[5-[2-(ethylcarbamoylamino)-7-(2-pyridyl)-1,3-benzothiazol-5-yl]pyrimidin-2-yl]-4-phenyl-piperidine-4-carboxamide;
186) 1-[5-[2-(ethylcarbamoylamino)-7-(2-pyridyl)-1,3-benzothiazol-5-yl]pyrimidin-2-yl]-4-methyl-N-methylsulfonyl-piperidine-4-carboxamide;
187) 1-[5-[2-(ethylcarbamoylamino)-7-[(E)-N-(2-hydroxyethoxy)-C-methyl-carbonimidoyl]-1,3-benzothiazol-5-yl]pyrimidin-2-yl]-4-methyl-piperidine-4-carboxylic acid;
188) 1-[5-[2-(ethylcarbamoylamino)-7-[(E)-N-(2-methoxyethoxy)-C-methyl-carbonimidoyl]-1,3-benzothiazol-5-yl]pyrimidin-2-yl]-4-methyl-piperidine-4-carboxylic acid;
189) 4-ethyl-1-[5-[2-(ethylcarbamoylamino)-7-[(E)-methoxyiminomethyl]-1,3-benzothiazol-5-yl]pyrimidin-2-yl]piperidine-4-carboxylic acid;
190) 7-[2-(ethylcarbamoylamino)-7-(2-pyridyl)-1,3-benzothiazol-5-yl]-2-methyl-3-oxo-4H-pyrido[3,2-b][1,4]oxazine-2-carboxylic acid;
191) ethyl 1-[5-[2-(ethylcarbamoylamino)-6-methoxy-1,3-benzothiazol-5-yl]pyrimidin-2-yl]-4-methyl-piperidine-4-carboxylate;
192) 1-[5-[2-(ethylcarbamoylamino)-7-(2-pyridyl)-1,3-benzothiazol-5-yl]pyrimidine-2-carbonyl]-4-methyl-piperidine-4-carboxylic acid;
193) 1-[5-[7-carbamoyl-2-(ethylcarbamoylamino)-1,3-benzothiazol-5-yl]pyrimidin-2-yl]-4-ethyl-piperidine-4-carboxylic acid;
194) 1-[5-[2-(ethylcarbamoylamino)-7-(2-methoxyethoxymethyl)-1,3-benzothiazol-5-yl]pyrimidin-2-yl]-4-methyl-piperidine-4-carboxylic acid;
195) 1-[5-[2-(ethylcarbamoylamino)-7-(2-pyridyl)-1,3-benzothiazol-5-yl]pyridazin-3-yl]-4-methyl-piperidine-4-carboxylic acid;
196) 5-[[5-[2-(ethylcarbamoylamino)-7-(2-pyridyl)-1,3-benzothiazol-5-yl]pyrimidin-2-yl]amino]-2-methyl-1,3-dioxane-2-carboxylic acid;
197) 1-[5-[7-(4,5-dihydroisoxazol-3-yl)-2-(ethylcarbamoylamino)-1,3-benzothiazol-5-yl]pyrimidin-2-yl]-4-ethyl-piperidine-4-carboxylic acid;
198) 1-[5-[2-(ethylcarbamoylamino)-6-methoxy-1,3-benzothiazol-5-yl]pyrimidin-2-yl]-4-methyl-piperidine-4-carboxylic acid;
199) 4-ethyl-1-[5-[2-(ethylcarbamoylamino)-7-formyl-1,3-benzothiazol-5-yl]pyrimidin-2-yl]piperidine-4-carboxylic acid;
200) 1-[5-[2-(ethylcarbamoylamino)-7-methoxy-1,3-benzothiazol-5-yl]pyrimidin-2-yl]-4-methyl-piperidine-4-carboxylic acid;
201) 1-[5-[2-(ethylcarbamoylamino)-7-(2-pyridyl)-1,3-benzothiazol-5-yl]pyrimidin-2-yl]-3-methyl-pyrrolidine-3-carboxylic acid;
202) 1-[5-[2-(ethylcarbamoylamino)-7-(methoxymethyl)-1,3-benzothiazol-5-yl]pyrimidin-2-yl]-4-methyl-piperidine-4-carboxylic acid;
203) 4-ethyl-1-[5-[2-(ethylcarbamoylamino)-7-(methoxymethyl)-1,3-benzothiazol-5-yl]pyrimidin-2-yl]piperidine-4-carboxylic acid;
204) 1-[5-[7-[(E)-ethoxyiminomethyl]-2-(ethylcarbamoylamino)-1,3-benzothiazol-5-yl]pyrimidin-2-yl]-4-methyl-piperidine-4-carboxylic acid;
205) 1-[5-[2-(ethylcarbamoylamino)-7-[5-(morpholinomethyl)-2-pyridyl]-1,3-benzothiazol-5-yl]pyrimidin-2-yl]-4-methyl-piperidine-4-carboxylic acid;
206) 3-ethyl-1-[5-[2-(ethylcarbamoylamino)-7-(2-pyridyl)-1,3-benzothiazol-5-yl]pyrimidin-2-yl]pyrrolidine-3-carboxylic acid;
207) 4-ethyl-1-[5-[2-(ethylcarbamoylamino)-7-propanoyl-1,3-benzothiazol-5-yl]pyrimidin-2-yl]piperidine-4-carboxylic acid;
208) 1-[5-[7-bromo-2-(ethylcarbamoylamino)-6-methoxy-1,3-benzothiazol-5-yl]pyrimidin-2-yl]-4-methyl-piperidine-4-carboxylic acid;
209) 4-ethyl-1-[4-[2-(ethylcarbamoylamino)-7-(2-pyridyl)-1,3-benzothiazol-5-yl]thiazol-2-yl]piperidine-4-carboxylic acid;
210) 4-ethyl-1-[5-[2-(ethylcarbamoylamino)-4-(2-pyridyl)thiazolo[5,4-c]pyridin-6-yl]pyrimidin-2-yl]piperidine-4-carboxylic acid;
211) 4-ethyl-1-[5-[2-(ethylcarbamoylamino)-7-[(Z)—C-ethyl-N-methoxy-carbonimidoyl]-1,3-benzothiazol-5-yl]pyrimidin-2-yl]piperidine-4-carboxylic acid;
212) 4-ethoxy-1-[5-[2-(ethylcarbamoylamino)-7-(2-pyridyl)-1,3-benzothiazol-5-yl]pyrimidin-2-yl]piperidine-4-carboxylic acid;
213) 1-[5-[2-(ethylcarbamoylamino)-7-(5-methyl-1,2,4-oxadiazol-3-yl)-1,3-benzothiazol-5-yl]pyrimidin-2-yl]-4-methyl-piperidine-4-carboxylic acid;
214) 1-[5-[2-(ethylcarbamoylamino)-7-(2-pyridyl)-1,3-benzothiazol-5-yl]-4-methyl-pyrimidin-2-yl]-4-methyl-piperidine-4-carboxylic acid;
215) 1-[5-[7-bromo-2-(ethylcarbamoylamino)-1,3-benzothiazol-5-yl]pyrimidin-2-yl]-4-methyl-piperidine-4-carboxylic acid;
216) ethyl 5-[[5-[2-(ethylcarbamoylamino)-7-(2-pyridyl)-1,3-benzothiazol-5-yl]pyrimidin-2-yl]amino]-2-methyl-1,3-dioxane-2-carboxylate;
217) 1-[5-[7-[5-[(4,4-difluoro-1-piperidyl)methyl]-2-pyridyl]-2-(ethylcarbamoylamino)-1,3-benzothiazol-5-yl]pyrimidin-2-yl]-4-methyl-piperidine-4-carboxylic acid;
218) 4-[[5-[2-(ethylcarbamoylamino)-7-(2-pyridyl)-1,3-benzothiazol-5-yl]pyrimidin-2-yl]-methyl-amino]-1-methyl-cyclohexanecarboxylic acid;
219) 2-ethyl-7-[5-[2-(ethylcarbamoylamino)-7-(2-pyridyl)-1,3-benzothiazol-5-yl]pyrimidin-2-yl]-7-azaspiro[3.5]nonane-2-carboxylic acid;
220) 1-[5-[2-(ethylcarbamoylamino)-7-(2-pyridyl)-1,3-benzothiazol-5-yl]-4-methoxy-pyrimidin-2-yl]-4-methyl-piperidine-4-carboxylic acid;
221) 4-Ethyl-1-[5-[2-(ethylcarbamoylamino)-7-(2-pyridyl)-1,3-benzothiazol-5-yl]thiazol-2-yl]piperidine-4-carboxylic acid;
222) 4-[[5-[2-(ethylcarbamoylamino)-7-(2-pyridyl)-1,3-benzothiazol-5-yl]pyrimidin-2-yl]amino]-1-methyl-cyclohexanecarboxylic acid;
223) 4-ethyl-1-[5-[2-(ethylcarbamoylamino)-7-(C-ethyl-N-methoxy-carbonimidoyl)-1,3-benzothiazol-5-yl]pyrimidin-2-yl]piperidine-4-carboxylic acid;
224) 4-ethyl-1-[5-[2-(ethylcarbamoylamino)-7-[(E)-C-ethyl-N-methoxy-carbonimidoyl]-1,3-benzothiazol-5-yl]pyrimidin-2-yl]piperidine-4-carboxylic acid;
225) 4-ethyl-1-[5-[2-(ethylcarbamoylamino)-7-[(Z)-methoxyiminomethyl]-1,3-benzothiazol-5-yl]pyrimidin-2-yl]piperidine-4-carboxylic acid;
226) 4-ethyl-1-[5-[2-(ethylcarbamoylamino)-7-(5-methylpyrimidin-2-yl)-1,3-benzothiazol-5-yl]pyrimidin-2-yl]piperidine-4-carboxylic acid;
227) 1-[5-[2-(ethylcarbamoylamino)-5-pyrazol-1-yl-[1,2,4]triazolo[1,5-a]pyridin-7-yl]pyrimidin-2-yl]-4-methyl-piperidine-4-carboxylic acid;
228) 4-ethyl-1-[5-[2-(ethylcarbamoylamino)-7-pyrimidin-2-yl-1,3-benzothiazol-5-yl]pyrimidin-2-yl]piperidine-4-carboxylic acid;
229) 4-ethyl-1-[5-[2-(ethylcarbamoylamino)-7-(2-pyridyl)-1H-benzimidazol-5-yl]pyrimidin-2-yl]piperidine-4-carboxylic acid;
230) 4-ethyl-1-[5-[2-(ethylcarbamoylamino)-5-pyrazol-1-yl-imidazo[1,2-a]pyridin-7-yl]pyrimidin-2-yl]piperidine-4-carboxylic acid;
231) 1-[5-[2-(ethylcarbamoylamino)-5-pyrazol-1-yl-imidazo[1,2-a]pyridin-7-yl]pyrimidin-2-yl]-4-methyl-piperidine-4-carboxylic acid;
232) 4-ethyl-1-(3-{2-[(ethylcarbamoyl)amino]-7-(pyridin-2-yl)-1,3-benzothiazol-5-yl}-1,2,4-thiadiazol-5-yl)piperidine-4-carboxylic acid;
233) 1-(5-{2-[(ethylcarbamoyl)amino]-7-(pyridin-2-yl)-1,3-benzothiazol-5-yl}-1,2,4-thiadiazol-3-yl)-4-methylpiperidine-4-carboxylic acid; and
234) Diethyl 1,1′-({2-[(ethylcarbamoyl)amino]-1,3-benzoxazole-5,7-diyl}dipyrimidine-5,2-diyl)bis(4-ethylpiperidine-4-carboxylate); and
   and; salts, racemates, diastereomers, enantiomers, deuterated forms, hydrates, solvates and prodrugs thereof.

In one embodiment of Formula (I):

Alk, Ring A, X₁, X₂ and X₃ are as previously defined;

Z₁ may be an alcohol containing group of general formula (CH₂)ₛC(OH)(R₆)(R₇) or an ester, carbamate, phosphate, sulfate or prodrug thereof; wherein the OH, R₆ and R₇ groups are each attached to the same carbon atom; and

s may be an integer selected from 0, 1, 2 and 3. For example, s may be 0 or 1. In one example, s may be 0.

R₆ may be H or may be selected from optionally substituted C₁₋₆alkyl, optionally substituted C₂₋₆alkenyl, optionally substituted C₂₋₆alkynyl, optionally substituted (CH₂)ₜOC₁₋₆alkyl, optionally substituted (CH₂)ₜOC(═O)C₁₋₆alkyl, optionally substituted (CH₂)ₜSC₁₋₆alkyl, optionally substituted (CH₂)ₜS(═O)C₁₋₆alkyl, halo, optionally substituted haloC₁₋₃alkyl and optionally substituted (CH₂)ₜNR^{a}R^{b}. For example, R₆ may be H or optionally substituted C₁₋₃alkyl, such as, methyl or ethyl.

R₇ may be selected from optionally substituted C₁₋₆alkyl, optionally substituted C₂₋₆alkenyl, optionally substituted C₂₋₆alkynyl, optionally substituted C₃₋₇cycloalkyl ring, optionally substituted phenyl, optionally substituted 4-6-membered heterocyclyl ring, optionally substituted 5-6-membered heteroaryl ring, optionally substituted (CH₂)ₜOC₁₋₆alkyl, optionally substituted (CH₂)ₜOC(═O)C₁₋₆alkyl, optionally substituted (CH₂)ₜSC₁₋₆alkyl, optionally substituted (CH₂)ₜS(═O)C₁₋₆alkyl, halo, optionally substituted haloC₁₋₃alkyl and optionally substituted (CH₂)ₜNR^{a}R^{b}. For example, R₇ may be selected from optionally substituted C₁₋₃alkyl, such as methyl or ethyl, optionally substituted haloC₁₋₃alkyl, such as CHF₂, CH₂CHF₂, CF₃ or CH₂CF₃, optionally substituted C₃₋₇cycloalkyl ring, such as cyclopropyl, cyclobutyl, cyclopentyl or cyclohexyl), optionally substituted 4-6-membered heterocyclyl ring, such as morpholinyl, optionally substituted 5-6-membered heteroaryl ring. For example R₇ may be an imidazolyl or pyridinyl.

t may be an integer selected from 1, 2, 3, 4, 5 and 6. For example, t may be an integer selected from 1, 2 or 3.

or R₆ and R₇ together with the carbon atom to which they are attached form an optionally substituted 4-6-membered heterocyclic ring or C₃₋₇cycloalkyl ring;

and further wherein the prodrug may be selected from an ester, carbamate, phosphate or sulfate formed from the hydroxyl moiety.

Suitable optional substituents for R₆ and R₇ may include but are not limited to, for example, one or more, for example 1 or 2, substituents independently selected from OH, C₁₋₃alkyl such as methyl, haloC₁₋₃alkyl such as CHF₂ and CF₃, CO₂H, CO₂C₁₋₄alkyl, C₁₋₃alkoxyl such as methoxy, oxo (═O), NH₂, NHC₁₋₃alkyl and N(C₁₋₃alkyl)₂.

Examples of compounds according to this embodiment include but are not limited to, any one of compound examples 1 to 202 as previously disclosed in WO2013/138860:
1) 1-ethyl-3-[5-[2-(1-hydroxy-1-methyl-ethyl)pyrimidin-5-yl]-7-(2-pyridyl)-1,3-benzothiazol-2-yl]urea;
2) 1-ethyl-3-[7-[4-[(3-hydroxy-3-methyl-azetidin-1-yl)methyl]-2-pyridyl]-5-[2-(1-hydroxy-1-methyl-ethyl)pyrimidin-5-yl]-1,3-benzothiazol-2-yl]urea;
3) 1-ethyl-3-[5-[2-(1-hydroxy-1-methyl-ethyl)pyrimidin-5-yl]-6-(tetrahydrofuran-2-ylmethoxy)-1,3-benzothiazol-2-yl]urea;
4) 1-ethyl-3-[6-fluoro-5-[6-[hydroxy(3-pyridyl)methyl]-3-pyridyl]-1,3-benzothiazol-2-yl]urea;
5) 1-(2-hydroxyethyl)-3-[5-[2-(1-hydroxy-1-methyl-ethyl)pyrimidin-5-yl]-7-(2-pyridyl)-1,3-benzothiazol-2-yl]urea;
6) 1-ethyl-3-[5-[5-(1-hydroxyethyl)pyrazin-2-yl]-7-(2-pyridyl)-1,3-benzothiazol-2-yl]urea;
7) 1-[5-[2-[(1S*,2R*)-1,2-dihydroxypropyl]pyrimidin-5-yl]-7-(2-pyridyl)-1,3-benzothiazol-2-yl]-3-ethyl-urea (mixture 1-[5-[2-[(1S,2R)-1,2-dihydroxypropyl]pyrimidin-5-yl]-7-(2-pyridyl)-1,3-benzothiazol-2-yl]-3-ethyl-urea; and 1-[5-[2-[(1R,2S)-1,2-dihydroxypropyl]pyrimidin-5-yl]-7-(2-pyridyl)-1,3-benzothiazol-2-yl]-3-ethyl-urea);
8) 1-[5-[2-[(3R*,4S*)-3,4-dihydroxytetrahydropyran-4-yl]pyrimidin-5-yl]-7-(2-pyridyl)-1,3-benzothiazol-2-yl]-3-ethyl-urea (mixture 1-[5-[2-[(3R,4S)-3,4-dihydroxytetrahydropyran-4-yl]pyrimidin-5-yl]-7-(2-pyridyl)-1,3-benzothiazol-2-yl]-3-ethyl-urea; and 1-[5-[2-[(3S,4R)-3,4-dihydroxytetrahydropyran-4-yl]pyrimidin-5-yl]-7-(2-pyridyl)-1,3-benzothiazol-2-yl]-3-ethyl-urea);
9) 1-ethyl-3-[5-[4-(1-hydroxyethyl)triazol-1-yl]-7-(2-pyridyl)-1,3-benzothiazol-2-yl]urea;
10) 1-ethyl-3-[5-[2-(1-hydroxy-1-methyl-ethyl)pyrimidin-5-yl]-7-pyrimidin-2-yl-1,3-benzothiazol-2-yl]urea;
11) 1-ethyl-3-[5-[4-(1-hydroxy-1-methyl-ethyl)imidazol-1-yl]-7-(2-pyridyl)-1,3-benzothiazol-2-yl]urea;
12) 1-ethyl-3-[5-[2-(1-hydroxy-1-methyl-ethyl)pyrimidin-5-yl]-7-methoxy-1,3-benzothiazol-2-yl]urea;
13) 1-ethyl-3-[5-[2-(1-hydroxy-1-methyl-ethyl)pyrimidin-5-yl]-7-(methoxymethyl)-1,3-benzothiazol-2-yl]urea;
14) 1-ethyl-3-[5-[2-(1-hydroxyethyl)pyrimidin-5-yl]-7-[(6-methyl-3-pyridyl)methoxy]-1,3-benzothiazol-2-yl]urea;
15) 1-ethyl-3-[5-[2-(1-hydroxyethyl)pyrimidin-5-yl]-7-[4-(methylsulfanylmethyl)-2-pyridyl]-1,3-benzothiazol-2-yl]urea;
16) 1-ethyl-3-[5-[2-(1-hydroxyethyl)pyrimidin-5-yl]-7-[4-(methylsulfinylmethyl)-2-pyridyl]-1,3-benzothiazol-2-yl]urea;
17) 1-ethyl-3-[5-[2-(1-hydroxyethyl)pyrimidin-5-yl]-7-(2-pyridyl)-1,3-benzothiazol-2-yl]urea;
18) 1-[5-[2-(ethylcarbamoylamino)-7-(2-pyridyl)-1,3-benzothiazol-5-yl]pyrimidin-2-yl]ethyl 4-methylpiperazine-1-carboxylate;
19) 4-[1-[5-[2-(ethylcarbamoylamino)-7-(2-pyridyl)-1,3-benzothiazol-5-yl]pyrimidin-2-yl]ethoxy]-4-oxo-butanoic acid;
20) O4-[1-[5-[2-(ethylcarbamoylamino)-7-(2-pyridyl)-1,3-benzothiazol-5-yl]pyrimidin-2-yl]-1-methyl-ethyl] O1-methyl butanedioate;
21) 4-[1-[5-[2-(ethylcarbamoylamino)-7-(2-pyridyl)-1,3-benzothiazol-5-yl]pyrimidin-2-yl]-1-methyl-ethoxy]-4-oxo-butanoic acid;
22) 1-ethyl-3-[5-[2-[(1R)-1-hydroxyethyl]pyrimidin-5-yl]-7-(2-pyridyl)-1,3-benzothiazol-2-yl]urea;
23) 1-ethyl-3-[5-[2-[(1S)-1-hydroxyethyl]pyrimidin-5-yl]-7-(2-pyridyl)-1,3-benzothiazol-2-yl]urea;
24) 1-ethyl-3-[5-[6-[hydroxy-(1-methylimidazol-2-yl)methyl]-3-pyridyl]-7-(2-pyridyl)-1,3-benzothiazol-2-yl]urea;
25) 1-ethyl-3-[5-[2-(1-hydroxy-1-methyl-ethyl)pyrimidin-5-yl]-7-[5-(morpholinomethyl)-2-pyridyl]-1,3-benzothiazol-2-yl]urea;
26) 1-ethyl-3-[5-[2-(1-hydroxy-1-methyl-ethyl)pyrimidin-5-yl]-7-[4-(pyrrolidin-1-ylmethyl)-2-pyridyl]-1,3-benzothiazol-2-yl]urea;
27) 1-ethyl-3-[5-[6-(1-hydroxy-1-methyl-ethyl)-3-pyridyl]-7-(2-pyridyl)-1,3-benzothiazol-2-yl]urea;
28) 1-ethyl-3-[5-[2-(1-hydroxy-1-methyl-ethyl)pyrimidin-5-yl]-7-[4-(morpholinomethyl)-2-pyridyl]-1,3-benzothiazol-2-yl]urea;
29) 1-ethyl-3-[5-[2-(1-hydroxy-1-methyl-ethyl)pyrimidin-5-yl]-7-[4-[(3-hydroxypyrrolidin-1-yl)methyl]-2-pyridyl]-1,3-benzothiazol-2-yl]urea;
30) 1-ethyl-3-[7-[4-[(3-hydroxyazetidin-1-yl)methyl]-2-pyridyl]-5-[2-(1-hydroxy-1-methyl-ethyl)pyrimidin-5-yl]-1,3-benzothiazol-2-yl]urea;
31) 1-ethyl-3-[5-[2-(1-hydroxy-1-methyl-ethyl)pyrimidin-5-yl]-7-[4-[(3-methoxyazetidin-1-yl)methyl]-2-pyridyl]-1,3-benzothiazol-2-yl]urea;
32) 1-ethyl-3-[5-[2-(1-hydroxy-1-methyl-ethyl)pyrimidin-5-yl]-7-[4-(2-morpholinoethoxy)-2-pyridyl]-1,3-benzothiazol-2-yl]urea;
33) 1-ethyl-3-[5-[2-(1-hydroxy-1-methyl-ethyl)pyrimidin-5-yl]-7-[4-(1-morpholinoethyl)-2-pyridyl]-1,3-benzothiazol-2-yl]urea;
34) 1-[7-[4-[(3,3-difluoropyrrolidin-1-yl)methyl]-2-pyridyl]-5-[2-(1-hydroxy-1-methyl-ethyl)pyrimidin-5-yl]-1,3-benzothiazol-2-yl]-3-ethyl-urea;
35) 1-ethyl-3-[7-[4-[[(3S)-3-fluoropyrrolidin-1-yl]methyl]-2-pyridyl]-5-[2-(1-hydroxy-1-methyl-ethyl)pyrimidin-5-yl]-1,3-benzothiazol-2-yl]urea;
36) 1-ethyl-3-[7-[4-[[(3R)-3-fluoropyrrolidin-1-yl]methyl]-2-pyridyl]-5-[2-(1-hydroxy-1-methyl-ethyl)pyrimidin-5-yl]-1,3-benzothiazol-2-yl]urea;
37) 1-[7-[4-[(3,3-difluoro-1-piperidyl)methyl]-2-pyridyl]-5-[2-(1-hydroxy-1-methyl-ethyl)pyrimidin-5-yl]-1,3-benzothiazol-2-yl]-3-ethyl-urea;
38) 1-ethyl-3-[5-[2-(1-hydroxy-1-methyl-ethyl)pyrimidin-5-yl]-7-[4-(3-morpholinopropoxy)-2-pyridyl]-1,3-benzothiazol-2-yl]urea;
39) 1-ethyl-3-[5-[2-(1-hydroxy-1-methyl-ethyl)pyrimidin-5-yl]-7-pyrazin-2-yl-1,3-benzothiazol-2-yl]urea;
40) 1-[5-[2-(1,2-dihydroxyethyl)pyrimidin-5-yl]-7-(2-pyridyl)-1,3-benzothiazol-2-yl]-3-ethyl-urea;
41) 1-[7-(dimethylaminomethyl)-6-hydroxy-5-[6-(1-hydroxy-1-methyl-ethyl)-3-pyridyl]-1,3-benzothiazol-2-yl]-3-ethyl-urea;
42) 1-ethyl-3-[5-[2-(1-hydroxy-1-methyl-ethyl)pyrimidin-5-yl]-7-[4-[[(3R)-3-methoxypyrrolidin-1-yl]methyl]-2-pyridyl]-1,3-benzothiazol-2-yl]urea;
43) 1-ethyl-3-[5-[2-(1-hydroxy-1-methyl-ethyl)pyrimidin-5-yl]-7-(6-methylpyrimidin-4-yl)-1,3-benzothiazol-2-yl]urea;
44) 1-ethyl-3-[6-hydroxy-5-[6-(1-hydroxy-1-methyl-ethyl)-3-pyridyl]-7-(morpholinomethyl)-1,3-benzothiazol-2-yl]urea;
45) 1-[6-[2-(ethylcarbamoylamino)-5-[2-(1-hydroxy-1-methyl-ethyl)pyrimidin-5-yl]-1,3-benzothiazol-7-yl]-3-pyridyl]-4-methyl-piperidine-4-carboxylic acid;
46) 2-[6-[2-(ethylcarbamoylamino)-5-[2-(1-hydroxy-1-methyl-ethyl)pyrimidin-5-yl]-1,3-benzothiazol-7-yl]-3-pyridyl]acetic acid;
47) 1-ethyl-3-[5-[2-(1-hydroxycyclohexyl)pyrimidin-5-yl]-7-(2-pyridyl)-1,3-benzothiazol-2-yl]urea;
48) 1-ethyl-3-[5-[2-(1-hydroxy-1-methyl-ethyl)thiazol-5-yl]-7-(2-pyridyl)-1,3-benzothiazol-2-yl]urea;
49) 1-ethyl-3-[5-[5-(1-hydroxy-1-methyl-ethyl)pyrazin-2-yl]-7-(2-pyridyl)-1,3-benzothiazol-2-yl]urea;
50) 1-[2-[2-(ethylcarbamoylamino)-5-[2-(1-hydroxy-1-methyl-ethyl)pyrimidin-5-yl]-1,3-benzothiazol-7-yl]-4-pyridyl]-4-methyl-piperidine-4-carboxylic acid;
51) 1-[6-[2-(ethylcarbamoylamino)-5-[2-(1-hydroxy-1-methyl-ethyl)pyrimidin-5-yl]-1,3-benzothiazol-7-yl]-2-pyridyl]-4-methyl-piperidine-4-carboxylic acid;
52) 1-[4-[2-(ethylcarbamoylamino)-5-[2-(1-hydroxy-1-methyl-ethyl)pyrimidin-5-yl]-1,3-benzothiazol-7-yl]pyrimidin-2-yl]-4-methyl-piperidine-4-carboxylic acid;
53) 1-[7-[4-[(cyclopropylamino)methyl]-2-pyridyl]-5-[2-(1-hydroxy-1-methyl-ethyl)pyrimidin-5-yl]-1,3-benzothiazol-2-yl]-3-ethyl-urea;
54) 4-[[2-[2-(ethylcarbamoylamino)-5-[2-(1-hydroxy-1-methyl-ethyl)pyrimidin-5-yl]-1,3-benzothiazol-7-yl]-4-pyridyl]amino]-1-methyl-cyclohexanecarboxylic acid;
55) 1-ethyl-3-[5-[2-(1-hydroxyethyl)pyrimidin-5-yl]-7-[4-(morpholinomethyl)-2-pyridyl]-1,3-benzothiazol-2-yl]urea;
56) 1-ethyl-3-[5-[2-(1-hydroxy-1-methyl-ethyl)pyrimidin-5-yl]-7-[5-(2-morpholinoethoxy)-2-pyridyl]-1,3-benzothiazol-2-yl]urea;
57) 1-[5-[2-(1-hydroxy-1-methyl-ethyl)pyrimidin-5-yl]-7-(2-pyridyl)-1,3-benzothiazol-2-yl]-3-propyl-urea;
58) 1-[5-[2-[cyclopropyl(hydroxy)methyl]pyrimidin-5-yl]-7-(2-pyridyl)-1,3-benzothiazol-2-yl]-3-ethyl-urea;
59) 1-ethyl-3-[5-[2-(1-hydroxypropyl)pyrimidin-5-yl]-7-(2-pyridyl)-1,3-benzothiazol-2-yl]urea;
60) 1-ethyl-3-[5-[2-(1-hydroxy-2,2-dimethyl-propyl)pyrimidin-5-yl]-7-(2-pyridyl)-1,3-benzothiazol-2-yl]urea;
61) 1-ethyl-3-[5-[2-(1-hydroxybutyl)pyrimidin-5-yl]-7-(2-pyridyl)-1,3-benzothiazol-2-yl]urea;
62) [(1R)-1-[5-[2-(ethylcarbamoylamino)-7-(2-pyridyl)-1,3-benzothiazol-5-yl]pyrimidin-2-yl]ethyl] (2R)-2-amino-3-methyl-butanoate;
63) [(1S)-1-[5-[2-(ethylcarbamoylamino)-7-(2-pyridyl)-1,3-benzothiazol-5-yl]pyrimidin-2-yl]ethyl] (2R)-2-amino-3-methyl-butanoate;
64) 1-ethyl-3-[7-[4-[[(3S)-3-fluoropyrrolidin-1-yl]methyl]-2-pyridyl]-5-[2-(1-hydroxyethyl)pyrimidin-5-yl]-1,3-benzothiazol-2-yl]urea;
65) 1-ethyl-3-[7-[4-[(3-hydroxyazetidin-1-yl)methyl]-2-pyridyl]-5-[2-(1-hydroxyethyl)pyrimidin-5-yl]-1,3-benzothiazol-2-yl]urea;
66) 1-ethyl-3-[5-[2-(1-hydroxyethyl)pyrimidin-5-yl]-7-[4-[(3-hydroxy-3-methyl-azetidin-1-yl)methyl]-2-pyridyl]-1,3-benzothiazol-2-yl]urea;
67) 1-ethyl-3-[5-[2-(1-hydroxy-1-methyl-ethyl)pyrimidin-5-yl]-7-[4-[(3-hydroxy-3-methyl-pyrrolidin-1-yl)methyl]-2-pyridyl]-1,3-benzothiazol-2-yl]urea;
68) 1-ethyl-3-[5-[2-(1-hydroxyethyl)pyrimidin-5-yl]-7-[4-[(3-methylmorpholin-4-yl)methyl]-2-pyridyl]-1,3-benzothiazol-2-yl]urea;
69) 1-[7-[4-[[(2R,6S)-2,6-dimethylmorpholin-4-yl]methyl]-2-pyridyl]-5-[2-(1-hydroxyethyl)pyrimidin-5-yl]-1,3-benzothiazol-2-yl]-3-ethyl-urea;
70) 1-ethyl-3-[5-[2-(1-hydroxyethyl)pyrimidin-5-yl]-7-[4-[(2,2,3,3,5,5,6,6-octadeuteriomorpholin-4-yl)methyl]-2-pyridyl]-1,3-benzothiazol-2-yl]urea;
71) 1-[7-[4-[(2,5-dimethylmorpholin-4-yl)methyl]-2-pyridyl]-5-[2-(1-hydroxyethyl)pyrimidin-5-yl]-1,3-benzothiazol-2-yl]-3-ethyl-urea;
72) (2S)-1-[[2-[2-(ethylcarbamoylamino)-5-[2-(1-hydroxyethyl)pyrimidin-5-yl]-1,3-benzothiazol-7-yl]-4-pyridyl]methyl]pyrrolidine-2-carboxylic acid;
73) 1-ethyl-3-[5-[2-(1-hydroxyethyl)pyrimidin-5-yl]-7-[4-(6-oxa-2-azaspiro[3.3]heptan-2-ylmethyl)-2-pyridyl]-1,3-benzothiazol-2-yl]urea;
74) 1-[5-[2-(ethylcarbamoylamino)-7-[4-(1-hydroxy-1-methyl-ethyl)-2-pyridyl]-1,3-benzothiazol-5-yl]pyrimidin-2-yl]-4-methyl-piperidine-4-carboxylic acid;
75) 1-ethyl-3-[5-[2-(1-hydroxyethyl)pyrimidin-5-yl]-7-[4-(2-morpholinoethoxy)-2-pyridyl]-1,3-benzothiazol-2-yl]urea;
76) 1-ethyl-3-[5-[2-(1-hydroxyethyl)pyrimidin-5-yl]-7-[4-[(3-methoxyazetidin-1-yl)methyl]-2-pyridyl]-1,3-benzothiazol-2-yl]urea;
77) 1-ethyl-3-[5-[2-(1-hydroxyethyl)pyrimidin-5-yl]-7-[4-[[(3R)-3-hydroxypyrrolidin-1-yl]methyl]-2-pyridyl]-1,3-benzothiazol-2-yl]urea;
78) 1-ethyl-3-[5-[2-(1-hydroxy-1-methyl-ethyl)pyrimidin-5-yl]-7-[4-(2-methoxyethylamino)pyrimidin-2-yl]-1,3-benzothiazol-2-yl]urea;
79) 1-ethyl-3-[7-[4-(4-ethylpiperazin-1-yl)pyrimidin-2-yl]-5-[2-(1-hydroxy-1-methyl-ethyl)pyrimidin-5-yl]-1,3-benzothiazol-2-yl]urea;
80) 1-ethyl-3-[5-[2-(1-hydroxy-1-methyl-ethyl)pyrimidin-5-yl]-7-[4-(2-methoxyethoxy)-2-pyridyl]-1,3-benzothiazol-2-yl]urea;
81) 1-ethyl-3-[5-[2-(1-hydroxy-2-morpholino-ethyl)pyrimidin-5-yl]-7-(2-pyridyl)-1,3-benzothiazol-2-yl]urea;
82) 1-[5-[2-(1-hydroxy-1-methyl-ethyl)pyrimidin-5-yl]-7-(2-pyridyl)-1,3-benzothiazol-2-yl]-3-methyl-urea;
83) 1-[5-[2-(1-hydroxyethyl)pyrimidin-5-yl]-7-(2-pyridyl)-1,3-benzothiazol-2-yl]-3-methyl-urea;
84) 1-ethyl-3-[5-[2-(1-hydroxyethyl)pyrimidin-5-yl]-7-[4-[(3-hydroxy-3-methyl-pyrrolidin-1-yl)methyl]-2-pyridyl]-1,3-benzothiazol-2-yl]urea;
85) 1-ethyl-3-[7-[4-(2-hydroxyethylamino)pyrimidin-2-yl]-5-[2-(1-hydroxy-1-methyl-ethyl)pyrimidin-5-yl]-1,3-benzothiazol-2-yl]urea;
86) 1-ethyl-3-[7-[4-(3-hydroxy-3-methyl-azetidin-1-yl)pyrimidin-2-yl]-5-[2-(1-hydroxy-1-methyl-ethyl)pyrimidin-5-yl]-1,3-benzothiazol-2-yl]urea;
87) 1-[6-[2-(ethylcarbamoylamino)-5-[2-(1-hydroxy-1-methyl-ethyl)pyrimidin-5-yl]-1,3-benzothiazol-7-yl]pyrimidin-4-yl]-4-methyl-piperidine-4-carboxylic acid;
88) 1-[5-[2-(ethylcarbamoylamino)-7-[4-(1-hydroxyethyl)-2-pyridyl]-1,3-benzothiazol-5-yl]pyrimidin-2-yl]-4-methyl-piperidine-4-carboxylic acid;
89) [1-[5-[2-(ethylcarbamoylamino)-7-(2-pyridyl)-1,3-benzothiazol-5-yl]pyrimidin-2-yl]-1-methyl-ethyl] 4-methylpiperazine-1-carboxylate;
90) 1-ethyl-3-[5-[2-(1-hydroxyethyl)pyrimidin-5-yl]-7-(4-hydroxy-2-pyridyl)-1,3-benzothiazol-2-yl]urea;
91) 1-ethyl-3-[5-[2-(1-hydroxy-1-methyl-ethyl)pyrimidin-5-yl]-7-[4-(2-morpholinoethylamino)pyrimidin-2-yl]-1,3-benzothiazol-2-yl]urea;
92) 1-ethyl-3-[5-[2-(1-hydroxyethyl)pyrimidin-5-yl]-7-[4-(2-methoxyethoxy)-2-pyridyl]-1,3-benzothiazol-2-yl]urea;
93) 1-ethyl-3-[5-[2-(1-hydroxyethyl)pyrimidin-5-yl]-7-[4-(3-methoxyazetidin-1-yl)pyrimidin-2-yl]-1,3-benzothiazol-2-yl]urea;
94) 1-ethyl-3-[5-[2-(1-hydroxyethyl)pyrimidin-5-yl]-7-(4-morpholinopyrimidin-2-yl)-1,3-benzothiazol-2-yl]urea;
95) 1-ethyl-3-[5-[6-(1-hydroxyethyl)-3-pyridyl]-7-(2-pyridyl)-1,3-benzothiazol-2-yl]urea;
96) 1-ethyl-3-[7-(2-pyridyl)-5-[6-(2,2,2-trifluoro-1-hydroxy-ethyl)-3-pyridyl]-1,3-benzothiazol-2-yl]urea;
97) 1-[2-[2-(ethylcarbamoylamino)-5-[2-(1-hydroxy-1-methyl-ethyl)pyrimidin-5-yl]-1,3-benzothiazol-7-yl]pyrimidin-4-yl]-4-methyl-piperidine-4-carboxylic acid;
98) 1-ethyl-3-[5-[2-(1-ethyl-1-hydroxy-propyl)pyrimidin-5-yl]-7-(2-pyridyl)-1,3-benzothiazol-2-yl]urea;
99) 1-[5-[2-(ethylcarbamoylamino)-7-[5-(1-hydroxy-1-methyl-ethyl)-2-pyridyl]-1,3-benzothiazol-5-yl]pyrimidin-2-yl]-4-methyl-piperidine-4-carboxylic acid;
100) 1-[7-[4-(diethoxyphosphorylmethyl)-2-pyridyl]-5-[2-(1-hydroxyethyl)pyrimidin-5-yl]-1,3-benzothiazol-2-yl]-3-ethyl-urea;
101) 1-ethyl-3-[5-[2-(1-hydroxy-1-methyl-ethyl)pyrimidin-5-yl]-7-[4-(morpholinomethyl)pyrimidin-2-yl]-1,3-benzothiazol-2-yl]urea;
102) 1-ethyl-3-[5-[2-(1-hydroxy-1-methyl-ethyl)pyrimidin-5-yl]-7-[6-(morpholinomethyl)pyrazin-2-yl]-1,3-benzothiazol-2-yl]urea;
103) 1-ethyl-3-[5-[2-(1-hydroxy-1-methyl-ethyl)pyrimidin-5-yl]-7-[4-[(2-hydroxy-2-methyl-propyl)amino]pyrimidin-2-yl]-1,3-benzothiazol-2-yl]urea;
104) 1-ethyl-3-[5-[2-(1-hydroxyethyl)pyrimidin-5-yl]-7-[4-(tetrahydrofuran-2-ylmethoxy)-2-pyridyl]-1,3-benzothiazol-2-yl]urea;
105) 1-ethyl-3-[7-[4-(3-hydroxyazetidin-1-yl)pyrimidin-2-yl]-5-[2-(1-hydroxyethyl)pyrimidin-5-yl]-1,3-benzothiazol-2-yl]urea;
106) 1-ethyl-3-[7-(5-fluoro-4-morpholino-pyrimidin-2-yl)-5-[2-(1-hydroxyethyl)pyrimidin-5-yl]-1,3-benzothiazol-2-yl]urea;
107) 1-ethyl-3-[5-[2-(1-hydroxy-1-methyl-ethyl)pyrimidin-5-yl]-7-[4-[4-(2-methoxyethyl)piperazin-1-yl]pyrimidin-2-yl]-1,3-benzothiazol-2-yl]urea;
108) 1-ethyl-3-[7-[4-(morpholinomethyl)-2-pyridyl]-5-[6-(2,2,2-trifluoro-1-hydroxy-ethyl)-3-pyridyl]-1,3-benzothiazol-2-yl]urea;
109) 1-[6-[2-(ethylcarbamoylamino)-5-[2-(1-hydroxyethyl)pyrimidin-5-yl]-1,3-benzothiazol-7-yl]pyrimidin-4-yl]-4-methyl-piperidine-4-carboxylic acid;
110) 1-[2-[2-(ethylcarbamoylamino)-5-[2-(1-hydroxy-1-methyl-ethyl)pyrimidin-5-yl]-1,3-benzothiazol-7-yl]-4-pyridyl]piperidine-4-carboxylic acid;
111) 1-ethyl-3-[5-[2-(4-hydroxytetrahydropyran-4-yl)pyrimidin-5-yl]-7-(2-pyridyl)-1,3-benzothiazol-2-yl]urea;
112) 1-ethyl-3-[5-[2-(1-hydroxyethyl)pyrimidin-5-yl]-7-[4-(2-oxa-7-azaspiro[3.5]nonan-7-ylmethyl)-2-pyridyl]-1,3-benzothiazol-2-yl]urea;
113) 1-ethyl-3-[5-[2-(3-hydroxyoxetan-3-yl)pyrimidin-5-yl]-7-(2-pyridyl)-1,3-benzothiazol-2-yl]urea;
114) 1-ethyl-3-[5-[2-(1-hydroxy-1-methyl-ethyl)pyrimidin-5-yl]-7-[4-[(3-methoxy-3-methyl-azetidin-1-yl)methyl]-2-pyridyl]-1,3-benzothiazol-2-yl]urea;
115) 1-ethyl-3-[5-[2-(1-hydroxyethyl)pyrimidin-5-yl]-7-pyrimidin-2-yl-1,3-benzothiazol-2-yl]urea;
116) 1-ethyl-3-[7-[4-(2-morpholinoethoxy)-2-pyridyl]-5-[6-(2,2,2-trifluoro-1-hydroxy-ethyl)-3-pyridyl]-1,3-benzothiazol-2-yl]urea;
117) 1-[2-[2-(ethylcarbamoylamino)-5-[2-(1-hydroxyethyl)pyrimidin-5-yl]-1,3-benzothiazol-7-yl]pyrimidin-4-yl]-4-methyl-piperidine-4-carboxylic acid;
118) 1-[5-[2-[(1R*,2R*)-1,2-dihydroxypropyl]pyrimidin-5-yl]-7-(2-pyridyl)-1,3-benzothiazol-2-yl]-3-ethyl-urea (mixture 1-[5-[2-[(1R,2R)-1,2-dihydroxypropyl]pyrimidin-5-yl]-7-(2-pyridyl)-1,3-benzothiazol-2-yl]-3-ethyl-urea; and
1-[5-[2-[(1S,2S)-1,2-dihydroxypropyl]pyrimidin-5-yl]-7-(2-pyridyl)-1,3-benzothiazol-2-yl]-3-ethyl-urea);
119) 1-ethyl-3-[5-[2-(1-hydroxycyclopentyl)pyrimidin-5-yl]-7-(2-pyridyl)-1,3-benzothiazol-2-yl]urea;
120) 1-ethyl-3-[5-[2-(1-hydroxy-1-methyl-ethyl)pyrimidin-5-yl]-7-[5-(morpholinomethyl)pyrimidin-2-yl]-1,3-benzothiazol-2-yl]urea;
121) 1-ethyl-3-[5-[2-[(1R)-1-hydroxyethyl]pyrimidin-5-yl]-7-[4-(morpholinomethyl)-2-pyridyl]-1,3-benzothiazol-2-yl]urea;
122) 1-ethyl-3-[5-[2-[(1S)-1-hydroxyethyl]pyrimidin-5-yl]-7-[4-(morpholinomethyl)-2-pyridyl]-1,3-benzothiazol-2-yl]urea;
123) 4-[3-[5-[2-(ethylcarbamoylamino)-7-(2-pyridyl)-1,3-benzothiazol-5-yl]pyrimidin-2-yl]oxetan-3-yl]oxy-4-oxo-butanoic acid;
124) 4-[2-[5-[2-(ethylcarbamoylamino)-7-(2-pyridyl)-1,3-benzothiazol-5-yl]pyrimidin-2-yl]-2-hydroxy-propoxy]-4-oxo-butanoic acid;
125) 1-ethyl-3-[5-[2-(4-hydroxytetrahydropyran-4-yl)pyrimidin-5-yl]-7-[4-(morpholinomethyl)-2-pyridyl]-1,3-benzothiazol-2-yl]urea;
126) 1-[5-[2-(ethylcarbamoylamino)-7-(2-pyridyl)-1,3-benzothiazol-5-yl]pyrimidin-2-yl]ethyl 2-aminoacetate;
127) 1-ethyl-3-[5-[2-(4-hydroxytetrahydrothiopyran-4-yl)pyrimidin-5-yl]-7-(2-pyridyl)-1,3-benzothiazol-2-yl]urea;
128) 1-ethyl-3-[5-[2-(4-hydroxy-1-methyl-4-piperidyl)pyrimidin-5-yl]-7-(2-pyridyl)-1,3-benzothiazol-2-yl]urea;
129) [1-[5-[2-(ethylcarbamoylamino)-7-(2-pyridyl)-1,3-benzothiazol-5-yl]pyrimidin-2-yl]-1-methyl-ethyl] 2-(2-aminoethylamino)acetate;
130) 1-[5-[2-[(1R*,2S*)-3,3-difluoro-1,2-dihydroxy-propyl]pyrimidin-5-yl]-7-(2-pyridyl)-1,3-benzothiazol-2-yl]-3-ethyl-urea (mixture 1-[5-[2-[(1R,2S)-3,3-difluoro-1,2-dihydroxy-propyl]pyrimidin-5-yl]-7-(2-pyridyl)-1,3-benzothiazol-2-yl]-3-ethyl-urea; and
1-[5-[2-[(1S,2R)-3,3-difluoro-1,2-dihydroxy-propyl]pyrimidin-5-yl]-7-(2-pyridyl)-1,3-benzothiazol-2-yl]-3-ethyl-urea);
131) 1-ethyl-3-[7-(2-pyridyl)-5-[2-[(1R*,2S*)-3,3,3-trifluoro-1,2-dihydroxy-propyl]pyrimidin-5-yl]-1,3-benzothiazol-2-yl]urea (mixture 1-ethyl-3-[7-(2-pyridyl)-5-[2-[(1R,2S)-3,3,3-trifluoro-1,2-dihydroxy-propyl]pyrimidin-5-yl]-1,3-benzothiazol-2-yl]urea; and 1-ethyl-3-[7-(2-pyridyl)-5-[2-[(1S,2R)-3,3,3-trifluoro-1,2-dihydroxy-propyl]pyrimidin-5-yl]-1,3-benzothiazol-2-yl]urea);
132) [1-[5-[2-(ethylcarbamoylamino)-7-(2-pyridyl)-1,3-benzothiazol-5-yl]pyrimidin-2-yl]-1-methyl-ethyl] (2S)-2-aminopropanoate;
133) 4-[(1S)-1-[5-[2-(ethylcarbamoylamino)-7-(2-pyridyl)-1,3-benzothiazol-5-yl]pyrimidin-2-yl]ethoxy]-4-oxo-butanoic acid;
134) 4-[(1R)-1-[5-[2-(ethylcarbamoylamino)-7-(2-pyridyl)-1,3-benzothiazol-5-yl]pyrimidin-2-yl]ethoxy]-4-oxo-butanoic acid;
135) 1-[5-[2-(1,2-dihydroxy-1-methyl-ethyl)pyrimidin-5-yl]-7-[4-(morpholinomethyl)-2-pyridyl]-1,3-benzothiazol-2-yl]-3-ethyl-urea;
136) 1-[5-[2-(ethylcarbamoylamino)-7-(2-pyridyl)-1,3-benzothiazol-5-yl]pyrimidin-2-yl]ethyl 2-amino-2-methyl-propanoate;
137) 1-[5-[2-(ethylcarbamoylamino)-7-(2-pyridyl)-1,3-benzothiazol-5-yl]pyrimidin-2-yl]ethyl 3-aminopropanoate;
138) tert-butyl 4-[5-[2-(ethylcarbamoylamino)-7-(2-pyridyl)-1,3-benzothiazol-5-yl]pyrimidin-2-yl]-4-hydroxy-piperidine-1-carboxylate;
139) 1-ethyl-3-[5-[2-(4-hydroxy-1-oxo-thian-4-yl)pyrimidin-5-yl]-7-(2-pyridyl)-1,3-benzothiazol-2-yl]urea;
140) [1-[5-[2-(ethylcarbamoylamino)-7-(2-pyridyl)-1,3-benzothiazol-5-yl]pyrimidin-2-yl]-1-methyl-ethyl] 2-(dimethylamino)acetate;
141) [1-[5-[2-(ethylcarbamoylamino)-7-(2-pyridyl)-1,3-benzothiazol-5-yl]pyrimidin-2-yl]-1-methyl-ethyl] 2-morpholinoacetate;
142) 1-[7-[4-[[(2R,6S)-2,6-dimethylmorpholin-4-yl]methyl]-2-pyridyl]-5-[2-[(1R)-1-hydroxyethyl]pyrimidin-5-yl]-1,3-benzothiazol-2-yl]-3-ethyl-urea;
143) 1-[7-[4-[[(2R,6S)-2,6-dimethylmorpholin-4-yl]methyl]-2-pyridyl]-5-[2-[(1S)-1-hydroxyethyl]pyrimidin-5-yl]-1,3-benzothiazol-2-yl]-3-ethyl-urea;
144) [1-[5-[2-(ethylcarbamoylamino)-7-(2-pyridyl)-1,3-benzothiazol-5-yl]pyrimidin-2-yl]-1-methyl-ethyl] 5-aminopentanoate;
145) [1-[5-[2-(ethylcarbamoylamino)-7-(2-pyridyl)-1,3-benzothiazol-5-yl]pyrimidin-2-yl]-1-methyl-ethyl] 5-(dimethylamino)pentanoate;
146) [1-[5-[2-(ethylcarbamoylamino)-7-(2-pyridyl)-1,3-benzothiazol-5-yl]pyrimidin-2-yl]-1-methyl-ethyl] 2-aminoacetate;
147) 1-[7-[4-[(3,3-difluoroazetidin-1-yl)methyl]-2-pyridyl]-5-[2-(1-hydroxy-1-methyl-ethyl)pyrimidin-5-yl]-1,3-benzothiazol-2-yl]-3-ethyl-urea;
148) 1-[7-[4-[[(2R,6S)-2,6-dimethylmorpholin-4-yl]methyl]-2-pyridyl]-5-[2-(3-hydroxyoxetan-3-yl)pyrimidin-5-yl]-1,3-benzothiazol-2-yl]-3-ethyl-urea;
149) 1-[5-[2-(ethylcarbamoylamino)-7-(2-pyridyl)-1,3-benzothiazol-5-yl]pyrimidin-2-yl]ethyl dihydrogen phosphate;
150) 1-ethyl-3-[5-[2-(1-hydroxy-1-methyl-ethyl)pyrimidin-5-yl]-6-(2-methoxyethylamino)-1,3-benzothiazol-2-yl]urea;
151) 1-ethyl-3-[5-[2-(1-hydroxy-1-methyl-ethyl)pyrimidin-5-yl]-7-morpholino-1,3-benzothiazol-2-yl]urea;
152) [(1R)-1-[5-[2-(ethylcarbamoylamino)-7-(2-pyridyl)-1,3-benzothiazol-5-yl]pyrimidin-2-yl]ethyl] dihydrogen phosphate;
153) [(1S)-1-[5-[2-(ethylcarbamoylamino)-7-(2-pyridyl)-1,3-benzothiazol-5-yl]pyrimidin-2-yl]ethyl] dihydrogen phosphate;
154) 1-ethyl-3-[5-[2-(3-hydroxyoxetan-3-yl)pyrimidin-5-yl]-7-[4-(morpholinomethyl)-2-pyridyl]-1,3-benzothiazol-2-yl]urea;
155) 1-ethyl-3-[7-[4-[(3-methoxyazetidin-1-yl)methyl]-2-pyridyl]-5-[6-[2,2,2-trifluoro-1-hydroxy-ethyl]-3-pyridyl]-1,3-benzothiazol-2-yl]urea;
156) 1-[7-[4-[(4,4-difluoro-1-piperidyl)methyl]-2-pyridyl]-5-[2-(1-hydroxy-1-methyl-ethyl)pyrimidin-5-yl]-1,3-benzothiazol-2-yl]-3-ethyl-urea;
157) 1-[5-[2-[1,2-dihydroxy-1-methyl-ethyl]pyrimidin-5-yl]-7-(2-pyridyl)-1,3-benzothiazol-2-yl]-3-ethyl-urea;
158) 1-ethyl-3-[5-[2-[1-hydroxyethyl]pyrimidin-5-yl]-7-(5-methyl-2-pyridyl)-1,3-benzothiazol-2-yl]urea;
159) 1-ethyl-3-[5-[2-(1-hydroxy-1-methyl-ethyl)pyrimidin-5-yl]-7-[4-[(4-methylpiperazin-1-yl)methyl]-2-pyridyl]-1,3-benzothiazol-2-yl]urea;
160) [(1S)-1-[5-[2-(ethylcarbamoylamino)-7-(2-pyridyl)-1,3-benzothiazol-5-yl]pyrimidin-2-yl]ethyl] (2S)-pyrrolidine-2-carboxylate;
161) [(1R)-1-[5-[2-(ethylcarbamoylamino)-7-(2-pyridyl)-1,3-benzothiazol-5-yl]pyrimidin-2-yl]ethyl] (2S)-pyrrolidine-2-carboxylate;
162) tert-butyl 3-[5-[2-(ethylcarbamoylamino)-7-(2-pyridyl)-1,3-benzothiazol-5-yl]pyrimidin-2-yl]-3-hydroxy-azetidine-1-carboxylate;
163) 1-ethyl-3-[5-[2-(3-hydroxyazetidin-3-yl)pyrimidin-5-yl]-7-(2-pyridyl)-1,3-benzothiazol-2-yl]urea;
164) 1-[7-[4-[[(2R,6S)-2,6-dimethylmorpholin-4-yl]methyl]-2-pyridyl]-5-[2-(1-hydroxy-1-methyl-ethyl)pyrimidin-5-yl]-1,3-benzothiazol-2-yl]-3-ethyl-urea;
165) [1-[5-[2-(ethylcarbamoylamino)-7-(2-pyridyl)-1,3-benzothiazol-5-yl]pyrimidin-2-yl]-1-methyl-ethyl] 2-[[(2S)-pyrrolidine-2-carbonyl]amino]acetate;
166) [1-[5-[2-(ethylcarbamoylamino)-7-(2-pyridyl)-1,3-benzothiazol-5-yl]pyrimidin-2-yl]-1-methyl-ethyl] (2S)-pyrrolidine-2-carboxylate;
167) 4-[3-[5-[7-[4-[[(2R,6S)-2,6-dimethylmorpholin-4-yl]methyl]-2-pyridyl]-2-(ethylcarbamoylamino)-1,3-benzothiazol-5-yl]pyrimidin-2-yl]oxetan-3-yl]oxy-4-oxo-butanoic acid;
168) 1-ethyl-3-[7-[5-(1-hydroxyethyl)-2-pyridyl]-5-[2-(1-hydroxyethyl)pyrimidin-5-yl]-1,3-benzothiazol-2-yl]urea;
169) [1-[5-[2-(ethylcarbamoylamino)-7-(2-pyridyl)-1,3-benzothiazol-5-yl]pyrimidin-2-yl]-1-methyl-ethyl] 2-(2-morpholinoethylamino)acetate;
170) 2-[[2-[1-[5-[2-(ethylcarbamoylamino)-7-(2-pyridyl)-1,3-benzothiazol-5-yl]pyrimidin-2-yl]-1-methyl-ethoxy]-2-oxo-ethyl]amino]acetic acid;
171) (2S)-2-amino-4-[1-[5-[2-(ethylcarbamoylamino)-7-(2-pyridyl)-1,3-benzothiazol-5-yl]pyrimidin-2-yl]-1-methyl-ethoxy]-4-oxo-butanoic acid;
172) [1-[5-[2-(ethylcarbamoylamino)-7-(2-pyridyl)-1,3-benzothiazol-5-yl]pyrimidin-2-yl]-1-methyl-ethyl] 4-aminobutanoate;
173) 1-ethyl-3-[5-[2-(4-hydroxy-1,1-dioxo-thian-4-yl)pyrimidin-5-yl]-7-(2-pyridyl)-1,3-benzothiazol-2-yl]urea;
174) 1-ethyl-3-[5-[2-(4-hydroxy-4-piperidyl)pyrimidin-5-yl]-7-(2-pyridyl)-1,3-benzothiazol-2-yl]urea;
175) 1-[7-[4-[(3-ethoxyazetidin-1-yl)methyl]-2-pyridyl]-5-[2-(1-hydroxy-1-methyl-ethyl)pyrimidin-5-yl]-1,3-benzothiazol-2-yl]-3-ethyl-urea;
176) 1-ethyl-3-[5-[2-(1-hydroxy-1-methyl-ethyl)pyrimidin-5-yl]-6-methoxy-1,3-benzothiazol-2-yl]urea;
177) 1-ethyl-3-[6-fluoro-5-[2-(1-hydroxy-1-methyl-ethyl)pyrimidin-5-yl]-1,3-benzothiazol-2-yl]urea;
178) 1-ethyl-3-[7-(3-fluoro-4-methoxy-2-pyridyl)-5-[2-[1-hydroxyethyl]pyrimidin-5-yl]-1,3-benzothiazol-2-yl]urea;
179) 3-[[2-[1-[5-[2-(ethylcarbamoylamino)-7-(2-pyridyl)-1,3-benzothiazol-5-yl]pyrimidin-2-yl]-1-methyl-ethoxy]-2-oxo-ethyl]amino]propanoic acid;
180) [1-[5-[2-(ethylcarbamoylamino)-7-(2-pyridyl)-1,3-benzothiazol-5-yl]pyrimidin-2-yl]-1-methyl-ethyl] (3R)-pyrrolidine-3-carboxylate;
181) [1-[5-[2-(ethylcarbamoylamino)-7-(2-pyridyl)-1,3-benzothiazol-5-yl]pyrimidin-2-yl]-1-methyl-ethyl] (3R)-morpholine-3-carboxylate;
182) 1-[6-(cyclopropylmethoxy)-5-[2-(1-hydroxy-1-methyl-ethyl)pyrimidin-5-yl]-1,3-benzothiazol-2-yl]-3-ethyl-urea;
183) 1-ethyl-3-[5-[2-(1-hydroxy-1-methyl-ethyl)pyrimidin-5-yl]-6-(2-methoxyethoxy)-1,3-benzothiazol-2-yl]urea;
184) [1-[5-[2-(ethylcarbamoylamino)-7-(2-pyridyl)-1,3-benzothiazol-5-yl]pyrimidin-2-yl]-1-methyl-ethyl] 2-(2-phosphonooxyethylamino)acetate;
185) 1-ethyl-3-[5-[2-[1-hydroxyethyl]pyrimidin-5-yl]-7-[4-(thiomorpholinomethyl)-2-pyridyl]-1,3-benzothiazol-2-yl]urea;
186) 1-ethyl-3-[6-(2-hydroxyethoxy)-5-[2-(1-hydroxy-1-methyl-ethyl)pyrimidin-5-yl]-1,3-benzothiazol-2-yl]urea;
187) 1-ethyl-3-[5-[2-(1-hydroxy-1-methyl-ethyl)pyrimidin-5-yl]-7-[4-(thiomorpholinomethyl)-2-pyridyl]-1,3-benzothiazol-2-yl]urea;
188) 1-ethyl-3-[5-[2-(1-hydroxy-1-methyl-ethyl)pyrimidin-5-yl]-7-[4-[(1-oxo-1,4-thiazinan-4-yl)methyl]-2-pyridyl]-1,3-benzothiazol-2-yl]urea;
189) 1-ethyl-3-[5-[2-(1-hydroxy-1-methyl-ethyl)pyrimidin-5-yl]-6-[2-methoxyethyl(methyl)amino]-1,3-benzothiazol-2-yl]urea;
190) [1-[5-[2-(ethylcarbamoylamino)-7-(2-pyridyl)-1,3-benzothiazol-5-yl]pyrimidin-2-yl]-1-methyl-ethyl] dihydrogen phosphate;
191) 1-[7-[4-[(1,1-dioxo-1,4-thiazinan-4-yl)methyl]-2-pyridyl]-5-[2-(1-hydroxy-1-methyl-ethyl)pyrimidin-5-yl]-1,3-benzothiazol-2-yl]-3-ethyl-urea;
192) 1-[6-[(3,4-dimethoxyphenyl)methoxy]-5-[2-(1-hydroxy-1-methyl-ethyl)pyrimidin-5-yl]-1,3-benzothiazol-2-yl]-3-ethyl-urea;
193) 1-ethyl-3-[5-[2-[1-hydroxyethyl]pyrimidin-5-yl]-6-(tetrahydrofuran-2-ylmethoxy)-1,3-benzothiazol-2-yl]urea;
194) 1-ethyl-3-[5-[2-[1-hydroxyethyl]pyrimidin-5-yl]-6-morpholino-1,3-benzothiazol-2-yl]urea;
195) 1-[7-[(3S)-3-aminopyrrolidin-1-yl]-5-[2-(1-hydroxy-1-methyl-ethyl)pyrimidin-5-yl]-1,3-benzothiazol-2-yl]-3-ethyl-urea;
196) 1-ethyl-3-[7-[4-[(2-hydroxyethylamino)methyl]-2-pyridyl]-5-[2-(1-hydroxy-1-methyl-ethyl)pyrimidin-5-yl]-1,3-benzothiazol-2-yl]urea;
197) 1-ethyl-3-[5-[5-(1-hydroxyethyl)-3-pyridyl]-7-(2-pyridyl)-1,3-benzothiazol-2-yl]urea;
198) 1-ethyl-3-[5-[2-(1-hydroxy-1-methyl-ethyl)pyrimidin-5-yl]-7-[4-[(2,2,3,3,5,5,6,6-octadeuteriomorpholin-4-yl)methyl]-2-pyridyl]-1,3-benzothiazol-2-yl]urea;
199) 1-ethyl-3-[5-[2-(1-hydroxy-1-methyl-ethyl)pyrimidin-5-yl]-6-(2-morpholinoethoxy)-1,3-benzothiazol-2-yl]urea;
200) 1-ethyl-3-[5-[2-(1-hydroxy-1-methyl-ethyl)pyrimidin-5-yl]-6-(2-methoxyethylsulfanyl)-1,3-benzothiazol-2-yl]urea;
201) 1-ethyl-3-[5-[2-(1-hydroxy-1-methyl-ethyl)pyrimidin-5-yl]-6-(2-methoxyethylsulfinyl)-1,3-benzothiazol-2-yl]urea;
202) 1-ethyl-3-[5-[2-[(1R)-1-hydroxyethyl]pyrimidin-5-yl]-7-(5-methoxy-2-pyridyl)-1,3-benzothiazol-2-yl]urea; and
   and; salts, racemates, diastereomers, enantiomers, deuterated forms, hydrates, solvates and prodrugs thereof.

Examples of compounds of Formula (I) wherein Z₁ is a sulfonamide containing group of general formula NRS(═O)₂R₈ or S(═O)₂NR₉R₁₀ or a sulfamide containing group of general formula NRS(═O)₂NR₉R₁₀ are believed to be novel.

Accordingly, in one embodiment, there is provided compound of Formula (II):

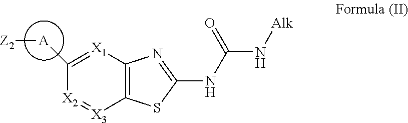

and; salts, racemates, diastereomers, enantiomers, deuterated forms, hydrates, solvates and prodrugs thereof;

wherein Alk, Ring A, X₁, X₂ and X₃ are as previously defined according to Formula (I) and embodiments thereof; and

Z₂ may be (CH₂)ᵥNRS(═O)₂R₈, (CH₂)ᵥS(═O)₂NR₉R₁₀ or (CH₂)ᵥNRS(═O)₂NR₉R₁₀; wherein

v is an integer 0, 1, 2 or 3;

R is H or an optionally substituted C₁₋₆alkyl; and

R₈, R₉ and R₁₀ may each be independently selected from H, C₁₋₆alkyl, C₂₋₆alkenyl, C₂₋₆alkynyl, C₃₋₇cycloalkyl, phenyl, benzyl, a 3-10-membered heterocyclic ring, a 5-10-membered heteroaryl ring and further wherein each C₁₋₆alkyl, C₂₋₆alkenyl, C₂₋₆alkynyl, C₃₋₇cycloalkyl, phenyl, benzyl, a 3-10-membered heterocyclic ring, a 5-10-membered heteroaryl ring may be optionally substituted;

or R₉ and R₁₀ may join to form an optionally substituted 3-6-membered heterocyclic ring together with the nitrogen to which they are attached.

Optional substituents for R₈, R₉ and R₁₀ may include but are not limited to one or more substituents independently selected from halo (for example Cl, Br, F, I), C₁₋₄alkyl (for example methyl, ethyl, propyl, iso-propyl, n-butyl, sec-butyl, tert-butyl), C₂₋₄alkenyl (for example ethenyl, propenyl, butenyl), C₂₋₄alkynyl (for example ethynyl, propynyl, butynyl), C₁₋₄alkylhalo (for example CH₂F, CF₃), OH, OC₁₋₄alkyl (for example OCH₃, OCH₂CH₃), C₁₋₄alkoxyl (for example CH₂OCH₃, CH₂CH₂OCH₃), C₁₋₄alkoxylhalo (for example OCH₂F, OCF₃), CN, NH₂, NH(C₁₋₄alkyl) (for example NHCH₃, NHCH₂CH₃), N(C₁₋₄alkyl)₂ (for example N(CH₃)₂, N(CH₃)CH₂CH₃, N(CH₂CH₃)₂), C(═O)OC₁₋₄alkyl (for example C(═O)OCH₃, C(═O)OCH₂CH₃), C₃₋₆ cycloalkyl (for example cyclopropyl, cyclobutyl, cyclopentyl, cyclohexyl), phenyl, benzyl, a 3-6-membered heterocyclic ring containing N and/or O heteroatoms (for example aziridine, oxirane, pyrrolidine, pyrazoline, imidazoline, pyrazolidine, imidazolidine, tetrahydrofuran, piperidine, tetrahydropyran, piperizine, morphline), or a 5-10-membered heteroaryl ring containing one or more N, O and/or S heteroatoms (for example 5-membered heteroaryl rings such as pyrrole, furan, thiophene, pyrazole, imidazole, oxazole, isoxazole, thiazole, isothiazole, triazole, oxadiazole, furazan, thiadiazole, tetrazole, and 6-membered heteroaryl rings such as pyridine, pyridazine, pyrimidine and pyrazine).

In one embodiment of Formula (II) Z₂ is NRS(═O)₂R₈.

In another embodiment of Formula (II) Z₂ is S(═O)₂NR₉R₁₀.

In another embodiment of Formula (II) Z₂ is NRS(═O)₂NR₉R₁₀.

Examples of compounds of Formula (II), include but is not limited to, any one of compound examples A-10 to A-21, A-24 to A-100, A-103 to A-107, A-110, A-111, and A-113 to A-115, as described in the Examples section which follows:
A-10) 2-[[5-[2-(Ethylcarbamoylamino)-7-(2-pyridyl)-1,3-benzothiazol-5-yl]pyrimidin-2-yl]sulfamoyl]benzoic acid;
A-11) 1-Ethyl-3-[7-(5-methyl-2-pyridyl)-5-[2-(pyrrolidin-1-ylsulfonylamino)pyrimidin-5-yl]-1,3-benzothiazol-2-yl]urea;
A-12) 1-Ethyl-3-[7-(5-methyl-2-pyridyl)-5-[2-(propylsulfonylamino)pyrimidin-5-yl]-1,3-benzothiazol-2-yl]urea;
A-13) 1-[5-[2-(tert-Butylsulfonylamino)pyrimidin-5-yl]-7-(5-methyl-2-pyridyl)-1,3-benzothiazol-2-yl]-3-ethyl-urea;
A-14) 1-Ethyl-3-[5-[2-[(2-hydroxy-1,1-dimethyl-ethyl)sulfonylamino]pyrimidin-5-yl]-7-(5-methyl-2-pyridyl)-1,3-benzothiazol-2-yl]urea;
A-15) 1-Ethyl-3-[5-[2-[[2-hydroxyethyl(methyl)sulfamoyl]amino]pyrimidin-5-yl]-7-(5-methyl-2-pyridyl)-1,3-benzothiazol-2-yl]urea;
A-16) Methyl 2-[[5-[2-(ethylcarbamoylamino)-7-(5-methyl-2-pyridyl)-1,3-benzothiazol-5-yl]pyrimidin-2-yl]sulfamoyl]acetate;
A-17) 1-[5-[2-(allylsulfonylamino)pyrimidin-5-yl]-7-(5-methyl-2-pyridyl)-1,3-benzothiazol-2-yl]-3-ethyl-urea;
A-18) 1-[5-[2-(tert-butylsulfonylamino)pyrimidin-5-yl]-7-[2-(dimethylamino)pyrimidin-5-yl]-1,3-benzothiazol-2-yl]-3-ethyl-urea;
A-19) 1-ethyl-3-[5-[2-(methanesulfonamidomethyl)pyrimidin-5-yl]-7-(5-methyl-2-pyridyl)-1,3-benzothiazol-2-yl]urea;
A-20) 1-[5-[2-(tert-butylsulfonylamino)pyrimidin-5-yl]-7-[4-(2-pyrrolidin-1-ylethyl)piperazin-1-yl]-1,3-benzothiazol-2-yl]-3-ethyl-urea;
A-21) 1-[5-[2-(cyclopentylsulfonylamino)pyrimidin-5-yl]-7-(5-methyl-2-pyridyl)-1,3-benzothiazol-2-yl]-3-ethyl-urea;
A-24) 1-(5-(2-(1,1-dioxido-1,2-thiazinan-2-yl)pyrimidin-5-yl)-7-(pyridin-2-yl)benzo[d]thiazol-2-yl)-3-ethylurea;
A-25) 1-[5-[6-(1,1-dioxo-1,2-thiazolidin-2-yl)-3-pyridyl]-7-(2-pyridyl)-1,3-benzothiazol-2-yl]-3-ethylurea;
A-26) 1-(5-(2-(1,1-dioxidoisothiazolidin-2-yl)pyrimidin-5-yl)-7-(pyridin-2-yl)benzo[d]thiazol-2-yl)-3-ethylurea;
A-27) 1-(5-(2-(1,1-dioxidoisothiazolidin-2-yl)pyrimidin-5-yl)-6-((tetrahydrofuran-2-yl)methoxy)benzo[d]thiazol-2-yl)-3-ethylurea;
A-28) 1-(5-(2-(1,1-dioxidoisothiazolidin-2-yl)pyrimidin-5-yl)-7-(5-methylpyridin-2-yl)benzo[d]thiazol-2-yl)-3-ethylurea;
A-29) N-(5-(2-(3-ethylureido)-7-(5-methylpyridin-2-yl)benzo[d]thiazol-5-yl)pyrimidin-2-yl)-N-methylmethanesulfonamide;
A-30) N-(5-(2-(3-ethylureido)-7-(5-methylpyridin-2-yl)benzo[d]thiazol-5-yl)pyrimidin-2-yl)methanesulfonamide;
A-31) N-(5-(2-(3-ethylureido)-7-(pyridin-2-yl)benzo[d]thiazol-5-yl)pyrimidin-2-yl)methanesulfonamide;
A-32) 1-[5-[2-(dimethylsulfamoylamino)pyrimidin-5-yl]-7-(2-pyridyl)-1,3-benzothiazol-2-yl]-3-ethyl urea;
A-33) N-(5-(2-(3-ethylureido)-7-(pyridin-2-yl)benzo[d]thiazol-5-yl)pyrimidin-2-yl)propane-1-sulfonamide;
A-34) N-(5-(2-(3-ethylureido)-7-(pyridin-2-yl)benzo[d]thiazol-5-yl)pyrimidin-2-yl)cyclopropanesulfonamide;
A-35) N-(5-(2-(3-ethylureido)-7-(pyridin-2-yl)benzo[d]thiazol-5-yl)pyrimidin-2-yl)cyclopentanesulfonamide;
A-36) N-(5-(2-(3-ethylureido)-7-(pyridin-2-yl)benzo[d]thiazol-5-yl)pyrimidin-2-yl)ethanesulfonamide;
A-37) 1-ethyl-3-[5-[2-(ethylsulfamoylamino)pyrimidin-5-yl]-7-(2-pyridyl)-1,3-benzothiazol-2-yl]urea;
A-38) 1-[5-[2-(dimethylsulfamoylamino)pyrimidin-5-yl]-7-(5-methyl-2-pyridyl)-1,3-benzothiazol-2-yl]-3-ethyl urea;
A-39) 1-ethyl-3-[5-[2-(ethylsulfamoylamino)pyrimidin-5-yl]-7-(5-methyl-2-pyridyl)-1,3-benzothiazol-2-yl]urea;
A-40) N-(5-(2-(3-ethylureido)-7-(5-methylpyridin-2-yl)benzo[d]thiazol-5-yl)pyrimidin-2-yl)cyclopropanesulfonamide;
A-41) 1-ethyl-3-[5-[2-(methylsulfamoylamino)pyrimidin-5-yl]-7-(2-pyridyl)-1,3-benzothiazol-2-yl]urea;
A-42) 1-ethyl-3-[7-(5-methyl-2-pyridyl)-5-[2-(methylsulfamoylamino)pyrimidin-5-yl]-1,3-benzothiazol-2-yl]urea;
A-43) N-(5-(2-(3-ethylureido)-7-(pyridin-2-yl)benzo[d]thiazol-5-yl)pyrimidin-2-yl)morpholine-4-sulfonamide;
A-44) N-(5-(2-(3-ethylureido)-7-(5-methylpyridin-2-yl)benzo[d]thiazol-5-yl)pyrimidin-2-yl)morpholine-4-sulfonamide;
A-45) N-(5-(2-(3-ethylureido)-7-(pyridin-2-yl)benzo[d]thiazol-5-yl)pyrimidin-2-yl)pyrrolidine-1-sulfonamide;
A-46) (S)-2-amino-N-(5-(2-(3-ethylureido)-7-(5-methylpyridin-2-yl)benzo[d]thiazol-5-yl)pyrimidin-2-yl)-3-phenylpropane-1-sulfonamide;
A-47) N-(5-(2-(3-ethylureido)-7-(5-methylpyridin-2-yl)benzo[d]thiazol-5-yl)pyrimidin-2-yl)ethane sulfonamide;
A-48) N-(5-(2-(3-ethylureido)-7-(5-methylpyridin-2-yl)benzo[d]thiazol-5-yl)pyrimidin-2-yl)propane-2-sulfonamide;
A-49) N-(5-(2-(3-ethylureido)-7-(pyridin-2-yl)benzo[d]thiazol-5-yl)pyrimidin-2-yl)-3-methoxyazetidine-1-sulfonamide;
A-50) N-(5-(2-(3-ethylureido)-7-(5-methylpyridin-2-yl)benzo[d]thiazol-5-yl)pyrimidin-2-yl)-3-methoxyazetidine-1-sulfonamide;
A-51) N-(5-(2-(3-ethylureido)-7-(5-methylpyridin-2-yl)benzo[d]thiazol-5-yl)pyrimidin-2-yl)benzenesulfonamide;
A-52) N-(5-(2-(3-ethylureido)-7-(5-methylpyridin-2-yl)benzo[d]thiazol-5-yl)pyrimidin-2-yl)-2-morpholinoethanesulfonamide;
A-53) N-(5-(2-(3-ethylureido)-7-(5-methylpyridin-2-yl)benzo[d]thiazol-5-yl)pyrimidin-2-yl)pyrrolidine-3-sulfonamide;
A-54) N-(5-(2-(3-ethylureido)-7-(5-methylpyridin-2-yl)benzo[d]thiazol-5-yl)pyrimidin-2-yl)-3-hydroxypyrrolidine-1-sulfonamide;
A-55) N-(5-(2-(3-ethylureido)-7-(5-methylpyridin-2-yl)benzo[d]thiazol-5-yl)pyrimidin-2-yl)-1-(hydroxymethyl)cyclopropane-1-sulfonamide;
A-56) (R)—N-(5-(2-(3-ethylureido)-7-(5-methylpyridin-2-yl)benzo[d]thiazol-5-yl)pyrimidin-2-yl)-3-morpholinopyrrolidine-1-sulfonamide;
A-57) (R)-3-(dimethylamino)-N-(5-(2-(3-ethylureido)-7-(5-methylpyridin-2-yl)benzo[d]thiazol-5-yl)pyrimidin-2-yl)pyrrolidine-1-sulfonamide;
A-58) 1-acetyl-N-(5-(2-(3-ethylureido)-7-(5-methylpyridin-2-yl)benzo[d]thiazol-5-yl)pyrimidin-2-yl)pyrrolidine-3-sulfonamide;
A-59) (R)—N-(5-(2-(3-ethylureido)-7-(5-methylpyridin-2-yl)benzo[d]thiazol-5-yl)pyrimidin-2-yl)-3-hydroxypyrrolidine-1-sulfonamide;
A-60) (S)—N-(5-(2-(3-ethylureido)-7-(5-methylpyridin-2-yl)benzo[d]thiazol-5-yl)pyrimidin-2-yl)-3-hydroxypyrrolidine-1-sulfonamide;
A-61) N-(5-(2-(3-ethylureido)-6-fluorobenzo[d]thiazol-5-yl)pyrimidin-2-yl)-2-methylpropane-2-sulfonamide;
A-62) (R)—N-(5-(2-(3-ethylureido)-7-(5-methylpyridin-2-yl)benzo[d]thiazol-5-yl)pyrimidin-2-yl)-2-(hydroxymethyl)pyrrolidine-1-sulfonamide;
A-63) (S)—N-(5-(2-(3-ethylureido)-7-(5-methylpyridin-2-yl)benzo[d]thiazol-5-yl)pyrimidin-2-yl)tetrahydrofuran-3-sulfonamide;
A-64) N-(5-(7-bromo-2-(3-ethylureido)benzo[d]thiazol-5-yl)pyrimidin-2-yl)-2-methylpropane-2-sulfonamide;
A-65) 1-[7-bromo-5-[6-(tert-butylsulfonylamino)-3-pyridyl]-1,3-benzothiazol-2-yl]-3-ethyl urea;
A-66) methyl 2-[[5-[2-(ethylcarbamoylamino)-7-(5-methyl-2-pyridyl)-1,3-benzothiazol-5-yl]pyrimidin-2-yl]sulfamoyl]acetate;
A-67) (R)—N-(5-(2-(3-ethylureido)-7-(5-methylpyridin-2-yl)benzo[d]thiazol-5-yl)pyrimidin-2-yl)-1-(tetrahydro-2H-pyran-2-yl)methane sulfonamide;
A-68) N-(5-(2-(3-ethylureido)-7-(5-methylpyridin-2-yl)benzo[d]thiazol-5-yl)pyrimidin-2-yl)-2-methoxyethanesulfonamide;
A-69) 1-[5-[6-(tert-butylsulfonylamino)-3-pyridyl]-7-(2-ethylthiazol-4-yl)-1,3-benzothiazol-2-yl]-3-ethyl urea;
A-70) N-(5-(2-(3-ethylureido)-7-(5-methylpyridin-2-yl)benzo[d]thiazol-5-yl)pyrimidin-2-yl)-2-hydroxyethanesulfonamide;
A-71) N-(5-(2-(3-ethylureido)-7-(1H-pyrazol-4-yl)benzo[d]thiazol-5-yl)pyrimidin-2-yl)-2-methylpropane-2-sulfonamide;
A-72) N-(5-(7-(3,5-dimethylisoxazol-4-yl)-2-(3-ethylureido)benzo[d]thiazol-5-yl)pyrimidin-2-yl)-2-methylpropane-2-sulfonamide;
A-73) N-(5-(2-(3-ethylureido)-7-(1-methyl-1H-pyrazol-4-yl)benzo[d]thiazol-5-yl)pyrimidin-2-yl)-2-methylpropane-2-sulfonamide;
A-74) N-(5-(2-(3-ethylureido)-7-((5-methylpyridin-2-yl)amino)benzo[d]thiazol-5-yl)pyrimidin-2-yl)-2-methylpropane-2-sulfonamide;
A-75) N-(5-(2-(3-ethylureido)-7-methylbenzo[d]thiazol-5-yl)pyrimidin-2-yl)-2-methylpropane-2-sulfonamide;
A-76) N-(5-(2-(3-ethylureido)benzo[d]thiazol-5-yl)pyrimidin-2-yl)-2-methylpropane-2-sulfonamide;
A-77) N-(5-(7-cyclopropyl-2-(3-ethylureido)benzo[d]thiazol-5-yl)pyrimidin-2-yl)-2-methylpropane-2-sulfonamide;
A-78) N-(5-(2-(3-ethylureido)-7-(tetrahydro-2H-pyran-4-yl)benzo[d]thiazol-5-yl)pyrimidin-2-yl)-2-methylpropane-2-sulfonamide;
A-79) N-(5-(2-(3-ethylureido)-7-(pyrrolidin-1-yl)benzo[d]thiazol-5-yl)pyrimidin-2-yl)-2-methylpropane-2-sulfonamide;
A-80) N-(5-(2-(3-ethylureido)-7-(piperazin-1-yl)benzo[d]thiazol-5-yl)pyrimidin-2-yl)-2-methylpropane-2-sulfonamide;
A-81) N-(5-(2-(3-ethylureido)-7-morpholinobenzo[d]thiazol-5-yl)pyrimidin-2-yl)-2-methylpropane-2-sulfonamide;
A-82) N-(5-(2-(3-ethylureido)-7-(4-methylpiperazin-1-yl)benzo[d]thiazol-5-yl)pyrimidin-2-yl)-2-methylpropane-2-sulfonamide;
A-83) N-(5-(7-(4-acetylpiperazin-1-yl)-2-(3-ethylureido)benzo[d]thiazol-5-yl)pyrimidin-2-yl)-2-methylpropane-2-sulfonamide;
A-84) N-(5-(2-(3-ethylureido)-7-(1-(2-morpholinoethyl)-1H-pyrazol-4-yl)benzo[d]thiazol-5-yl)pyrimidin-2-yl)-2-methylpropane-2-sulfonamide;
A-85) N-(5-(2-(3-ethylureido)-7-(4-methylpyridin-2-yl)benzo[d]thiazol-5-yl)pyrimidin-2-yl)-2-methylpropane-2-sulfonamide;
A-86) N-(5-(2-(3-ethylureido)-7-(5-(morpholinomethyl)pyridin-2-yl)benzo[d]thiazol-5-yl)pyrimidin-2-yl)-2-methylpropane-2-sulfonamide;
A-87) N-(5-(2-(3-ethylureido)-7-(2-(3-hydroxypyrrolidin-1-yl)thiazol-4-yl)benzo[d]thiazol-5-yl)pyrimidin-2-yl)-2-methylpropane-2-sulfonamide;
A-88) 1-[5-[2-(tert-butylsulfonylamino)pyrimidin-5-yl]-7-(2-hydroxy-4-pyridyl)-1,3-benzothiazol-2-yl]-3-ethyl-urea;
A-89) N-(5-(2-(3-ethylureido)-7-(1-methyl-2-oxo-1,2-dihydropyridin-4-yl)benzo[d]thiazol-5-yl)pyrimidin-2-yl)-2-methylpropane-2-sulfonamide;
A-90) 2-(4-(5-(2-(1,1-dimethylethylsulfonamido)pyrimidin-5-yl)-2-(3-ethylureido)benzo[d]thiazol-7-yl)piperazin-1-yl)-N-methylacetamide;
A-91)ethyl 2-(4-(5-(2-(1,1-dimethylethylsulfonamido)pyrimidin-5-yl)-2-(3-ethylureido)benzo[d]thiazol-7-yl)piperazin-1-yl)acetate;
A-92) N-(5-(2-(3-ethylureido)-7-(2-(piperazin-1-yl)thiazol-4-yl)benzo[d]thiazol-5-yl)pyrimidin-2-yl)-2-methylpropane-2-sulfonamide;
A-93) N-(5-(2-(3-ethylureido)-7-(6-methylpyridin-2-yl)benzo[d]thiazol-5-yl)pyrimidin-2-yl)-2-methylpropane-2-sulfonamide;
A-94) N-(5-(7-(2-aminopyridin-3-yl)-2-(3-ethylureido)benzo[d]thiazol-5-yl)pyrimidin-2-yl)-2-methylpropane-2-sulfonamide;
A-95) N-(5-(7-(5-aminopyridin-3-yl)-2-(3-ethylureido)benzo[d]thiazol-5-yl)pyrimidin-2-yl)-2-methylpropane-2-sulfonamide;
A-96) N-(5-(2-(3-ethylureido)-7-(4-(2-methoxyethyl)piperazin-1-yl)benzo[d]thiazol-5-yl)pyrimidin-2-yl)-2-methylpropane-2-sulfonamide;
A-97) N-(5-(2-(3-ethylureido)-7-(piperidin-1-yl)benzo[d]thiazol-5-yl)pyrimidin-2-yl)-2-methylpropane-2-sulfonamide;
A-98) N-(5-(7-(5-(aminomethyl)-2-fluorophenyl)-2-(3-ethylureido)benzo[d]thiazol-5-yl)pyrimidin-2-yl)-2-methylpropane-2-sulfonamide;
A-99) N-(5-(2-(3-ethylureido)-7-[2-dimethylaminoethyl(methyl)amino]benzo[d]thiazol-5-yl)pyrimidin-2-yl)-2-methylpropane-2-sulfonamide;
A-100) N-(5-(2-(3-ethylureido)-7-(pyridin-2-yl)benzo[d]thiazol-5-yl)pyrimidin-2-yl)-2-methylpropane-2-sulfonamide;
A-103) 1-[6-[2-(tert-butylsulfonylamino)pyrimidin-5-yl]-5-methoxy-thiazolo[5,4-b]pyridin-2-yl]-3-ethylurea;
A-104) 1-[5-[2-(tert-butylsulfonylamino)pyrimidin-5-yl]-7-(5-hydroxy-2-pyridyl)-1,3-benzothiazol-2-yl]-3-ethylurea;
A-105) 1-[5-[2-(tert-butylsulfonylamino)pyrimidin-5-yl]-7-[4-[(cyclopropylamino)methyl]-2-pyridyl]-1,3-benzothiazol-2-yl]-3-ethylurea;
A-106) 1-[7-(5-amino-2-pyridyl)-5-[2-(tert-butylsulfonylamino)pyrimidin-5-yl]-1,3-benzothiazol-2-yl]-3-ethylurea;
A-107) 1-[5-[2-(tert-butylsulfonylamino)pyrimidin-5-yl]-7-(3-methyl-2-pyridyl)-1,3-benzothiazol-2-yl]-3-ethyl-urea;
A-110) 1-[7-(4-amino-2-pyridyl)-5-[2-(tert-butylsulfonylamino)pyrimidin-5-yl]-1,3-benzothiazol-2-yl]-3-ethylurea;
A-111) 1-[5-[2-(2,3-dihydroxypropylsulfonylamino)pyrimidin-5-yl]-7-(5-methyl-2-pyridyl)-1,3-benzothiazol-2-yl]-3-ethylurea;
A-113) 1-[5-[2-(tert-butylsulfonylamino)pyrimidin-5-yl]-7-(4,5-dimethyl-2-pyridyl)-1,3-benzothiazol-2-yl]-3-ethylurea;
A-114) 1-[5-[2-(tert-butylsulfonylamino)pyrimidin-5-yl]-7-[(3S,4R,5R,6R)-3,4,5,6-tetrahydroxycyclohexen-1-yl]-1,3-benzothiazol-2-yl]-3-ethylurea;
A-115) 1-[7-[(3aR,6aR)-2,3,3a,4,6,6a-hexahydro-1H-pyrrolo[2,3-c]pyrrol-5-yl]-5-[2-(tert-butylsulfonylamino)pyrimidin-5-yl]-1,3-benzothiazol-2-yl]-3-ethyl-urea;
   and; salts, racemates, diastereomers, enantiomers, deuterated forms, hydrates, solvates and prodrugs thereof.

In a further embodiment, there is provided compound of Formula (III):

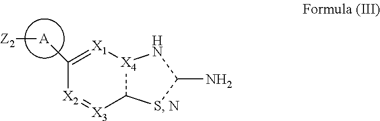

and; salts, racemates, diastereomers, enantiomers, deuterated forms, hydrates, solvates and prodrugs thereof;

wherein Ring A, X₁, X₂ and X₃ are as previously defined according to Formula (I) and embodiments thereof; X₄ is C or N; and

Z₂ may be (CH₂)ᵥNRS(═O)₂R₈, (CH₂)ᵥS(═O)₂NR₉R₁₀ or (CH₂)ᵥNRS(═O)₂NR₉R₁₀; wherein

v is an integer 0, 1, 2 or 3;

R is H or an optionally substituted C₁₋₆alkyl; and

R₈, R₉ and R₁₀ may each be independently selected from H, C₁₋₆alkyl, C₂₋₆alkenyl, C₂₋₆alkynyl, C₃₋₇cycloalkyl, phenyl, benzyl, a 3-10-membered heterocyclic ring, a 5-10-membered heteroaryl ring and further wherein each C₁₋₆alkyl, C₂₋₆alkenyl, C₂₋₆alkynyl, C₃₋₇cycloalkyl, phenyl, benzyl, a 3-10-membered heterocyclic ring, a 5-10-membered heteroaryl ring may be optionally substituted;

or R₉ and R₁₀ may join to form an optionally substituted 3-6-membered heterocyclic ring together with the nitrogen to which they are attached.

Optional substituents for R₈, R₉ and R₁₀ may include but are not limited to one or more substituents independently selected from halo (for example Cl, Br, F, I), C₁₋₄alkyl (for example methyl, ethyl, propyl, iso-propyl, n-butyl, sec-butyl, tert-butyl), C₂₋₄alkenyl (for example ethenyl, propenyl, butenyl), C₂₋₄alkynyl (for example ethynyl, propynyl, butynyl), C₁₋₄alkylhalo (for example CH₂F, CF₃), OH, OC₁₋₄alkyl (for example OCH₃, OCH₂CH₃), C₁₋₄alkoxyl (for example CH₂OCH₃, CH₂CH₂OCH₃), C₁₋₄alkoxylhalo (for example OCH₂F, OCF₃), CN, NH₂, NH(C₁₋₄alkyl) (for example NHCH₃, NHCH₂CH₃), N(C₁₋₄alkyl)₂ (for example N(CH₃)₂, N(CH₃)CH₂CH₃, N(CH₂CH₃)₂), C(═O)OC₁₋₄alkyl (for example C(═O)OCH₃, C(═O)OCH₂CH₃), C₃₋₆ cycloalkyl (for example cyclopropyl, cyclobutyl, cyclopentyl, cyclohexyl), phenyl, benzyl, a 3-6-membered heterocyclic ring containing N and/or O heteroatoms (for example aziridine, oxirane, pyrrolidine, pyrazoline, imidazoline, pyrazolidine, imidazolidine, tetrahydrofuran, piperidine, tetrahydropyran, piperizine, morphline), or a 5-10-membered heteroaryl ring containing one or more N, O and/or S heteroatoms (for example 5-membered heteroaryl rings such as pyrrole, furan, thiophene, pyrazole, imidazole, oxazole, isoxazole, thiazole, isothiazole, triazole, oxadiazole, furazan, thiadiazole, tetrazole, and 6-membered heteroaryl rings such as pyridine, pyridazine, pyrimidine and pyrazine).

In one embodiment of Formula (III) Z₂ is NRS(═O)₂R₈.

In another embodiment of Formula (III) Z₂ is S(═O)₂NR₉R₁₀.

In another embodiment of Formula (III) Z₂ is NRS(═O)₂NR₉R₁₀.

Examples of compounds of Formula (III), include but are not limited to, any one of compound examples A-102, A-108, and A-112 as described in the Examples section which follows:

A-102) N-[5-(2-amino-7-bromo-1,3-benzothiazol-5-yl)pyrimidin-2-yl]-2-methyl-propane-2-sulfonamide;

A-108) N-[5-[2-amino-4-(5-methyl-2-pyridyl)pyrazolo[1,5-a]pyridin-6-yl]pyrimidin-2-yl]-2-methyl-propane-2-sulfonamide;

A-112) N-[5-(2-amino-1,3-benzothiazol-5-yl)pyrimidin-2-yl]-2-methyl-propane-2-sulfonamide;

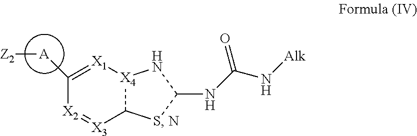

and; salts, racemates, diastereomers, enantiomers, deuterated forms, hydrates, solvates and prodrugs thereof;

wherein Alk, Ring A, X₁, X₂ and X₃ are as previously defined according to Formula (I) and embodiments thereof; X₄ is C or N; and

Z₂ may be (CH₂)ᵥNRS(═O)₂R₈, (CH₂)ᵥS(═O)₂NR₉R₁₀ or (CH₂)ᵥNRS(═O)₂NR₉R₁₀; wherein

v is an integer 0, 1, 2 or 3;

R is H or an optionally substituted C₁₋₆alkyl; and

R₈, R₉ and R₁₀ may each be independently selected from H, C₁₋₆alkyl, C₂₋₆alkenyl, C₂₋₆alkynyl, C₃₋₇cycloalkyl, phenyl, benzyl, a 3-10-membered heterocyclic ring, a 5-10-membered heteroaryl ring and further wherein each C₁₋₆alkyl, C₂₋₆alkenyl, C₂₋₆alkynyl, C₃₋₇cycloalkyl, phenyl, benzyl, a 3-10-membered heterocyclic ring, a 5-10-membered heteroaryl ring may be optionally substituted;

or R₉ and R₁₀ may join to form an optionally substituted 3-6-membered heterocyclic ring together with the nitrogen to which they are attached.

Optional substituents for R₈, R₉ and R₁₀ may include but are not limited to one or more substituents independently selected from halo (for example Cl, Br, F, I), C₁₋₄alkyl (for example methyl, ethyl, propyl, iso-propyl, n-butyl, sec-butyl, tert-butyl), C₂₋₄alkenyl (for example ethenyl, propenyl, butenyl), C₂₋₄alkynyl (for example ethynyl, propynyl, butynyl), C₁₋₄alkylhalo (for example CH₂F, CF₃), OH, OC₁₋₄alkyl (for example OCH₃, OCH₂CH₃), C₁₋₄alkoxyl (for example CH₂OCH₃, CH₂CH₂OCH₃), C₁₋₄alkoxylhalo (for example OCH₂F, OCF₃), CN, NH₂, NH(C₁₋₄alkyl) (for example NHCH₃, NHCH₂CH₃), N(C₁₋₄alkyl)₂ (for example N(CH₃)₂, N(CH₃)CH₂CH₃, N(CH₂CH₃)₂), C(═O)OC₁₋₄alkyl (for example C(═O)OCH₃, C(═O)OCH₂CH₃), C₃₋₆cycloalkyl (for example cyclopropyl, cyclobutyl, cyclopentyl, cyclohexyl), phenyl, benzyl, a 3-6-membered heterocyclic ring containing N and/or O heteroatoms (for example aziridine, oxirane, pyrrolidine, pyrazoline, imidazoline, pyrazolidine, imidazolidine, tetrahydrofuran, piperidine, tetrahydropyran, piperizine, morphline), or a 5-10-membered heteroaryl ring containing one or more N, O and/or S heteroatoms (for example 5-membered heteroaryl rings such as pyrrole, furan, thiophene, pyrazole, imidazole, oxazole, isoxazole, thiazole, isothiazole, triazole, oxadiazole, furazan, thiadiazole, tetrazole, and 6-membered heteroaryl rings such as pyridine, pyridazine, pyrimidine and pyrazine).

In one embodiment of Formula (IV) Z₂ is NRS(═O)₂R₈.

In another embodiment of Formula (IV) Z₂ is S(═O)₂NR₉R₁₀.

In another embodiment of Formula (IV) Z₂ is NRS(═O)₂NR₉R₁₀.

Examples of compounds of Formula (IV), include but is not limited to, any one of compound examples A-101 and A-109 as described in the Examples section which follows:
A-101) 1-[6-[2-(tert-butylsulfonylamino)pyrimidin-5-yl]-8-(5-methyl-2-pyridyl)-[1,2,4]triazolo[1,5-a]pyridin-2-yl]-3-ethyl-urea;
A-109) 1-[4-bromo-6-[2-(tert-butylsulfonylamino)pyrimidin-5-yl]pyrazolo[1,5-a]pyridin-2-yl]-3-ethylurea;

The compounds of Formula (II), Formula (III), and Formula (IV) are useful when used in the novel combination of the present disclosure.

In one embodiment, the bacterial type II topoisomerase inhibitor for use in combination with a polymyxin or polymyxin derivative may be selected from the group consisting of:
1-ethyl-3-[5-(1-methyl-2-oxo-4-pyridyl)-7-(2-pyridyl)-1,3-benzothiazol-2-yl]urea;
1-[7-(3-amino-2-pyridyl)-5-(3-pyridyl)-1,3-benzothiazol-2-yl]-3-ethyl-urea;
1-ethyl-3-[7-(2-pyridyl)-5-(3-pyridyl)-1,3-benzothiazol-2-yl]urea;
1-ethyl-3-[5-(3-pyridyl)-1,3-benzothiazol-2-yl]urea;
1-[5-[2-(ethylcarbamoylamino)-7-(2-pyridyl)-1,3-benzothiazol-5-yl]pyrimidin-2-yl]-4-methyl-piperidine-4-carboxylic acid;
1-ethyl-3-[5-[2-[(1R)-1-hydroxyethyl]pyrimidin-5-yl]-7-(2-pyridyl)-1,3-benzothiazol-2-yl]urea;
1-ethyl-3-[5-[2-(1-hydroxy-1-methyl-ethyl)pyrimidin-5-yl]-7-[4-(morpholinomethyl)-2-pyridyl]-1,3-benzothiazol-2-yl]urea;
[(1R)-1-[5-[2-(ethylcarbamoylamino)-7-(2-pyridyl)-1,3-benzothiazol-5-yl]pyrimidin-2-yl]ethyl]dihydrogen phosphate;
(3R)—N-[5-[2-(Ethylcarbamoylamino)-7-(5-methyl-2-pyridyl)-1,3-benzothiazol-5-yl]pyrimidin-2-yl]-3-hydroxy-pyrrolidine-1-carboxamide;
1-Ethyl-3-[5-[2-methyl-1-[(6-methyl-2-pyridyl)methyl]-6-oxo-4-pyridyl]-7-(2-pyridyl)-1,3-benzothiazol-2-yl]urea;
1-Ethyl-3-[5-(4-methylimidazol-1-yl)-7-(2-pyridyl)-1,3-benzothiazol-2-yl]urea;
1-Ethyl-3-[5-[2-methyl-1-[1-(6-methyl-3-pyridyl)ethyl]-6-oxo-4-pyridyl]-7-(2-pyridyl)-1,3-benzothiazol-2-yl]urea;
Methyl N-[5-[2-(ethylcarbamoylamino)-7-(2-pyridyl)-1,3-benzothiazol-5-yl]pyrimidin-2-yl]carbamate;
N-[5-[2-(Ethylcarbamoylamino)-7-[4-(morpholinomethyl)-2-pyridyl]-1,3-benzothiazol-5-yl]pyrimidin-2-yl]pyrrolidine-1-carboxamide;
1-[5-[2-(1,2-Dihydroxy-1-methyl-ethyl)pyrimidin-5-yl]-1,3-benzothiazol-2-yl]-3-ethyl-urea;
1-[5-[2-(1,2-Dihydroxy-1-methyl-ethyl)pyrimidin-5-yl]-7-(5-methylpyrimidin-2-yl)-1,3-benzothiazol-2-yl]-3-ethyl-urea;
1-[5-[2-(1,2-Dihydroxy-1-methyl-ethyl)pyrimidin-5-yl]-7-methyl-1,3-benzothiazol-2-yl]-3-ethyl-urea;
2-[[5-[2-(Ethylcarbamoylamino)-7-(2-pyridyl)-1,3-benzothiazol-5-yl]pyrimidin-2-yl]sulfamoyl]benzoic acid;
1-Ethyl-3-[7-(5-methyl-2-pyridyl)-5-[2-(pyrrolidin-1-ylsulfonylamino)pyrimidin-5-yl]-1,3-benzothiazol-2-yl]urea;
1-Ethyl-3-[7-(5-methyl-2-pyridyl)-5-[2-(propylsulfonylamino)pyrimidin-5-yl]-1,3-benzothiazol-2-yl]urea;
1-[5-[2-(tert-Butylsulfonylamino)pyrimidin-5-yl]-7-(5-methyl-2-pyridyl)-1,3-benzothiazol-2-yl]-3-ethyl-urea;
1-Ethyl-3-[5-[2-[(2-hydroxy-1,1-dimethyl-ethyl)sulfonylamino]pyrimidin-5-yl]-7-(5-methyl-2-pyridyl)-1,3-benzothiazol-2-yl]urea;
1-Ethyl-3-[5-[2-[[2-hydroxyethyl(methyl)sulfamoyl]amino]pyrimidin-5-yl]-7-(5-methyl-2-pyridyl)-1,3-benzothiazol-2-yl]urea;
Methyl 2-[[5-[2-(ethylcarbamoylamino)-7-(5-methyl-2-pyridyl)-1,3-benzothiazol-5-yl]pyrimidin-2-yl]sulfamoyl]acetate;
1-[5-[2-(allylsulfonylamino)pyrimidin-5-yl]-7-(5-methyl-2-pyridyl)-1,3-benzothiazol-2-yl]-3-ethyl-urea;
1-[5-[2-(tert-butylsulfonylamino)pyrimidin-5-yl]-7-[2-(dimethylamino)pyrimidin-5-yl]-1,3-benzothiazol-2-yl]-3-ethyl-urea;
1-ethyl-3-[5-[2-(methanesulfonamidomethyl)pyrimidin-5-yl]-7-(5-methyl-2-pyridyl)-1,3-benzothiazol-2-yl]urea;
1-[5-[2-(tert-butylsulfonylamino)pyrimidin-5-yl]-7-[4-(2-pyrrolidin-1-ylethyl)piperazin-1-yl]-1,3-benzothiazol-2-yl]-3-ethyl-urea;
1-[5-[2-(cyclopentylsulfonylamino)pyrimidin-5-yl]-7-(5-methyl-2-pyridyl)-1,3-benzothiazol-2-yl]-3-ethyl-urea;
N-[5-(2-amino-7-bromo-1,3-benzothiazol-5-yl)pyrimidin-2-yl]-2-methyl-propane-2-sulfonamide;
1-[6-[2-(tert-butylsulfonylamino)pyrimidin-5-yl]-8-(5-methyl-2-pyridyl)-[1,2,4]triazolo[1,5-a]pyridin-2-yl]-3-ethyl-urea; and
   salts, racemates, diastereomers, enantiomers, esters, carbamates, phosphates, sulfates, deuterated forms and prodrugs thereof.

### Compositions

There is also provided a composition comprising a bacterial type II topoisomerase inhibitor and a polymyxin or polymyxin derivative, wherein the bacterial type II topoisomerase inhibitor has on-target enzyme activity against DNA gyrase and optionally on-target enzyme activity against topoisomerase IV.

In one embodiment the composition optionally comprises a carrier, diluent or excipient.

In another embodiment the composition is a pharmaceutical composition and the carrier, diluent or excipient is pharmaceutically acceptable.

In one embodiment, the bacterial type II topoisomerase inhibitor is a compound of Formula (I), its salts, isomers, racemates, diastereomers, enantiomers and prodrugs thereof as previously defined herein.

In another embodiment, the bacterial type II topoisomerase inhibitor is a compound of Formula (II), its salts, isomers, racemates, diastereomers, enantiomers and prodrugs thereof as previously defined herein.

In one embodiment the polymyxin or polymyxin derivative is provided in a therapeutically effective antibacterial amount or dosage.

In an alternative embodiment the polymyxin or polymyxin derivative is provided in a sub-inhibitory MIC amount or dosage, that is, a non-therapeutically effective antibacterial amount or dosage.

In one embodiment the composition comprises a bacterial type II topoisomerase inhibitor as previously defined and a polymyxin.

In one embodiment the polymyxin may be colistin (Polymyxin E) or polymyxin B (PMB).

In one embodiment the polymyxin may be colistin (Polymyxin E). In a further embodiment colistin may be administered in an antibacterially effective amount or dosage. In another embodiment colistin may be administered in a non-antibacterially effective amount or dosage.

In another embodiment the polymyxin may be Polymyxin B (PMB). In another embodiment PMB may be administered in an antibacterially effective amount or dosage. In another embodiment PMB is administered in a non-antibacterially effective amount or dosage.

In one embodiment the composition comprises a bacterial type II topoisomerase inhibitor as previously defined and a polymyxin derivative.

In one embodiment the polymyxin derivative may be polymyxin B nonapeptide (PMBN) or colistin methanesulfonate (CMS).

In a particular embodiment the polymyxin derivative may be a prodrug of colistin. In a further embodiment the prodrug of colistin may be administered in an amount or dosage to provide an antibacterially effective amount or dosage of colistin.

In another embodiment the prodrug of colistin may be administered in an amount or dosage to provide a non-antibacterially effective amount or dosage of colistin.

In a further embodiment the prodrug of colistin may be colistin methanesulfonate (CMS).

In another particular embodiment the polymyxin derivative may be Polymyxin B nonapeptide (PMBN).

The compositions of the present disclosure may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Pharmaceutical compositions include those for oral, rectal, nasal, topical (including buccal and sub-lingual), vaginal or parenteral (including intramuscular, sub-cutaneous and intravenous) administration or in a form suitable for administration by inhalation or insufflation. The compounds of the disclosure, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, in the form of suppositories for rectal administration; or in the form of sterile injectable solutions for parenteral (including subcutaneous) use.

In one embodiment the compositions of the present disclosure are formulated for oral administration and/or intravenous (IV) administration.

In another embodiment the compositions of the present disclosure may be administered in combination with or additionally comprise another antibacterial agent. Suitable antibacterial agents will be familiar to those in the art and may include penicillins, cephalosporins, carbapenems, monobactams, beta-lactams, glycopeptides, aminoglycosides, tetracyclines, macrolides, ketolides, quinolones, fluoroquinolones, oxazolidinones, coumarins, cyclothialidines, vancomycin and derivatives thereof.

The pharmaceutical compositions for the administration of the compounds of the present disclosure may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

### Methods of Treatment

There is provided a method for the treatment or prevention of a bacterial infection comprising administration of a bacterial type II topoisomerase inhibitor in combination with a polymyxin or polymyxin derivative to a subject suffering from infection or at risk of infection, wherein the bacterial infection is caused by one or more Gram-negative bacteria or drug resistant Gram-negative bacteria.

In one embodiment, the method is for the treatment of a bacterial infection wherein the subject is suffering from said infection as defined herein.

In another embodiment, the method is for the prevention of a bacterial infection wherein the subject is at risk of said infection as defined herein. Subjects at risk of infection include, for example, a patient, particularly a human patient, who is about to undergo surgery.

In one embodiment the subject is a human. In another embodiment, the subject is a non-human animal.

In one embodiment the combination may be administered concurrently, sequentially or separately to a patient suffering from infection or at risk of infection.

In one embodiment, the bacterial type II topoisomerase inhibitor may be a compound of Formula (I) as defined herein, or a salt, racemate, diastereomer, enantiomer, ester, carbamate, phosphate, sulfate, deuterated form or prodrug thereof.

In one embodiment, the bacterial type II topoisomerase inhibitor may be a compound of Formula (II) as defined herein, or a salt, racemate, diastereomer, enantiomer, ester, carbamate, phosphate, sulfate, deuterated form or prodrug thereof.

In one embodiment the Gram-negative bacteria or drug resistant Gram-negative bacteria comprises a lipopolysaccharide (LPS) layer. Gram-negative pathogens which comprise an LPS layer include, but are not limited to bacterial strains that may be selected from the group comprising E. coli, K pneumoniae, A. baumannii, P. aeruginosa, Enterobacter spp

In one embodiment, the Gram-negative pathogen is one or more bacterial strains selected from the group E. coli, K pneumoniae, A. baumannii, P. aeruginosa, and Enterobacter spp or drug resistant strain thereof.

In one embodiment, the bacterial infection may be caused by an E. coli bacterial strain or drug resistant strain thereof.

In one embodiment, the bacterial infection may be caused by a K. pneumoniae bacterial strain or drug resistant strain thereof.

In one embodiment, the bacterial infection may be caused by an A. baumannii bacterial strain of drug resistant strain thereof.

In one embodiment, the bacterial infection may be caused by a P. aeruginosa bacterial strain or drug resistant strain thereof.

In one embodiment, the bacterial infection may be caused by an Enterobacter spp bacterial strain or drug resistant strain thereof.

In another embodiment, the Gram-negative bacteria or drug resistant Gram-negative bacteria comprises a lipooligosaccharide (LOS) layer. Gram-negative pathogens which comprise an LOS layer include, but are not limited to bacterial strains selected from the group comprising Moraxella catarrhalis and members of the genera Neisseria, Haemophilus and Bordetella, such as Neisseria gonorrhoeae and Haemophilus influenza and drug resistant strain thereof.

In one embodiment, the bacterial infection may be caused by an H. influenzae bacterial strain or drug resistant strain thereof.

In one embodiment, the bacterial infection may be caused by an N. gonorrhoeae bacterial strain or drug resistant strain thereof.

In one embodiment, the bacterial infection may be caused by an M. catarrhalis bacterial strain or drug resistant strain thereof.

Other Gram-negative pathogens include but are not limited to bacterial strains selected from the group comprising Legionella pneumoniae, Chlamydia trachomatis and Chlamydophila pneumoniae and Chlamydophila pneumoniae, and biodefence pathogens such as Yersinia pestis, Francisella species, eg F. tularensis, Burkholderia species, eg B. pseudomallei, Burkholderia mallei, Coxiella burnetii, Brucella species, Chlamydia psittaci and Rickettsia prowazekii.

Clinical manifestations which commonly result from infections caused by Gram-negative bacterial pathogens or drug resistant Gram-negative bacteria include conditions such as intra-abdominal infections (IAI), hospital acquired pneumonias (HAP), ventilator-associated pneumonia (VAP), urinary tract infection (UTI), bacteremias, community acquired bacterial pneumonia (CABP), gonococcal infection (GI), wound or surgical site infections, endocarditis, otitis media, cystic fibrosis and meningitis.

Accordingly, in one embodiment according to the method, the combination to be administered to the subject is wherein the subject is suffering from or at risk of an intra-abdominal infection (IAI), hospital acquired pneumonia (HAP), ventilator-associated pneumonia (VAP), urinary tract infection (UTI), bacteremias, community acquired bacterial pneumonia (CABP), gonococcal infection (GI), wound or surgical site infections, endocarditis, otitis media, cystic fibrosis or meningitis.

The compounds of the combination or composition may be administered by any suitable means, for example, orally, parenterally, such as by subcutaneous, intravenous, intramuscular, or intracisternal injection or infusion techniques (e.g., as sterile injectable aqueous or non-aqueous solutions or suspensions).

In the treatment or prevention of bacterial infections, an appropriate dosage level will generally be about 0.01 to 500 mg per kg patient body weight per day which can be administered in single or multiple doses. For oral administration, the compositions are preferably provided in the form of tablets containing 1.0 to 1000 milligrams of the active ingredient.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In addition to primates, such as humans, a variety of other mammals may be treated according to the method of the present disclosure. For instance, mammals including, but not limited to, pigs, cows, sheep, goats, horses, dogs, cats, guinea pigs, rats or other porcine, bovine, ovine, caprine, equine, canine, feline, rodent or murine species can be treated. However, the method may also be practiced in other species, such as avian species (e.g., chickens).

The subjects which may treated in the above method are mammals, including, but not limited to, pigs, cows, sheep, goats, horses, dogs, cats, guinea pigs, rats or other porcine, bovine, ovine, caprine, equine, canine, feline, rodent or murine species, and preferably a human being, male or female.

### DEFINITIONS

Unless otherwise herein defined, the following terms will be understood to have the general meanings which follow. The term “effective amount” means the amount of the subject composition that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts.

By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the disclosure to the individual in need of treatment.

The term “C₁₋₆alkyl” encompasses optionally substituted straight chain or branched chain hydrocarbon groups having from 1, 2, 3, 4, 5 or 6 carbon atoms or a range comprising any of two of those integers. Examples include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “C₁₋₆alkyl” also encompasses alkyl groups containing one less hydrogen atom such that the group is attached via two positions i.e. divalent. Such groups are also referred to as “C₁₋₆alkylene” groups. For example, C₁₋₃alkyl and C₁₋₃alkylene groups.

The term “C₂₋₆alkenyl” refers to optionally substituted straight chain or branched chain hydrocarbon groups having at least one double bond of either E or Z stereochemistry where applicable and 2, 3, 4, 5 or 6 carbon atoms or a range comprising any of two of those integers. Examples include vinyl, 1-propenyl, 1- and 2-butenyl, 2-methyl-2-propenyl, hexenyl, butadienyl, hexadienyl, hexatrienyl and the like. Unless the context requires otherwise, the term “C₁₋₆alkenyl” also encompasses alkenyl groups containing one less hydrogen atom such that the group is attached via two positions i.e. divalent. Such groups are also referred to as “C₂₋₆alkenylene” groups. For example, C₂₋₃alkenyl and C₂₋₃alkenylene groups.

The term “C₂₋₆alkynyl” refers to optionally substituted straight chain or branched chain hydrocarbon groups having at least one triple bond and 2, 3, 4, 5 or 6 carbon atoms or a range comprising any of two of those integers. Examples include ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl, 5-hexynyl and the like. Unless the context indicates otherwise, the term “C₂₋₆alkynyl” also encompasses alkynyl groups containing one less hydrogen atom such that the group is attached via two positions i.e. divalent. Such groups are also referred to as “C₂₋₆alkynylene” groups. For example, C₂₋₃alkynyl and C₂₋₃alkynylene groups.

The term “C₃₋₈cycloalkyl” refers to non-aromatic cyclic hydrocarbon groups having from 3, 4, 5, 6, 7 or 8 carbon atoms or a range comprising any of two of those integers, including cyclopropyl, cyclobutyl, cyclopentyl, cyclohexyl, cyclohexenyl, cycloheptyl and the like. It will be understood that cycloalkyl groups may be saturated such as cyclohexyl or unsaturated such as cyclohexenyl. For example, C₃₋₆cycloalkyl.

The terms “hydroxy” and “hydroxyl” refer to the group —OH.

The term “oxo” refers to the group ═O.

The term “C₁₋₆alkoxyl” refers to the group OC₁₋₆alkyl. Examples include methoxy, ethoxy, propoxy, isoproxy, butoxy, tert-butoxy, pentoxy and the like. The oxygen atom may be located along the hydrocarbon chain, and need not be the atom linking the group to the remainder of the compound. For example, C₁₋₃alkoxyl groups.

The term “aryloxy” refers to the group —Oaryl and may include variations thereof such as “alkoxyaryl”, wherein aryl is defined herein. Examples include, but are not limited to, phenoxy and naphthoxy and benzyloxy.

The terms “halo”, “halogen”, “halogenated” and similar terms refers to fluoro, chloro, bromo and iodo (F, Cl, Br, I).

The term “C₁₋₆alkylhalo” refers to a C₁₋₆alkyl which is substituted with one or more halogens. For example, C₁₋₃alkylhalo groups, such as for example —CHF₂ and —CF₃.

The term “C₁₋₆alkoxylhalo” refers to a C₁₋₆alkoxyl which is substituted with one or more halogens. For example, C₁₋₃alkoxylhalo groups, such as for example, —OCHF₂ and —OCF₃.

The term “carboxylate” or “carboxyl” refers to the group —COO⁻ or —COOH.

The term “ester” refers to a carboxyl group having the hydrogen replaced with, for example a C₁₋₆alkyl group (“carboxylC₁₋₆alkyl” or “alkylester”), an aryl or aralkyl group (“arylester” or “aralkylester”) and so on. Examples include but are not limited to CO₂C₁₋₃alkyl, such as for example, methylester (CO₂Me), ethylester (CO₂Et) and propylester (CO₂Pr) and includes reverse esters thereof (e.g. —OCOMe, —OCOEt and —OCOPr).

The term “cyano” refers to the group —CN.

The term “nitro” refers to the group —NO₂.

The term “amino” refers to the group —NH₂.

The term “substituted amino” or “secondary amino” refers to an amino group having a hydrogen replaced with, for example a C₁₋₆alkyl group (“C₁₋₆alkylamino”), an aryl or aralkyl group (“arylamino”, “aralkylamino”) and so on. For example, C₁₋₃alkylamino groups, such as for example, methylamino (NHMe), ethylamino (NHEt) and propylamino (NHPr).

The term “disubstituted amino” or “tertiary amino” refers to an amino group having the two hydrogens replaced with, for example a C₁₋₆alkyl group, which may be the same or different (“dialkylamino”), an aryl and alkyl group (“aryl(alkyl)amino”) and so on. For example, di(C₁₋₃alkyl)amino groups, such as for example, dimethylamino (NMe₂), diethylamino (NEt₂), dipropylamino (NPr₂) and variations thereof (e.g. N(Me)(Et) and so on).

The term “acyl” or “aldehyde” refers to the group —C(═O)H.

The term “substituted acyl” or “ketone” refers to an acyl group having a hydrogen replaced with, for example a C₁₋₆alkyl group (“C₁₋₆alkylacyl” or “alkylketone” or “ketoalkyl”), an aryl group (“arylketone”), an aralkyl group (“aralkylketone”) and so on. For example, C₁₋₃alkylacyl groups

The term “amido” or “amide” refers to the group —C(O)NH₂.

The term “aminoacyl” refers to the group —NHC(O)H.

The term “substituted amido” or “substituted amide” refers to an amido group having a hydrogen replaced with, for example a C₁₋₆alkyl group (“C₁₋₆alkylamido” or “C₁₋₆alkylamide”), an aryl (“arylamido”), aralkyl group (“aralkylamido”) and so on. For example, C₁₋₃alkylamide groups, such as for example, methylamide (—C(O)NHMe), ethylamide (—C(O)NHEt) and propylamide (—C(O)NHPr) and includes reverse amides thereof (e.g. —NHMeC(O)—, —NHEtC(O)— and —NHPrC(O)—).

The term “disubstituted amido” or “disubstituted amide” refers to an amido group having the two hydrogens replaced with, for example a C₁₋₆alkyl group (“di(C₁₋₆alkyl)amido”) or “di(C₁₋₆alkyl)amide”), an aralkyl and alkyl group (“alkyl(aralkyl)amido”) and so on. For example, di(C₁₋₃alkyl)amide groups, such as for example, dimethylamide (—C(O)NMe₂), diethylamide (—C(O)NEt₂) and dipropylamide (—C(O)NPr₂) and variations thereof (e.g. —C(O)N(Me)Et and so on) and includes reverse amides thereof.

The term “carbamic acid” refers to the group NH₂CO₂H.

The term “carbamate” refers to a carbamic acid group having one or both amino hydrogens independently replaced with, for example a C₁₋₆alkyl group (“C₁₋₆alkyl carbamate”), an aryl (“arylcarbamate”), aralkyl group (“aralkylcarbamate”) and so on.

The term “thiol” refers to the group —SH.

The term “C₁₋₆alkylthio” refers to a thiol group having the hydrogen replaced with a C₁₋₆alkyl group. For example, C₁₋₃alkylthio groups, such as for example, thiolmethyl, thiolethyl and thiolpropyl.

The term “thioxo” refers to the group ═S.

The term “sulfinyl” refers to the group —S(═O)H.

The term “substituted sulfinyl” or “sulfoxide” refers to a sulfinyl group having the hydrogen replaced with, for example a C₁₋₆alkyl group (“C₁₋₆alkylsulfinyl” or “C₁₋₆alkylsulfoxide”), an aryl (“arylsulfinyl”), an aralkyl (“aralkyl sulfinyl”) and so on. For example, C₁₋₃alkylsulfinyl groups, such as for example, —SOmethyl, —SOethyl and —SOpropyl.

The term “sulfonyl” refers to the group —SO₂H.

The term “substituted sulfonyl” refers to a sulfonyl group having the hydrogen replaced with, for example a C₁₋₆alkyl group (“sulfonylC₁₋₆alkyl”), an aryl (“arylsulfonyl”), an aralkyl (“aralkylsulfonyl”) and so on. For example, sulfonylC₁₋₃alkyl groups, such as for example, —SO₂Me, —SO₂Et and —SO₂Pr.

The term “sulfonylamido” or “sulfonamide” refers to the group —SO₂NH₂.

The term “substituted sulfonamido” or “substituted sulfonamide” refers to an sulfonylamido group having a hydrogen replaced with, for example a C₁₋₆alkyl group (“sulfonylamidoC₁₋₆alkyl”), an aryl (“arylsulfonamide”), aralkyl (“aralkylsulfonamide”) and so on. For example, sulfonylamidoC₁₋₃alkyl groups, such as for example, —SO₂NHMe, —SO₂NHEt and —SO₂NHPr and includes reverse sulfonamides thereof (e.g. —NHSO₂Me, —NHSO₂Et and —NHSO₂Pr).

The term “disubstituted sulfonamido” or “disubstituted sulfonamide” refers to a sulfonylamido group having the two hydrogens replaced with, for example a C₁₋₆alkyl group, which may be the same or different (“sulfonylamidodi(C₁₋₆alkyl)”), an aralkyl and alkyl group (“sulfonamido(aralkyl)alkyl”) and so on. For example, sulfonylamidodi(C₁₋₃alkyl) groups, such as for example, —SO₂NMe₂, —SO₂NEt₂ and —SO₂NPr₂ and variations thereof (e.g. —SO₂N(Me)Et and so on) and includes reverse sulfonamides thereof.

The term “sulfate” refers to the group OS(O)₂OH and includes groups having the hydrogen replaced with, for example a C₁₋₆alkyl group (“alkylsulfates”), an aryl (“arylsulfate”), an aralkyl (“aralkylsulfate”) and so on. For example, C₁₋₃sulfates, such as for example, OS(O)₂OMe, OS(O)₂OEt and OS(O)₂OPr.

The term “sulfonate” refers to the group SO₃H and includes groups having the hydrogen replaced with, for example a C₁₋₆alkyl group (“alkylsulfonate”), an aryl (“arylsulfonate”), an aralkyl (“aralkylsulfonate”) and so on. For example, C₁₋₃sulfonates, such as for example, SO₃Me, SO₃Et and SO₃Pr.

The term “phosphate” refers to a group —OP(O)(OH)₂ and includes groups having each hydrogen independently replaced with, for example a C₁₋₆alkyl group (“alkylphosphate”), an aryl (“arylphosphate”), an aralkyl (“aralkylphosphate”) and so on.

The term “phosphonate” refers to a group —P(O)(OH)₂ and includes groups having each hydrogen independently replaced with, for example a C₁₋₆alkyl group (“alkylphosphonate”), an aryl (“arylphosphonate”), an aralkyl (“aralkylphosphpmate”) and so on.

The term “aryl” refers to any group containing a carbocyclic (non-heterocyclic) aromatic ring and may be a mono-, bi- or tri-cyclic ring system. The aromatic ring or ring system is generally composed of 6 or 10 carbon atoms. Such groups may contain fused ring systems (such as naphthyl, tetrahydronaphthyl, fluorenyl, indenyl, azulenyl, anthracenyl and the like), linked ring systems (such as biphenyl groups), and may be substituted or unsubstituted. Examples of aryl groups include, but are not limited to, phenyl, biphenyl, naphthyl and tetrahydronaphthyl. For example, phenyl.

The term “aralkyl” refers to an aryl group substituted with a C₁₋₆alkyl group. Examples include benzyl and phenethyl.

The term “heterocyclyl” refers to a moiety obtained by removing a hydrogen atom from a ring atom of a heterocyclic compound which moiety has from 3 to 10 ring atoms (unless otherwise specified), of which 1, 2, 3 or 4 are ring heteroatoms each heteroatom being independently selected from O, S and N.

In this context, the prefixes 3-, 4-, 5-, 6-, 7-, 8-, 9- and 10-membered denote the number of ring atoms, or range of ring atoms, whether carbon atoms or heteroatoms. For example, the term “3-10 membered heterocyclyl”, as used herein, pertains to a heterocyclyl group having 3, 4, 5, 6, 7, 8, 9 or 10 ring atoms or a range comprising any of two of those integers. Examples of heterocyclyl groups include 5-6-membered monocyclic heterocyclyls and 9-10 membered fused bicyclic heterocyclyls.

Examples of monocyclic heterocyclyl groups include, but are not limited to, those containing one nitrogen atom such as aziridine (3-membered ring), azetidine (4-membered ring), pyrrolidine (tetrahydropyrrole), pyrroline (e.g., 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), imidazoline, pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring).

Heterocyclyls also encompass aromatic heterocyclyls and non-aromatic heterocyclyls. Such groups may be substituted or unsubstituted.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O.

“Heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to about four heteroatoms typically selected from nitrogen, sulfur and oxygen. The heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered heteroaryl groups containing nitrogen include pyridyl (1 nitrogen), pyrazinyl, pyrimidinyl and pyridazinyl (2 nitrogens). It will be understood that, such as in the case of pyridyl when substituted with an oxo (═O) substituted the group may be interchangeably referred to as a pyridinone group.

Aromatic heterocyclyl groups may also be bicyclic or polycyclic heteroaromatic ring systems such as fused ring systems (including purine, pteridinyl, napthyridinyl, 1H thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, naphthyl, indenyl, azulenyl, fluorenyl, anthracenyl and the like, such as 5- or 6-membered aromatic heterocyclyls fused to a phenyl ring including 5-membered aromatic heterocyclyls containing nitrogen fused to a phenyl ring, 5-membered aromatic heterocyclyls containing 1 or 2 nitrogens fused to a phenyl ring and such as 5- or 6-membered aromatic heteroaryls fused to a 6-membered aromatic or non-aromatic heterocyclyls.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f) an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; 1) a furan ring fused to a 5- or 6-membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms.

Particular examples of bicyclic heteroaryl groups containing a five membered ring fused to another five membered ring i.e. 8-membered fused bicyclic rings include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring i.e. 9-membered fused bicyclic rings include but are not limited to benzofuran, benzothiophene, benzimidazole, benzoxazole, isobenzoxazole, benzisoxazole, benzothiazole, benzisothiazole, isobenzofuran, indole, isoindole, indolizine, indoline, isoindoline, purine (e.g. adenine, guanine), indazole, imidazopyridine (e.g. imidazo[1,2-a]pyridine and imidazo[4,5-b]pyridine], pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings i.e. 10-membered fused bicyclic rings include but are not limited to quinoline, isoquinoline, chroman, thiochroman, chromene (including optionally substituted with oxo (═O) i.e. oxochromene), isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoline, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “non-aromatic heterocyclyl” encompasses optionally substituted saturated and unsaturated rings which contain at least one heteroatom selected from the group consisting of N, S and O.

Non-aromatic heterocyclyls may be 3-7 membered mono-cyclic rings. The term “3-7 membered monocyclic”, as used herein, pertains to a mono-cyclic group having 3, 4, 5, 6 or 7 ring atoms or a range comprising any of two of those integers. Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihyrdopyranyl, tetrahydropyranyl, 2H pyranyl, 4H pyranyl, thianyl, thianyl oxide, thianyl dioxide, piperazinyl, diozanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like.

Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to carbocyclic aromatic rings such as phenyl, naphthyl, indenyl, azulenyl, fluorenyl, anthracenyl and the like. Examples of non-aromatic 5-membered, 6-membered or 7-membered heterocyclyls fused to carbocyclic aromatic rings include indolinyl, benzodiazepinyl, benzazepinyl, dihydrobenzofuranyl and the like.

The term “spiro ring system” means a bicyclic ring system in which the rings are connected via a single shared atom or “spiroatom”. For example, a quaternary carbon (“spiro carbon”) and encompasses spiro bicyclic 7-11-membered carbocyclic rings and spiro bicyclic 7-11-membered heterocyclic rings containing one, two, three or four heteroatoms independently selected from O, N and S.

The term “derived from an amino acid” refers to any side chain that may be present in natural (L-) or unnatural (D-) amino acids. Examples of amino acid side chain moieties derived from natural amino acids, with the amino acids from which they are derived shown in brackets, are —H (Glycine), —CH₃ (Alanine), —CH(CH₃)₂ (Valine), —CH₂CH(CH₃)₂ (Leucine), —CH(CH₃)CH₂CH₃ (Isoleucine), —(CH₂)₄NH₂ (Lysine), —(CH₂)₃NHC(═NH)NH₂ (Arginine), —CH₂— (5-1H-imidazolyl) (Histidine), —CH₂CONH₂ (Asparagine), —CH₂CH₂CONH₂ (Glutamine), —CH₂COOH (Aspartic acid), —CH₂CH₂COOH (Glutamic acid), —CH₂-phenyl (Phenylalanine), —CH₂-(4-OH-phenyl) (Tyrosine), —CH₂-(3-1H-indolyl) (Tryptophan), —CH₂SH (Cysteine), —CH₂CH₂SCH₃ (Methioine), —CH₂OH (Serine), —CH(OH)CH₃ (Threonine) and the cyclic side chain pyrrolidinyl (Proline) whereby the covalent bond between the nitrogen and carbon in the pyrrolidinyl ring forms the backbone. Examples of amino acid side chain moieties derived from unnatural amino acids, with the amino acids from which they are derived shown in brackets, are —(CH₂)₂—C(O)—O—C(CH₃)₃ (glutamic acid t-butyl ester), —(CH₂)₄—NH—C(O)—O—C(CH₃)₃ (Nₑ-(tert-butoxycarbonyl)-lysine), —(CH₂)₃—NH—C(O)NH₂ (citrulline), —CH₂—CH₂OH (homoserine) and —(CH₂)₂—CH₂NH₂ (ornithine). Examples can also include alkyl, alkenyl, alkynyl, aryl, saturated and unsaturated heterocycles (functionalized and unfunctionalized). The term “amino-acid side chain moiety” can also include a number of unnatural amide and sulfonamide, aryl and heteroaryl side chains.

Unless otherwise defined, the term “optionally substituted” or “optional substituent” as used herein refers to a group which may or may not be further substituted with 1, 2, 3, 4; 1, 2 or 3; or 1 or 2 groups selected from the group consisting of C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃ and CHF₂), C₁₋₆alkoxyhalo (such as OCF₃ and OCHF₂), pentafluorosulfanyl (SF₅), carboxylic acid, carboxyl, esters, cyano, nitro, amino, mono substituted amino, disubstituted amino, acyl, ketones, amides, aminoacyl, substituted amides, disubstituted amides, carbamic acid, carbamates, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, phosphates, phosphonates, aryl, arC₁₋₆alkyl, heterocyclyl, heteroaryl and spiro ring systems wherein each alkyl, alkenyl, alkynyl, cycloalkyl, aryl, heterocyclyl, heteroaryl and spiro ring system and groups containing them may be further optionally substituted. Unless otherwise defined, examples of optional substituents in one embodiment of the invention include 1, 2, 3 or 4, e.g. 1 or 2 substituents each independently selected from the group consisting of C₁₋₄alkyl (e.g. methyl), halo (e.g. F), haloC₁₋₃alkyl (e.g. CHF₂ and CF₃), OH, C₁₋₄alkoxyl (e.g. OCH₃), CO₂H, CO₂C₁₋₄alkyl (e.g. CO₂CH₃), NH₂, NHC₁₋₄alkyl (e.g. NHCH₃), N(C₁₋₄alkyl)₂ (e.g. N(CH₃)₂), NHC(═O)C₁₋₄alkyl, NHC(═O)-4-6-membered heterocyclyl, OP(═O)(OR)₂ (where each R is independently H or C₁ alkyl), P(═O)(OR)₂ (where each R is independently H or C₁₋₄alkyl), C₃₋₆cycloalkyl (e.g. cyclopropyl, cyclobutyl, cyclopenyl and cyclohexyl), phenyl, 4-6-membered heterocyclyl (e.g. oxetanyl, azetidinyl, tetrahydrofuranyl, pyrrolidinyl, tetrahydropyranyl, morpholinyl, thiomorpholinyl, oxothiazinyl, dioxothiazinyl, thianyl (also known as tetrahydrothiopyranyl), oxothianyl, dioxothianyl, piperidinyl, and piperazinyl) and further where C₁₋₄alkyl either alone or as part of a substituent group includes methyl, ethyl, n-propyl, iso-propyl, n-butyl, iso-butyl, sec-butyl and tert-butyl and may be further optionally substituted.

Optional substituents in the case of heterocycles, heteroaryls and spiro bicyclic heterocyclic ring systems containing N may also include but are not limited to alkyl i.e. N—C₁₋₃alkyl. For example, methyl. In one example, N-methyl.

It will be understood that suitable derivatives of aromatic heterocyclyls containing nitrogen include N-oxides thereof.

### Methods of Preparation

Generally, the compounds of the present disclosure may be prepared according to the methods previously described in applicant's earlier filed applications WO2007/148093, WO2009/074812, WO2009/074810, WO2012/045124 and WO2013/138860.

Compounds of general Formula (II) which are not previously disclosed in applicant's early filed applications, may be generally prepared as follows:

### General Method A

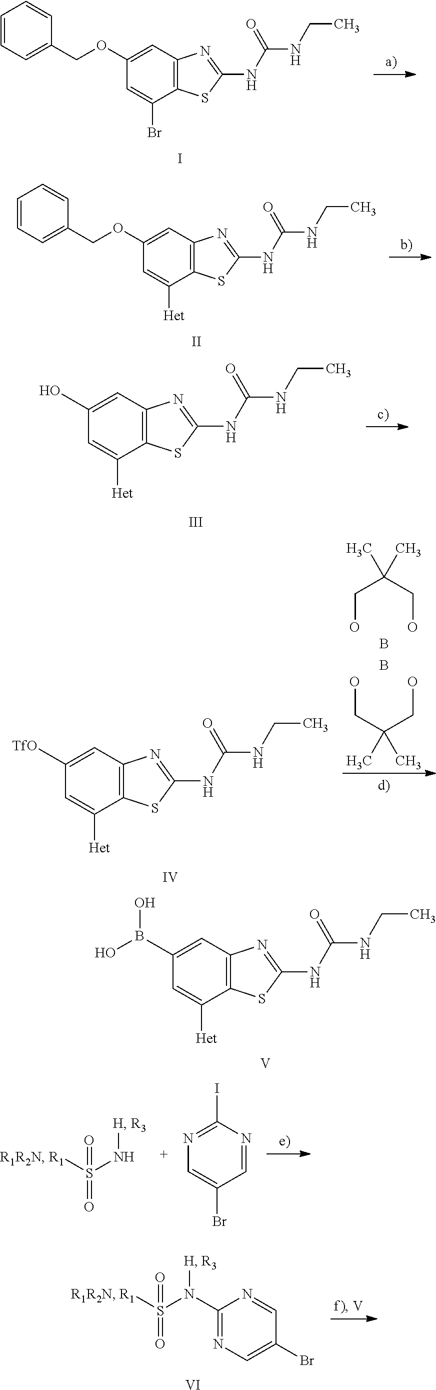

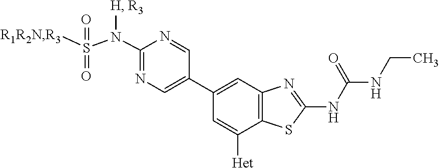

a) Coupling (e.g. Het-SnBu₃, Pd(PPh₃)₄, DMF); b) Protecting group removal (e.g. MsOH, DCM); c) Triflate formation (e.g. (CF₃SO₂)₂NPh, DIPEA, DMF); d) Boronic acid formation (e.g. Pd(dppf)Cl₂.DCM, KOAc, DMSO); e) Coupling (e.g. Pd₂(dba)₃, Xantphos, Cs₂CO₃, dioxane); f) Coupling (e.g. Pd(dppf)Cl₂.DCM, aq. Na₂CO₃ or aq. K₃PO₄, Pd(PPh₃)₂Cl₂, in DMF or dioxane).

Intermediate I was subjected to a cross coupling reaction such as Stille coupling with a heterocyclic stannane (e.g. 2-pyridyltributyltin) in the presence of a Pd catalyst in a solvent such as DMF, resulting in C₇-substituted intermediate II. Cleavage of a protecting group (such as removal of a benzylic ether using excess methanesulfonic acid in dichloromethane) gave III which was activated by conversion to a halide or triflate IV (e.g. triflate formed by reaction with bis(trifluorophenylsulfonyl)anilide in the presence of Hunig's base in DMF). This intermediate was converted to the boronic acid or boronic ester (for example by Pd-mediated cross-coupling with bis-glycolatodiboron) giving V. Formation of the C₅ moiety was achieved by Buchwald-type coupling for example between an appropriately substituted sulfonamide or sulfamide and a heterocycle such 2-iodo-5-bromopyrimidine to generate intermediates VI, which were then installed onto core intermediate V via a Suzuki coupling or similar method.

### Variations to General Method A

### Routes A1 and A2

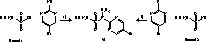

a) Sulfonyl chloride formation and sulfonylation e.g. (COCl)₂, DMF (cat), dicloromethance then KO^{t}Bu; b) Displacement reaction e.g. K₂CO₃, DMF.

Alternative preparations of sulfonamide/sulfamide building blocks VI could be accomplished via Route A1, wherein sulfonic acids were converted to the sulfonyl chlorides by means of Vilsmeier-type conditions (e.g. with oxalyl chloride/catalytic DMF) and then trapping with an amino heterocycle such as 5-bromo-2-aminopyrimidine. Intermediates of type VI can also be formed via Route A2, where a direct S_{N}Ar reaction takes place between a sulfonamide/sulfamide and an appropriate halogenated heterocycle, for example 5-bromo-2-fluoropyrimidine. Alternatively, in Route A3, pyridine intermediates were formed by Buchwald-type coupling between the sulfonamide/sulfamide and an appropriately substituted halogenated pyridine such as 2,5-dibromopyridine. In Route A4, intermediates of type VI were prepared by sulfonylation of 5-bromo-2-aminopyrimidine with sulfonyl chlorides in the presence of a strong base. In Route A5, heterocyclic-methylpyridones were prepared by opening of the pyranone ring with a substituted aminomethylheterocycle, such as 2-aminomethyl-6-methylpyridine. Triflate formation and Suzuki coupling afforded the final products.

### Route A3

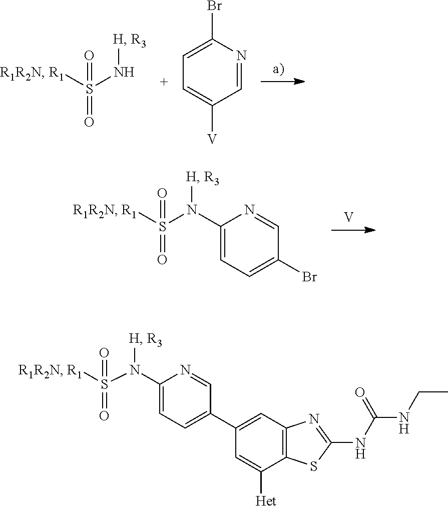

a) Coupling reaction e.g. Pd₂(dba)₃, Xantphos, Cs₂CO₃, Dioxane

### Route A4

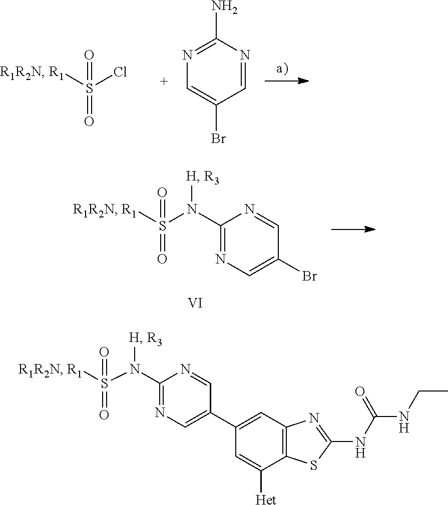

a) Sulfonylation reaction e.g. KO^{t}Bu, THF

### General Method B

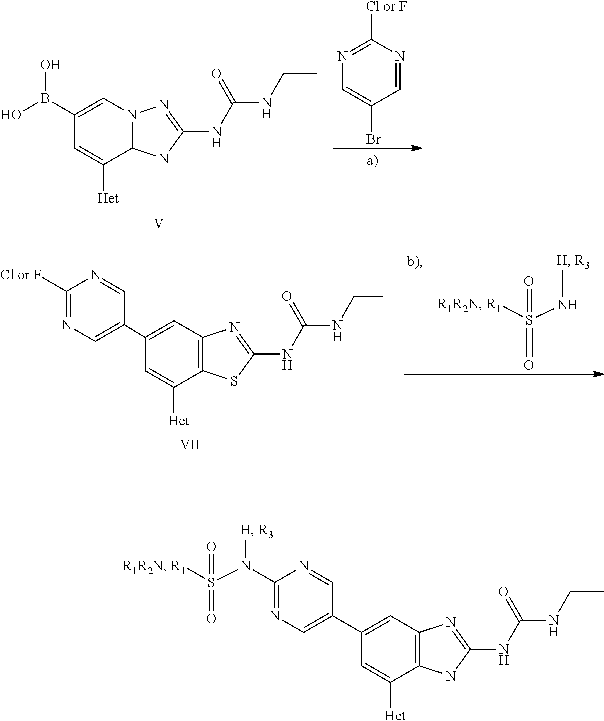

a) Coupling reaction e.g. Pd(dppf)Cl₂.DCM, aq. Cs₂CO₃, dioxane; b) Coupling reaction e.g. Pd₂(dba)₃, Xantphos, Cs₂CO₃, dioxane, microwave, or displacement e.g. Cs₂CO₃, dimethylacetamide.

In General Method B an appropriately substituted halogenated heterocycle (e.g. 5-bromo-2-chloro pyrimidine or 5-bromo-2-fluoropyrimidine) is first added to intermediate V via a coupling reaction such as Suzuki coupling to form VII which is subsequently coupled to a sulfonamide or sulfamide by either Buchwald-type coupling reaction or direct S_{N}Ar displacement of the halogen.

### General Method C

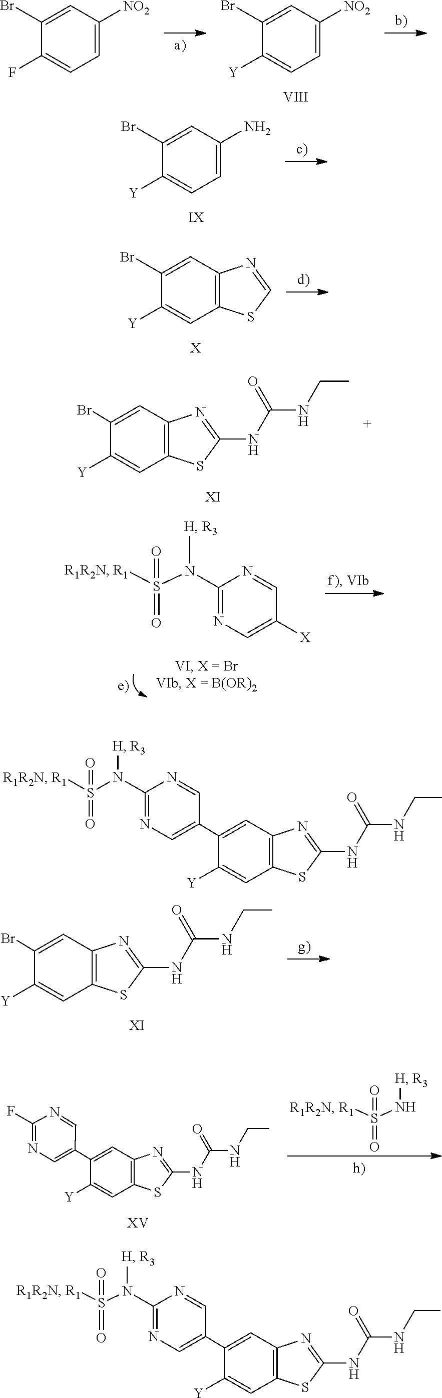

a) Displacement e.g. NaOH, DMSO; b) Reduction e.g. SnCl₂.2H₂O, THF; c) Cyclization: i) NH₄SCN, ii) Br₂, AcOH; d) Urea formation e.g. EtNCO, dioxane; e) Coupling e.g. B₂(OR)₂, PCy₃, Pd₂(dba)₃, dioxane; f) Coupling e.g. Pd(dppf)Cl₂.DCM, K₃PO₄, aq. dioxane; g) i) Coupling: B₂(pin)₂, Pd(dppf)Cl₂.DCM, KOAc, DMSO; ii) 5-bromo-2-fluoropyrimidine, Pd(dppf)Cl₂.DCM, aq. NaHCO₃, dioxane; h) Displacement e.g. Cs₂CO₃, dimethylacetamide.

Preparation of C₆-substituted benzothiazoles was accomplished according to General Method C. Displacement of 3-bromo-4-fluoronitrobenzene with nucleophiles (for example Y=ether groups) afforded the appropriately substituted nitrobenzenes VIII, which upon reduction with tin dichloride, afforded anilines IX. Cyclization to the aminobenzimidazoles X followed in two steps: condensation with ammonium thiocyanate, thereafter oxidative cyclization for example with bromine in acetic acid. Urea formation in the presence of ethyl isocyanate gave intermediate XI. Cross-coupling then took place with boronate esters (or acids) VIb, derived from the 5-bromopyrimidines VI via standard borylation procedures. Alternatively, 6-fluorobenzothiazolesulfonamides were prepared in similar fashion, except that the bromofluorobenzothiazole intermediate XIV was coupled with the boronate ester derived from a halogenated heterocycle such as 5-bromo-2-fluoropyridine. Direct S_{N}Ar displacement of the fluorine by reaction with sulfonamides then delivered the target products.

### General Method D

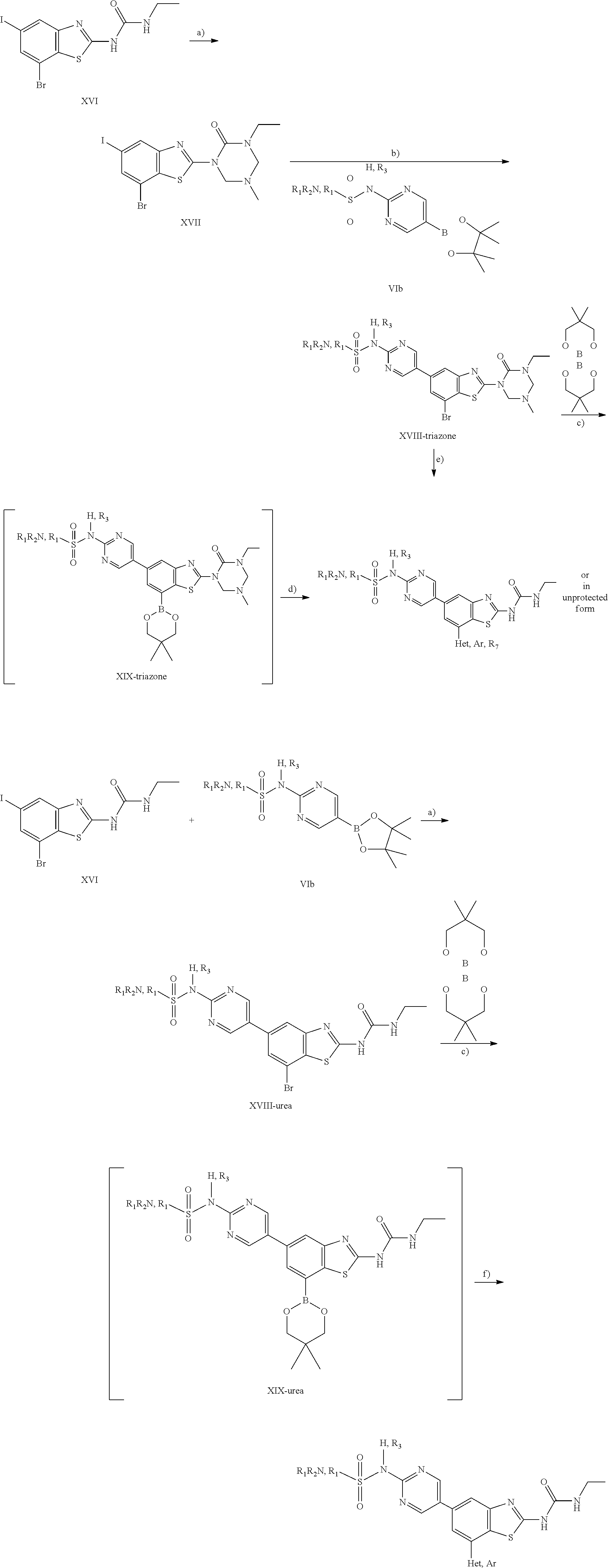

a) Protection (CH₂O)ₙ, MeNH₂/THF, MeOH, NMM; b) Coupling Pd(dppf)Cl₂.DCM, 2M aq. Na₂CO₃ or aq. K₃PO₄, Pd(PPh₃)₂Cl₂, solvent such as DMF, dioxane, 53 dioxane/MeOH; c) Coupling Pd(dppf)Cl₂.DCM, KOAc, toluene, thermal or microwave; d) Coupling i) Ar-Br/Het-Br, Pd(dppf)Cl₂.DCM, aq. Cs₂CO₃, dioxane; Deprotection ii) 4M HCl/dioxane. e) i) Coupling Trialkylboroxine, Pd(dppf)Cl₂.DCM, aq. Cs₂CO₃, dioxane; or heteroarylstannane, Pd(PPh₃)₄, DMF ii) Deprotection 4M HCl/dioxane f) Coupling Het-Br/Ar—Br, Pd(PPh₃)₄, aq. Cs₂CO₃, dioxane.

A suitable benzothiazole such as the 5-iodo-7-bromobenzothiazole XVI (see WO2012045124) can be protected as the triazone XVII for example by treatment with paraformaldehyde, methylamine, and N-methylmorpholine. Coupling with a boronate ester VIb prepared as described in Route C, under Suzuki conditions, selectively favors replacement of the iodine at the 5 position of the ring, giving XVIII-triazone. Installation of the C₇ heterocycle proceeded first by replacement of the C₇ bromide with the glycolatoboron esters XIX-triazone, which were not isolated, but coupled directly under Suzuki conditions with heteroaryl bromides (Het-Br) and subsequently deprotected under acidic conditions to liberate the free ureas. Use of non-aryl boronates such as trimethylboroxine permitted direct conversion of XVIII-triazone to C7-alkyl substituted examples, which upon acidic deprotection resulted in the target compounds. Alternatively, direct coupling of XVIII-triazone with heteroarylstannanes under Stille conditions, afforded target C7-heteroaryl compounds.

In unprotected form, XVI can be coupled directly with sulfonamide/sulfamide boronates VIb and then subjected to one-pot boronate formation, giving boronate intermediates XIX-urea, which were then cross-coupled under Suzuki conditions to give the final products.

### General Method E

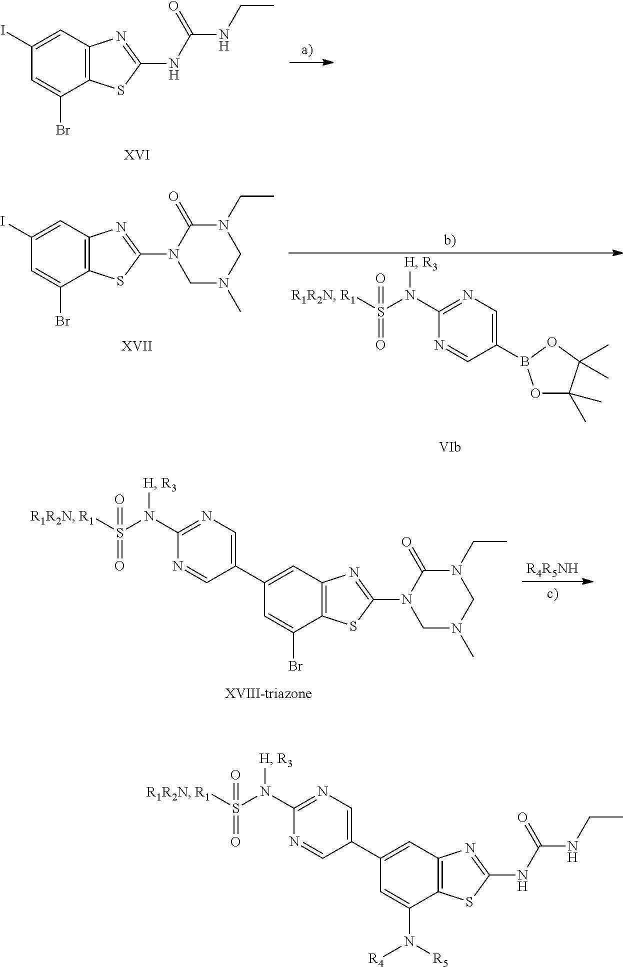

A suitable benzothiazole such as the 5-iodo-7-bromobenzothiazole XVI (see WO2012045124) was protected as the triazone XVII (see Route D). Cross-coupling with sulfonamide or sulfamide boronates VIb under Suzuki conditions afforded C₇-bromobenzothiazoles XVIII-triazone, from which C₇-aminosubstituted benzothiazoles were prepared by means of Buchwald-type coupling with amines R₄R₅NH in the presence of palladium catalysts, under microwave radiation. During the course of the reaction, the triazone group was cleaved, permitting the urea-based products to be isolated directly.

### General Method F

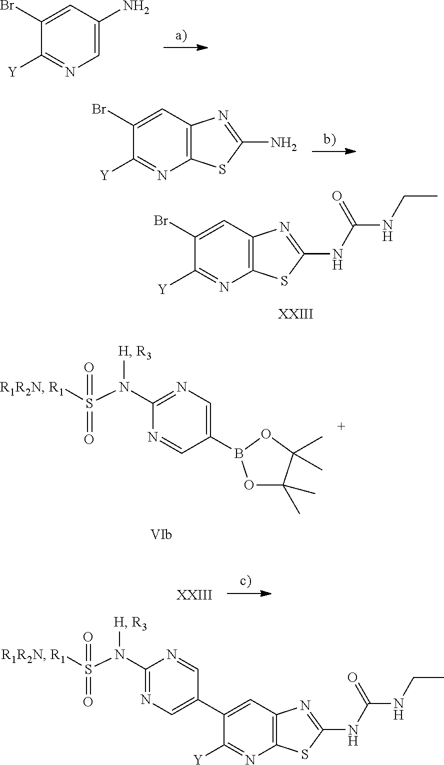

a) Oxidative cyclization e.g. KSCN, Br₂, HOAc; b) Urea formation EtNCO, Et₃N, DME/THF; c) Pd(PPh₃)₄, NaHCO₃. DME, H₂O

Oxidative cyclization of substituted pyridines, (such as Y=MeO; 5-bromo-6-methoxy-pyridin-3-amine) followed by urea formation (ethyl isocyanate/triethylamine in dimethoxyethane/THF) afforded thiazolopyridines XXIII, which were then subjected to Suzuki coupling with sulfonamide boronates VIb to deliver the 6-substituted thiazolopyridine derivatives.

### General Method G

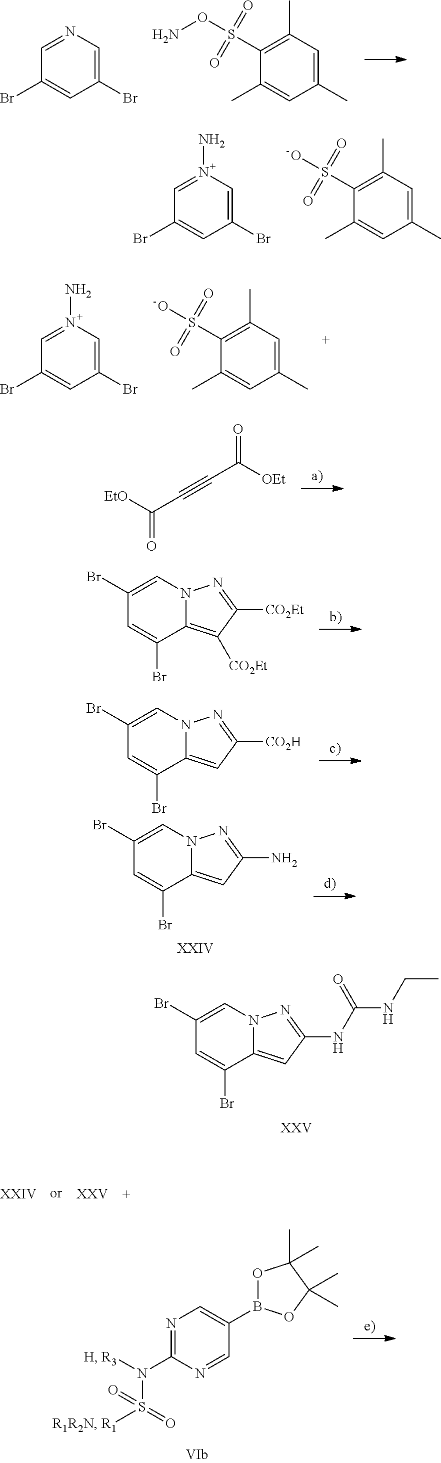

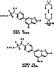

a) K₂CO₃, DMF, RT; b) H₂SO₄, 120-130° C.; c) DPPA, Et₃N, t-BuOH, then TFA/DCM; d) EtNCO, Bu₂Sn(OAc)₂, toluene, reflux; e) Pd(dppf)Cl₂.DCM, Cs₂CO₃, dioxane/H₂O; f) Coupling Pd(dppf)Cl₂.DCM, KOAc, toluene, thermal or microwave; g) Coupling Het-Br/Ar—Br, Pd(PPh₃)₄, aq. Cs₂CO₃, dioxane.

N-amination of 3,5-dibromopyridine with mesitylenehydroxylamine (Mendiola, J. et al. Org. Process R&D (2009) 13, 263-267), followed by cyclization with diethylacetylene dicarboxylate formed the pyrazolopyridines, which were then subjected to saponification and thermal decarboxylation to give the 2-carboxylate derivatives. Curtius rearrangement and cleavage of the Boc groups delivered the 2-aminopyrazolopyridines XXIV. Acylation with ethyl isocyanate formed ureas XXV. Either XXIV or XXV were then subjected to Suzuki-type coupling with boronates VIb selectively at the C5 position to give XXVI or XXVII respectively. Finally, derivatives could be formed at the C7 position via one-pot Suzuki-type coupling to give XXVIII (aminopyrazolopyridines) or XXIX (aminopyrazolopyridine ureas).

### General Method H

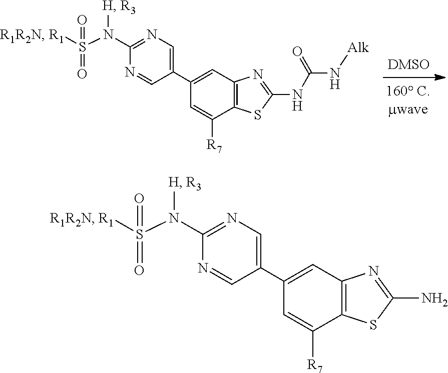

Deacylation of aminobenzothiazole alkyl ureas is accomplished by thermal decomposition under microwave conditions, to form the 2-aminobenzothiazoles as shown.

### General Method I

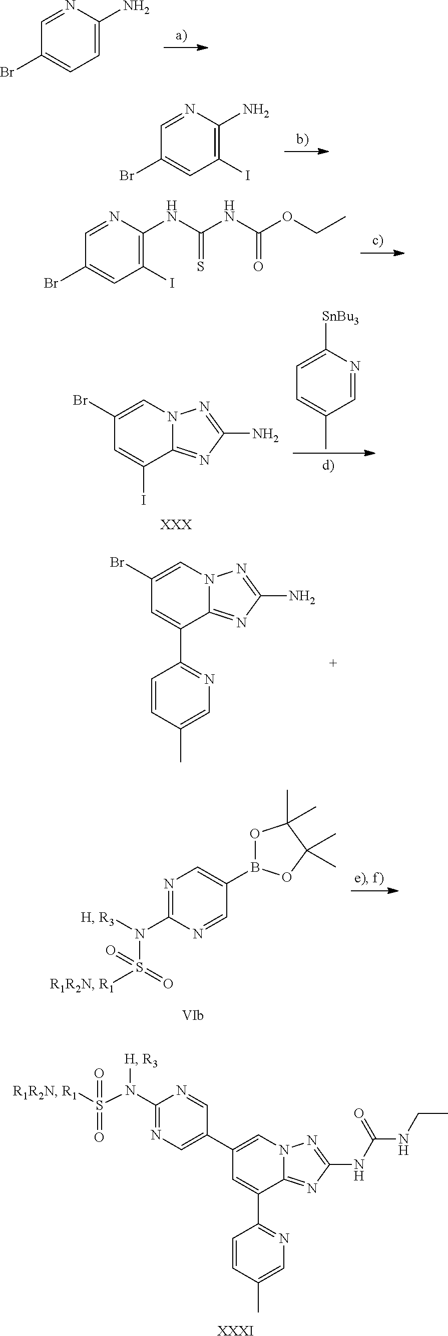

a) NIS, TFA, DMF; b) EtOOCNCS, dioxane; c) HONH₂.HCl, DIPEA, MeOH; d) Pd(PPh₃)₄, DMF; e) Pd(dppf)Cl₂. CH₂Cl₂, Cs₂CO₃, aq. dioxane; f) EtNCO, Bu₂Sn(OAc)₂, toluene.

Regioselective iodination of 2-amino-5-bromopyridine with N-iodosuccinimide, followed by N-carbamoylthiourea formation mediated by the addition of ethoxycarbonyl isothiocyanate, was followed by ring closure to form 2-aminotriazolopyridine XXX in the presence of hydroxylamine at 50° C. Stille coupling proceeded selectively at the iodo group, following which, Suzuki coupling with boronates VIb completed the synthesis of disubstituted triazolopyridine ureas XXXI.

### Salt Formation Method(s)

Salts of the compounds of the present disclosure may be formed using conditions familiar to those in the art, for example, as follows.

### General Salt Formation Conditions

The free base material is dissolved or suspended in an organic solvent, organic solvent mixture or organic solvent water mixture (for example; DCM, THF, THF/MeOH, EtOH) and a solution/suspension of the acid in the same organic solvent or organic solvent mixture in molar equivalents of 1 or greater than 1 is added. The acid may also be added neat. The salt product may precipitate at room temperature or alternatively the addition may be done at a higher temperature with subsequent cooling to enable precipitation of the salt product. An antisolvent (for example; hexanes, n-heptane, Isopropyl acetate) may be added after the addition of acid to enable precipitation of the salt product which is collected by vacuum filtration and washed with an appropriate organic solvent.

### Example Hydrochloride Salts

Hydrochloride salts can be made, for example, by suspending the compound in a suitable solvent, such as acetonitrile, and adding aqueous 2M hydrochloric acid solution. Dilution of the mixture with water and then removal of the solvent gives the hydrochloride salt of the compound.

### Example Methanesulfonic Acid Salts

Methanesulfonic acid salts can be made, for example, by suspending the compound in a suitable solvent, such as acetonitrile, and adding 1 equivalent of methanesulfonic acid in water. Removal of the solvent gives the methanesulfonic acid salt of the compound.

### Chiral Separation Method(s) and Synthesis

Compounds of the present disclosure may be separated into their diastereoisomers or enantiomers under chiral HPLC conditions familiar to those in the art. Alternatively, chiral precursor moieties may be resolved from their racemates via derivatization with a chiral auxiliary, such as a blocked amino acid, e,g, Boc-valine, separated by fractional crystallization of diastereomers from a suitable solvent, such as heptane, and reconstitution of the enantiomeric precursors through cleavage of the auxiliary, such as base-mediated cleavage on resin or in solution. Mitsonobu-type inversion of enantiomerically enriched mixtures of chiral alcohols can also be accomplished by coupling the alcohol with the chiral auxiliary, such as an amino acid, e.g. Boc-valine, in the presence of trialkylated phosphines, such as triphenylphosphine, and dialkylazodicarboxylates, and fractionally crystallizing the enriched diastereomeric mixture as above.

### Protecting Groups

During the reactions a number of the moieties may need to be protected. Suitable protecting groups are well known in industry and have been described in many references such as Protecting Groups in Organic Synthesis, Greene T W, Wiley-Interscience, New York, 1981. It will be understood that in addition to protecting groups such as hydroxyl and amino groups during the course of reaction, the urea moiety may require protection under any of the reactions conditions described herein, for example, as a 5-methyl-1,3,5-triazinan-2-one.

### Functional Group Interconversions

Further, it will be understood that compounds of the present disclosure produced under any of the reaction conditions described herein may undergo further functionalisation under suitable conditions familiar to those in the art. That is, the skilled person will appreciate that a wide diversity of compounds may be provided by functional group interconversions of hydroxyls and carboxylates including but not limited to halogens, ethers, ketones, carboxylic acids, esters, carbonates, amines, amides, ureas, carbamates, sulfates, sulfonamides, phosphates, heterocycles, heteroaryls, optionally substituted alkyl chain extensions and so on.

### EXAMPLES

Those skilled in the art will appreciate that the present disclosure described herein is susceptible to variations and modifications other than those specifically described. The invention will now be described without limitation by reference to the examples which follow.

The abbreviations used in the Examples are as follows unless indicated otherwise.

### Abbreviations

Ac: acetyl
ACN: acetonitrile
cfus: colony forming units
DCM: dichloromethane

### DIPEA: N,N-diisopropylethylamine

DMAP: N,N-dimethylpyridin-4-amine

### DMF: N,N-dimethylformamide

DMSO: dimethylsulfoxide
EtOAc: ethyl acetate
Et₂O: diethyl ether
EtOH: ethanol
g: gram(s)
h: hour(s)

### H₂O: Water

HPLC: high performance liquid chromatography
IPA: propan-2-ol
kg: kilogram(s)
L: litre(s)
LCMS: liquid chromatography coupled mass spectrometry
LDA: lithium diisopropylamide
M: molar
mg: milligram(s)
MIC: minimum inhibitory concentration
min minute(s)
mL: millilitre(s)
MeOH: methanol
mol: mole(s)
mmol: millimole(s)
MS: mass spectrometry

### NB S: N-bromosuccinimide

NMP: 1-methylpyrrolidin-2-one
NMR: nuclear magnetic resonance
Pd(dppf)Cl₂: [1,1′-Bis(diphenylphosphino)ferrocene]dichloropalladium(II), DCM adduct
Pd₂(dba)₃ Tris(dibenzylideneacetone)dipalladium(0)
Pd(PPh₃)₄ Tetrakis(triphenylphosphine)palladium(0)
RT: room temperature
THF: tetrahydrofuran
TLC: thin-layer chromatography

### Compound Synthesis

¹H NMR spectra were recorded on either a Brüker Avance DRX 400, AC 200 or AM 300 spectrometer. Spectra were recorded in deuterated solvents (CDCl₃, MeOD, DMSO-d₆, CD₃CN, or Acetone-d₆) using the residual solvent peak as a reference. Chemical shifts are reported on the 6 scale in parts per million (ppm) using the following conventions to assign the multiplicity: s (singlet), d (doublet), t (triplet), q (quartet), p (pentet), m (multiplet) and prefixed br (broad). Mass spectra (ESI) were recorded on either a Micromass Platform QMS or Thermo Finnigan LCQ Advantage spectrometer. Flash chromatography was performed on 40-63 μm silica gel 60 (Merck No. 9385). Automated flash chromatography was performed either on a Combi-Flash™ purification system using Combi-Flash™ silica gel columns or on a Biotage SP4 purification system using either GraceResolv™ silica gel cartridges, Grace Reveleris™ C-18 reverse phase silica gel cartridges or Biotage SNAP™ C-18 reverse phase silica gel cartridges. Preparative HPLC was carried out using either a Gilson 322 pump with a Gilson 215 liquid handler and a HP1100 PDA detector or an Agilent 1200 Series mass detected preparative LCMS using a Varian XRs C-18 100×21.2 mm column Unless otherwise specified, the HPLC systems employed Phenomenex C8(2) columns using either acetonitrile or acetonitrile containing 0.06% TFA in water, water containing 0.1% TFA or water containing 0.1% formic acid.

### Example 1

Compounds A-1, A-2, A-3, A-4, A-5 and A-6 of Formula (I) were prepared with reference to the methods described in applicant's earlier filed application WO2007/148093.

### (3R)—N-[5-[2-(Ethylcarbamoylamino)-7-(5-methyl-2-pyridyl)-1,3-benzothiazol-5-yl]pyrimidin-2-yl]-3-hydroxy-pyrrolidine-1-carboxamide (A-1)

Compound synthesised and characterised: m/z: 519.2 [M+H]⁺; and ¹H NMR (400 MHz, DMSO-d₆) δ 10.65 (s, 1H), 9.38 (s, 1H), 9.11 (s, 2H), 8.67 (dt, J=2.2, 0.8 Hz, 1H), 8.43 (d, J=8.3 Hz, 1H), 8.29 (d, J=1.7 Hz, 1H), 8.06 (d, J=1.6 Hz, 1H), 7.85 (ddd, J=8.1, 2.3, 0.9 Hz, 1H), 7.00 (t, J=5.6 Hz, 1H), 5.00 (d, J=3.4 Hz, 1H), 4.38-4.26 (m, 1H), 3.66-3.45 (m, 2H), 3.39-3.17 (m, 4H), 2.42 (s, 3H), 2.04-1.76 (m, 2H), 1.14 (t, J=7.2 Hz, 3H).

### 1-Ethyl-3-[5-[2-methyl-1-[(6-methyl-2-pyridyl)methyl]-6-oxo-4-pyridyl]-7-(2-pyridyl)-1,3-benzothiazol-2-yl]urea (A-2)

Compound synthesised and characterised: m/z: 511 [M+H]⁺; and ¹H NMR (400 MHz, DMSO-d₆) δ 10.69 (s, 1H); 8.86 (d, J=4 Hz, 1H), 8.56 (d, J=8 Hz, 1H), 8.34 (d, J=1.2 Hz, 1H), 8.09 (s, 1H); 8.04 (dt, J=6, 1.2 Hz, 1H), 7.69 (t, J=8 Hz, 1H), 7.50 (m, 1H), 7.19 (d, J=7.6 Hz, 1H), 6.97 (d, J=8 Hz, 1H), 6.93 (s, 1H), 6.89 (d, J=1.2 Hz, 2H), 5.37 (s, 2H), 3.25 (m, 2H), 2.49-2.55 (m*+DMSO-d₆), 1.16 (t, J=7.2 Hz, 3H).

### 1-Ethyl-3-[5-(4-methylimidazol-1-yl)-7-(2-pyridyl)-1,3-benzothiazol-2-yl]urea (A-3)

Compound synthesised and characterised: m/z: 379.04 [M+H]⁺; and ¹H NMR (400 MHz, CD₃OD): δ 9.51 (s, 1H); 8.87 (d, 1H); 8.39 (d, 1H); 8.28 (s, 1H); 8.18 (t, 1H); 8.06 (s, 1H); 7.98 (s, 1H); 7.62 (m, 1H); 3.35 (m*, 2H); 2.72 (s, 6H); 2.52 (s, 3H); 1.22 (t, J=7.2 Hz, 3H).

### 1-Ethyl-3-[5-[2-methyl-1-[1-(6-methyl-3-pyridyl)ethyl]-6-oxo-4-pyridyl]-7-(2-pyridyl)-1,3-benzothiazol-2-yl]urea (A-4)

Compound synthesised and characterised: m/z: 525 [M+H]⁺; and ¹H NMR (400 MHz, DMSO-d₆) δ 10.67 (s, 1H); 8.86 (d, J=3.6 Hz, 1H), 8.53 (d, J=8.4 Hz, 1H), 8.40 (s, 1H), 8.38 (s, 1H), 8.03 (m, 2H), 7.59 (d, J=8 Hz, 1H), 7.50 (m, 1H), 7.26 (d, J=8.4 Hz, 1H), 6.88 (s, 1H), 6.76 (s*, 2H), 3.64 (m, 1H), 3.27 (m, 2H), 2.49-2.55 (s*+DMSO-d₆, 3H), 1.96 (d, J=6.8 Hz, 3H), 1.16 (t, J=7.2 Hz, 3H).

### Methyl N-[5-[2-(ethylcarbamoylamino)-7-(2-pyridyl)-1,3-benzothiazol-5-yl]pyrimidin-2-yl]carbamate (A-5)

Compound synthesised and characterised: m/z: 450.27 [M+H]⁺; and ¹H NMR (400 MHz, DMSO-d₆) δ 10.59 (s, 1H), 10.57 (s, 1H), 9.18 (s, 2H), 8.82 (d, J=3.6 Hz, 1H), 8.51 (d, J=8 Hz, 1H), 8.35 (s, 1H), 8.38 (s, 1H), 8.10 (s, 1H), 8.02 (m, 1H), 7.46 (m, 1H), 6.86 (m, 1H), 3.70 (s, 3H), 3.22 (m, 2H), 1.12 (t, J=7.2 Hz, 3H).

### N-[5-[2-(Ethylcarbamoylamino)-7-[4-(morpholinomethyl)-2-pyridyl]-1,3-benzothiazol-5-yl]pyrimidin-2-yl]pyrrolidine-1-carboxamide (A-6)

Compound synthesised and characterised: m/z: 588.35 [M+H]⁺; and ¹H NMR (400 MHz, DMSO-d₆) δ 10.58 (s, 1H), 9.34 (s, 1H), 9.09 (s, 2H), 8.75 (d, J=5.0 Hz, 1H), 8.39 (s, 1H), 8.30 (d, J=1.7 Hz, 1H), 8.15-7.89 (m, 1H), 7.45-7.41 (m, 1H), 6.87 (t, J=5.6 Hz, 1H), 3.65 (s, 2H), 3.63-3.59 (m, 4H), 3.47-3.39 (m, 4H), 3.26-3.17 (m, 2H), 2.47-2.41 (m, 4H), 1.89-1.82 (m, 4H), 1.12 (t, J=7.1 Hz, 3H).

### Example 2

Compounds A-7, A-8 and A-9 of Formula (I) were prepared with reference to the methods described in applicant's earlier filed application WO2013/138860.

### 1-[5-[2-(1,2-Dihydroxy-1-methyl-ethyl)pyrimidin-5-yl]-1,3-benzothiazol-2-yl]-3-ethyl-urea (A-7)

Compound synthesised and characterised: m/z: 374.1 [M+H]⁺; and ¹H NMR (400 MHz, DMSO-d₆) δ 10.77 (s, 1H), 9.18 (s, 2H), 8.04 (d, J=9 Hz, 2H), 7.63 (d, J=8 Hz, 1H), 6.76 (s, 1H), 5.01 (s, 1H), 4.64 (t, J=6 Hz, 1H), 3.69 (d, J=4.8 Hz, 2H), 3.20 (d, J=5.6 Hz, 2H), 1.47 (s, 3H), 1.12 (t, J=7.2 Hz, 3H).

### 1-[5-[2-(1,2-Dihydroxy-1-methyl-ethyl)pyrimidin-5-yl]-7-(5-methylpyrimidin-2-yl)-1,3-benzothiazol-2-yl]-3-ethyl-urea (A-8)

Compound synthesised and characterised: m/z: 466.15 [M+H]⁺; and ¹H NMR (400 MHz, DMSO-d₆) δ 10.69 (br s, 1H), 9.24 (s, 2H), 8.92 (s, 2H), 8.66 (s, 1H), 8.19 (s, 1H), 6.83 (m, 1H), 5.03 (s, 1H), 4.65 (t, J=6.0 Hz, 1H), 3.64-3.75 (m, 2H), 3.16-3.23 (m, 2H), 2.38 (s, 3H), 1.49 (s, 3H) and 1.08 (m, 3H).

### 1-[5-[2-(1,2-Dihydroxy-1-methyl-ethyl)pyrimidin-5-yl]-7-methyl-1,3-benzothiazol-2-yl]-3-ethyl-urea (A-9)

Compound synthesised and characterised: m/z: 388.12 [M+H]⁺; and ¹H NMR (400 MHz, DMSO-d₆) δ 10.41 (br s, 1H), 9.16 (s, 2H), 7.89 (s, 1H), 7.48 (s, 1H), 6.83 (m, 1H), 5.0 (s, 1H), 4.64 (t, J=6.0 Hz, 1H), 3.67-3.72 (m, 2H), 3.16-3.23 (m, 2H), 2.54 (s, 3H), 1.46 (s, 3H) and 1.09 (t, J=7.20 Hz, 3H).

### Example 3

Compounds A-10, A-11, A-12, A-13, A-14, A-15, A-16, A-17, A-18, A-19, A-20, A-21, A-24, A-25, A-26, A-27, A-28, A-29, A-30, A-31, A-32, A-33, A-34, A-35, A-36, A-37, A-38, A-39, A-40, A-41, A-42, A-43, A-44, A-45, A-46, A-47, A-48, A-49, A-50, A-51, A-52, A-53, A-54, A-55, A-56, A-57, A-58, A-59, A-60, A-61, A-62, A-63, A-64, A-65, A-66, A-67, A-68, A-69, A-70, A-71, A-72, A-73, A-74, A-75, A-76, A-77, A-78, A-79, A-80, A-81, A-82, A-83, A-84, A-85, A-86, A-87, A-88, A-89, A-90, A-91, A-92, A-93, A-94, A-95, A-96, A-97, A-98, A-99, A-100, A-103, A-104, A-105, A-106, A-107, A-110, A-111, A-113, A-114, A-115 of Formula (II) were prepared according to the General Methods described herein as follows.

### 2-[[5-[2-(Ethylcarbamoylamino)-7-(2-pyridyl)-1,3-benzothiazol-5-yl]pyrimidin-2-yl]sulfamoyl]benzoic acid (A-10)

Compound synthesised according to General Method A and characterised: m/z: 576.01 [M+H]⁺; and ¹H NMR (400 MHz, DMSO) δ 9.05 (s, 2H), 8.83 (d, J=4.3 Hz, 1H), 8.53 (d, J=8.0 Hz, 1H), 8.30 (s, 1H), 8.13 (s, 1H), 8.00 (t, J=7.2 Hz, 1H), 7.95 (d, J=7.2 Hz, 1H), 7.72 (br s, 1H), 7.66-7.41 (m, 5H), 1.17 (t, J=7.1 Hz, 3H).

### 1-Ethyl-3-[7-(5-methyl-2-pyridyl)-5-[2-(pyrrolidin-1-ylsulfonylamino)pyrimidin-5-yl]-1,3-benzothiazol-2-yl]urea (A-11)

Compound synthesised according to General Method B and characterised: m/z: 539.18 [M+H]⁺; and ¹H NMR (400 MHz, DMSO) δ 11.12 (s, 1H), 9.30 (s, 2H), 8.66 (dd, J=1.2, 0.7 Hz, 1H), 8.44 (d, J=8.3 Hz, 1H), 8.31 (s, 1H), 8.12 (s, 1H), 7.85-7.80 (m, 1H), 7.42-7.32 (m, 1H), 3.46-3.34 (m, 4H), 3.30-3.23 (m, 2H), 2.41 (s, 3H), 1.78 (s, 4H), 1.14 (t, J=7.1 Hz, 3H).

### 1-Ethyl-3-[7-(5-methyl-2-pyridyl)-5-[2-(propylsulfonylamino)pyrimidin-5-yl]-1,3-benzothiazol-2-yl]urea (A-12)

Compound synthesised according to General Method B and characterised: m/z: 512.15 [M+H]⁺; and ¹H NMR (400 MHz, DMSO-d₆) δ 11.39 (brs, 1H), 10.59 (brs, 1H), 9.17 (s, 2H), 8.71-8.58 (m, 1H), 8.41 (d, J=8.3 Hz, 1H), 8.30 (d, J=1.3 Hz, 1H), 8.12-8.01 (m, 1H), 7.87-7.76 (m, 1H), 6.87 (brs, 1H), 3.59 (t, J=7.6 Hz, 2H), 3.26-3.17 (m, 2H), 2.40 (s, 3H), 1.86-1.70 (m, 2H), 1.12 (t, J=7.2 Hz, 3H), 1.01 (t, J=7.4 Hz, 3H).

### 1-[5-[2-(tert-Butylsulfonylamino)pyrimidin-5-yl]-7-(5-methyl-2-pyridyl)-1,3-benzothiazol-2-yl]-3-ethyl-urea (A-13)

Compound synthesised according to General Method B and characterised: m/z: 526.17 [M+H]⁺; and ¹H NMR (400 MHz, DMSO) δ 13.10 (s, 1H), 9.20 (s, 2H), 8.59 (dd, J=1.4, 0.7 Hz, 1H), 8.36 (d, J=8.3 Hz, 1H), 8.22 (s, 1H), 8.07 (s, 1H), 7.79-7.72 (m, 1H), 3.22 (m, 2H), 2.34 (s, 3H), 1.28 (s, 9H), 1.08 (t, J=7.1 Hz, 3H).

### 1-Ethyl-3-[5-[2-[(2-hydroxy-1,1-dimethyl-ethyl)sulfonylamino]pyrimidin-5-yl]-7-(5-methyl-2-pyridyl)-1,3-benzothiazol-2-yl]urea (A-14)

Compound synthesised according to General Method B and characterised: m/z: 542.20 [M+H]⁺; and ¹H NMR (400 MHz, DMSO-d₆) δ 10.61 (s, 1H), 9.13 (s, 2H), 8.68-8.63 (m, 1H), 8.42 (d, J=8.3 Hz, 1H), 8.29 (d, J=1.7 Hz, 1H), 8.05 (d, J=1.6 Hz, 1H), 7.86-7.79 (m, 1H), 6.89 (brs, 1H), 3.68 (s, 2H), 3.27-3.14 (m, 2H), 2.40 (s, 3H), 1.37 (s, 6H), 1.12 (t, J=7.2 Hz, 3H).

### 1-Ethyl-3-[5-[2-[[2-hydroxyethyl(methyl)sulfamoyl]amino]pyrimidin-5-yl]-7-(5-methyl-2-pyridyl)-1,3-benzothiazol-2-yl]urea (A-15)

Compound synthesised according to General Method B and characterised: m/z: 543.18 [M+H]⁺; and ¹H NMR (400 MHz, DMSO-d₆) δ 11.13 (brs, 1H), 10.59 (brs, 1H), 9.12 (s, 2H), 8.67-8.63 (m, 1H), 8.41 (d, J=8.2 Hz, 1H), 8.29 (d, J=1.7 Hz, 1H), 8.04 (d, J=1.6 Hz, 1H), 7.85-7.80 (m, 1H), 6.87 (brs, 1H), 4.81 (brs, 1H), 3.57 (t, J=6.2 Hz, 2H), 3.39 (t, J=6.2 Hz, 2H), 3.26-3.18 (m, 2H), 2.96 (s, 3H), 2.40 (s, 3H), 1.12 (t, J=7.2 Hz, 3H).

### Methyl 2-[[5-[2-(ethylcarbamoylamino)-7-(5-methyl-2-pyridyl)-1,3-benzothiazol-5-yl]pyrimidin-2-yl]sulfamoyl]acetate (A-16)

Compound synthesised according to General Method B and characterised: m/z: 542.0 [M+H]⁺; and ¹H NMR (400 MHz, DMSO-d₆) δ 13.62 (brs, 1H), 9.58 (s, 2H), 9.29 (brs, 1H), 8.70 (d, J=2.1 Hz, 1H), 8.50 (d, J=8.3 Hz, 1H), 8.41 (d, J=1.6 Hz, 1H), 8.28 (d, J=1.6 Hz, 1H), 7.86 (dd, J=8.3, 2.0 Hz, 1H), 4.40 (s, 2H), 3.64 (s, 3H), 3.41-3.25 (m, 2H), 2.44 (s, 3H), 1.18 (t, J=7.1 Hz, 3H).

### 1-[5-[2-(allylsulfonylamino)pyrimidin-5-yl]-7-(5-methyl-2-pyridyl)-1,3-benzothiazol-2-yl]-3-ethyl-urea (A-17)

Compound synthesised according to General Method B and characterised: m/z: 510.0 [M+H]⁺; and ¹H NMR (400 MHz, DMSO-d₆) δ 13.83 (brs, 1H), 9.56 (s, 3H), 8.74-8.66 (m, 1H), 8.50 (d, J=8.3 Hz, 1H), 8.40 (d, J=1.6 Hz, 1H), 8.26 (d, J=1.6 Hz, 1H), 7.91-7.81 (m, 1H), 5.91 (ddt, J=16.4, 10.7, 7.2 Hz, 1H), 5.24-5.20 (m, 1H), 5.18 (d, J=1.2 Hz, 1H), 4.13 (d, J=7.2 Hz, 2H), 3.39-3.27 (m, 2H), 2.44 (s, 3H), 1.18 (t, J=7.0 Hz, 3H).

### 1-[5-[2-(tert-butylsulfonylamino)pyrimidin-5-yl]-7-[2-(dimethylamino)pyrimidin-5-yl]-1,3-benzothiazol-2-yl]-3-ethyl-urea (A-18)

Compound synthesised according to General Method A and characterised: m/z: 556.15 [M+H]⁺; and ¹H NMR (400 MHz, DMSO-d₆) δ 10.94 (br, 2H), 9.06 (s, 2H), 8.80 (s, 2H), 7.99 (s, 1H), 7.67 (s, 1H), 6.79 (br, 1H), 3.49-3.29 (m incl. water obscuring multiple signals), 3.24 (s, 2H), 1.43 (s, 9H), 1.11 (t, J=7.2 Hz, 3H).

### 1-ethyl-3-[5-[2-(methanesulfonamidomethyl)pyrimidin-5-yl]-7-(5-methyl-2-pyridyl)-1,3-benzothiazol-2-yl]urea (A-19)

Compound synthesised according to General Method A and characterised: m/z: 498.1 [M+H]⁺; and ¹H NMR (400 MHz, DMSO-d₆) δ 10.57 (brs, 1H), 9.24 (s, 2H), 8.55 (dt, J=2.4, 0.9 Hz, 1H), 8.31 (d, J=8.3 Hz, 1H), 8.23 (d, J=1.7 Hz, 1H), 8.00 (d, J=1.6 Hz, 1H), 7.72 (ddd, J=8.3, 2.2, 0.9 Hz, 1H), 7.58 (t, J=6.0 Hz, 1H), 6.83 (t, J=5.2 Hz, 1H), 4.36 (d, J=4.5 Hz, 2H), 3.14-3.06 (m, 2H), 2.90 (s, 3H), 2.29 (s, 3H), 1.01 (t, J=7.2 Hz, 3H).

### 1-[5-[2-(tert-butylsulfonylamino)pyrimidin-5-yl]-7-[4-(2-pyrrolidin-1-ylethyl)piperazin-1-yl]-1,3-benzothiazol-2-yl]-3-ethyl-urea (A-20)

Compound synthesised according to General Method E and characterised: m/z: 616.3 [M+H]⁺; and ¹H NMR (400 MHz, DMSO-d₆) δ 8.96 (s, 2H), 7.64 (d, J=1.4 Hz, 1H), 7.08 (d, J=1.6 Hz, 1H), 3.23-3.16 (m, 8H), 2.75 (q, J=6.0, 5.1 Hz, 2H), 2.69-2.61 (m, 6H), 2.57 (dd, J=7.6, 5.5 Hz, 2H), 1.73 (m, 4H), 1.39 (s, 9H), 1.10 (t, J=7.1 Hz, 3H).

### 1-[5-[2-(cyclopentylsulfonylamino)pyrimidin-5-yl]-7-(5-methyl-2-pyridyl)-1,3-benzothiazol-2-yl]-3-ethyl-urea (A-21)

Compound synthesised according to General Method B and characterised: m/z: 538.2 [M+H]+; and ¹H NMR (400 MHz, DMSO-d₆) δ 11.32 (brs, 1H), 10.58 (brs, 1H), 9.15 (s, 2H), 8.68-8.63 (m, 1H), 8.40 (d, J=8.3 Hz, 1H), 8.28 (s, 1H), 8.04 (s, 1H), 7.90-7.73 (m, 1H), 6.86 (t, J=5.6 Hz, 1H), 4.40 (ddd, J=15.5, 8.8, 6.6 Hz, 1H), 3.28-3.12 (m, 2H), 2.40 (s, 3H), 2.10-1.86 (m, 4H), 1.83-1.67 (m, 2H), 1.67-1.50 (m, 2H), 1.12 (t, J=7.2 Hz, 3H).

### 1-(5-(2-(1,1-dioxido-1,2-thiazinan-2-yl)pyrimidin-5-yl)-7-(pyridin-2-yl)benzo[d]thiazol-2-yl)-3-ethyl-urea (A-24)

Compound synthesised according to General Method A and characterised: m/z: 508.27 [M−H]+; and ¹H NMR (400 MHz, DMSO-d6): δ 10.62 (br s, 1H), 9.30 (s, 2H), 8.81 (d, J=4.80 Hz, 1H), 8.52 (d, J=8.40 Hz, 1H), 8.39 (s, 1H), 8.13 (s, 1H), 8.01 (t, J=8.0 Hz, 1H), 7.50 (m, 1H), 6.85 (m, 1H), 4.15 (m, 2H), 3.45 (m, 2H), 3.20 (m, 2H), 2.18 (m, 2H), 1.76 (m, 2H) and 1.11 (t, J=7.20 Hz, 3H).

### 1-[5-[6-(1,1-dioxo-1,2-thiazolidin-2-yl)-3-pyridyl]-7-(2-pyridyl)-1,3-benzothiazol-2-yl]-3-ethylurea (A-25)

Compound synthesised according to General Method A and characterised: m/z: 495.15 [M+H]+; and ¹H NMR (400 MHz, DMSO-d6): δ 10.59 (s, 1H), 8.88 (d, J=2.40 Hz, 1H), 8.81 (d, J=4.0 Hz, 1H), 8.50 (d, J=8.40 Hz, 1H), 8.34 (dd, J=2.40 and 8.80 Hz respectively, 1H), 8.28 (s, 1H), 7.98-8.02 (m, 2H), 7.45 (m, 1H), 7.30 (d, J=8.80 Hz, 1H), 6.85 (m, 1H), 3.98 (t, J=6.80 Hz, 2H), 3.61 (t, J=7.20 Hz, 2H), 3.21 (m, 2H), 2.41 (m, 2H) and 1.08 (t, J=7.20 Hz, 3H).

### 1-(5-(2-(1,1-dioxidoisothiazolidin-2-yl)pyrimidin-5-yl)-7-(pyridin-2-yl)benzo[d]thiazol-2-yl)-3-ethylurea (A-26)

Compound synthesised according to General Method A and characterised: m/z: 496.18 [M+H]+; ¹H NMR (400 MHz, DMSO-d6): δ 10.61 (br s, 1H), 9.19 (s, 2H), 8.82 (d, J=4.40 Hz, 1H), 8.50 (d, J=8.0 Hz, 1H), 8.34 (s, 1H), 8.09 (s, 1H), 8.01 (m, 1H), 7.46 (m, 1H), 6.87 (m, 1H), 4.02 (t, J=6.40 Hz, 2H), 3.59 (m, 2H), 3.21 (m, 2H), 2.41 (m, 2H) and 1.11 (t, J=7.20 Hz, 3H)

### 1-(5-(2-(1,1-dioxidoisothiazolidin-2-yl)pyrimidin-5-yl)-6-((tetrahydrofuran-2-yl)methoxy)benzo[d]thiazol-2-yl)-3-ethylurea (A-27)

Compound synthesised according to General Method C and characterised: m/z: 519.16 [M+H]+; ¹H NMR (400 MHz, DMSO-d6) δ 10.61 (s, 1H), 8.84 (s, 2H), 7.70 (s, 1H), 7.66 (s, 1H), 6.86 (m, 1H), 4.15 (m, 1H), 3.96-4.04 (m, 4H), 3.65-3.73 (m, 2H), 3.56-3.59 (m, 2H), 3.16-3.19 (m, 2H), 2.37 (t, J=6.80 Hz, 2H), 1.93-1.79 (m, 1H), 1.77-1.81 (m, 2H), 1.60-1.65 (m, 1H) and 1.09 (t, J=6.80 Hz, 3H)

### 1-(5-(2-(1,1-dioxidoisothiazolidin-2-yl)pyrimidin-5-yl)-7-(5-methylpyridin-2-yl)benzo[d]thiazol-2-yl)-3-ethylurea (A-28)

Compound synthesised according to General Method A and characterised: m/z: 508.11 [M−H]+; ¹H NMR (400 MHz, DMSO-d6): δ 10.58 (s, 1H), 9.18 (s, 2H), 8.65 (s, 1H), 8.42 (d, J=8.40 Hz, 1H), 8.30 (s, 1H), 8.06 (s, 1H), 7.82 (m, 1H), 6.86 (m, 1H), 4.01 (t, J=6.80 Hz, 2H), 3.59 (t, J=6.80 Hz, 2H), 3.18-3.23 (m, 2H), 2.38-2.41 (m, 5H) and 1.11 (t, J=7.20 Hz, 3H).

### N-(5-(2-(3-ethylureido)-7-(5-methylpyridin-2-yl)benzo[d]thiazol-5-yl)pyrimidin-2-yl)-N-methylmethanesulfonamide (A-29)

Compound synthesised according to General Method A and characterised: m/z: 498.17 [M−H]+; ¹H NMR (400 MHz, DMSO) δ 10.59 (br s, 1H), 9.25 (s, 2H), 8.65 (s, 1H), 8.43 (d, J=7.60 Hz, 1H), 8.32 (s, 1H), 8.07 (s, 1H), 7.83 (m, 1H), 6.85 (m, 1H), 3.58 (s, 3H), 3.49 (s, 3H), 3.21-3.23 (m, 2H), 2.40 (s, 3H) and 1.11 (t, J=7.20 Hz, 3H).

### N-(5-(2-(3-ethylureido)-7-(5-methylpyridin-2-yl)benzo[d]thiazol-5-yl)pyrimidin-2-yl)methanesulfonamide (A-30)

Compound synthesised according to General Method D and characterised: m/z: 484.31 [M+H]+; ¹H NMR (400 MHz, DMSO) δ 11.45 (br s, 1H), 10.59 (br s, 1H), 9.18 (s, 2H), 8.65 (s, 1H), 8.42 (d, J=8.0 Hz, 1H), 8.29 (s, 1H), 8.06 (s, 1H), 7.83 (d, J=7.60 Hz, 1H), 6.88 (m, 1H), 3.41 (s, 3H), 3.16-3.23 (m, 2H), 2.40 (s, 3H) and 1.10 (t, J=7.20 Hz, 3H).

### N-(5-(2-(3-ethylureido)-7-(pyridin-2-yl)benzo[d]thiazol-5-yl)pyrimidin-2-yl)methanesulfonamide (A-31)

Compound synthesised according to General Method B and characterised: m/z: 470.02 [M+H]+; ¹H NMR (400 MHz, DMSO) δ 11.53 (s, 1H), 10.67 (s, 1H), 9.26 (s, 2H), 8.85 (d, J=4.4 Hz, 1H), 8.55 (d, J=8.2 Hz, 1H), 8.38 (s, 1H), 8.15 (m, 1H), 8.03 (td, J=7.6, 1.8 Hz, 1H), 7.47 (ddd, J=7.6, 4.4, 0.6 Hz, 1H), 6.90 (s, 1H), 3.35 (s, 2H), 3.30-3.24 (m, 2H), 1.15 (t, J=7.1 Hz, 3H).

### 1-[5-[2-(dimethylsulfamoylamino)pyrimidin-5-yl]-7-(2-pyridyl)-1,3-benzothiazol-2-yl]-3-ethyl urea (A-32)

Compound synthesised according to General Method B and characterised: m/z: 499.00 [M+H]+; ¹H NMR (400 MHz, DMSO) δ 10.8 (br s, 2H), 9.23 (s, 2H), 8.84 (ddd, J=4.8, 1.7, 0.8 Hz, 1H), 8.55 (d, J=8.0 Hz, 1H), 8.37 (d, J=1.1 Hz, 1H), 8.14 (s, 1H), 8.03 (dt, J=8.0, 1.7 Hz, 1H), 7.47 (ddd, J=7.4, 4.8, 0.8 Hz, 1H), 7.07 (s, 1H), 3.26 (m, 2H), 2.88 (s, 6H), 1.15 (t, J=7.1 Hz, 3H).

### N-(5-(2-(3-ethylureido)-7-(pyridin-2-yl)benzo[d]thiazol-5-yl)pyrimidin-2-yl)propane-1-sulfonamide (A-33)

Compound synthesised according to General Method B and characterised: m/z: 498.00 [M+H]+; ¹H NMR (400 MHz, DMSO-d6) δ 11.39 (brs, 1H), 10.64 (brs, 1H), 9.17 (s, 2H), 8.82 (d, J=4.8 Hz, 1H), 8.52 (d, J=8.2 Hz, 1H), 8.35 (s, 1H), 8.09 (s, 1H), 8.07-7.95 (m, 1H), 7.53-7.38 (m, 1H), 6.88 (t, J=5.6 Hz, 1H), 3.28-3.15 (m, 2H), 1.86-1.67 (m, 2H), 1.12 (t, J=7.2 Hz, 3H), 1.01 (t, J=7.5 Hz, 3H).

### N-(5-(2-(3-ethylureido)-7-(pyridin-2-yl)benzo[d]thiazol-5-yl)pyrimidin-2-yl)cyclopropanesulfonamide (A-34)

Compound synthesised according to General Method A and characterised: m/z: 495.98 [M+H]+; ¹H NMR (400 MHz, DMSO-d6) δ11.42 (brs, 1H), 10.64 (brs, 1H), 9.17 (s, 2H), 8.88-8.75 (m, 1H), 8.52 (d, J=8.2 Hz, 1H), 8.34 (d, J=1.6 Hz, 1H), 8.09 (d, J=1.6 Hz, 1H), 8.06-7.93 (m, 1H), 7.50-7.36 (m, 1H), 6.88 (t, J=5.7 Hz, 1H), 3.33-3.25 (m, 1H), 3.27-3.16 (m, 2H), 1.20-1.14 (m, 2H), 1.14-1.04 (m, 5H).

### N-(5-(2-(3-ethylureido)-7-(pyridin-2-yl)benzo[d]thiazol-5-yl)pyrimidin-2-yl)cyclopentanesulfonamide (A-35)

Compound synthesised according to General Method B and characterised: m/z: 524.02 [M+H]+; ¹H NMR (400 MHz, DMSO-d6) δ11.37 (brs, 1H), 10.62 (brs, 1H), 9.16 (s, 2H), 8.82 (d, J=4.8 Hz, 1H), 8.51 (d, J=8.3 Hz, 1H), 8.34 (s, 1H), 8.08 (s, 1H), 8.06-7.91 (m, 1H), 7.57-7.34 (m, 1H), 6.86 (brs, 1H), 4.40 (p, J=8.1 Hz, 1H), 3.28-3.12 (m, 2H), 2.17-1.85 (m, 4H), 1.84-1.67 (m, 2H), 1.67-1.49 (m, 2H), 1.12 (t, J=7.2 Hz, 3H).

### N-(5-(2-(3-ethylureido)-7-(pyridin-2-yl)benzo[d]thiazol-5-yl)pyrimidin-2-yl)ethanesulfonamide (A-36)

Compound synthesised according to General Method B and characterised: m/z: 483.98 [M+H]+; ¹H NMR (400 MHz, DMSO-d6) δ11.38 (brs, 1H), 10.63 (brs, 1H), 9.17 (s, 2H), 8.82 (d, J=4.3 Hz, 1H), 8.52 (d, J=8.2 Hz, 1H), 8.34 (s, 1H), 8.09 (s, 1H), 8.05-7.92 (m, 1H), 7.56-7.36 (m, 1H), 6.86 (brs, 1H), 3.72-3.52 (m, 2H), 3.28-3.16 (m, 2H), 1.27 (t, J=7.3 Hz, 3H), 1.12 (t, J=7.1 Hz, 3H).

### 1-ethyl-3-[5-[2-(ethylsulfamoylamino)pyrimidin-5-yl]-7-(2-pyridyl)-1,3-benzothiazol-2-yl]urea (A-37)

Compound synthesised according to General Method B and characterised: 498.99 [M+H]+; ¹H NMR (400 MHz, DMSO) δ 13.74 (s, 1H), 9.75 (s, 1H), 9.41 (s, 2H), 8.78 (dq, J=5.0, 0.7 Hz, 1H), 8.50 (d, J=8.0 Hz, 1H), 8.32 (s, 1H), 8.18 (s, 1H), 7.95 (td, J=8.0, 1.8 Hz, 1H), 7.39 (dd, J=7.1, 5.0 Hz, 1H), 7.31 (s, 1H), 5.62 (t, J=7.1 Hz, 1H), 3.19 (m, 2H), 2.76 (p, J=7.1 Hz, 2H), 1.11 (t, J=7.1 Hz, 3H), 0.98 (t, J=7.1 Hz, 3H).

### 1-[5-[2-(dimethylsulfamoylamino)pyrimidin-5-yl]-7-(5-methyl-2-pyridyl)-1,3-benzothiazol-2-yl]-3-ethyl urea (A-38)

Compound synthesised according to General Method B and characterised: 513.01 [M+H]+; ¹H NMR (400 MHz, DMSO) δ 13.66 (s, 1H), 9.43 (s, 2H), 8.69 (d, J=1.8 Hz, 1H), 8.47 (d, J=8.3 Hz, 1H), 8.36 (s, 1H), 8.22 (s, 1H), 7.85 (dd, J=8.3, 1.8 Hz, 1H), 3.32-3.28 (m, 2H), 2.77 (s, 6H), 2.44 (s, 3H), 1.18 (t, J=7.1 Hz, 3H).

### 1-ethyl-3-[5-[2-(ethylsulfamoylamino)pyrimidin-5-yl]-7-(5-methyl-2-pyridyl)-1,3-benzothiazol-2-yl]urea (A-39)

Compound synthesised according to General Method B and characterised: m/z: 512.97 [M+H]+; ¹H NMR (400 MHz, DMSO) δ 13.72 (s, 1H), 9.46 (s, 2H), 8.69 (d, J=1.9 Hz, 1H), 8.47 (d, J=8.3 Hz, 1H), 8.36 (s, 1H), 8.22 (s, 1H), 7.85 (dd, J=8.3, 1.9 Hz, 1H), 5.76 (s, 1H), 3.31-3.28 (m, 2H), 2.91-2.81 (qn, J=7.0 Hz, 2H), 2.44 (s, 3H), 1.19 (t, J=7.0 Hz, 3H), 1.06 (t, J=7.2 Hz, 3H).

### N-(5-(2-(3-ethylureido)-7-(5-methylpyridin-2-yl)benzo[d]thiazol-5-yl)pyrimidin-2-yl)cyclopropanesulfonamide (A-40)

Compound synthesised according to General Method B and characterised: m/z: 510.02 [M+H]+; ¹H NMR (400 MHz, DMSO-d6) δ11.40 (brs, 1H), 10.59 (brs, 1H), 9.16 (s, 2H), 8.68-8.59 (m, 1H), 8.41 (d, J=8.2 Hz, 1H), 8.29 (d, J=1.7 Hz, 1H), 8.05 (d, J=1.6 Hz, 1H), 7.87-7.77 (m, 1H), 6.87 (t, J=5.7 Hz, 1H), 3.28-3.16 (m, 2H), 2.40 (s, 3H), 1.20-1.05 (m, 7H).

### 1-ethyl-3-[5-[2-(methylsulfamoylamino)pyrimidin-5-yl]-7-(2-pyridyl)-1,3-benzothiazol-2-yl]urea (A-41)

Compound synthesised according to General Method B and characterised: m/z: 485.06 [M+H]+; ¹H NMR (400 MHz, DMSO) δ 13.69 (s, 1H), 9.44 (s, 2H), 8.86 (ddd, J=4.9, 1.8, 0.8 Hz, 1H), 8.56 (d, J=8.3 Hz, 1H), 8.39 (s, 1H), 8.24 (s, 1H), 8.03 (dt, J=7.6, 1.8 Hz, 1H), 7.48 (ddd, J=7.6, 4.9, 0.8 Hz, 1H), 5.69 (s, 1H), 3.32-3.24 (m, 2H), 2.47 (d, J=5.6 Hz, 3H), 1.19 (t, J=7.1 Hz, 3H).

### 1-ethyl-3-[7-(5-methyl-2-pyridyl)-5-[2-(methylsulfamoylamino)pyrimidin-5-yl]-1,3-benzothiazol-2-yl]urea (A-42)

Compound synthesised according to General Method B and characterised: m/z: 499.06 [M+H]+; ¹H NMR (400 MHz, DMSO) δ 13.64 (s, 1H), 9.42 (s, 2H), 8.69 (dd, J=1.4, 0.7 Hz, 1H), 8.45 (d, J=8.3 Hz, 1H), 8.34 (s, 1H), 8.21 (s, 1H), 7.89-7.82 (m, 1H), 5.69 (s, 1H), 3.32-3.24 (m, 2H), 2.46 (d, J=5.5 Hz, 3H), 2.44 (s, 3H), 1.19 (t, J=7.1 Hz, 3H).

### N-(5-(2-(3-ethylureido)-7-(pyridin-2-yl)benzo[d]thiazol-5-yl)pyrimidin-2-yl)morpholine-4-sulfonamide (A-43)

Compound synthesised according to General Method B and characterised: m/z: 499.06 [M+H]+; ¹H NMR (400 MHz, DMSO) δ 13.64 (s, 1H), 9.42 (s, 2H), 8.69 (dd, J=1.4, 0.7 Hz, 1H), 8.45 (d, J=8.3 Hz, 1H), 8.34 (s, 1H), 8.21 (s, 1H), 7.89-7.82 (m, 1H), 5.69 (s, 1H), 3.32-3.24 (m, 2H), 2.46 (d, J=5.5 Hz, 3H), 2.44 (s, 3H), 1.19 (t, J=7.1 Hz, 3H).

### N-(5-(2-(3-ethylureido)-7-(5-methylpyridin-2-yl)benzo[d]thiazol-5-yl)pyrimidin-2-yl)morpholine-4-sulfonamide (A-44)

Compound synthesised according to General Method B and characterised: m/z: 555.18 [M+H]+; ¹H NMR (400 MHz, DMSO) δ 13.27 (s, 1H), 9.37 (s, 2H), 8.69 (dd, J=1.5, 0.7 Hz, 1H), 8.48 (d, J=8.3 Hz, 1H), 8.35 (s, 1H), 8.18 (s, 1H), 7.84 (dd, J=8.3, 1.5 Hz, 1H), 3.64-3.61 (m, 4H), 3.28 (m, 2H), 3.23-3.19 (m, 4H), 2.43 (s, 3H), 1.17 (t, J=7.1 Hz, 3H).

### N-(5-(2-(3-ethylureido)-7-(pyridin-2-yl)benzo[d]thiazol-5-yl)pyrimidin-2-yl)pyrrolidine-1-sulfonamide (A-45)

Compound synthesised according to General Method B and characterised: m/z: 525.16 [M+H]+; ¹H NMR (400 MHz, DMSO) δ 13.84 (s, 1H), 11.13 (s, 1H), 9.28 (s, 2H), 8.85 (dd, J=4.8, 0.8 Hz, 1H), 8.56 (d, J=8.2 Hz, 1H), 8.37 (s, 1H), 8.17 (s, 1H), 8.07-7.99 (m, 1H), 7.47 (ddd, J=7.5, 4.8, 0.8 Hz, 1H), 7.15-6.68 (m, 1H), 3.47-3.39 (m, 4H), 3.30-3.25 (m, 2H), 1.84-1.78 (m, 4H), 1.16 (t, J=7.1 Hz, 3H).

### (S)-2-amino-N-(5-(2-(3-ethylureido)-7-(5-methylpyridin-2-yl)benzo[d]thiazol-5-yl)pyrimidin-2-yl)-3-phenylpropane-1-sulfonamide (A-46)

Compound synthesised according to General Method B and characterised: m/z: 603.36 [M+H]+; 1H NMR (400 MHz, DMSO) δ 11.64 (br s, 1H), 10.60 (br s, 1H), 9.19 (br s, 2H), 8.66 (d, J=1.2 Hz, 1H), 8.40 (d, J=8.4 Hz, 1H), 8.29 (s, 1H), 8.07 (s, 1H), 7.84 (dd, J=8.4, 2 Hz, 1H), 6.88 (br s, 1H), 4.65 (m, 1H), 3.91-3.45 (m, 5H), 3.24 (m*, 2H), 2.41 (s, 3H), 1.97, 1.94 (2xs, 3H), 1.13 (t, J=7.2 Hz, 3H).

### N-(5-(2-(3-ethylureido)-7-(5-methylpyridin-2-yl)benzo[d]thiazol-5-yl)pyrimidin-2-yl)ethanesulfonamide (A-47)

Compound synthesised according to General Method B and characterised: m/z: 498.07 [M+H]+; ¹H NMR (400 MHz, DMSO-d6) δ11.36 (brs, 1H), 10.60 (brs, 1H), 9.16 (s, 2H), 8.67-8.63 (m, 1H), 8.40 (d, J=8.3 Hz, 1H), 8.29 (d, J=1.7 Hz, 1H), 8.11-7.98 (m, 1H), 7.84-7.79 (m, 1H), 6.88 (brs, 1H), 3.71-3.50 (m, 2H), 3.27-3.12 (m, 2H), 2.40 (s, 3H), 1.28 (t, J=7.3 Hz, 3H), 1.12 (t, J=7.2 Hz, 3H).

### N-(5-(2-(3-ethylureido)-7-(5-methylpyridin-2-yl)benzo[d]thiazol-5-yl)pyrimidin-2-yl)propane-2-sulfonamide (A-48)

Compound synthesised according to General Method B and characterised: m/z: 512.15 [M+H]+; ¹H NMR (400 MHz, DMSO-d6) δ11.28 (brs, 1H), 10.58 (brs, 1H), 9.15 (s, 2H), 8.71-8.59 (m, 1H), 8.39 (d, J=8.3 Hz, 1H), 8.28 (d, J=1.4 Hz, 1H), 8.04 (d, J=1.4 Hz, 1H), 7.82 (dd, J=8.3, 2.2 Hz, 1H), 6.87 (t, J=5.6 Hz, 1H), 4.08 (p, J=6.9 Hz, 1H), 3.28-3.15 (m, 2H), 2.40 (s, 3H), 1.34 (d, J=6.9 Hz, 6H), 1.12 (t, J=7.2 Hz, 3H).

### N-(5-(2-(3-ethylureido)-7-(pyridin-2-yl)benzo[d]thiazol-5-yl)pyrimidin-2-yl)-3-methoxyazetidine-1-sulfonamide (A-49)

Compound synthesised according to General Method B and characterised: m/z: 541.19 [M+H]+; 1H NMR (400 MHz, DMSO) δ 10.61 (s, 1H), 9.22 (s, 2H), 8.86-8.81 (m, 1H), 8.54 (d, J=8.3 Hz, 1H), 8.38 (d, J=1.3 Hz, 1H), 8.12 (d, J=1.3 Hz, 1H), 8.03 (dt, J=7.6, 1.7 Hz, 1H), 8.00 (dd, J=15.3, 7.6 Hz, 1H), 7.48 (ddd, J=7.6, 4.9, 0.8 Hz, 1H), 6.87 (t, J=5.1 Hz, 1H), 4.62 (dd, J=14.0, 4.6 Hz, 1H), 4.06 (dd, J=14.0, 3.1 Hz, 1H), 3.85-3.76 (m, 1H), 3.70-3.56 (m, 2H), 3.39 (s, 3H), 3.28-3.20 (m, 2H), 1.14 (t, J=7.2 Hz, 3H).

### N-(5-(2-(3-ethylureido)-7-(5-methylpyridin-2-yl)benzo[d]thiazol-5-yl)pyrimidin-2-yl)-3-methoxyazetidine-1-sulfonamide (A-50)

Compound synthesised according to General Method B and characterised: m/z: 555.17 [M+H]+; ¹H NMR (400 MHz, DMSO) δ 10.59 (s, 1H), 9.21 (s, 2H), 8.67 (dd, J=1.5, 0.7 Hz, 1H), 8.44 (d, J=8.4 Hz, 1H), 8.33 (d, J=1.3 Hz, 1H), 8.09 (d, J=1.3 Hz, 1H), 7.99 (t, J=7.5 Hz, 1H), 7.85 (dd, J=8.4, 1.5 Hz, 1H), 6.88 (t, J=5.3 Hz, 1H), 4.61 (dd, J=14.0, 4.6 Hz, 1H), 4.06 (dd, J=14.0, 3.1 Hz, 1H), 3.84-3.75 (m, 1H), 3.64 (ddd, J=14.7, 7.5, 5.4 Hz, 1H), 3.58-3.44 (m, 1H), 3.39 (s, 3H), 3.27-3.19 (m, 2H), 2.42 (s, 3H), 1.14 (t, J=7.2 Hz, 3H).

### N-(5-(2-(3-ethylureido)-7-(5-methylpyridin-2-yl)benzo[d]thiazol-5-yl)pyrimidin-2-yl)benzenesulfonamide (A-51)

Compound synthesised according to General Method B and characterised: m/z: 546.05 [M+H]+; ¹H NMR (400 MHz, DMSO) δ 13.74 (s, 1H), 9.38 (s, 2H), 8.68 (dd, J=1.5, 0.7 Hz, 1H), 8.47 (d, J=8.2 Hz, 1H), 8.32 (s, 1H), 8.18 (s, 1H), 7.96 (m, 2H), 7.82 (dd, J=8.2, 1.5 Hz, 1H), 7.46 (m, 3H), 3.45-3.36 (m, 2H), 2.43 (s, 3H), 1.23 (t, J=7.0 Hz, 3H).

### N-(5-(2-(3-ethylureido)-7-(5-methylpyridin-2-yl)benzo[d]thiazol-5-yl)pyrimidin-2-yl)-2-morpholinoethanesulfonamide (A-52)

Compound synthesised according to General Method B and characterised: m/z: 583.17 [M+H]+; ¹H NMR (400 MHz, DMSO-d6) δ 11.59 (brs, 1H), 9.19 (s, 2H), 8.71-8.59 (m, 1H), 8.40 (d, J=8.3 Hz, 1H), 8.29 (d, J=1.3 Hz, 1H), 8.07 (d, J=1.5 Hz, 1H), 7.86 (dd, J=8.2, 2.1 Hz, 1H), 7.22 (t, J=5.7 Hz, 1H), 4.32-4.17 (m, 2H), 3.83-3.71 (m, partially obscured by water peak, assume 2H), 3.62-3.53 (m, 2H), 3.53-3.45 (m, 2H), 3.27-2.97 (m, 4H), 2.41 (s, 3H), 1.11 (t, J=7.2 Hz, 3H).

### N-(5-(2-(3-ethylureido)-7-(5-methylpyridin-2-yl)benzo[d]thiazol-5-yl)pyrimidin-2-yl)pyrrolidine-3-sulfonamide (A-53)

Compound synthesised according to General Method B and characterised: m/z: 539.25 [M+H]+; ¹H NMR (400 MHz, DMSO-d6) δ 9.88-9.64 (m, 1H), 9.65-9.45 (m, 1H), 9.19 (s, 2H), 8.71-8.60 (m, 1H), 8.40 (d, J=8.2 Hz, 1H), 8.28 (d, J=1.6 Hz, 1H), 8.06 (d, J=1.6 Hz, 1H), 7.91-7.78 (m, 1H), 7.24 (t, J=5.7 Hz, 1H), 4.78-4.65 (m, 1H), 3.76-3.62 (m, 1H), 3.63-3.49 (m, 1H), 3.39-3.24 (m, 2H), 3.26-3.14 (m, 2H), 2.45-2.34 (m, 5H), 1.11 (t, J=7.2 Hz, 3H).

### N-(5-(2-(3-ethylureido)-7-(5-methylpyridin-2-yl)benzo[d]thiazol-5-yl)pyrimidin-2-yl)-3-hydroxypyrrolidine-1-sulfonamide (A-54)

Compound synthesised according to General Method B and characterised: m/z: 555.18 [M+H]+; ¹H NMR (400 MHz, DMSO-d6) δ 11.05 (brs, 1H), 10.59 (s, 1H), 9.12 (s, 2H), 8.65 (d, J=2.2 Hz, 1H), 8.41 (d, J=8.3 Hz, 1H), 8.28 (d, J=1.7 Hz, 1H), 8.04 (d, J=1.6 Hz, 1H), 7.83 (dd, J=8.4, 2.3 Hz, 1H), 7.02-6.73 (m, 1H), 5.45-4.55 (m, 1H), 4.37-4.17 (m, 1H), 3.72-3.63 (m, 1H), 3.63-3.54 (m, 2H), 3.29-3.15 (m, 3H), 2.40 (s, 3H), 1.97-1.82 (m, 1H), 1.82-1.62 (m, 1H), 1.12 (t, J=7.2 Hz, 3H).

### N-(5-(2-(3-ethylureido)-7-(5-methylpyridin-2-yl)benzo[d]thiazol-5-yl)pyrimidin-2-yl)-1-(hydroxymethyl)cyclopropane-1-sulfonamide (A-55)

Compound synthesised according to General Method B and characterised: m/z: 540.12 [M+H]+; ¹H NMR (400 MHz, DMSO-d6) δ 10.63 (brs, 1H), 9.13 (s, 2H), 8.68-8.62 (m, 1H), 8.42 (d, J=8.3 Hz, 1H), 8.29 (d, J=1.7 Hz, 1H), 8.05 (d, J=1.5 Hz, 1H), 7.90-7.76 (m, 1H), 6.92 (brs, 1H), 3.85 (s, 2H), 3.26-3.18 (m, 2H), 2.41 (s, 3H), 1.57-1.38 (m, 2H), 1.12 (t, J=7.2 Hz, 3H), 1.09-0.98 (m, 2H).

### (R)—N-(5-(2-(3-ethylureido)-7-(5-methylpyridin-2-yl)benzo[d]thiazol-5-yl)pyrimidin-2-yl)-3-morpholinopyrrolidine-1-sulfonamide (A-56)

Compound synthesised according to General Method B and characterised: m/z: 624.13 [M+H]+; ¹H NMR (400 MHz, DMSO-d6) δ 11.97 (s, 1H), 9.19 (s, 2H), 8.66 (d, J=2.1 Hz, 1H), 8.42 (d, J=8.3 Hz, 1H), 8.31 (s, 1H), 8.07 (s, 1H), 7.86 (dd, J=8.3, 2.2 Hz, 1H), 7.29-7.05 (m, 1H), 4.26-4.13 (m, 1H), 3.47-3.32 (m, 3H), 3.32-3.06 (m, 4H), 2.41 (s, 3H), 2.38-2.27 (m, 2H), 1.11 (t, J=7.2 Hz, 3H). Note: 7 protons obscured by water peak, and 1 exchangeable proton not showing.

### (R)-3-(dimethylamino)-N-(5-(2-(3-ethylureido)-7-(5-methylpyridin-2-yl)benzo[d]thiazol-5-yl)pyrimidin-2-yl)pyrrolidine-1-sulfonamide (A-57)

Compound synthesised according to General Method B and characterised: m/z: 582.10 [M+H]+; ¹H NMR (400 MHz, DMSO-d6) δ 11.32 (s, 1H), 9.19 (s, 2H), 8.66 (s, 1H), 8.43 (d, J=8.2 Hz, 1H), 8.31 (s, 1H), 8.07 (s, 1H), 7.86 (d, J=8.0 Hz, 1H), 7.35-7.00 (m, 1H), 4.18-4.06 (m, 1H), 3.50-3.35 (m, 1H), 3.29-3.09 (m, 2H), 2.77 (dd, J=12.8, 4.6 Hz, 6H), 2.41 (s, 3H), 2.38-2.28 (m, 1H), 2.28-2.16 (m, 1H), 1.11 (t, J=7.1 Hz, 3H). Note: 3 protons obscured by water peak.

### 1-acetyl-N-(5-(2-(3-ethylureido)-7-(5-methylpyridin-2-yl)benzo[d]thiazol-5-yl)pyrimidin-2-yl)pyrrolidine-3-sulfonamide (A-58)

Compound synthesised according to General Method B and characterised: m/z: 581.31 [M+H]+; ¹H NMR (400 MHz, DMSO-d6) δ 12.91 (br s, 1H); 9.24 (s, 2H), 8.69 (d, J=2 Hz, 2H), 8.69 (d, J=8.4 Hz, 1H), 8.27 (s, 1H), 8.18 (s, 1H), 8.14 (s, 1H), 7.86 (dd, J=8.4, 1.6 Hz, 1H), 7.20-7.27 (m, 4H), 7.14 (t, J=7.2 Hz, 1H), 3.76 (m, 1H), 3.67 (dd, J=14, 2.8 Hz, 2H), 3.04 (dd, J=13.6, 5.2 Hz, 2H), 2.87 (dd, J=13.6, 8 Hz, 2H), 2.43 (s, 3H), 1.16 (t, J=7.2 Hz, 3H).

### (R)—N-(5-(2-(3-ethylureido)-7-(5-methylpyridin-2-yl)benzo[d]thiazol-5-yl)pyrimidin-2-yl)-3-hydroxypyrrolidine-1-sulfonamide (A-59)

Compound synthesised according to General Method B and characterised: m/z: 555.11 [M+H]+; ¹H NMR (400 MHz, DMSO-d6) δ 10.61 (s, 1H), 9.12 (s, 2H), 8.68-8.63 (m, 1H), 8.41 (d, J=8.2 Hz, 1H), 8.28 (d, J=1.6 Hz, 1H), 8.04 (d, J=1.6 Hz, 1H), 7.83 (dd, J=8.4, 2.3 Hz, 1H), 7.00-6.80 (m, 1H), 4.34-4.22 (m, 1H), 3.73-3.63 (m, 1H), 3.64-3.54 (m, 2H), 3.28-3.17 (m, 3H), 2.40 (s, 3H), 1.98-1.84 (m, 1H), 1.80-1.70 (m, 1H), 1.12 (t, J=7.2 Hz, 3H).

### (S)—N-(5-(2-(3-ethylureido)-7-(5-methylpyridin-2-yl)benzo[d]thiazol-5-yl)pyrimidin-2-yl)-3-hydroxypyrrolidine-1-sulfonamide (A-60)

Compound synthesised according to General Method B and characterised: m/z: 555.2 [M+H]+; ¹H NMR (400 MHz, DMSO-d6) δ 10.78 (brs, 1H), 9.14 (s, 2H), 8.68-8.63 (m, 1H), 8.41 (d, J=8.2 Hz, 1H), 8.28 (d, J=1.7 Hz, 1H), 8.05 (d, J=1.6 Hz, 1H), 7.86-7.80 (m, 1H), 7.08 (brs, 1H), 4.31-4.24 (m, 1H), 3.69-3.60 (m, 1H), 3.60-3.52 (m, 2H), 3.27-3.18 (m, 3H), 2.40 (s, 3H), 1.95-1.83 (m, 1H), 1.78-1.69 (m, 1H), 1.12 (t, J=7.1 Hz, 3H).

### N-(5-(2-(3-ethylureido)-6-fluorobenzo[d]thiazol-5-yl)pyrimidin-2-yl)-2-methylpropane-2-sulfonamide (A-61)

Compound synthesised according to General Method C and characterised: m/z: 453.12 [M+H]+; ¹H NMR (400 MHz, DMSO-d6) δ 10.99 (s, 1H), 10.80 (s, 1H), 8.87-8.74 (m, 2H), 7.96 (d, J=10.3 Hz, 1H), 7.84 (d, J=6.8 Hz, 1H), 6.71 (t, J=5.5 Hz, 1H), 3.19 (qd, J=7.2, 5.6 Hz, 2H), 1.41 (s, 9H), 1.09 (t, J=7.2 Hz, 3H).

### (R)—N-(5-(2-(3-ethylureido)-7-(5-methylpyridin-2-yl)benzo[d]thiazol-5-yl)pyrimidin-2-yl)-2-(hydroxymethyl)pyrrolidine-1-sulfonamide (A-62)

Compound synthesised according to General Method B and characterised: m/z: 569.2 [M+H]+; ¹H NMR (400 MHz, DMSO-d6) δ 10.60 (brs, 1H), 9.13 (brs, 2H), 8.69-8.61 (m, 1H), 8.42 (d, J=8.3 Hz, 1H), 8.29 (d, J=1.7 Hz, 1H), 8.06 (s, 1H), 7.83 (dd, J=8.4, 2.2 Hz, 1H), 7.60-7.43 (m, 1H), 6.88 (s, 1H), 4.30 (s, 1H), 3.50 (ddd, J=51.9, 12.3, 5.8 Hz, 4H), 3.25-3.18 (m, 3H), 2.41 (s, 3H), 2.00-1.59 (m, 4H), 1.12 (t, J=7.2 Hz, 3H).

### (S)—N-(5-(2-(3-ethylureido)-7-(5-methylpyridin-2-yl)benzo[d]thiazol-5-yl)pyrimidin-2-yl)tetrahydrofuran-3-sulfonamide (A-63)

Compound synthesised according to General Method C (C₅ moiety) and General Method A (C₇, core) and characterised: m/z: 540.0 [M+H]+; ¹H NMR (400 MHz, DMSO-d6) δ 13.75 (brs, 1H), 9.70-9.26 (m, 3H), 8.70 (d, J=1.9 Hz, 1H), 8.47 (d, J=8.3 Hz, 1H), 8.37 (s, 1H), 8.24 (s, 1H), 7.86 (dd, J=8.3, 1.8 Hz, 1H), 4.63 (ddd, J=14.9, 9.0, 6.2 Hz, 1H), 4.07-3.89 (m, 2H), 3.89-3.79 (m, 1H), 3.78-3.68 (m, 1H), 3.42-3.27 (m, 2H), 2.44 (s, 3H), 2.34-2.22 (m, 1H), 2.19-2.06 (m, 1H), 1.18 (t, J=7.1 Hz, 3H).

### N-(5-(7-bromo-2-(3-ethylureido)benzo[d]thiazol-5-yl)pyrimidin-2-yl)-2-methylpropane-2-sulfonamide (A-64)

Compound synthesised according to General Method B and characterised: m/z: 513, 514.81 [M+H]+; ¹H NMR (400 MHz, DMSO-d6) δ 8.98 (s, 2H), 7.99 (d, J=1.5 Hz, 1H), 7.81 (d, J=1.5 Hz, 1H), 3.27-3.13 (m, 2H), 1.40 (s, 9H), 1.10 (t, J=7.1 Hz, 3H).

### 1-[7-bromo-5-[6-(tert-butylsulfonylamino)-3-pyridyl]-1,3-benzothiazol-2-yl]-3-ethyl urea (A-65)

Compound synthesised according to General Method A and characterised: m/z: 513.84 [M+H]+; ¹H NMR (400 MHz, DMSO-d6) δ 8.60 (s, OH), 8.16-7.83 (m, 2H), 7.70 (d, J=1.5 Hz, 1H), 7.48 (d, J=9.2 Hz, 1H), 3.21 (q, J=7.1 Hz, 2H), 1.28 (s, 9H), 1.09 (t, J=7.1 Hz, 3H).

### methyl 2-[[5-[2-(ethylcarbamoylamino)-7-(5-methyl-2-pyridyl)-1,3-benzothiazol-5-yl]pyrimidin-2-yl]sulfamoyl]acetate (A-66)

Compound synthesised according to General Method C (C₅ moiety) and General Method A (C₇, core) and characterised: m/z: 542.0 [M+H]+; ¹H NMR (400 MHz, DMSO-d6) δ 13.62 (brs, 1H), 9.58 (s, 2H), 9.29 (brs, 1H), 8.70 (d, J=2.1 Hz, 1H), 8.50 (d, J=8.3 Hz, 1H), 8.41 (d, J=1.6 Hz, 1H), 8.28 (d, J=1.6 Hz, 1H), 7.86 (dd, J=8.3, 2.0 Hz, 1H), 4.40 (s, 2H), 3.64 (s, 3H), 3.41-3.25 (m, 2H), 2.44 (s, 3H), 1.18 (t, J=7.1 Hz, 3H).

### (R)—N-(5-(2-(3-ethylureido)-7-(5-methylpyridin-2-yl)benzo[d]thiazol-5-yl)pyrimidin-2-yl)-1-(tetrahydro-2H-pyran-2-yl)methanesulfonamide (A-67)

Compound synthesised according to General Method C (C₅ moiety) and General Method A (C₇, core) and characterised: m/z: 568.1 [M+H]+; ¹H NMR (400 MHz, DMSO-d6) δ 13.82 (brs, 1H), 9.54 (brs, 3H), 8.70 (d, J=2.2 Hz, 1H), 8.49 (d, J=8.3 Hz, 1H), 8.40 (s, 1H), 8.26 (s, 1H), 7.91-7.80 (m, 1H), 3.89-3.80 (m, 1H), 3.80-3.71 (m, 1H), 3.59 (dd, J=13.6, 3.9 Hz, 1H), 3.46-3.27 (m, 4H), 2.44 (s, 3H), 2.04 (d, J=13.2 Hz, 1H), 1.83-1.68 (m, 1H), 1.53-1.39 (m, 3H), 1.33-1.13 (m, 4H).

### N-(5-(2-(3-ethylureido)-7-(5-methylpyridin-2-yl)benzo[d]thiazol-5-yl)pyrimidin-2-yl)-2-methoxyethanesulfonamide (A-68)

Compound synthesised according to General Method C (C₅ moiety) and General Method A (C₇, core) and characterised: m/z: 528.0 [M+H]+; ¹H NMR (400 MHz, DMSO-d6) δ 13.76 (brs, 1H), 9.51 (brs, 3H), 8.69 (d, J=2.2 Hz, 1H), 8.48 (d, J=8.3 Hz, 1H), 8.39 (s, 1H), 8.24 (s, 1H), 7.92-7.80 (m, 1H), 3.81-3.58 (m, 4H), 3.43-3.28 (m, 2H), 3.25 (s, 3H), 2.44 (s, 3H), 1.18 (t, J=7.1 Hz, 3H).

### 1-[5-[6-(tert-butylsulfonylamino)-3-pyridyl]-7-(2-ethylthiazol-4-yl)-1,3-benzothiazol-2-yl]-3-ethyl urea (A-69)

Compound synthesised according to General Method D and characterised: m/z: 546.00 [M+H]+; ¹H NMR (400 MHz, DMF-d7) δ 9.39 (s, 2H), 8.45 (s, 1H), 8.29 (d, J=1.7 Hz, 1H), 8.24 (s, 1H), 3.43 (q, J=7.1 Hz, 2H), 3.22 (q, J=7.5 Hz, 2H), 1.53 (t, J=7.5 Hz, 4H), 1.48 (s, 9H), 1.23 (t, J=7.1 Hz, 4H).

### N-(5-(2-(3-ethylureido)-7-(5-methylpyridin-2-yl)benzo[d]thiazol-5-yl)pyrimidin-2-yl)-2-hydroxyethane sulfonamide (A-70)

Compound synthesised according to General Method C (C₅ moiety) and General Method A (C₇, core) and characterised: m/z: 514.0 [M+H]+; ¹H NMR (400 MHz, DMSO-d6) δ 13.77 (brs, 1H), 9.55 (brs, 3H), 8.70 (s, 1H), 8.49 (d, J=8.2 Hz, 1H), 8.39 (s, 1H), 8.26 (s, 1H), 7.95-7.76 (m, 1H), 4.65 (t, J=5.7 Hz, 1H), 3.85-3.68 (m, 2H), 3.60-3.47 (m, 2H), 3.38-3.30 (m, 2H), 2.44 (s, 3H), 1.19 (t, J=7.0 Hz, 3H).

### N-(5-(2-(3-ethylureido)-7-(1H-pyrazol-4-yl)benzo[d]thiazol-5-yl)pyrimidin-2-yl)-2-methylpropane-2-sulfonamide (A-71)

Compound synthesised according to General Method D and characterised: m/z: 500.96 [M+H]+; ¹H NMR (400 MHz, DMSO-d6) δ 9.18 (s, 2H), 8.22 (s, 2H), 7.92 (s, 1H), 7.78 (s, 1H), 3.30-3.22 (m, 2H), 1.33 (s, 9H), 1.13 (t, J=7.1 Hz, 3H).

### N-(5-(7-(3,5-dimethylisoxazol-4-yl)-2-(3-ethylureido)benzo[d]thiazol-5-yl)pyrimidin-2-yl)-2-methylpropane-2-sulfonamide (A-72)

Compound synthesised according to General Method D and characterised: m/z: 529.94 [M+H]+; ¹H NMR (400 MHz, DMSO-d6) δ 8.89 (s, 2H), 7.97 (d, J=1.6 Hz, 1H), 7.47 (d, J=1.7 Hz, 1H), 3.14 (q, J=7.2 Hz, 2H), 2.31 (s, 3H), 2.14 (s, 3H), 1.36 (s, 9H), 1.05 (t, J=7.2 Hz, 3H).

### N-(5-(2-(3-ethylureido)-7-(1-methyl-1H-pyrazol-4-yl)benzo[d]thiazol-5-yl)pyrimidin-2-yl)-2-methylpropane-2-sulfonamide (A-73)

Compound synthesised according to General Method D and characterised: m/z: 514.96 [M+H]+; ¹H NMR (400 MHz, DMSO-d6) δ 9.21 (s, 2H), 8.30 (s, 1H), 8.08 (s, 1H), 7.94 (s, 1H), 7.77 (s, 1H), 3.98 (s, 3H), 3.29-3.23 (m, 2H), 1.34 (s, 9H), 1.13 (t, J=7.1 Hz, 3H).

### N-(5-(2-(3-ethylureido)-7-((5-methylpyridin-2-yl)amino)benzo[d]thiazol-5-yl)pyrimidin-2-yl)-2-methylpropane-2-sulfonamide (A-74)

Compound synthesised according to General Method E and characterised: m/z: 541.2 [M+H]+; ¹H NMR (400 MHz, DMSO-d6) δ 10.78 (s, 1H), 8.89 (m, 3H), 8.17 (s, 1H), 7.98 (d, J=2.4 Hz, 1H), 7.79 (d, J=1.7 Hz, 1H), 7.64 (d, J=1.7 Hz, 1H), 7.44 (dd, J=8.5, 2.4 Hz, 1H), 6.85 (d, J=8.4 Hz, 2H), 3.24-3.12 (m, 2H), 2.19 (s, 3H), 1.40 (s, 9H), 1.09 (t, J=7.2 Hz, 3H).

### N-(5-(2-(3-ethylureido)-7-methylbenzo[d]thiazol-5-yl)pyrimidin-2-yl)-2-methylpropane-2-sulfonamide (A-75)

Compound synthesised according to General Method D and characterised: m/z: 449.1 [M+H]+; ¹H NMR (400 MHz, DMSO-d6) δ 10.79 (s, 1H), 8.95 (s, 2H), 7.83 (d, J=1.6 Hz, 1H), 7.49-7.30 (m, 1H), 6.83 (t, J=5.6 Hz, 1H), 3.25-3.15 (m, 2H), 2.52 (s, 3H), 1.41 (s, 9H), 1.10 (t, J=7.2 Hz, 3H).

### N-(5-(2-(3-ethylureido)benzo[d]thiazol-5-yl)pyrimidin-2-yl)-2-methylpropane-2-sulfonamide (A-76)

Compound synthesised according to General Method D (including hydrogenolysis of the C₇-bromide under Pd-catalyzed conditions after step b)) and characterised: m/z: 435.1 [M+H]+; ¹H NMR (400 MHz, DMSO-d6) δ 10.88 (s, 1H), 10.75 (s, 1H), 8.97 (s, 2H), 8.06-7.89 (m, 2H), 7.57 (dd, J=8.3, 1.8 Hz, 1H), 6.74 (t, J=5.6 Hz, 1H), 3.22-3.15 (m, 2H), 1.41 (s, 9H), 1.10 (t, J=7.2 Hz, 3H).

### N-(5-(7-cyclopropyl-2-(3-ethylureido)benzo[d]thiazol-5-yl)pyrimidin-2-yl)-2-methylpropane-2-sulfonamide (A-77)

Compound synthesised according to General Method D and characterised: m/z: 475.1 [M+H]+; ¹H NMR (400 MHz, Methanol-d4) δ 8.79 (s, 2H), 7.70 (d, J=1.6 Hz, 1H), 7.15 (dd, J=1.6, 0.8 Hz, 1H), 3.35 (m, 2H), 2.10 (tt, J=8.4, 5.2 Hz, 1H), 1.50 (s, 9H), 1.22 (t, J=7.2 Hz, 3H), 1.14-1.04 (m, 2H), 0.95-0.84 (m, 2H).

### N-(5-(2-(3-ethylureido)-7-(tetrahydro-2H-pyran-4-yl)benzo[d]thiazol-5-yl)pyrimidin-2-yl)-2-methylpropane-2-sulfonamide (A-78)

Compound synthesised according to General Method D and characterised: m/z: 519.01 [M+H]+; ¹H NMR (400 MHz, DMSO-d6) δ 10.66 (s, 1H), 9.27 (s, 1H), 7.91 (s, 1H), 7.37 (s, 1H), 7.17 (s, 1H), 6.86 (s, 1H), 6.74 (s, 1H), 6.26 (s, 1H), 4.29 (s, 4H), 3.86 (s, 1H), 3.17 (s, 2H), 2.50 (s*(+DMSO), 4H), 1.29 (s, 9H), 1.08 (s, 3H).

### N-(5-(2-(3-ethylureido)-7-(pyrrolidin-1-yl)benzo[d]thiazol-5-yl)pyrimidin-2-yl)-2-methylpropane-2-sulfonamide (A-79)

Compound synthesised according to General Method E and characterised: m/z: 504.2 [M+H]+; ¹H NMR (400 MHz, DMSO-d6) δ 9.06 (s, 2H), 7.43 (s, 1H), 6.63 (s, 1H), 3.60 (t, J=6.1 Hz, 4H), 3.28-3.22 (m, 2H), 2.03-1.97 (m, 4H), 1.31 (s, 9H), 1.11 (t, J=7.1 Hz, 3H).

### N-(5-(2-(3-ethylureido)-7-(piperazin-1-yl)benzo[d]thiazol-5-yl)pyrimidin-2-yl)-2-methylpropane-2-sulfonamide (A-80)

Compound synthesised according to General Method E and characterised: m/z: 519.3 [M+H]+; ¹H NMR (400 MHz, DMSO-d6) δ 9.00 (s, 2H), 8.21 (s, 1H), 7.67 (d, J=1.4 Hz, 1H), 7.11 (d, J=1.6 Hz, 1H), 3.28-3.17 (m, 7H), 3.13-3.02 (m, 4H), 1.39 (s, 9H), 1.10 (t, J=7.1 Hz, 3H).

### N-(5-(2-(3-ethylureido)-7-morpholinobenzo[d]thiazol-5-yl)pyrimidin-2-yl)-2-methylpropane-2-sulfonamide (A-81)

Compound synthesised according to General Method E and characterised: m/z: 520.1 [M+H]+; ¹H NMR (400 MHz, DMSO-d6) δ 10.92 (s, 1H), 8.97 (s, 2H), 7.66 (s, 1H), 7.12 (d, J=1.6 Hz, 1H), 6.89 (d, J=24.9 Hz, 1H), 3.87-3.76 (m, 4H), 3.24-3.16 (m, 6H), 1.40 (s, 9H), 1.10 (t, J=7.1 Hz, 3H).

### N-(5-(2-(3-ethylureido)-7-(4-methylpiperazin-1-yl)benzo[d]thiazol-5-yl)pyrimidin-2-yl)-2-methylpropane-2-sulfonamide (A-82)

Compound synthesised according to General Method E and characterised: m/z: 533.4 [M+H]+; ¹H NMR (400 MHz, DMSO-d6) δ 8.95 (s, 2H), 7.62 (d, J=1.5 Hz, 1H), 7.09 (d, J=1.6 Hz, 1H), 6.80 (s, 1H), 3.25-3.13 (m, 10H), 2.59-2.53 (m, 4H), 2.28 (s, 3H), 1.41 (s, 9H), 1.10 (t, J=7.2 Hz, 3H).

### N-(5-(7-(4-acetylpiperazin-1-yl)-2-(3-ethylureido)benzo[d]thiazol-5-yl)pyrimidin-2-yl)-2-methylpropane-2-sulfonamide (A-83)

Compound synthesised according to General Method E and characterised: m/z: 561.0 [M+H]+; ¹H NMR (400 MHz, DMSO-d6) δ 8.95 (s, 3H), 7.66 (s, 1H), 7.12 (s, 1H), 3.65 (m, 4H), 3.17 (m, 6H), 2.07 (s, 3H), 1.40 (s, 9H), 1.10 (t, J=7.1 Hz, 3H).

### N-(5-(2-(3-ethylureido)-7-(1-(2-morpholinoethyl)-1H-pyrazol-4-yl)benzo[d]thiazol-5-yl)pyrimidin-2-yl)-2-methylpropane-2-sulfonamide (A-84)

Compound synthesised according to General Method D and characterised: m/z: 614.30 [M+H]+; ¹H NMR (400 MHz, DMSO-d6) δ 10.96 (br s, 1H), 10.37 (br s, 1H), 9.06 (s, 2H), 8.45 (s, 1H), 8.25 (s, 1H), 7.92 (s, 1H), 7.84 (s, 1H), 6.93 (m, 1H), 4.74 (t, J=6.40 Hz, 2H), 3.97 (m, 2H), 3.69-3.75 (m, 5H), 3.13-3.23 (m, 5H), 1.42 (s, 9H), 1.10 (t, J=7.20 Hz, 3H).

### N-(5-(2-(3-ethylureido)-7-(4-methylpyridin-2-yl)benzo[d]thiazol-5-yl)pyrimidin-2-yl)-2-methylpropane-2-sulfonamide (A-85)

Compound synthesised according to General Method D and characterised: m/z: 526.50 [M+H]+; ¹H-NMR (DMSO-d6, 400 MHz): 10.93 (br s, 1H), 10.60 (br s, 1H), 9.13 (s, 2H), 8.66 (d, J=4.80 Hz, 1H), 8.39 (s, 1H), 8.33 (s, 1H), 8.06 (s, 1H), 7.29 (s, 1H), 6.87 (m, 1H), 3.18-3.24 (m, 2H), 2.46 (s, 3H), 1.42 (s, 9H), 1.11 (t, J=6.80 Hz, 3H).

### N-(5-(2-(3-ethylureido)-7-(5-(morpholinomethyl)pyridin-2-yl)benzo[d]thiazol-5-yl)pyrimidin-2-yl)-2-methylpropane-2-sulfonamide (A-86)

Compound synthesised according to General Method D and characterised: m/z: 611.57 [M+H]+; ¹H-NMR (DMSO-d6, 400 MHz): δ 10.96 (br s, 1H), 10.61 (br s, 1H), 9.13 (s, 2H), 8.72 (s, 1H), 8.49 (d, J=8.40 Hz, 1H), 8.32 (s, 1H), 8.07 (s, 1H), 7.92 (dd, J=2.0, 8.40 Hz respectively, 1H), 6.85 (m, 1H), 3.60 (m, 6H), 3.21 (m, 2H), 2.42 (br s, 4H), 1.43 (s, 9H), 1.11 (t, J=7.20 Hz, 3H).

### N-(5-(2-(3-ethylureido)-7-(2-(3-hydroxypyrrolidin-1-yl)thiazol-4-yl)benzo[d]thiazol-5-yl)pyrimidin-2-yl)-2-methylpropane-2-sulfonamide (A-87)

Compound synthesised according to General Method D and characterised: m/z: 603.40 [M+H]+; ¹H-NMR (DMSO-d6, 400 MHz): δ 10.92 (br s, 1H), 10.55 (br s, 1H), 9.05 (s, 2H), 8.03 (s, 1H), 7.90 (s, 1H), 7.55 (s, 1H), 6.83 (m, 1H), 5.17 (d, J=3.60 Hz, 1H), 4.48 (br s, 1H), 3.56-3.67 (m, 3H), 3.39-3.45 (m, 1H), 3.17-3.24 (m, 2H), 2.0-2.19 (m, 2H), 1.41 (s, 9H), 1.12 (t, J=7.20 Hz, 3H).

### 1-[5-[2-(tert-butylsulfonylamino)pyrimidin-5-yl]-7-(2-hydroxy-4-pyridyl)-1,3-benzothiazol-2-yl]-3-ethyl-urea (A-88)

Compound synthesised according to General Method D and characterised: m/z: 528.33 [M+H]+; ¹H-NMR (DMSO-d6, 400 MHz): δ 9.04 (s, 2H), 8.05 (s, 1H), 7.69 (s, 1H), 7.57 (d, J=6.0 Hz, 1H), 6.71 (s, 1H), 6.62 (m, 1H), 3.23 (m, 2H), 1.39 (s, 9H), 1.09 (t, J=7.20 Hz, 3H).

### N-(5-(2-(3-ethylureido)-7-(1-methyl-2-oxo-1,2-dihydropyridin-4-yl)benzo[d]thiazol-5-yl)pyrimidin-2-yl)-2-methylpropane-2-sulfonamide (A-89)

Compound synthesised according to General Method D and characterised: m/z: 542.30 [M+H]+; ¹H-NMR (DMSO-d6, 400 MHz): δ 10.89-10.93 (m, 2H), 9.07 (s, 2H), 8.07 (s, 1H), 7.90 (s, 1H), 7.71 (s, 1H), 6.66-6.79 (m, 3H), 3.50 (s, 3H), 3.15-3.20 (m, 2H), 1.41 (s, 9H), 1.09 (t, J=7.20 Hz, 3H).

### 2-(4-(5-(2-(1,1-Dimethylethylsulfonamido)pyrimidin-5-yl)-2-(3-ethylureido)benzo[d]thiazol-7-yl)piperazin-1-yl)-N-methylacetamide (A-90)

Compound synthesised according to General Method E and characterised: m/z: 590.6 [M+H]+; ¹H NMR (400 MHz, DMSO-d6) δ 10.81 (s, 1H), 8.95 (s, 2H), 7.73 (m, 1H), 7.63 (d, J=1.5 Hz, 1H), 7.09 (d, J=1.6 Hz, 1H), 6.80 (s, 1H), 3.25 (d, J=9.7 Hz, 5H), 3.19 (dd, J=7.3, 5.6 Hz, 2H), 3.02 (s, 2H), 2.65 (dd, J=10.7, 5.0 Hz, 6H), 1.41 (s, 9H), 1.10 (t, J=7.1 Hz, 3H).

### Ethyl 2-(4-(5-(2-(1,1-dimethylethylsulfonamido)pyrimidin-5-yl)-2-(3-ethylureido)benzo[d]thiazol-7-yl)piperazin-1-yl)acetate (A-91)

Compound synthesised according to General Method E and characterised: m/z: 605.7 [M+H]+; ¹H NMR (400 MHz, DMSO-d6) δ 10.85 (s, 1H), 8.96 (s, 2H), 7.64 (s, 1H), 7.10 (d, J=1.6 Hz, 1H), 4.12 (q, J=7.1 Hz, 2H), 3.25-3.14 (m, 7H), 2.75 (t, J=4.6 Hz, 5H), 1.40 (s, 9H), 1.22 (t, J=7.1 Hz, 4H), 1.10 (t, J=7.1 Hz, 3H).

### N-(5-(2-(3-ethylureido)-7-(2-(piperazin-1-yl)thiazol-4-yl)benzo[d]thiazol-5-yl)pyrimidin-2-yl)-2-methylpropane-2-sulfonamide (A-92)

Compound synthesised according to General Method D and characterised: m/z: 602.09 [M+H]+; ¹H NMR (400 MHz, DMSO-d6) δ 10.93 (br s, 1H), 10.71 (br s, 1H), 9.14 (br s, 1H), 9.07 (s, 2H), 8.06 (s, 1H), 7.95 (s, 1H), 7.79 (s, 1H), 6.93 (m, 1H), 3.80 (br s, 4H), 3.33 (br s, 4H), 3.17-3.24 (m, 2H), 1.42 (s, 9H), 1.10 (t, J=7.20 Hz, 3H).

### N-(5-(2-(3-ethyl N-(5-(2-(3-ethylureido)-7-(6-methylpyridin-2-yl)benzo[d]thiazol-5-yl)pyrimidin-2-yl)-2-methylpropane-2-sulfonamide (A-93)

Compound synthesised according to General Method D and characterised: m/z: 526.15 [M+H]+; ¹H NMR (400 MHz, DMSO-d6) δ 11.05 (br s, 1H, D₂O exchangeable), 10.59 (br s, 1H, D20 exchangeable), 9.12 (s, 2H), 8.32 (m, 2H), 8.06 (s, 1H), 7.86-7.90 (m, 1H), 7.31 (d, J=7.60 Hz, 1H), 6.95 (m, 1H, D20 exchangeable), 3.20-3.26 (m, 2H), 2.66 (s, 3H), 1.41 (s, 9H), 1.10 (t, J=7.20 Hz, 3H).

### N-(5-(7-(2-aminopyridin-3-yl)-2-(3-ethylureido)benzo[d]thiazol-5-yl)pyrimidin-2-yl)-2-methylpropane-2-sulfonamide (A-94)

Compound synthesised according to General Method D and characterised: m/z: 527.03 [M+H]+; ¹H NMR (400 MHz, DMSO-d6) δ 10.96 (br s, 1H), 10.78 (br s, 1H), 9.01 (s, 2H), 8.06 (d, J=4.0 Hz, 1H), 7.99 (s, 1H), 7.51-7.53 (m, 2H), 6.68-6.74 (m, 2H), 5.72 (br s, 2H), 3.12-3.19 (m, 2H), 1.41 (s, 9H), 1.07 (t, J=7.20 Hz, 3H).

### N-(5-(7-(5-aminopyridin-3-yl)-2-(3-ethylureido)benzo[d]thiazol-5-yl)pyrimidin-2-yl)-2-methylpropane-2-sulfonamide (A-95)

Compound synthesised according to General Method D and characterised: m/z: 527.09 [M+H]+; ¹H NMR (400 MHz, DMSO-d6) δ 10.98 (br s, 1H), 10.84 (br s, 1H), 9.04 (s, 2H), 8.10 (s, 1H), 8.01 (br s, 2H), 7.62 (s, 1H), 7.28 (s, 1H), 6.74 (m, 1H), 5.58 (br s, 2H), 3.17 (m, 2H), 1.41 (s, 9H), 1.08 (t, J=6.80 Hz, 3H).

### N-(5-(2-(3-ethylureido)-7-(4-(2-methoxyethyl)piperazin-1-yl)benzo[d]thiazol-5-yl)pyrimidin-2-yl)-2-methylpropane-2-sulfonamide (A-96)

Compound synthesised according to General Method E and characterised: m/z: 577.6 [M+H]+; ¹H NMR (400 MHz, DMSO-d6) δ 10.83 (s, 1H), 8.95 (s, 2H), 7.62 (d, J=1.4 Hz, 1H), 7.08 (d, J=1.6 Hz, 1H), 6.83 (s, 1H), 3.49 (t, J=5.8 Hz, 2H), 3.26 (s, 3H), 3.23-3.16 (m, 6H), 2.65 (t, J=4.5 Hz, 4H), 2.58 (t, J=5.7 Hz, 2H), 1.41 (s, 9H), 1.10 (t, J=7.2 Hz, 3H).

### N-(5-(2-(3-ethylureido)-7-(piperidin-1-yl)benzo[d]thiazol-5-yl)pyrimidin-2-yl)-2-methylpropane-2-sulfonamide (A-97)

Compound synthesised according to General Method E and characterised: m/z: 518.21 [M+H]+; ¹H NMR (400 MHz, CDCl3) δ 8.90 (s, 2H), 8.00 (s, 1H), 6.94 (d, J=1.6 Hz, 1H), 3.44 (p, J=7.0 Hz, 2H), 3.19 (t, J=5.3 Hz, 4H), 1.77 (q, J=10.0, 7.9 Hz, 4H), 1.56 (s, 11H), 1.27 (t, J=7.2 Hz, 3H).

### N-(5-(7-(5-(aminomethyl)-2-fluorophenyl)-2-(3-ethylureido)benzo[d]thiazol-5-yl)pyrimidin-2-yl)-2-methylpropane-2-sulfonamide (A-98)

Compound synthesised according to General Method D and characterised: m/z: 558.10 [M+H]+; ¹H NMR (400 MHz, DMSO-d6+TFA-d): d 9.02 (s, 2H), 8.20 (br s, 3H), 8.08 (s, 1H), 7.78 (d, J=5.60 Hz, 1H), 7.62 (m, 1H), 7.56 (s, 1H), 7.48-7.52 (m, 1H), 6.81 (m, 1H), 4.13 (m, 2H), 3.15 (m, 2H), 1.41 (s, 9H), 1.06 (t, J=7.20 Hz, 3H).

### N-(5-(2-(3-ethylureido)-7-[2-dimethylaminoethyl(methyl)amino]benzo[d]thiazol-5-yl)pyrimidin-2-yl)-2-methylpropane-2-sulfonamide (A-99)

Compound synthesised according to General Method E and characterised: m/z: 535.16 [M+H]+; ¹H NMR (400 MHz, DMSO-d6) δ 8.93 (s, 2H), 7.52 (d, J=1.5 Hz, 1H), 6.99 (d, J=1.6 Hz, 1H), 3.45-3.39 (m, 2H), 3.23-3.17 (m, 2H), 2.99 (d, J=4.9 Hz, 3H), 2.50 (m, 2H), 2.18 (d, J=3.0 Hz, 6H), 1.40 (d, J=2.1 Hz, 9H), 1.10 (t, J=7.2 Hz, 3H).

### N-(5-(2-(3-ethylureido)-7-(pyridin-2-yl)benzo[d]thiazol-5-yl)pyrimidin-2-yl)-2-methylpropane-2-sulfonamide (A-100)

Compound synthesised according to General Method D and characterised: m/z: 512.11 [M+H]+; ¹H NMR (400 MHz, DMSO-d6) δ 10.93 (br s, 1H), 10.63 (br s, 1H), 9.14 (s, 2H), 8.82 (m, 1H), 8.53 (d, J=8.0 Hz, 1H), 8.34 (s, 1H), 8.08 (s, 1H), 8.0 (t, J=7.20 Hz, 1H), 7.45 (m, 1H), 6.86 (m, 1H), 3.16-3.22 (m, 2H), 1.43 (s, 9H), 1.11 (t, J=7.20 Hz, 3H).

### 1-[6-[2-(tert-butylsulfonylamino)pyrimidin-5-yl]-5-methoxy-thiazolo[5,4-b]pyridin-2-yl]-3-ethylurea (A-103)

Compound synthesised according to General Method F and characterised: m/z: 466.09 [M+H]+; ¹H NMR (400 MHz, DMSO-d6) δ 10.97 (s, 1H), 10.77 (s, 1H), 8.79 (s, 2H), 8.11 (s, 1H), 6.71 (s, 1H), 3.95 (s, 3H), 3.25-3.13 (m, 2H), 1.41 (s, 9H), 1.09 (t, J=7.1 Hz, 3H).

### 1-[5-[2-(tert-butylsulfonylamino)pyrimidin-5-yl]-7-(5-hydroxy-2-pyridyl)-1,3-benzothiazol-2-yl]-3-ethylurea (A-104)

Compound synthesised according to General Method D and characterised: m/z: 528.11 [M+H]+; ¹H NMR (400 MHz, DMSO-d6) δ 10.92 (br s, 1H), 10.57 (br s, 1H), 10.29 (s, 1H), 9.11 (s, 2H), 8.36 (m, 2H), 8.17 (s, 1H), 7.98 (s, 1H), 7.36 (m, 1H), 6.85 (m, 1H), 3.21 (m, 2H), 1.42 (s, 9H), 1.11 (t, J=6.80 Hz, 3H).

### 1-[5-[2-(tert-butylsulfonylamino)pyrimidin-5-yl]-7-[4-[(cyclopropylamino)methyl]-2-pyridyl]-1,3-benzothiazol-2-yl]-3-ethylurea (A-105)

Compound synthesised according to General Method D and characterised: m/z: 581.17 [M+H]+; 1H NMR (400 MHz, DMSO-d6) δ 10.60 (br s, 1H), 9.14 (s, 2H), 8.73 (d, J=5.20 Hz, 1H), 8.45 (s, 1H), 8.40 (s, 1H), 8.14 (s, 1H), 7.48 (m, 1H), 6.85 (m, 1H), 3.91 (s, 2H), 3.19-3.24 (m, 2H), 2.08 (m, 1H), 1.43 (s, 9H), 1.11 (t, J=7.20 Hz, 3H), 0.40 (m, 2H), 0.33 (m, 2H).

### 1-[7-(5-amino-2-pyridyl)-5-[2-(tert-butylsulfonylamino)pyrimidin-5-yl]-1,3-benzothiazol-2-yl]-3-ethylurea (A-106)

Compound synthesised according to General Method D and characterised: m/z: 527.12 [M+H]+; ¹H NMR (400 MHz, DMSO-d6) δ 10.91 (br s, 1H), 10.51 (br s, 1H), 9.09 (s, 2H), 8.07-8.17 (m, 3H), 7.90 (s, 1H), 7.11 (m, 1H), 6.88 (m, 1H), 5.68 (br s, 2H), 3.17-3.24 (m, 2H), 1.42 (s, 9H), 1.11 (t, J=7.20 Hz, 3H).

### 1-[5-[2-(tert-butylsulfonylamino)pyrimidin-5-yl]-7-(3-methyl-2-pyridyl)-1,3-benzothiazol-2-yl]-3-ethyl-urea (A-107)

Compound synthesised according to General Method D and characterised: m/z: 526.11 [M+H]+; ¹H NMR (400 MHz, DMSO-d6) δ 10.92 (br s, 1H), 10.73 (br s, 1H), 9.01 (s, 2H), 8.58 (d, J=4.0 Hz, 1H), 8.03 (s, 1H), 7.86 (d, J=7.60 Hz, 1H), 7.74 (s, 1H), 7.39-7.42 (m, 1H), 6.77 (m, 1H), 3.13-3.19 (m, 2H), 2.42 (s, 3H), 1.41 (s, 9H), 1.07 (t, J=7.20 Hz, 3H).

### 1-[7-(4-amino-2-pyridyl)-5-[2-(tert-butylsulfonylamino)pyrimidin-5-yl]-1,3-benzothiazol-2-yl]-3-ethylurea (A-110)

Compound synthesised according to General Method D and characterised: m/z: 527.19 [M+H]+; ¹H NMR (400 MHz, DMSO-d6) δ 10.93 (br s, 1H), 10.54 (br s, 1H), 9.06 (s, 2H), 8.22 (d, J=5.60 Hz, 1H), 7.99 (br s, 2H), 7.47 (s, 1H), 6.87 (m, 1H), 6.55 (s, 1H), 6.12 (br s, 2H), 3.14-3.23 (m, 2H), 1.42 (s, 9H), 1.10 (t, J=7.20 Hz, 3H).

### 1-[5-[2-(2,3-dihydroxypropylsulfonylamino)pyrimidin-5-yl]-7-(5-methyl-2-pyridyl)-1,3-benzothiazol-2-yl]-3-ethylurea (A-111)

Compound synthesised according to General Method D and characterised: m/z: 544.12 [M+H]+; ¹H NMR (400 MHz, DMSO-d6) δ 11.38 (br s, 1H), 10.59 (br s, 1H), 9.15 (s, 2H), 8.65 (s, 1H), 8.43 (d, J=8.0 Hz, 1H), 8.29 (s, 1H), 8.05 (s, 1H), 7.84 (d, J=8.0 Hz, 1H), 6.85 (m, 1H), 5.01 (d, J=4.80 Hz, 1H), 4.80 (t, J=6.40 Hz, 1H), 3.94-4.02 (m, 1H), 3.61-3.78 (m, 2H), 3.40 (m, 2H), 3.20-3.32 (m, 2H), 2.40 (s, 3H), 1.11 (t, J=7.20 Hz, 3H).

### 1-[5-[2-(tert-butylsulfonylamino)pyrimidin-5-yl]-7-(4,5-dimethyl-2-pyridyl)-1,3-benzothiazol-2-yl]-3-ethylurea (A-113)

Compound synthesised according to General Method D and characterised: m/z: 540.14 [M+H]+; ¹H NMR (400 MHz, DMSO-d6) δ 10.93 (m, 2H), 9.02 (s, 2H), 8.51 (s, 1H), 8.09 (s, 1H), 7.67 (s, 1H), 7.47 (m, 1H), 6.82 (m, 1H), 3.15 (m, 2H), 2.44 (s, 3H), 2.24 (s, 3H), 1.41 (s, 9H), 1.06 (t, J=7.20 Hz, 3H).

### 1-[5-[2-(tert-butylsulfonylamino)pyrimidin-5-yl]-7-[(3S,4R,5R,6R)-3,4,5,6-tetrahydroxycyclohexen-1-yl]-1,3-benzothiazol-2-yl]-3-ethylurea (A-114)

Compound synthesised according to General Method D and characterised: m/z: 579.12 [M+H]+; ¹H NMR (400 MHz, DMSO-d6) δ 9.07 (s, 2H), 7.97 (s, 1H), 7.74 (s, 1H), 6.10 (d, J=2.5 Hz, 1H), 5.21 (d, J=5.7 Hz, 1H), 5.12 (d, J=5.4 Hz, 1H), 4.77 (d, J=4.5 Hz, 1H), 4.64 (d, J=5.8 Hz, 1H), 4.56 (dd, J=5.4, 3.7 Hz, 1H), 4.02 (ddd, J=8.0, 5.9, 2.5 Hz, 1H), 3.69 (ddd, J=10.4, 7.5, 4.3 Hz, 1H), 3.46-3.41 (m, 1H), 3.27-3.23 (m, 2H), 1.33 (s, 9H), 1.12 (t, J=7.0 Hz, 3H).

### 1-[7-[(3aR,6aR)-2,3,3a,4,6,6a-hexahydro-1H-pyrrolo[2,3-c]pyrrol-5-yl]-5-[2-(tert-butylsulfonylamino)pyrimidin-5-yl]-1,3-benzothiazol-2-yl]-3-ethyl-urea (A-115)

Compound synthesised according to General Method E and characterised: m/z: 545.50 [M+H]+; ¹H NMR (400 MHz, DMSO-d6) δ 8.89 (s, 2H), 7.50 (d, J=1.4 Hz, 1H), 6.81 (d, J=1.5 Hz, 1H), 4.28-4.18 (m, 1H), 3.55 (dt, J=10.4, 5.0 Hz, 3H), 3.37 (dd, J=9.7, 7.7 Hz, 1H), 3.24-3.07 (m, 5H), 2.23-2.14 (m, 1H), 1.93-1.82 (m, 1H), 1.35 (s, 9H), 1.08 (t, J=7.1 Hz, 3H).

### Example 4

Compounds A-102, A-108, and A-112 of Formula (III) were prepared according to the General Methods described herein as follows.

### N-[5-(2-amino-7-bromo-1,3-benzothiazol-5-yl)pyrimidin-2-yl]-2-methyl-propane-2-sulfonamide (A-102)

Compound synthesised according to General Method H and characterised: m/z: 442.1, 444.1 [M+H]+; ¹H NMR (400 MHz, DMSO-d6) δ 8.68 (s, 2H), 7.77 (s, 2H), 7.58 (s, 1H), 7.49 (s, 1H), 1.33 (s, 9H).

### N-[5-[2-amino-4-(5-methyl-2-pyridyl)pyrazolo[1,5-a]pyridin-6-yl]pyrimidin-2-yl]-2-methyl-propane-2-sulfonamide (A-108)

Compound synthesised according to General Method G and characterised: m/z: 438.20 [M+H]+; ¹H NMR (400 MHz, DMSO-d6) δ 8.98 (s, 1H), 8.87 (s, 2H), 8.47 (d, J=2.4 Hz, 1H), 7.97 (d, J=8.0 Hz, 1H), 7.86 (s, 1H), 7.68 (dd, J=8.5, 2.8 Hz, 1H), 5.82 (d, J=0.8 Hz, 1H), 5.54 (s, 2H), 2.33 (s, 3H), 1.41 (s, 9H).

### N-[5-(2-amino-1,3-benzothiazol-5-yl)pyrimidin-2-yl]-2-methyl-propane-2-sulfonamide (A-112)

Compound synthesised according to General Method H and characterised: m/z: 364.07 [M+H]+; ¹H NMR (400 MHz, DMSO-d6) δ 10.87 (br s, 1H), 8.92 (s, 2H), 7.77 (d, J=8.0 Hz, 1H), 7.69 (s, 1H), 7.58 (br s, 2H), 7.37 (d, J=8.40 Hz, 1H), 1.40 (s, 9H).

### Example 5

Compounds A-101 and A-109 of Formula (IV) were prepared according to the General Methods described herein as follows.

### 1-[6-[2-(tert-butylsulfonylamino)pyrimidin-5-yl]-8-(5-methyl-2-pyridyl)-[1,2,4]triazolo[1,5-a]pyridin-2-yl]-3-ethyl-urea (A-101)

Compound synthesised according to General Method I and characterised: m/z: 510.2 [M+H]+; ¹H NMR (400 MHz, DMSO-d6) δ 11.03 (s, 1H), 10.06 (s, 1H), 9.35 (d, J=1.8 Hz, 1H), 9.02 (s, 2H), 8.74-8.59 (m, 3H), 8.10 (t, J=5.3 Hz, 1H), 7.84 (ddd, J=8.2, 2.3, 0.9 Hz, 1H), 3.31-3.26 (m, 2H), 2.41 (s, 3H), 1.42 (s, 9H), 1.19 (t, J=7.2 Hz, 3H).

### 1-[4-bromo-6-[2-(tert-butylsulfonylamino)pyrimidin-5-yl]pyrazolo[1,5-a]pyridin-2-yl]-3-ethylurea (A-109)

Compound synthesised according to General Method G and characterised: m/z: 495.9/497.9 [M+H]+; ¹H NMR (400 MHz, DMSO-d6) δ 9.22 (s, 1H), 8.81 (s, 1H), 8.67 (s, 2H), 7.41 (s, 1H), 6.66 (s, 1H), 6.61 (t, J=5.5 Hz, 1H), 3.14 (dt, J=13.6, 7.1 Hz, 2H), 1.36 (s, 9H), 1.07 (t, J=7.2 Hz, 3H).

### Example 6

Examples of other bacterial type II topoisomerase inhibitor that have on-target enzyme activity against DNA gyrase and optionally on-target enzyme activity against topoisomerase IV which may also be suitable for use in combination with polymyxin or a polymyxin derivative include, but are not limited to the following.

### Methyl 2-(ethylcarbamoylamino)-6-(3-pyridyl)-3H-benzimidazole-4-carboxylate (A-22)

Compound class generally described in WO2003/105846 (Vertex Pharmaceuticals Incorporated, Charifson, P. et. al.).

### 6-Fluoro-N-methyl-2-[(6-methyl-3-pyridyl)oxy]-4-pyrrolidin-1-yl-9H-pyrimido[4, 5-b]indol-8-amine (A-23)

Compound class generally described in WO2012/125746 (Trius Therapeutics Inc., Bensen, D. et. al.).

### ADME Assays

The following assay(s) may be used to assess the properties of a prodrug and the potential suitability of the prodrug to deliver a compound in vivo.

### Chemical Stability Assay

Compounds may be tested for chemical stability across four pH values i.e. 2.1, 4.5, 7.4 and 9.1 and in water. Stress solutions are prepared with 10% acetonitrile and samples introduced from DMSO stocks (2 mM) to give a final concentration of 16 μM. Test samples are analysed by HPLC using a C8 reverse phase column (Phenomenex Kinetex™ 2.6 μm C8 100 Å LC Column 50×3 mm or similar) with an elution gradient of 5-100% acetonitrile:water+0.1% formic acid. The assay is conducted over 24 hrs with 2-hourly injections. Data analysis is performed using peak areas at 254 nm and LCMS for mass determination.

In an alternative method, compounds are dissolved in DMSO are diluted to produce triplicate 10 μM solutions in HEPES pH 7.4 containing 5% DMSO. The samples are incubated for 24 h at 37° C. and analysed by LCMS following the addition of 2 volumes of methanol. The percentage of compound remaining is determined by comparing peak areas to a T0 sample.

### Thermodynamic Solubility

In a 1.5 mL eppendorf tube, approximately 2 mg of compound is resuspended in a volume of HEPES buffer pH 7.4 to achieve a 5 mg/mL suspension. The tubes are placed on an orbital shaker at room temperature and following shaking for 24 hours the tubes are spun at 1400 rpm in a bench top centrifuge to pellet the undissolved compound. Duplicate 150 μL aliquots of the supernatant are then transferred into two ultracentrifuge tubes and spun at 357440 g for 4 hours at 20° C. 50 μL of the supernatant from each tube is then diluted with 100 μL of methanol and analysed by HPLC or LCMS to determine the concentration of compound in solution by comparing to a standard curve.

### Microsomal Stability

In a 96 well polypropylene plate, 10 μM compound is prepared in 100 mM KPO₄ buffer pH 7.4, 5 mM MgCl₂, 25 μg/ml Alamethicin, 1 mg/ml (protein) liver microsomes (mouse) and a final DMSO concentration of 0.1% in 100 μl in duplicate. The plate is pre-incubated at 37° C. for 10 minutes after which reactions are initiated by the addition of NADPH and UDPGA to a final concentration of 1 mM and 5 mM respectively. Reactions are incubated at 37° C. and terminated by the addition of 100 μl DMSO at 0, 10, 30 and 60 minutes. Samples of 100 μl are withdrawn and added to 50 μl ice cold methanol and mixed on an orbital shaker for 10 minutes to precipitate the proteins. The samples are then centrifuged at 4000 rpm and 10° C. for 30 minutes and supernatants are analysed by LCMS. The T1/2 and clearance are determined by linear regression from the peak areas.

### Plasma Stability

Compounds dissolved in DMSO are diluted to produce duplicate 50 μl aliquots of 10 μM solutions in neat plasma containing 1% DMSO in 96 well polypropylene plates. Following incubation at 37° C. for 5 hours, 100 μl ice cold acetonitrile is added to the samples and mixed on an orbital shaker for 10 minutes to precipitate the proteins. The samples are then centrifuged at 4000 rpm and 10° C. for 30 minutes and supernatants are analysed by LCMS. The recovery and percentage of compound remaining is determined by comparing peak areas to a DMSO stock and T0 sample respectively.

### Biological Data

The in vitro and in vivo antiviral activity of the compounds of the present disclosure may be determined using the following protocols.

### On-Target Enzyme Assay: Determination of ATPase Activity

The bacterial type II topoisomerases, DNA gyrase and topoisomerase IV, convert ATP into ADP and inorganic phosphate. The released phosphate can be detected by the addition of malachite green solution and measured by monitoring the increase in absorbance at 600 nm. The DNA gyrase ATPase assay is carried out in 25 μl of a buffer containing 16 nM DNA Gyrase enzyme (A₂B₂ complex from Escherichia coli), 10 μg/mL stDNA, 80 mM Tris pH 7.5, 100 mM potassium glutamate, 20 mM magnesium acetate, 10 mM DTT, 0.2 mg/mL BSA and 1% DMSO solution containing the inhibitor. The topoisomerase IV ATPase assay is carried out in 25 μl of a buffer containing 10 nM topoisomerase IV enzyme (C₂E₂ complex from Escherichia coli), 100 stDNA, 80 mM Tris pH 7.5, 100 mM potassium glutamate, 20 mM magnesium acetate, 10 mM DTT, 0.2 mg/mL BSA and 1% DMSO solution containing the inhibitor. The reactions are started by adding ATP to a final concentration of 1 mM (DNA gyrase) or 0.5 mM (topoisomerase IV) and allowed to incubate at 30° C. for 60 minutes. The reactions are stopped by adding 200 μL of malachite green solution (0.034% malachite green, 10 mM ammonium molybdate, 1 M HCl, 3.4% ethanol, 0.01% tween 20). Colour is allowed to develop for 5 minutes and the absorbance at 600 nm is measured spectrophotometrically. The IC₅₀ values are determined from the absorbance readings using no compound and no enzyme controls.

The compounds of the present disclosure demonstrated on target enzyme activity with the majority of compounds tested showing Gyrase ATPase activity IC₅₀ values less than 1 μg/mL, with most of these being less than 0.1 μg/mL.

### Bacterial Assays: Determination of Antibacterial Activity

Compounds of the present disclosure were tested for antimicrobial activity by susceptibility testing in liquid or on solid media. MICs for compounds against each strain were determined by the broth microdilution or agar dilution method according to the guidelines of the Clinical Laboratories and Standards Institute, formerly the National Committee for Clinical Laboratory Standards (Clinical Laboratories and Standards Institute. Methods for Dilution Antimicrobial Susceptibility Tests for Bacteria That Grow Aerobically; Approved Standard-Seventh Edition. Document M7-A7. CLSI, Wayne, Pa., 2006; Clinical Laboratories and Standards Institute. Gram-positive bacterial strains tested include E. faecalis (Enteroccocus faecalis (Isolate ID ATCC 29212) and S. aureus (Staphylococcus aureus (Isolate ID ATCC 29213). Gram-negative bacterial strains tested include A. baumannii (Acinetobacter baumannii (Isolate ID ATCC 19606)), E. coli (Escherichia coli (Isolate ID ATCC 25922)), K. pneumoniae (Klebsiella pneumoniae (Isolate ID ATCC 13882)), P. aeruginosa (Pseudomonas aeruginosa (Isolate ID ATCC 27853)), E. cloaceae (Enterobacter cloacae (Isolate ID ATCC 13047)), B. cepacia (Burkholderia cepacia (Isolate ID ATCC 25416)), F. philomiragia (Francisella philomiragia (Isolate ID ATCC 25015)), N. gonorrhoeae (Neisseria gonorrhoeae (Isolate ID ATCC 49226)), and H. influenzae (Haemophilus influenzae (Isolate ID ATCC 49247)).

### Gram-Positive Antibacterial Activity

Selected compounds of the present disclosure demonstrated antibacterial activity against the Gram-positive bacterial strains E. faecalis (ATCC 29212) and S. aureus (ATCC 29213) with the majority of the compounds of Formula (II), Formula (III) and Formula (IV) tested showing activity less than 4 μg/mL, with most of these being less than 0.25 μg/mL.

### Gram-Negative Antibacterial Activity

Selected compounds of Formula (I), Formula (II), Formula (III) and Formula (IV) were tested alone and in combination with a polymyxin or polymyxin derivative for activity against Gram-negative bacterial pathogens and drug resistant clinical isolates thereof.

The compounds of the present disclosure demonstrated antibacterial activity against the Gram-negative bacterial strains N. gonorrhoeae (ATCC 49226) and H. influenzae (ATCC 49247) with the majority of compounds tested showing activity less than 4 μg/mL, with most of these being less than 0.1 μg/mL.

In one experiment, selected compounds of Formula (I), Formula (II), Formula (III), and Formula (IV) and a comparative compound example, ofloxacin, were tested for activity against E. coli (ATCC 25922) alone and in combination with a polymyxin derivative, polymyxin B nonapeptide (PMBN). In contrast to the results obtained for compounds of Formula (I), Formula (II), Formula (III), and Formula (IV) in the presence of PMBN which showed an improvement or enhancement of activity when compared to the activity of these compounds alone, no effect was observed for the comparative compound, ofloxacin, in the presence of PMBN.

TABLE 1
Antibacterial activity against Gram-negative bacterial type strain:
E. coli (ATCC 25922)

| | | MIC | MIC of Compound |
|---|---|---|---|
| | | PMBN | (μg/ml) in the |
| | MIC Compound | alone | presence of PMBN |
| Compound Example | alone (μg/ml) | (μg/ml) | (128 μg/ml) |
| Example 51 | 16 | >128^{(a)} | 0.06 |
| (WO2007/148093) | | | |
| Example 94 | >64 | >128^{(a)} | 32 |
| (WO2007/148093) | | | |
| Example 163 | >64 | >128^{(a)} | 0.25 |
| (WO2007/148093) | | | |
| Example 1 Compound | >64 | >128^{(a)} | 8 |
| VI(a) | | | |
| (WO2009/074812) | | | |
| Example 1 | >64 | >128^{(a)} | 0.5 |
| (WO2012/045124) | | | |
| Example 23 | 4 | >128^{(a)} | 0.06 |
| (WO2013/138860) | | | |
| Example 28 | >64 | >128^{(a)} | 0.5 |
| (WO2013/138860) | | | |
| A-1 | >64 | >128^{(a)} | 0.12 |
| A-2 | >64 | >128^{(a)} | <=.12 |
| A-3 | >64 | >128^{(a)} | <=1 |
| A-4 | >64 | >128^{(a)} | 0.5 |
| A-5 | >64 | >128^{(a)} | 0.008 |
| A-6 | >64 | >128^{(a)} | 0.25 |
| A-7 | 32 | >128^{(a)} | 4 |
| A-8 | 4 | >128^{(a)} | 0.06 |
| A-9 | 32 | >128^{(a)} | 2 |
| A-10 | >64 | >128^{(a)} | 4 |
| A-11 | 2 | >128^{(a)} | 0.015 |
| A-12 | 2 | >128^{(a)} | 0.008 |
| A-13 | 1 | >128^{(a)} | 0.008 |
| A-14 | 4 | >128^{(a)} | 0.03 |
| A-15 | 4 | >128^{(a)} | 0.03 |
| A-16 | >64 | >128^{(a)} | 0.25 |
| A-17 | 2 | >128^{(a)} | 0.03 |
| A-18 | >64 | >128^{(a)} | 0.12 |
| A-19 | >64 | >128^{(a)} | <=.12 |
| A-20 | >64 | >128^{(a)} | 32 |
| A-22 | 8 | >128^{(a)} | 0.5 |
| A-23 | 1 | >128^{(a)} | 0.06 |
| A-101 | >64 | >128^{(a)} | 2 |
| A-102 | >64 | >128^{(a)} | 2 |
| Ofloxacin | 0.03 | >128^{(a)} | 0.03^{(b)} |

^{(a)}No measurable antibacterial activity
^{(b)}No effect

In another experiment, selected compounds of Formula (I) and Formula (II) were tested for activity against E. coli (ATCC 25922) alone and in combination with the polymyxin colistin, an approved drug product. The amount of colistin used in this experiment is considered to be sub-inhibitory, that is, one dilution below the antibacterial MIC for colistin. The results are presented in Table 2 and show an improvement or enhancement of activity in the presence of Colistin when compared to the activity of these compounds alone. In a study by Gunderson, B. W., et. al. (Antimicrob. Agents Chemother. March 2003 Vol. 47. No. 3:905-909) involving a comparative bacterial type II topoisomerase inhibitor, ciprofloxacin, was found not to enhance the activity of colistin when tested against two clinical isolates of P. aeruginosa. This lack of synergy observed in the original study is presented in a recent review article by Biswas, S., et. al. (Expert Rev. Anti. Infect. Ther. 2012 Vol. 10 No. 8: 917-934).

TABLE 2
Antibacterial activity against Gram-negative bacterial type strain:
E. coli (ATCC 25922)

| | | MIC | MIC of Compound |
|---|---|---|---|
| | | Colistin | (μg/ml) in the |
| | MIC Compound | alone | presence of |
| Compound Example | alone (μg/ml) | (μg/ml) | Colistin (1 μg/ml) |
| Example 23 | 4 | 2 | 1 |
| (WO2013/138860) | | | |
| A-3 | >64 | 2 | ≦1 |
| A-13 | 1 | 2 | 0.25 |

In another experiment, selected compounds of Formula (I) and Formula (II) were tested for activity against E. coli (ATCC 25922) alone and in combination with polymyxin B (PMB), a polymyxin that is an approved drug product. The amount of PMB used in this experiment is considered to be sub-inhibitory, that is, one dilution below the antibacterial MIC for PMB. The results are presented in Table 3.

TABLE 3
Antibacterial activity against Gram-negative bacterial type strain:
E. coli (ATCC 25922)

| | | | MIC of |
|---|---|---|---|
| | | | Compound |
| | | MIC PMB | (μg/ml) in the |
| | MIC Compound | alone | presence of |
| Compound Example | alone (μg/ml) | (μg/ml) | PMB (0.5 μg/ml) |
| Example 23 | 4 | 1 | 1 |
| (WO2013/138860) | | | |
| A-3 | >64 | 1 | ≦1 |
| A-13 | 1 | 1 | 0.12 |

In another experiment, selected compounds of Formula (I) and Formula (II) were tested for activity against A. baumannii (ATCC 19606) alone and in combination with a polymyxin derivative, PMBN. The results are presented in Table 4.

TABLE 4
Antibacterial activity against Gram-negative bacterial type strain:
A. baumannii (ATCC 19606)

| | | MIC | MIC of Compound |
|---|---|---|---|
| | | PMBN | (μg/ml) in the |
| | MIC Compound | alone | presence of PMBN |
| Compound Example | alone (μg/ml) | (μg/ml) | (128 μg/ml) |
| Example 51 | 4 | >128^{(a)} | 0.5 |
| WO2007/148093 | | | |
| Example 23 | 4 | >128^{(a)} | 0.25 |
| WO2013/138860 | | | |
| A-8 | 2 | >128^{(a)} | 0.25 |
| A-13 | 1 | >128^{(a)} | 0.03 |
| A-21 | 1 | >128^{(a)} | 0.03 |

^{(a)}No measurable antibacterial activity

In another experiment, selected compounds of Formula (I) and Formula (II) were tested for activity against P. aeruginosa (ATCC 27853) alone and in combination with a polymyxin derivative, PMBN. The results are presented in Table 5.

TABLE 5
Antibacterial activity against Gram-negative bacterial type strain:
P. aeruginosa (ATCC 27853)

| | | MIC | MIC of Compound |
|---|---|---|---|
| | | PMBN | (μg/ml) in the |
| | MIC Compound | alone | presence of PMBN |
| Compound Example | alone (μg/ml) | (μg/ml) | (128 μg/ml) |
| Example 23 | >64 | >128^{(a)} | <=0.008 |
| WO2013/138860 | | | |
| A-13 | 4 | >128^{(a)} | <=0.008 |

^{(a)}No measurable antibacterial activity

In another experiment, selected compounds of Formula (I) and Formula (II) were tested for activity against E. cloaceae (ATCC 13047) alone and in combination with a polymyxin derivative, PMBN. The results are presented in Table 6.

TABLE 6
Antibacterial activity against Gram-negative bacterial type strain:
E. cloaceae (ATCC 13047)

| | | MIC | MIC of Compound |
|---|---|---|---|
| | | PMBN | (μg/ml) in the |
| | MIC Compound | alone | presence of PMBN |
| Compound Example | alone (μg/ml) | (μg/ml) | (128 μg/ml) |
| Example 23 | >64 | >128^{(a)} | 0.5 |
| WO2013/138860 | | | |
| A-13 | >64 | >128^{(a)} | 0.25 |

^{(a)}No measurable antibacterial activity

In another experiment, a selected compound of Formula (I) was tested for activity against E. cloaceae (ATCC 13047) alone and in combination with the polymyxin colistin, an approved drug product. The amount of colistin used in this experiment is considered to be non-inhibitory. The results are presented in Table 7 and show an improvement or enhancement of activity in the presence of colistin when compared to the activity of this compound alone.

TABLE 7
Antibacterial activity against Gram-negative bacterial type strain:
E. cloaceae (ATCC 13047)

| | | MIC | MIC of Compound |
|---|---|---|---|
| | | colistin | (μg/ml) in the |
| | MIC Compound | alone | presence of |
| Compound Example | alone (μg/ml) | (μg/ml) | colistin (2 μg/ml) |
| Example 23 | >64 | >64^{(a)} | <=0.12 |
| WO2013/138860 | | | |

^{(a)}No measurable antibacterial activity

In another experiment, a selected compound of Formula (I) was tested for activity against B. cepacia (ATCC 25416) alone and in combination with the polymyxin colistin, an approved drug product. The amount of colistin used in this experiment is considered to be non-inhibitory. The results are presented in Table 8 and show an improvement or enhancement of activity in the presence of Colistin when compared to the activity of this compound alone.

TABLE 8
Antibacterial activity against Gram-negative bacterial type strain:
B. cepacia (ATCC 25416)

| | | MIC | MIC of Compound |
|---|---|---|---|
| | | colistin | (μg/ml) in the |
| | MIC Compound | alone | presence of |
| Compound Example | alone (μg/ml) | (μg/ml) | colistin (2 μg/ml) |
| Example 23 | >64 | >64^{(a)} | 8 |
| WO2013/138860 | | | |

^{(a)}No measurable antibacterial activity

In another experiment, a selected compound of Formula (I) was tested for activity against Francisella sp. F. philomiragia (ATCC 25015) alone and in combination with a polymyxin derivative, PMBN. The results are presented in Table 9.

TABLE 9
Antibacterial activity against Gram-negative bacterial type strain:
F. philomiragia (ATCC 25015)

| | | MIC | MIC of Compound |
|---|---|---|---|
| | | PMBN | (μg/ml) in the |
| | MIC Compound | alone | presence of |
| Compound Example | alone (μg/ml) | (μg/ml) | PMBN (32 μg/ml) |
| Example 23 | >16 | >32^{(a)} | 2 |
| WO2013/138860 | | | |

^{(a)}No measurable antibacterial activity

In another experiment, the activity of selected compounds of Formula (I) and Formula (II) were tested for activity against a panel of drug resistant clinical isolates alone and in combination with a polymyxin derivative, PMBN. The results are presented in Table 10. It will be understood that reference to drug resistant clinical isolates in this experiment, may be more generally described as drug resistant Gram-negative bacteria.

TABLE 10
Antibacterial activity against Gram-negative bacterial
drug resistant clinical isolates

| | | | MIC of |
|---|---|---|---|
| | | | Compound |
| | | MIC | (μg/ml) |
| | | Com- | in the |
| | | pound | presence of |
| Compound | Drug Resistant Clinical | alone | PMBN |
| Example | Isolate ID | (μg/ml) | (128 μg/ml) |
| Example 23 | E. coli (MMX 1312) | >64 | ≦0.008 |
| WO2013/138860 | | | |
| Example 23 | E. coli (MMX 2232) | >64 | ≦0.008 |
| WO2013/138860 | | | |
| Example 23 | P. aeruginosa (NCTC13437) | >64 | 0.25 |
| WO2013/138860 | | | |
| Example 23 | P. aeruginosa (MMX3007) | >64 | 0.25 |
| WO2013/138860 | | | |
| Example 23 | P. aeruginosa (MMX3022) | >64 | 0.25 |
| WO2013/138860 | | | |
| Example 23 | P. aeruginosa (MMX3025) | >64 | 0.12 |
| WO2013/138860 | | | |
| Example 23 | A. baumannii (MMX 6331) | >64 | 0.25 |
| WO2013/138860 | | | |
| Example 23 | A. baumannii (MMX 4454) | 2 | 0.5 |
| WO2013/138860 | | | |
| Example 23 | E. cloaceae (MMX 6087) | >64 | 2 |
| WO2013/138860 | | | |
| Example 23 | E. cloaceae (MMX 6093) | >16 | 0.5 |
| WO2013/138860 | | | |
| Example 23 | E. cloaceae (MMX 6095) | >64 | 8 |
| WO2013/138860 | | | |
| Example 23 | E. cloaceae (MMX 6304) | >16 | 1 |
| WO2013/138860 | | | |
| A-13 | E. coli (BAA200) | >4 | ≦0.008 |
| A-13 | E. coli (NCTC13476) | 8 | ≦0.12 |
| A-13 | E. coli (NCTC11954) | 2 | ≦0.008 |
| A-13 | E. coli (NCTC13352) | 4 | ≦0.008 |
| A-13 | E. coli (NCTC13353) | 1 | ≦0.008 |
| A-13 | E. coli (NCTC13400) | >4 | ≦0.008 |
| A-13 | E. coli (NCTC13462) | >4 | ≦0.008 |
| A-13 | E. coli (NCTC13463) | 2 | ≦0.008 |
| A-13 | E. coli (MMX 5743) | 4 | ≦0.008 |
| A-13 | E. coli (NCTC13351) | >64 | ≦0.12 |
| A-13 | E. coli (NCTC13441) | >64 | ≦0.12 |
| A-13 | E. coli (NCTC13450) | >64 | ≦0.12 |
| A-13 | E. coli (NCTC13461) | >64 | ≦0.12 |
| A-13 | E. coli (MMX 6413) | >64 | ≦0.12 |
| A-13 | E. coli (MMX 5771) | >64 | ≦0.12 |
| A-13 | E. coli (MMX 1312) | 16 | ≦0.008 |
| A-13 | E. coli (MMX 2232) | 16 | ≦0.008 |
| A-13 | P. aeruginosa (NCTC13437) | 16 | 0.016 |
| A-13 | P. aeruginosa (MMX3007) | >16 | 0.03 |
| A-13 | P. aeruginosa (MMX3022) | 8 | 0.03 |
| A-13 | P. aeruginosa (MMX3025) | >16 | 0.03 |
| A-13 | P. aeruginosa (MMX3026) | >16 | ≦0.008 |
| A-13 | P. aeruginosa (MMX4700) | 16 | 0.12 |
| A-13 | A. baumannii (MMX 6331) | 4 | 0.06 |
| A-13 | A. baumannii (MMX 4454) | 1 | 0.12 |
| A-13 | A. baumannii (NCTC13301) | 4 | 0.12 |
| A-13 | A. baumannii (NCTC13302) | 1 | 0.03 |
| A-13 | A. baumannii (NCTC13303) | 2 | 0.06 |
| A-13 | A. baumannii (NCTC13304) | 1 | 0.03 |
| A-13 | A. baumannii (NCTC13305) | 2 | 0.12 |
| A-13 | A. baumannii (NCTC13421) | 2 | 0.03 |
| A-13 | A. baumannii (NCTC13422) | 4 | 0.03 |
| A-13 | A. baumannii (NCTC13424) | 1 | 0.03 |
| A-13 | A. baumannii (NCTC 13420) | 1 | 0.03 |
| A-13 | A. baumannii (MMX 2600) | 2 | 0.06 |
| A-13 | A. baumannii (MMX 2585) | 4 | 0.12 |
| A-13 | A. baumannii (MMX 4405) | 4 | 0.12 |
| A-13 | A. baumannii (MMX 2598) | 2 | 0.06 |
| A-13 | E. cloaceae (MMX 6087) | >64 | 0.25 |
| A-13 | E. cloaceae (MMX 6093) | 8 | 0.12 |
| A-13 | E. cloaceae (MMX 6095) | >64 | 1 |
| A-13 | E. cloaceae (MMX 6304) | >16 | 0.25 |

In another experiment, a selected compound of Formula (I) was tested against a panel of colistin resistant Gram-negative strains. The results are presented in Table 11. Unexpectedly, the combination was shown to be active against all of the strains tested.

TABLE 11
Antibacterial activity against colistin-resistant Gram-negative strains

| | | | MIC of |
|---|---|---|---|
| | | | Compound |
| | | | (μg/ml) |
| | | MIC | in the |
| | | Compound | presence |
| Compound | | alone | of colistin |
| Example | Colistin-Resistant Strain | (μg/ml) | (2 μg/ml) |
| Example 23 | P. aeruginosa (MMX | >64 | 4 |
| WO2013/138860 | 1497) | | |
| Example 23 | K. pneumonia colistin- | 1 | 0.25 |
| WO2013/138860 | resistant strain #1^{(a)} | | |
| Example 23 | K. pneumonia colistin- | 1 | 0.25 |
| WO2013/138860 | resistant strain #2^{(a)} | | |
| Example 23 | K. pneumonia colistin- | 2 | 0.12 |
| WO2013/138860 | resistant strain #3^{(a)} | | |
| Example 23 | E. coli colistin-resistant | 4 | 2 |
| WO2013/138860 | strain #1^{(a)} | | |
| Example 23 | E. coli colistin-resistant | 4 | 1 |
| WO2013/138860 | strain #2^{(a)} | | |
| Example 23 | E. coli colistin-resistant | 8 | 1 |
| WO2013/138860 | strain #3^{(a)} | | |
| Example 23 | E. coli colistin-resistant | 8 | 1 |
| WO2013/138860 | strain #26^{(a)} | | |
| Example 23 | E. coli colistin-resistant | 4 | 1 |
| WO2013/138860 | strain #43^{(a)} | | |
| Example 23 | A. baumannii colistin- | <=0.03 | 0.015 |
| WO2013/138860 | resistant strain #1^{(a)} | | |
| Example 23 | A. baumannii colistin- | 4 | 0.03 |
| WO2013/138860 | resistant strain #2^{(a)} | | |
| Example 23 | A. baumannii colistin- | <=0.03 | <=0.008 |
| WO2013/138860 | resistant strain #3^{(a)} | | |

^{(a)}Raised by selection with 2 or 4 μg/mL of colistin on MH agar: MIC (colistin) = 16-32 μg/mL

In another experiment, a selected compound of Formula (I) and a panel of comparative antibiotics were tested for activity against E. coli (ATCC 25922) alone and in combination with a polymyxin derivative, polymyxin B nonapeptide (PMBN). The results are presented in Table 12. The comparative compounds showed no effect or only a very modest improvement in activity in the presence of PMBN.

TABLE 12
Antibacterial activity against Gram-negative bacterial type strain:
E. coli (ATCC 25922)

| | | | MIC of |
|---|---|---|---|
| | | | Compound |
| | | | (μg/ml) |
| | | | in the |
| | MIC | | presence |
| | Compound | MIC PMBN alone | of PMBN |
| Compound Example | alone (μg/ml) | (μg/ml) | (32 μg/ml) |
| Example 23 | >2 | >128^{(a)} | 0.12 |
| (WO2013/138860) | | | |
| Ofloxacin | 0.03 | >128^{(a)} | 0.03^{(b)} |
| Piperacillin | 2 | >128^{(a)} | 0.25 |
| Tetracycline | 1 | >128^{(a)} | 1^{(b)} |
| Trimethoprim | 0.5 | >128^{(a)} | 2^{(b)} |
| Ceftriaxone | 0.06 | >128^{(a)} | 0.06^{(b)} |
| Imipenem | 0.5 | >128^{(a)} | 0.5^{(b)} |
| Meropenem | 0.5 | >128^{(a)} | 0.5^{(b)} |
| Carbenicillin | 64 | >128^{(a)} | 32^{(b)} |
| Ceftazidime | 0.6 | >128^{(a)} | 0.15 |
| Gentamicin | 8 | >128^{(a)} | 8^{(b)} |
| Chloramphenicol | 4 | >128^{(a)} | 1 |
| Levofloxacin | 0.008 | >128^{(a)} | 0.008^{(b)} |
| Enoxacin | 0.06 | >128^{(a)} | 0.03^{(b)} |
| Kanamycin | 8 | >128^{(a)} | 4^{(b)} |
| Bacitracin | >64 | >128^{(a)} | >64^{(b)} |
| Daptomycin | >64 | >128^{(a)} | >64^{(b)} |
| Colistin | 4 | >128^{(a)} | 4^{(b)} |
| Amoxicillin | 32 | >128^{(a)} | 8 |
| Tigecyclin | ≦0.25 | >128^{(a)} | ≦0.25^{(b)} |

^{(a)}No measurable antibacterial activity.
^{(b)}No significant effect

In another experiment, selected compounds were tested for activity against Gram-positive strains S. aureus (ATCC 29213) and E. faecalis (ATCC 29212) and Gram-negative strains H. influenzae (ATCC 49247) and N. gonorrheae (ATCC 49226). The results are presented in Table 13.

TABLE 13
Antibacterial activity against Gram-positive bacterial type strains:
S. aureus (ATCC 29212), E. faecalis (ATCC 29213); and
Gram-negative bacterial type strains: H. influenzae (ATCC 49247).
N. gonorrheae (ATCC 49226)

| | MIC vs. | MIC vs. | | |
|---|---|---|---|---|
| | S. aureus | E. faecalis | MIC vs. | MIC vs. |
| | (ATCC | (ATCC | H. influenzae | N. gonorrheae |
| Compound | 29213) | 29212) | (ATCC | (ATCC 49226) |
| Example | (μg/ml) | (μg/ml) | 49247) (μg/ml) | (μg/ml) |
| A-10 | 2 | 0.5 | 4 | 0.25 |
| A-11 | 0.004 | <=0.008 | 0.06 | 0.015 |
| A-12 | <=0.002 | <=0.008 | 0.015 | <=0.008 |
| A-13 | <=0.008 | <=0.008 | 0.015 | <=0.008 |
| A-14 | 0.03 | <=0.008 | 0.015 | 0.015 |
| A-15 | 0.12 | 0.015 | 0.03 | <=0.008 |
| A-21 | <=0.002 | <=0.008 | 0.03 | <=0.008 |
| A-24 | 0.06 | 0.03 | 0.5 | 0.12 |
| A-25 | 0.06 | 0.06 | 1 | 0.5 |
| A-26 | 0.06 | 0.06 | 0.5 | 0.25 |
| A-28 | 0.03 | 0.015 | 0.12 | 0.03 |
| A-29 | 0.25 | 0.06 | 2 | 0.25 |
| A-30 | 0.06 | <=0.008 | 0.008 | <=0.008 |
| A-31 | 2 | 0.03 | 0.015 | 0.03 |
| A-32 | 0.12 | 0.015 | 0.015 | 0.03 |
| A-33 | 0.25 | 0.03 | 0.12 | 0.03 |
| A-34 | 0.25 | 0.015 | 0.12 | 0.015 |
| A-35 | <=0.008 | <=0.008 | 0.03 | <=0.008 |
| A-36 | 0.25 | <=0.008 | 0.03 | <=0.008 |
| A-37 | 0.25 | 0.015 | 0.12 | 0.03 |
| A-38 | 0.015 | <=0.008 | 0.03 | <=0.008 |
| A-39 | 0.015 | <=0.008 | 0.03 | 0.015 |
| A-40 | 0.015 | <=0.008 | 0.015 | <=0.008 |
| A-41 | 0.5 | <=0.008 | 0.06 | 0.015 |
| A-42 | 0.06 | <=0.008 | 0.03 | <=0.008 |
| A-43 | 0.12 | <=0.008 | 0.03 | <=0.008 |
| A-44 | 0.03 | <=0.008 | 0.03 | <=0.008 |
| A-45 | 0.015 | <=0.008 | 0.06 | <=0.008 |
| A-46 | 1 | 0.25 | 2 | 0.06 |
| A-47 | 0.015 | <=0.008 | <=0.008 | <=0.008 |
| A-48 | 0.015 | <=0.008 | 0.03 | <=0.008 |
| A-49 | 1 | 0.25 | 2 | 0.25 |
| A-50 | 0.5 | 0.03 | 4 | 0.25 |
| A-51 | 0.015 | 0.008 | 0.015 | <=0.008 |
| A-52 | 0.5 | 0.12 | 0.12 | 0.03 |
| A-53 | >64 | 0.5 | >64 | 2 |
| A-54 | 0.12 | <=0.008 | 0.03 | <=0.008 |
| A-55 | 0.12 | <=0.008 | 0.03 | <=0.008 |
| A-56 | 0.06 | 0.015 | 0.12 | 0.06 |
| A-57 | 0.25 | 0.03 | 0.5 | 0.12 |
| A-58 | 2 | 0.25 | 1 | 0.03 |
| A-59 | 0.12 | 0.015 | 0.03 | 0.015 |
| A-60 | 0.12 | 0.06 | 0.03 | <=0.008 |
| A-61 | 0.06 | 0.06 | 0.12 | 0.25 |

### Pharmacokinetic Assays: Determination of PK Profile

The pharmacokinetic profiles of compounds are determined by measuring the compound concentration in plasma by LC/MS/MS following a single intravenous or peroral administration of the compounds at a dose of 1 or 3 mg/kg individually or in a cassette of up to 5 compounds. The concentrations are described as the mean plasma concentrations at each time point from three animals. Intravenous dose formulation is administered as a single bolus dose through the tail vein. Oral dose formulation is administered to animals by an oral gavage needle. In both cases the dose volume is 5.0 mL/kg. Blood is collected from rats using a jugular vein catheter and from anesthetized mice through a capillary guided into the retro-orbital plexus. The collected blood is then centrifuged to obtain plasma and the compounds extracted into methanol prior to determining the compound concentration by LC/MS/MS.

### Animal Models of Infection

Suitable models of infection will be familiar to those skilled in the art and include the following suitable for intravenous (IV) or oral (PO) dosing.

### Thigh Infection Model(s)

Mouse: The thighs of mice, rendered neutropenic by the intraperitoneal administration of cyclophosphamide (150 mg/kg at day −4 and 100 mg/kg at day −1), are inoculated with a bacterial suspension prepared from a fresh overnight culture. Compounds are administered at various times and the cfus enumerated at various times post dosing by harvesting the thighs, homogenising in saline on ice and plating serial dilutions onto charcoal containing plates for growth overnight and colony counting.

Rat: The thighs of Sprague-Dawley rats, rendered neutropenic by the intraperitoneal administration of cyclophosphamide (75 mg/kg on days −4 and −1), are inoculated with a bacterial suspension. Compounds are administered at various times and the cfus enumerated at various times post dosing by harvesting the thighs, homogenising in PBS on ice and plating serial dilutions onto CLED agar plates for growth at 37° C. and colony counting.

### Lung Infection Model(s)

Mouse:

Anaesthetised mice are inoculated intranasally with a bacterial suspension prepared from a fresh overnight culture by placing 50 μl of inoculum on the nares and allowing the mice to inhale. Compounds are administered at various times and the cfus enumerated at 48 hours post inoculation by harvesting the lungs, homogenising in PBS on ice and plating serial dilutions onto bacterial growth medium for colony counting.

Rat:

Anaesthetised Sprague-Dawley rats, rendered neutropenic by the intraperitoneal administration of cyclophosphamide, are inoculated intratracheally with a bacterial suspension prepared from a fresh culture by delivering 0.5 ml of inoculum in molten agar. Compounds are administered at various times and the cfus enumerated at 96 hours post inoculation by harvesting the lungs, homogenising in PBS on ice and plating serial dilutions onto bacterial growth medium for cfu determination

Mouse Survival Model:

Mice are inoculated intranasally with a bacterial suspension prepared from a fresh overnight culture by placing 50 μl of inoculum on the nares and allowing the mice to inhale. Compounds are administered at various times post inoculation and the mice monitored for survival after infection.

### Skin Infection Model(s)

Mouse:

An area of the skin is stripped from the back dorsal surface of anaesthetised mice by abrading with a fine emery board following removal of the fur by shaving. An infection is initiated by placing 5 μl of bacterial suspension prepared from a fresh overnight culture, onto the damaged skin Compounds are administered at various times and the cfus enumerated after 5 days post inoculation by harvesting the wounds, homogenising in PBS on ice and plating serial dilutions onto charcoal containing plates for growth overnight and colony counting.

### Septicaemia Infection Model(s)

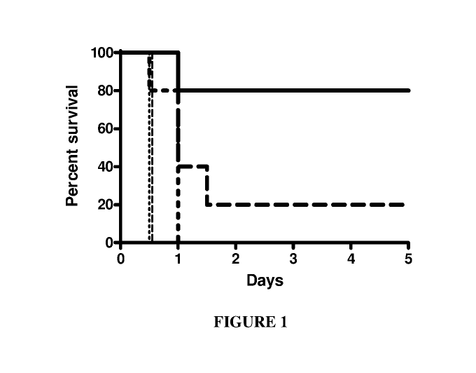

Mouse: Female CD-1 mice (18-22 g) were inoculated intraperitoneally with a bacterial suspension of 1.35×10⁶ cfus of E. coli (NDM-1; CTX-M15) prepared from a fresh culture suspended in 5% hog gastric mucin. Compounds are administered as indicated at 1 or 1 and 3 hours post infection and the mice monitored for survival for 5 days after infection. For example, FIG. 1 shows that 20% and 80% of mice survived at the end of 5 days following treatment with one or two doses respectively of the combination of polymyxin B nonapeptide (PMBN) administered subcutaneously (SC) at 50 mg/kg and a Compound of Formula (I) (Example 152 of WO2013/138860) administered intravenously (IV) at 100 mg/kg. No mice survived in the control group or following two doses of polymyxin B nonapeptide (PMBN) administered subcutaneously (SC) at 50 mg/kg or a Compound of Formula (I) (Example 152 of WO2013/138860) administered intravenously (IV) at 100 mg/kg alone.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication, or information derived from it, or to any matter which is known, is not, and should not be taken as an acknowledgement or admission or any form of suggestion that that prior publication, or information derived from it, or known matter forms part of the common general knowledge in the field of endeavour to which this specification relates.

1. A method for the treatment or prevention of a bacterial infection comprising administration of a bacterial type II topoisomerase inhibitor in combination with a polymyxin or polymyxin derivative to a subject suffering from the bacterial infection or at risk of the bacterial infection, wherein the bacterial infection is caused by a Gram-negative bacteria or drug resistant Gram-negative bacteria and the bacterial type II topoisomerase inhibitor has on-target enzyme activity against DNA gyrase and optionally on-target enzyme activity against topoisomerase IV.

2. The method according to claim 1 wherein the bacterial type II topoisomerase inhibitor has on-target enzyme activity against DNA gyrase and on-target enzyme activity against topoisomerase IV.

3. The method according to claim 1 wherein the bacterial type II topoisomerase inhibitor is a GyrB/ParE inhibitor.

4. The method according to claim 1 wherein the bacterial type II topoisomerase inhibitor is a compound of Formula (I): 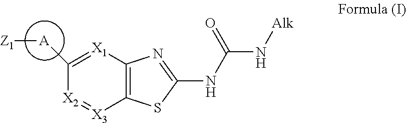
and; salts, racemates, diastereomers, enantiomers, deuterated forms, hydrates, solvates and prodrugs thereof
wherein:
Alk is an optionally substituted C₁₋₆alkyl, C₂₋₆alkenyl, C₂₋₆alkynyl, or C₃₋₆cycloalkyl;
A represents “Ring A” which is selected from saturated or unsaturated monocyclic C₃₋₇cycloalkyl, saturated or unsaturated monocyclic 3-7 membered heterocyclyl, saturated or unsaturated fused bicyclic C₈₋₁₀cycloalkyl, saturated or unsaturated fused bicyclic 8-10 membered-heterocyclyl, C₆₋₁₀aryl and 5-10 membered heteroaryl and may be optionally substituted;
X₁ is CH, —N═ or C—R₁, where R₁ is selected from OH, optionally substituted C₁₋₃alkyl, optionally substituted C₂₋₃alkenyl, optionally substituted C₂₋₃alkynyl, optionally substituted C₁₋₃alkoxyl, halo, haloC₁₋₃alkyl, NH₂, optionally substituted NHC₁₋₃alkyl, optionally substituted N(C₁₋₃ alkyl)₂, optionally substituted SC₁₋₃alkyl and CN;
X₂ is CH, —N═ or C—R₂, where R₂ is selected from OH, optionally substituted C₁₋₆alkyl, optionally substituted C₂₋₆alkenyl, optionally substituted C₂₋₆alkynyl, optionally substituted (CH₂)ₘOC₁₋₆ alkyl, optionally substituted (CH₂)ₘSC₁₋₆alkyl, optionally substituted (CH₂)ₘS(═O)C₁₋₆alkyl, optionally substituted (CH₂)ₘO(CH₂)ₛC₃₋₇cycloalkyl, optionally substituted (CH₂)ₘC₃₋₇cycloalkyl, optionally substituted (CH₂)ₘO(CH₂)ₘphenyl, optionally substituted (CH₂)ₘphenyl, optionally substituted (CH₂)ₘO(CH₂)ₘ-5-10-membered heterocycle, optionally substituted (CH₂)ₘ-5-10-membered heterocyclyl, halo, optionally substituted haloC₁₋₃alkyl, CN and optionally substituted (CH₂)ₘNR^{a}R^{b};
X₃ is CH, —N═ or C—R₃, where R₃ is selected from OH, optionally substituted C₁₋₆alkyl, optionally substituted C₂₋₆alkenyl, optionally substituted C₂₋₆alkynyl, optionally substituted (CH₂)ₘOC₁₋₆ alkyl, optionally substituted (CH₂)ₘSC₁₋₆ alkyl, optionally substituted (CH₂)ₘS(═O)C₁₋₆alkyl, optionally substituted (CH₂)ₘO(CH₂)ₘC₃₋₇cycloalkyl, optionally substituted (CH₂)ₘC₃₋₇cycloalkyl, optionally substituted (CH₂)ₘO(CH₂)ₘphenyl, optionally substituted (CH₂)ₘphenyl, optionally substituted (CH₂)ₘO(CH₂)ₘ-5-10-membered heterocycle, optionally substituted (CH₂)ₘ-5-10-membered heterocyclyl, halo, optionally substituted haloC₁₋₃alkyl, CN and optionally substituted (CH₂)ₘNR^{a}R^{b};
each R^{a} and R^{b} is independently selected from H, optionally substituted C₁₋₆alkyl, optionally substituted C₃₋₆cycloalkyl and optionally substituted 4-6-membered heterocyclyl or R^{a} and R^{b} join together to form an optionally substituted 4-6-membered heterocyclyl;
each m is an integer independently selected from 0, 1, 2 and 3;
Z₁ is selected from H, halo, C₁₋₆alkyl, a 5-membered heterocyclic ring, a 6-membered heterocyclic ring, OH, OC₁₋₆alkyl, C₁₋₆alkoxyl, cyano (CN), a carbonyl moiety (═O), C(═O)OC₁₋₆alkyl, NH₂, NH—C₁₋₆alkyl, N(C₁₋₆alkyl)₂, and C(═O)NH—C₁₋₆alkyl;
or Z₁ is a carbonyl containing group of general formula —(Y)_{q}B(R₄)—C(═O)—W—R₅
wherein:
q is an integer 0 or 1;
Y is attached to Ring A and when q is 0 then Y is a covalent bond, a spiro ring centre, or a fused ring bond; or when q is 1 then Y is selected from optionally substituted C₁₋₃alkylene, optionally substituted C₂₋₃alkenylene and optionally substituted C₂₋₃alkynylene and wherein each carbon atom in C₁₋₃alkylene may be optionally replaced by an oxygen or nitrogen heteroatom or C(═O);
B represents “Ring B” and is selected from saturated or unsaturated monocyclic C₃₋₇cycloalkyl, saturated or unsaturated monocyclic 3-7 membered heterocycle, saturated or unsaturated fused bicyclic C₈₋₁₀cycloalkyl, saturated or unsaturated fused bicyclic 8-12 membered heterocyclyl, C₆₋₁₀aryl, 5-10 membered heteroaryl, and a spiro bicyclic 8-12 membered heterocyclic ring system; and further Ring B may be optionally substituted; or Ring B may join together with Ring A to form a saturated or unsaturated fused bicyclic C₈₋₁₀cycloalkyl, a saturated or unsaturated fused bicyclic 8-10 membered heterocyclyl and a spiro bicyclic 8-12 membered heterocyclic ring system;
R₄ is joined to the same Ring B atom as the —C(═O)—W—R₅ moiety and is selected from C₁₋₆alkyl, C₂₋₆alkenyl, C₂₋₆alkynyl, (C₁₋₆alkyl)ₜC₃₋₇cycloalkyl, (C₁₋₆alkyl)ₜaryl, (C₁₋₆ alkyl)ₜheterocyclyl, (C₁₋₆alkyl)ₜheteroaryl, NH₂, NH(C₁₋₆alkyl), N(C₁₋₆alkyl)₂, CN, OH, C₁₋₆alkoxy, SO₂H, SO₂C₁₋₆alkyl, SH, SC₁₋₆alkyl, halo, haloC₁₋₆alkyl, —NH(C═O)OC₁₋₆alkyl, —NH(C═O)OC(C₁₋₃alkyl)₃, and wherein C₁₋₃alkyl, C₁₋₆alkyl, C₂₋₆alkenyl, C₂₋₆alkynyl, C₃₋₇cycloalkyl, aryl and heterocyclyl in each case may be further optionally substituted or R₄ is a chain of 3 or 4 carbon atoms or carbon and heteroatoms which joins with an adjacent B ring atom to form a fused carbocyclylic or heterocyclic ring which is optionally further substituted;
the —C(═O)—W—R₅ moiety is joined to the same Ring B atom as R₄ wherein:
W is O, NH or N(C₁₋₆alkyl);
R₅ is selected from H, C₁₋₆alkyl, C₂₋₆alkenyl, C₂₋₆alkynyl, S(O)₂OH, S(O)₂—C₁₋₆alkyl, or M where M represents a monovalent or divalent cation selected from the group comprising pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids;
or Z₁ is an alcohol containing group of general formula (CH₂)ₛC(OH)(R₆)(R₇) or an ester, carbamate, phosphate, sulfate or prodrug thereof wherein the OH, R₆ and R₇ groups are each attached to the same carbon atom; and
wherein:
s is an integer selected from 0, 1, 2 and 3;
R₆ is H or is selected from optionally substituted C₁₋₆alkyl, optionally substituted C₂₋₆alkenyl, optionally substituted C₂₋₆alkynyl, optionally substituted (CH₂)ₜOC₁₋₆alkyl, optionally substituted (CH₂)ₜOC(═O)C₁₋₆alkyl, optionally substituted (CH₂)ₜSC₁₋₆alkyl, optionally substituted (CH₂)ₜS(═O)C₁₋₆alkyl, halo, optionally substituted haloC₁₋₃ alkyl and optionally substituted (CH₂)ₜNR^{a}R^{b};
R₇ is selected from optionally substituted C₁₋₆alkyl, optionally substituted C₂₋₆alkenyl, optionally substituted C₂₋₆alkynyl, optionally substituted C₃₋₇cycloalkyl ring, optionally substituted phenyl, optionally substituted 4-6-membered heterocyclyl ring, optionally substituted 5-6-membered heteroaryl ring, optionally substituted (CH₂)ₜOC₁₋₆alkyl, optionally substituted (CH₂)ₜOC(═O)C₁₋₆alkyl, optionally substituted (CH₂)ₜSC₁₋₆alkyl, optionally substituted (CH₂)ₜS(═O)C₁₋₆alkyl, halo, optionally substituted haloC₁₋₃alkyl and optionally substituted (CH₂)ₜNR^{a}R^{b};
t is an integer selected from 1, 2, 3, 4, 5 and 6;
or R₆ and R₇ together with the carbon atom to which they are attached form an optionally substituted 4-6-membered heterocyclic ring or C₃₋₇cycloalkyl ring;
and further wherein the prodrug is selected from an ester, carbamate, phosphate or sulfate formed from the hydroxyl moiety;
or Z₁ a sulfonamide containing group of general formula (CH₂)ᵥNRS(═O)₂R₈ or (CH₂)ᵥS(═O)₂NR₉R₁₀ or a sulfamide containing group of general formula (CH₂)ᵥNRS(═O)₂NR₉R₁₀ wherein:
v is an integer 0, 1, 2 or 3;
R is H or an optionally substituted C₁₋₆alkyl; and
R₈, R₉ and R₁₀ are each independently selected from H, C₁₋₆alkyl, C₂₋₆alkenyl, C₂₋₆alkynyl, C₃₋₇cycloalkyl, phenyl, benzyl, a 3-10-membered heterocyclic ring, a 5-10-membered heteroaryl ring and further wherein each C₁₋₆alkyl, C₂₋₆alkenyl, C₂₋₆alkynyl, C₃₋₇cycloalkyl, phenyl, benzyl, a 3-10-membered heterocyclic ring, a 5-10-membered heteroaryl ring may be optionally substituted;
or R₉ and R₁₀ may join to form an optionally substituted 3-6-membered heterocyclic ring together with the nitrogen to which they are attached.

5. The method according to claim 1 wherein the bacterial type II topoisomerase inhibitor is a compound of Formula (II): 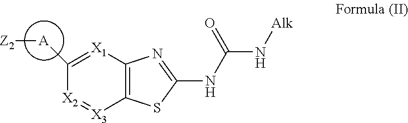
and; salts, racemates, diastereomers, enantiomers, deuterated forms, hydrates, solvates and prodrugs thereof:
wherein Alk, Ring A, X₁, X₂ and X₃ are according to claim 4; and
Z₂ is (CH₂)ᵥNRS(═O)₂R₈, (CH₂)ᵥS(═O)₂NR₉R₁₀ or (CH₂)ᵥNRS(═O)₂NR₉R₁₀;
wherein
v is an integer 0, 1, 2 or 3;
R is H or an optionally substituted C₁₋₆alkyl; and
R₈, R₉ and R₁₀ are each independently selected from H, C₁₋₆alkyl, C₂₋₆alkenyl, C₂₋₆alkynyl, C₃₋₇cycloalkyl, phenyl, benzyl, a 3-10-membered heterocyclic ring, or a 5-10-membered heteroaryl ring and further wherein each C₁₋₆alkyl, C₂₋₆alkenyl, C₂₋₆alkynyl, C₃₋₇cycloalkyl, phenyl, benzyl, 3-10-membered heterocyclic ring and 5-10-membered heteroaryl ring may be optionally substituted;
or R₉ and R₁₀ may join to form an optionally substituted 3-6-membered heterocyclic ring together with the nitrogen to which they are attached.

6. The method according to claim 1 wherein the polymyxin and polymyxin derivative is selected from an antibacterial polymyxin, an antibacterial polymyxin derivative, a non-antibacterial polymyxin, and a non-antibacterial polymyxin derivative.

7. The method according to claim 1 wherein the polymyxin is Polymyxin B or colistin.

8. The method according to claim 1 wherein the polymyxin derivative is Polymyxin B nonapeptide or a prodrug of colistin.

9. The method according to claim 1 wherein the polymyxin or polymyxin derivative is provided in a therapeutically effective antibacterial amount or dosage.

10. The method according to claim 1 wherein the polymyxin or polymyxin derivative is provided in a sub-inhibitory antibacterial minimum inhibitory concentration amount or dosage.

11. The method according to claim 1 wherein the combination may be administered concurrently, sequentially or separately to a patient suffering from infection or at risk of infection.

12. The method according to claim 1 wherein the Gram-negative bacteria or drug resistant Gram-negative bacteria comprises a lipopolysaccharide layer.

13. The method according to claim 1 wherein the Gram-negative bacteria or drug resistant Gram-negative bacteria comprises a lipooligosaccharide layer.

14. The method according to claim 1 wherein the Gram-negative bacteria is one or more bacterial strains selected from the group comprising E. coli, K pneumoniae, A. baumannii, P. aeruginosa, and Enterobacter spp and drug resistant strains thereof.

15. The method according to claim 1 wherein the Gram-negative pathogen is one or more bacterial strains selected from the group comprising M. catarrhalis, Neisseria, Haemophilus and Bordetella and drug resistant strains thereof.

16. The method according to claim 1 wherein the Gram-negative pathogen is one or more bacterial strains selected from the group comprising L. pneumoniae, C. trachomatis, C. pneumonia, Y. pestis, F. tularensis, B. pseudomallei, C. burnetii, Brucella species, B. mallei, C. psittaci and R. prowazekii.

17. The method according to claim 1 wherein the subject is suffering from or at risk of an intra-abdominal infection, hospital acquired pneumonia, ventilator-associated pneumonia, urinary tract infection, bacteremias, community acquired bacterial pneumonia, gonococcal infection, wound or surgical site infections, endocarditis, otitis media, cystic fibrosis or meningitis.

18. Use of a bacterial type II topoisomerase inhibitor in combination with a polymyxin or polymyxin derivative in the treatment or prevention of a bacterial infection wherein the bacterial type II topoisomerase inhibitor has on-target enzyme activity against DNA gyrase and optionally on-target enzyme activity against topoisomerase IV and wherein the bacterial infection is caused by a Gram-negative bacteria or drug resistant Gram-negative bacteria.

19-20. (canceled)

21. A method of improving the antibacterial efficacy of a bacterial type II topoisomerase inhibitor wherein the method comprises the step of administration of a bacterial type II topoisomerase inhibitor with a polymyxin or polymyxin derivative to a subject suffering from a bacterial infection or at risk of a bacterial infection wherein the bacterial type II topoisomerase inhibitor has on-target enzyme activity against DNA gyrase and optionally on-target enzyme activity against topoisomerase IV and wherein the bacterial infection is caused by a Gram-negative bacteria or drug resistant Gram-negative bacteria.

22-25. (canceled)

26. The method according to claim 5 wherein the bacterial type II topoisomerase inhibitor is a compound selected from the group consisting of:
A-10) 2-[[5-[2-(Ethylcarbamoylamino)-7-(2-pyridyl)-1,3-benzothiazol-5-yl]pyrimidin-2-yl]sulfamoyl]benzoic acid;
A-11) 1-Ethyl-3-[7-(5-methyl-2-pyridyl)-5-[2-(pyrrolidin-1-ylsulfonylamino)pyrimidin-5-yl]-1,3-benzothiazol-2-yl]urea;
A-12) 1-Ethyl-3-[7-(5-methyl-2-pyridyl)-5-[2-(propylsulfonylamino)pyrimidin-5-yl]-1,3-benzothiazol-2-yl]urea;
A-13) 1-[5-[2-(tert-Butylsulfonylamino)pyrimidin-5-yl]-7-(5-methyl-2-pyridyl)-1,3-benzothiazol-2-yl]-3-ethyl-urea;
A-14) 1-Ethyl-3-[5-[2-[(2-hydroxy-1,1-dimethyl-ethyl) sulfonylamino]pyrimidin-5-yl]-7-(5-methyl-2-pyridyl)-1,3-benzothiazol-2-yl]urea;
A-15) 1-Ethyl-3-[5-[2-[[2-hydroxyethyl(methyl)sulfamoyl]amino]pyrimidin-5-yl]-7-(5-methyl-2-pyridyl)-1,3-benzothiazol-2-yl]urea;
A-16) Methyl 2-[[5-[2-(ethylcarbamoylamino)-7-(5-methyl-2-pyridyl)-1,3-benzothiazol-5-yl]pyrimidin-2-yl]sulfamoyl]acetate;
A-17) 1-[5-[2-(allylsulfonylamino)pyrimidin-5-yl]-7-(5-methyl-2-pyridyl)-1,3-benzothiazol-2-yl]-3-ethyl-urea;
A-18) 1-[5-[2-(tert-butylsulfonylamino)pyrimidin-5-yl]-7-[2-(dimethylamino)pyrimidin-5-yl]-1,3-benzothiazol-2-yl]-3-ethyl-urea;
A-19) 1-ethyl-3-[5-[2-(methane sulfonamidomethyl)pyrimidin-5-yl]-7-(5-methyl-2-pyridyl)-1,3-benzothiazol-2-yl]urea;
A-20) 1-[5-[2-(tert-butylsulfonylamino)pyrimidin-5-yl]-7-[4-(2-pyrrolidin-1-ylethyl)piperazin-1-yl]-1,3-benzothiazol-2-yl]-3-ethyl-urea;
A-21) 1-[5-[2-(cyclopentylsulfonylamino)pyrimidin-5-yl]-7-(5-methyl-2-pyridyl)-1,3-benzothiazol-2-yl]-3-ethyl-urea;
A-24) 1-(5-(2-(1,1-dioxido-1,2-thiazinan-2-yl)pyrimidin-5-yl)-7-(pyridin-2-yl)benzo[d]thiazol-2-yl)-3-ethylurea;
A-25) 1-[5-[6-(1,1-dioxo-1,2-thiazolidin-2-yl)-3-pyridyl]-7-(2-pyridyl)-1,3-benzothiazol-2-yl]-3-ethylurea;
A-26) 1-(5-(2-(1,1-dioxidoisothiazolidin-2-yl)pyrimidin-5-yl)-7-(pyridin-2-yl)benzo[d]thiazol-2-yl)-3-ethylurea;
A-27) 1-(5-(2-(1,1-dioxidoisothiazolidin-2-yl)pyrimidin-5-yl)-6-((tetrahydrofuran-2-yl)methoxy)benzo[d]thiazol-2-yl)-3-ethylurea;
A-28) 1-(5-(2-(1,1-dioxidoisothiazolidin-2-yl)pyrimidin-5-yl)-7-(5-methylpyridin-2-yl)benzo[d]thiazol-2-yl)-3-ethylurea;
A-29) N-(5-(2-(3-ethylureido)-7-(5-methylpyridin-2-yl)benzo[d]thiazol-5-yl)pyrimidin-2-yl)-N-methylmethane sulfonamide;
A-30) N-(5-(2-(3-ethylureido)-7-(5-methylpyridin-2-yl)benzo[d]thiazol-5-yl)pyrimidin-2-yl)methanesulfonamide;
A-31) N-(5-(2-(3-ethylureido)-7-(pyridin-2-yl)benzo[d]thiazol-5-yl)pyrimidin-2-yl)methanesulfonamide;
A-32) 1-[5-[2-(dimethylsulfamoylamino)pyrimidin-5-yl]-7-(2-pyridyl)-1,3-benzothiazol-2-yl]-3-ethyl urea;
A-33) N-(5-(2-(3-ethylureido)-7-(pyridin-2-yl)benzo[d]thiazol-5-yl)pyrimidin-2-yl)propane-1-sulfonamide;
A-34) N-(5-(2-(3-ethylureido)-7-(pyridin-2-yl)benzo[d]thiazol-5-yl)pyrimidin-2-yl)cyclopropanesulfonamide;
A-35) N-(5-(2-(3-ethylureido)-7-(pyridin-2-yl)benzo[d]thiazol-5-yl)pyrimidin-2-yl)cyclopentanesulfonamide;
A-36) N-(5-(2-(3-ethylureido)-7-(pyridin-2-yl)benzo[d]thiazol-5-yl)pyrimidin-2-yl)ethanesulfonamide;
A-37) 1-ethyl-3-[5-[2-(ethylsulfamoylamino)pyrimidin-5-yl]-7-(2-pyridyl)-1,3-benzothiazol-2-yl]urea;
A-38) 1-[5-[2-(dimethylsulfamoylamino)pyrimidin-5-yl]-7-(5-methyl-2-pyridyl)-1,3-benzothiazol-2-yl]-3-ethyl urea;
A-39) 1-ethyl-3-[5-[2-(ethylsulfamoylamino)pyrimidin-5-yl]-7-(5-methyl-2-pyridyl)-1,3-benzothiazol-2-yl]urea;
A-40) N-(5-(2-(3-ethylureido)-7-(5-methylpyridin-2-yl)benzo[d]thiazol-5-yl)pyrimidin-2-yl)cyclopropanesulfonamide;
A-41) 1-ethyl-3-[5-[2-(methylsulfamoylamino)pyrimidin-5-yl]-7-(2-pyridyl)-1,3-benzothiazol-2-yl]urea;
A-42) 1-ethyl-3-[7-(5-methyl-2-pyridyl)-5-[2-(methylsulfamoylamino)pyrimidin-5-yl]-1,3-benzothiazol-2-yl]urea;
A-43) N-(5-(2-(3-ethylureido)-7-(pyridin-2-yl)benzo[d]thiazol-5-yl)pyrimidin-2-yl)morpholine-4-sulfonamide;
A-44) N-(5-(2-(3-ethylureido)-7-(5-methylpyridin-2-yl)benzo[d]thiazol-5-yl)pyrimidin-2-yl)morpholine-4-sulfonamide;
A-45) N-(5-(2-(3-ethylureido)-7-(pyridin-2-yl)benzo[d]thiazol-5-yl)pyrimidin-2-yl)pyrrolidine-1-sulfonamide;
A-46) (S)-2-amino-N-(5-(2-(3-ethylureido)-7-(5-methylpyridin-2-yl)benzo[d]thiazol-5-yl)pyrimidin-2-yl)-3-phenylpropane-1-sulfonamide;
A-47) N-(5-(2-(3-ethylureido)-7-(5-methylpyridin-2-yl)benzo[d]thiazol-5-yl)pyrimidin-2-yl)ethanesulfonamide;
A-48) N-(5-(2-(3-ethylureido)-7-(5-methylpyridin-2-yl)benzo[d]thiazol-5-yl)pyrimidin-2-yl)propane-2-sulfonamide;
A-49) N-(5-(2-(3-ethylureido)-7-(pyridin-2-yl)benzo[d]thiazol-5-yl)pyrimidin-2-yl)-3-methoxyazetidine-1-sulfonamide;
A-50) N-(5-(2-(3-ethylureido)-7-(5-methylpyridin-2-yl)benzo[d]thiazol-5-yl)pyrimidin-2-yl)-3-methoxyazetidine-1-sulfonamide;
A-51) N-(5-(2-(3-ethylureido)-7-(5-methylpyridin-2-yl)benzo[d]thiazol-5-yl)pyrimidin-2-yl)benzenesulfonamide;
A-52) N-(5-(2-(3-ethylureido)-7-(5-methylpyridin-2-yl)benzo[d]thiazol-5-yl)pyrimidin-2-yl)-2-morpholinoethanesulfonamide;
A-53) N-(5-(2-(3-ethylureido)-7-(5-methylpyridin-2-yl)benzo[d]thiazol-5-yl)pyrimidin-2-yl)pyrrolidine-3-sulfonamide;
A-54) N-(5-(2-(3-ethylureido)-7-(5-methylpyridin-2-yl)benzo[d]thiazol-5-yl)pyrimidin-2-yl)-3-hydroxypyrrolidine-1-sulfonamide;
A-55) N-(5-(2-(3-ethylureido)-7-(5-methylpyridin-2-yl)benzo[d]thiazol-5-yl)pyrimidin-2-yl)-1-(hydroxymethyl)cyclopropane-1-sulfonamide;
A-56) (R)—N-(5-(2-(3-ethylureido)-7-(5-methylpyridin-2-yl)benzo[d]thiazol-5-yl)pyrimidin-2-yl)-3-morpholinopyrrolidine-1-sulfonamide;
A-57) (R)-3-(dimethylamino)-N-(5-(2-(3-ethylureido)-7-(5-methylpyridin-2-yl)benzo[d]thiazol-5-yl)pyrimidin-2-yl)pyrrolidine-1-sulfonamide;
A-58) 1-acetyl-N-(5-(2-(3-ethylureido)-7-(5-methylpyridin-2-yl)benzo[d]thiazol-5-yl)pyrimidin-2-yl)pyrrolidine-3-sulfonamide;
A-59) (R)—N-(5-(2-(3-ethylureido)-7-(5-methylpyridin-2-yl)benzo[d]thiazol-5-yl)pyrimidin-2-yl)-3-hydroxypyrrolidine-1-sulfonamide;
A-60) (S)—N-(5-(2-(3-ethylureido)-7-(5-methylpyridin-2-yl)benzo[d]thiazol-5-yl)pyrimidin-2-yl)-3-hydroxypyrrolidine-1-sulfonamide;
A-61) N-(5-(2-(3-ethylureido)-6-fluorobenzo[d]thiazol-5-yl)pyrimidin-2-yl)-2-methylpropane-2-sulfonamide;
A-62) (R)—N-(5-(2-(3-ethylureido)-7-(5-methylpyridin-2-yl)benzo[d]thiazol-5-yl)pyrimidin-2-yl)-2-(hydroxymethyl)pyrrolidine-1-sulfonamide;
A-63) (S)—N-(5-(2-(3-ethylureido)-7-(5-methylpyridin-2-yl)benzo[d]thiazol-5-yl)pyrimidin-2-yl)tetrahydrofuran-3-sulfonamide;
A-64) N-(5-(7-bromo-2-(3-ethylureido)benzo[d]thiazol-5-yl)pyrimidin-2-yl)-2-methylpropane-2-sulfonamide;
A-65) 1-[7-bromo-5-[6-(tert-butyl sulfonylamino)-3-pyridyl]-1,3-benzothiazol-2-yl]-3-ethyl urea;
A-66) methyl 2-[[5-[2-(ethylcarbamoylamino)-7-(5-methyl-2-pyridyl)-1,3-benzothiazol-5-yl]pyrimidin-2-yl]sulfamoyl]acetate;
A-67) (R)—N-(5-(2-(3-ethylureido)-7-(5-methylpyridin-2-yl)benzo[d]thiazol-5-yl)pyrimidin-2-yl)-1-(tetrahydro-2H-pyran-2-yl)methanesulfonamide;
A-68) N-(5-(2-(3-ethylureido)-7-(5-methylpyridin-2-yl)benzo[d]thiazol-5-yl)pyrimidin-2-yl)-2-methoxyethanesulfonamide;
A-69) 1-[5-[6-(tert-butylsulfonylamino)-3-pyridyl]-7-(2-ethylthiazol-4-yl)-1,3-benzothiazol-2-yl]-3-ethyl urea;
A-70) N-(5-(2-(3-ethylureido)-7-(5-methylpyridin-2-yl)benzo[d]thiazol-5-yl)pyrimidin-2-yl)-2-hydroxyethanesulfonamide;
A-71) N-(5-(2-(3-ethylureido)-7-(1H-pyrazol-4-yl)benzo[d]thiazol-5-yl)pyrimidin-2-yl)-2-methylpropane-2-sulfonamide;
A-72) N-(5-(7-(3,5-dimethylisoxazol-4-yl)-2-(3-ethylureido)benzo[d]thiazol-5-yl)pyrimidin-2-yl)-2-methylpropane-2-sulfonamide;
A-73) N-(5-(2-(3-ethylureido)-7-(1-methyl-1H-pyrazol-4-yl)benzo[d]thiazol-5-yl)pyrimidin-2-yl)-2-methylpropane-2-sulfonamide;
A-74) N-(5-(2-(3-ethylureido)-7-((5-methylpyridin-2-yl)amino)benzo[d]thiazol-5-yl)pyrimidin-2-yl)-2-methylpropane-2-sulfonamide;
A-75) N-(5-(2-(3-ethylureido)-7-methylbenzo[d]thiazol-5-yl)pyrimidin-2-yl)-2-methylpropane-2-sulfonamide;
A-76) N-(5-(2-(3-ethylureido)benzo[d]thiazol-5-yl)pyrimidin-2-yl)-2-methylpropane-2-sulfonamide;
A-77) N-(5-(7-cyclopropyl-2-(3-ethylureido)benzo[d]thiazol-5-yl)pyrimidin-2-yl)-2-methylpropane-2-sulfonamide;
A-78) N-(5-(2-(3-ethylureido)-7-(tetrahydro-2H-pyran-4-yl)benzo[d]thiazol-5-yl)pyrimidin-2-yl)-2-methylpropane-2-sulfonamide;
A-79) N-(5-(2-(3-ethylureido)-7-(pyrrolidin-1-yl)benzo[d]thiazol-5-yl)pyrimidin-2-yl)-2-methylpropane-2-sulfonamide;
A-80) N-(5-(2-(3-ethylureido)-7-(piperazin-1-yl)benzo[d]thiazol-5-yl)pyrimidin-2-yl)-2-methylpropane-2-sulfonamide;
A-81) N-(5-(2-(3-ethylureido)-7-morpholinobenzo[d]thiazol-5-yl)pyrimidin-2-yl)-2-methylpropane-2-sulfonamide;
A-82) N-(5-(2-(3-ethylureido)-7-(4-methylpiperazin-1-yl)benzo[d]thiazol-5-yl)pyrimidin-2-yl)-2-methylpropane-2-sulfonamide;
A-83) N-(5-(7-(4-acetylpiperazin-1-yl)-2-(3-ethylureido)benzo[d]thiazol-5-yl)pyrimidin-2-yl)-2-methylpropane-2-sulfonamide;
A-84) N-(5-(2-(3-ethylureido)-7-(1-(2-morpholinoethyl)-1H-pyrazol-4-yl)benzo[d]thiazol-5-yl)pyrimidin-2-yl)-2-methylpropane-2-sulfonamide;
A-85) N-(5-(2-(3-ethylureido)-7-(4-methylpyridin-2-yl)benzo[d]thiazol-5-yl)pyrimidin-2-yl)-2-methylpropane-2-sulfonamide;
A-86) N-(5-(2-(3-ethylureido)-7-(5-(morpholinomethyl)pyridin-2-yl)benzo[d]thiazol-5-yl)pyrimidin-2-yl)-2-methylpropane-2-sulfonamide;
A-87) N-(5-(2-(3-ethylureido)-7-(2-(3-hydroxypyrrolidin-1-yl)thiazol-4-yl)benzo[d]thiazol-5-yl)pyrimidin-2-yl)-2-methylpropane-2-sulfonamide;
A-88) 1-[5-[2-(tert-butyl sulfonylamino)pyrimidin-5-yl]-7-(2-hydroxy-4-pyridyl)-1,3-benzothiazol-2-yl]-3-ethyl-urea;
A-89) N-(5-(2-(3-ethylureido)-7-(1-methyl-2-oxo-1, 2-dihydropyridin-4-yl)benzo[d]thiazol-5-yl)pyrimidin-2-yl)-2-methylpropane-2-sulfonamide;
A-90) 2-(4-(5-(2-(1,1-dimethylethylsulfonamido)pyrimidin-5-yl)-2-(3-ethylureido)benzo[d]thiazol-7-yl)piperazin-1-yl)-N-methylacetamide;
A-91) ethyl 2-(4-(5-(2-(1,1-dimethylethylsulfonamido)pyrimidin-5-yl)-2-(3-ethylureido)benzo[d]thiazol-7-yl)piperazin-1-yl)acetate;
A-92) N-(5-(2-(3-ethylureido)-7-(2-(piperazin-1-yl)thiazol-4-yl)benzo[d]thiazol-5-yl)pyrimidin-2-yl)-2-methylpropane-2-sulfonamide;
A-93) N-(5-(2-(3-ethylureido)-7-(6-methylpyridin-2-yl)benzo[d]thiazol-5-yl)pyrimidin-2-yl)-2-methylpropane-2-sulfonamide;
A-94) N-(5-(7-(2-aminopyridin-3-yl)-2-(3-ethylureido)benzo[d]thiazol-5-yl)pyrimidin-2-yl)-2-methylpropane-2-sulfonamide;
A-95) N-(5-(7-(5-aminopyridin-3-yl)-2-(3-ethylureido)benzo[d]thiazol-5-yl)pyrimidin-2-yl)-2-methylpropane-2-sulfonamide;
A-96) N-(5-(2-(3-ethylureido)-7-(4-(2-methoxyethyl)piperazin-1-yl)benzo[d]thiazol-5-yl)pyrimidin-2-yl)-2-methylpropane-2-sulfonamide;
A-97) N-(5-(2-(3-ethylureido)-7-(piperidin-1-yl)benzo[d]thiazol-5-yl)pyrimidin-2-yl)-2-methylpropane-2-sulfonamide;
A-98) N-(5-(7-(5-(aminomethyl)-2-fluorophenyl)-2-(3-ethylureido)benzo[d]thiazol-5-yl)pyrimidin-2-yl)-2-methylpropane-2-sulfonamide;
A-99) N-(5-(2-(3-ethylureido)-7-[2-dimethylaminoethyl(methyl)amino]benzo[d]thiazol-5-yl)pyrimidin-2-yl)-2-methylpropane-2-sulfonamide;
A-100) N-(5-(2-(3-ethylureido)-7-(pyridin-2-yl)benzo[d]thiazol-5-yl)pyrimidin-2-yl)-2-methylpropane-2-sulfonamide;
A-103) 1-[6-[2-(tert-butylsulfonylamino)pyrimidin-5-yl]-5-methoxy-thiazolo[5,4-b]pyridin-2-yl]-3-ethylurea;
A-104) 1-[5-[2-(tert-butylsulfonylamino)pyrimidin-5-yl]-7-(5-hydroxy-2-pyridyl)-1,3-benzothiazol-2-yl]-3-ethylurea;
A-105) 1-[5-[2-(tert-butylsulfonylamino)pyrimidin-5-yl]-7-[4-[(cyclopropylamino)methyl]-2-pyridyl]-1,3-benzothiazol-2-yl]-3-ethylurea;
A-106) 1-[7-(5-amino-2-pyridyl)-5-[2-(tert-butylsulfonylamino)pyrimidin-5-yl]-1,3-benzothiazol-2-yl]-3-ethylurea;
A-107) 1-[5-[2-(tert-butylsulfonylamino)pyrimidin-5-yl]-7-(3-methyl-2-pyridyl)-1,3-benzothiazol-2-yl]-3-ethyl-urea;
A-110) 1-[7-(4-amino-2-pyridyl)-5-[2-(tert-butylsulfonylamino)pyrimidin-5-yl]-1,3-benzothiazol-2-yl]-3-ethylurea;
A-111) 1-[5-[2-(2,3-dihydroxypropyl sulfonylamino)pyrimidin-5-yl]-7-(5-methyl-2-pyridyl)-1,3-benzothiazol-2-yl]-3-ethylurea;
A-113) 1-[5-[2-(tert-butylsulfonylamino)pyrimidin-5-yl]-7-(4,5-dimethyl-2-pyridyl)-1,3-benzothiazol-2-yl]-3-ethylurea;
A-114) 1-[5-[2-(tert-butylsulfonylamino)pyrimidin-5-yl]-7-[(3S,4R,5R,6R)-3,4,5,6-tetrahydroxycyclohexen-1-yl]-1,3-benzothiazol-2-yl]-3-ethylurea; and
A-115) 1-[7-[(3 aR,6aR)-2,3,3a,4,6,6a-hexahydro-1H-pyrrolo[2,3-c]pyrrol-5-yl]-5-[2-(tert-butyl sulfonylamino)pyrimidin-5-yl]-1,3-benzothiazol-2-yl]-3-ethyl-urea; or
a salt, racemate, diastereomer, enantiomer, ester, carbamate, phosphate, sulfate, deuterated form or prodrug thereof.

27. The method according to claim 4 wherein the bacterial type II topoisomerase inhibitor is a compound selected from the group consisting of:
1) 1-ethyl-3-[5-[2-(1-hydroxy-1-methyl-ethyl)pyrimidin-5-yl]-7-(2-pyridyl)-1,3-benzothiazol-2-yl]urea;
2) 1-ethyl-3-[7-[4-[(3-hydroxy-3-methyl-azetidin-1-yl)methyl]-2-pyridyl]-5-[2-(1-hydroxy-1-methyl-ethyl)pyrimidin-5-yl]-1,3-benzothiazol-2-yl]urea;
5) 1-(2-hydroxyethyl)-3-[5-[2-(1-hydroxy-1-methyl-ethyl)pyrimidin-5-yl]-7-(2-pyridyl)-1,3-benzothiazol-2-yl]urea;
10) 1-ethyl-3-[5-[2-(1-hydroxy-1-methyl-ethyl)pyrimidin-5-yl]-7-pyrimidin-2-yl-1,3-benzothiazol-2-yl]urea;
12) 1-ethyl-3-[5-[2-(1-hydroxy-1-methyl-ethyl)pyrimidin-5-yl]-7-methoxy-1,3-benzothiazol-2-yl]urea;
13) 1-ethyl-3-[5-[2-(1-hydroxy-1-methyl-ethyl)pyrimidin-5-yl]-7-(methoxymethyl)-1,3-benzothiazol-2-yl]urea;
14) 1-ethyl-3-[5-[2-(1-hydroxyethyl)pyrimidin-5-yl]-7-[(6-methyl-3-pyridyl)methoxy]-1,3-benzothiazol-2-yl]urea;
15) 1-ethyl-3-[5-[2-(1-hydroxyethyl)pyrimidin-5-yl]-7-[4-(methylsulfanylmethyl)-2-pyridyl]-1,3-benzothiazol-2-yl]urea;
16) 1-ethyl-3-[5-[2-(1-hydroxyethyl)pyrimidin-5-yl]-7-[4-(methylsulfinylmethyl)-2-pyridyl]-1,3-benzothiazol-2-yl]urea;
17) 1-ethyl-3-[5-[2-(1-hydroxyethyl)pyrimidin-5-yl]-7-(2-pyridyl)-1,3-benzothiazol-2-yl]urea;
26) 1-ethyl-3-[5-[2-(1-hydroxy-1-methyl-ethyl)pyrimidin-5-yl]-7-[4-(pyrrolidin-1-ylmethyl)-2-pyridyl]-1,3-benzothiazol-2-yl]urea;
27) 1-ethyl-3-[5-[6-(1-hydroxy-1-methyl-ethyl)-3-pyridyl]-7-(2-pyridyl)-1,3-benzothiazol-2-yl]urea;
39) 1-ethyl-3-[5-[2-(1-hydroxy-1-methyl-ethyl)pyrimidin-5-yl]-7-pyrazin-2-yl-1,3-benzothiazol-2-yl]urea;
40) 1-[5-[2-(1,2-dihydroxyethyl)pyrimidin-5-yl]-7-(2-pyridyl)-1,3-benzothiazol-2-yl]-3-ethyl-urea;
41) 1-[7-(dimethylaminomethyl)-6-hydroxy-5-[6-(1-hydroxy-1-methyl-ethyl)-3-pyridyl]-1,3-benzothiazol-2-yl]-3-ethyl-urea;
43) 1-ethyl-3-[5-[2-(1-hydroxy-1-methyl-ethyl)pyrimidin-5-yl]-7-(6-methylpyrimidin-4-yl)-1,3-benzothiazol-2-yl]urea;
47) 1-ethyl-3-[5-[2-(1-hydroxycyclohexyl)pyrimidin-5-yl]-7-(2-pyridyl)-1,3-benzothiazol-2-yl]urea;
55) 1-ethyl-3-[5-[2-(1-hydroxyethyl)pyrimidin-5-yl]-7-[4-(morpholinomethyl)-2-pyridyl]-1,3-benzothiazol-2-yl]urea;
56) 1-ethyl-3-[5-[2-(1-hydroxy-1-methyl-ethyl)pyrimidin-5-yl]-7-[5-(2-morpholinoethoxy)-2-pyridyl]-1,3-benzothiazol-2-yl]urea;
57) 1-[5-[2-(1-hydroxy-1-methyl-ethyl)pyrimidin-5-yl]-7-(2-pyridyl)-1,3-benzothiazol-2-yl]-3-propyl-urea;
58) 1-[5-[2-[cyclopropyl(hydroxy)methyl]pyrimidin-5-yl]-7-(2-pyridyl)-1,3-benzothiazol-2-yl]-3-ethyl-urea;
59) 1-ethyl-3-[5-[2-(1-hydroxypropyl)pyrimidin-5-yl]-7-(2-pyridyl)-1,3-benzothiazol-2-yl]urea;
60) 1-ethyl-3-[5-[2-(1-hydroxy-2,2-dimethyl-propyl)pyrimidin-5-yl]-7-(2-pyridyl)-1,3-benzothiazol-2-yl]urea;
61) 1-ethyl-3-[5-[2-(1-hydroxybutyl)pyrimidin-5-yl]-7-(2-pyridyl)-1,3-benzothiazol-2-yl]urea;
68) 1-ethyl-3-[5-[2-(1-hydroxyethyl)pyrimidin-5-yl]-7-[4-[(3-methylmorpholin-4-yl)methyl]-2-pyridyl]-1,3-benzothiazol-2-yl]urea;
81) 1-ethyl-3-[5-[2-(1-hydroxy-2-morpholino-ethyl)pyrimidin-5-yl]-7-(2-pyridyl)-1,3-benzothiazol-2-yl]urea;
100) 1-[7-[4-(diethoxyphosphorylmethyl)-2-pyridyl]-5-[2-(1-hydroxyethyl)pyrimidin-5-yl]-1,3-benzothiazol-2-yl]-3-ethyl-urea;
103) 1-ethyl-3-[5-[2-(1-hydroxy-1-methyl-ethyl)pyrimidin-5-yl]-7-[4-[(2-hydroxy-2-methyl-propyl)amino]pyrimidin-2-yl]-1,3-benzothiazol-2-yl]urea;
115) 1-ethyl-3-[5-[2-(1-hydroxyethyl)pyrimidin-5-yl]-7-pyrimidin-2-yl-1,3-benzothiazol-2-yl]urea;
119) 1-ethyl-3-[5-[2-(1-hydroxycyclopentyl)pyrimidin-5-yl]-7-(2-pyridyl)-1,3-benzothiazol-2-yl]urea;
149) 1-[5-[2-(ethylcarbamoylamino)-7-(2-pyridyl)-1,3-benzothiazol-5-yl]pyrimidin-2-yl]ethyl dihydrogen phosphate;
150) 1-ethyl-3-[5-[2-(1-hydroxy-1-methyl-ethyl)pyrimidin-5-yl]-6-(2-methoxyethylamino)-1,3-benzothiazol-2-yl]urea;
152) [(1R)-1-[5-[2-(ethylcarbamoylamino)-7-(2-pyridyl)-1,3-benzothiazol-5-yl]pyrimidin-2-yl]ethyl] dihydrogen phosphate;
153) [(1S)-1-[5-[2-(ethylcarbamoylamino)-7-(2-pyridyl)-1,3-benzothiazol-5-yl]pyrimidin-2-yl]ethyl] dihydrogen phosphate;
157) 1-[5-[2-[1,2-dihydroxy-1-methyl-ethyl]pyrimidin-5-yl]-7-(2-pyridyl)-1,3-benzothiazol-2-yl]-3-ethyl-urea;
158) 1-ethyl-3-[5-[2-[1-hydroxyethyl]pyrimidin-5-yl]-7-(5-methyl-2-pyridyl)-1,3-benzothiazol-2-yl]urea;
182) 1-[6-(cyclopropylmethoxy)-5-[2-(1-hydroxy-1-methyl-ethyl)pyrimidin-5-yl]-1,3-benzothiazol-2-yl]-3-ethyl-urea;
183) 1-ethyl-3-[5-[2-(1-hydroxy-1-methyl-ethyl)pyrimidin-5-yl]-6-(2-methoxyethoxy)-1,3-benzothiazol-2-yl]urea;
184) [1-[5-[2-(ethylcarbamoylamino)-7-(2-pyridyl)-1,3-benzothiazol-5-yl]pyrimidin-2-yl]-1-methyl-ethyl] 2-(2-phosphonooxyethylamino)acetate;
185) 1-ethyl-3-[5-[2-[1-hydroxyethyl]pyrimidin-5-yl]-7-[4-(thiomorpholinomethyl)-2-pyridyl]-1,3-benzothiazol-2-yl]urea;
186) 1-ethyl-3-[6-(2-hydroxyethoxy)-5-[2-(1-hydroxy-1-methyl-ethyl)pyrimidin-5-yl]-1,3-benzothiazol-2-yl]urea;
187) 1-ethyl-3-[5-[2-(1-hydroxy-1-methyl-ethyl)pyrimidin-5-yl]-7-[4-(thiomorpholinomethyl)-2-pyridyl]-1,3-benzothiazol-2-yl]urea;
188) 1-ethyl-3-[5-[2-(1-hydroxy-1-methyl-ethyl)pyrimidin-5-yl]-7-[4-[(1-oxo-1,4-thiazinan-4-yl)methyl]-2-pyridyl]-1,3-benzothiazol-2-yl]urea;
189) 1-ethyl-3-[5-[2-(1-hydroxy-1-methyl-ethyl)pyrimidin-5-yl]-6-[2-methoxyethyl(methyl)amino]-1,3-benzothiazol-2-yl]urea;
190) [1-[5-[2-(ethylcarbamoylamino)-7-(2-pyridyl)-1,3-benzothiazol-5-yl]pyrimidin-2-yl]-1-methyl-ethyl] dihydrogen phosphate;
191) 1-[7-[4-[(1,1-dioxo-1,4-thiazinan-4-yl)methyl]-2-pyridyl]-5-[2-(1-hydroxy-1-methyl-ethyl)pyrimidin-5-yl]-1,3-benzothiazol-2-yl]-3-ethyl-urea;
192) 1-[6-[(3,4-dimethoxyphenyl)methoxy]-5-[2-(1-hydroxy-1-methyl-ethyl)pyrimidin-5-yl]-1,3-benzothiazol-2-yl]-3-ethyl-urea;
193) 1-ethyl-3-[5-[2-[1-hydroxyethyl]pyrimidin-5-yl]-6-(tetrahydrofuran-2-ylmethoxy)-1,3-benzothiazol-2-yl]urea;
194) 1-ethyl-3-[5-[2-[1-hydroxyethyl]pyrimidin-5-yl]-6-morpholino-1,3-benzothiazol-2-yl]urea;
195) 1-[7-[(3 S)-3-aminopyrrolidin-1-yl]-5-[2-(1-hydroxy-1-methyl-ethyl)pyrimidin-5-yl]-1,3-benzothiazol-2-yl]-3-ethyl-urea;
196) 1-ethyl-3-[7-[4-[(2-hydroxyethylamino)methyl]-2-pyridyl]-5-[2-(1-hydroxy-1-methyl-ethyl)pyrimidin-5-yl]-1,3-benzothiazol-2-yl]urea;
197) 1-ethyl-3-[5-[5-(1-hydroxyethyl)-3-pyridyl]-7-(2-pyridyl)-1,3-benzothiazol-2-yl]urea;
198) 1-ethyl-3-[5-[2-(1-hydroxy-1-methyl-ethyl)pyrimidin-5-yl]-7-[4-[(2,2,3,3,5,5,6,6-octadeuteriomorpholin-4-yl)methyl]-2-pyridyl]-1,3-benzothiazol-2-yl]urea;
199) 1-ethyl-3-[5-[2-(1-hydroxy-1-methyl-ethyl)pyrimidin-5-yl]-6-(2-morpholinoethoxy)-1,3-benzothiazol-2-yl]urea;
200) 1-ethyl-3-[5-[2-(1-hydroxy-1-methyl-ethyl)pyrimidin-5-yl]-6-(2-methoxyethylsulfanyl)-1,3-benzothiazol-2-yl]urea;
201) 1-ethyl-3-[5-[2-(1-hydroxy-1-methyl-ethyl)pyrimidin-5-yl]-6-(2-methoxyethylsulfinyl)-1,3-benzothiazol-2-yl]urea;
and; salts, racemates, diastereomers, enantiomers, deuterated forms, hydrates, solvates and prodrugs thereof.
